(19) United States
(12) Patent Application Publication
(10) Pub. No.: US 2023/0382979 A1
(43) Pub. Date: Nov. 30, 2023

(54) ANTI-SARS-COV-2 ANTIGEN ANTIBODIES AND RELATED COMPOSITIONS AND METHODS

(71) Applicant: Adaptive Biotechnologies Corporation, Seattle, WA (US)

(72) Inventors: Erik Yusko, Seattle, WA (US); Peter J. R. Ebert, Seattle, WA (US); Amy Gilbert, Seattle, WA (US); Gladys Keitany, Seattle, WA (US); Mark Klinger, Seattle, WA (US); Ben Rubin, Seattle, WA (US)

(21) Appl. No.: 18/034,134

(22) PCT Filed: Oct. 29, 2021
(86) PCT No.: PCT/US2021/057452

§ 371 Date: Apr. 27, 2023

Related U.S. Application Data
(60) Provisional application No. 63/190,097, filed on May 18, 2021.
(60) Provisional application No. 63/112,096, filed on Nov. 10, 2020.
(60) Provisional application No. 63/108,791, filed on Nov. 2, 2020.
(60) Provisional application No. 63/108,158, filed on Oct. 30, 2020.

(51) Int. Cl. C07K 16/10; A61P 31/14

(52) U.S. Cl. CPC C07K 16/1003; A61P 31/14; A61K 2039/505

(57) ABSTRACT

Provided are antibodies that specifically bind a Severe Acute Respiratory Syndrome Coronavirus 2 (SARS-CoV-2) antigen. Nucleic acids that encode one or both of the variable chain polypeptides of an antibody of the present disclosure are also provided, as are cells that include such nucleic acids. Also provided are compositions that include the antibodies of the present disclosure, including in some instances, pharmaceutical compositions. Methods of making and using the antibodies of the present disclosure are also provided. In certain aspects, provided are methods that include administering to an individual in need thereof, e.g., an individual having or suspected of having a SARS-CoV-2 infection, a therapeutically effective amount of an antibody of the present disclosure.

### CROSS-REFERENCE TO RELATED APPLICATIONS

This application claims the benefit of U.S. Provisional Patent Application No. 63/190,097, filed May 18, 2021, U.S. Provisional Patent Application No. 63/112,096, filed Nov. 10, 2020, U.S. Provisional Patent Application No. 63/108,791, filed Nov. 2, 2020, and U.S. Provisional Patent Application No. 63/108,158, filed Oct. 30, 2020, which applications are incorporated herein by reference in their entireties.

### INTRODUCTION

A novel coronavirus, Severe Acute Respiratory Syndrome Coronavirus 2 (SARS-CoV-2), was first identified in December 2019 as the cause of a respiratory illness designated coronavirus disease 2019, or COVID-19. A new clinical syndrome, COVID-19 is characterized by respiratory symptoms with varying degrees of severity, from mild upper respiratory illness to severe interstitial pneumonia and acute respiratory distress syndrome, aggravated by thrombosis in the pulmonary microcirculation. Its clinical evolution is characterized by three main phases—early infection phase, pulmonary phase, and hyperinflammation phase—with clinical features ranging from mild or no symptoms to acute respiratory distress syndrome and multi-organ failure.

SARS-CoV-2 is a positive-sense single-stranded RNA virus that belongs to the (3-coronaviruse family along with SARS and MERS. The SARS-CoV-2 genome contains five genes that code for four structural proteins-spike (S), envelope (E), membrane (M) and nucleocapsid (N)- and 16 non-structural proteins. Viral entry into human cells is mediated by an interaction between the S glycoprotein and the Angiotensin-Converting Enzyme 2 (ACE2) receptor. ACE2 is a metalloprotease that lowers blood pressure by catalyzing the hydrolyses of angiotensin II. ACE2 enzymatic activity is not related, or needed, in SARS-CoV-2 entry into the host cells.

A number of investigational agents and drugs that are approved for other indications are currently being evaluated in clinical trials for the treatment of COVID-19 and associated complications. Data from randomized controlled trials, prospective and retrospective observational cohorts, and case series studies are rapidly emerging. Remdesivir (GS-5734), an inhibitor of the viral RNA-dependent, RNA polymerase with in vitro inhibitory activity against SARS-CoV-1 and the Middle East respiratory syndrome (MERS-CoV), was identified early as a promising therapeutic candidate for COVID-19 because of its ability to inhibit SARS-CoV-2 in vitro. The U.S. Food and Drug Administration (FDA) recently approved remdesivir for the treatment of patients with COVID-19 requiring hospitalization. However, a study of more than 11,000 people in 30 countries sponsored by the World Health Organization found that remdesivir had little or no effect on hospitalized COVID-19, as indicated by overall mortality, initiation of ventilation and duration of hospital stay. As such, there remains a need for effective therapeutics for prevention and treatment of SARS-CoV-2 infection and COVID-19.

### SUMMARY

Provided are antibodies that specifically bind Severe Acute Respiratory Syndrome Coronavirus 2 (SARS-CoV-2) antigens. Nucleic acids that encode one or both of the variable chain polypeptides of an antibody of the present disclosure are also provided, as are cells that include such nucleic acids. Also provided are compositions that include the antibodies of the present disclosure, including in some instances, pharmaceutical compositions. Methods of making and using the antibodies of the present disclosure are also provided. In certain aspects, provided are methods that include administering to an individual in need thereof a therapeutically effective amount of an antibody of the present disclosure.

### BRIEF DESCRIPTION OF THE FIGURES

FIG. 1: Process diagram for the discovery of fully human SARS-CoV-2 neutralizing antibodies from the blood of infected individuals. Antibodies were isolated from patient memory B cells and plasmablasts and sequenced using ImmunoSEQ (heavy chain) and pairSEQ (corresponding paired light chains) in steps 1-3. Following sequencing, antibodies were recombinantly expressed and evaluated for their ability to specifically bind to SARS-CoV-2 by ELISA and capacity to neutralize the virus using pseudo and authentic live virus assays against multiple variants in steps 4-6.

FIG. 2A: Antibodies react to RBD domain of the spike protein but do not bind to S2 domain or the nucleocapsid. Black bars in both graphs represent positive control.

FIG. 2B: ELISA data of non-RBD/non-S2 antibodies. The antibodies bound to either Trimer alone or Trimer and S1 but not RBD, S2 or nucleocapsid suggesting they are specific to N-terminal domain. Black bars in both graphs represent positive control.

FIG. 3A: Anti-RBD antibody candidates display pM affinity by ELISA. Representative graphs of a dose response ELISA with RBD specific antibodies.

FIG. 3B: Representative sensorgrams of anti RBD antibodies confirming high affinity to RBD protein.

FIG. 3C: Summary table with pM binding affinities of RBD antibodies by ELISA and Biocore.

FIG. 4: ELISA screening data of antibodies isolated from a patient during acute phase of immune response. The majority of the antibodies reacted to trimer and S2 but not RBD, S1 or nucleocapsid. Two antibodies did not react spike or nucleocapsid.

FIG. 5: Selected anti-S2 antibodies show high affinity binding by ELISA. The table shows a summary of EC50 in pM.

FIG. 6A: Dose response ELISA assay comparing reactivity of class 1 antibodies to RBD protein expressed by WA01/2020 SARs-CoV2 (WT) compared to those in the Beta variant (K417N, E484K and N501Y).

FIG. 6B: Dose response ELISA assay comparing reactivity of class 3 antibodies to RBD protein expressed by WA01/2020 SARs-CoV2 (WT) compared to those in the Beta variant (K417N, E484K and N501Y).

FIG. 7A: Dose response ELISA assay comparing reactivity of class 1 antibodies to RBD protein expressed by WA01/2020 SARs-CoV2 (WT) compared to that in the delta variant (L452R).

FIG. 7B: Dose response ELISA assay comparing reactivity of class 3 antibodies to RBD protein expressed by WA01/2020 SARs-CoV2 (WT) compared to that in the delta variant (L452R).

FIG. 8A: Dose response ELISA assay comparing reactivity of class 1 antibodies to RBD protein expressed by WA01/2020 SARs-CoV2 (WT) compared to that in the delta variant (T4781).

FIG. 8B: Dose response ELISA assay comparing reactivity of class 3 antibodies to RBD protein expressed by WA01/2020 SARs-CoV2 (WT) compared to that in the delta variant (T4781).

FIG. 9A: Dose response ELISA assay comparing reactivity of selected anti-RBD antibodies of Spike expressed by WA01/2020 SARs-CoV2 (WT) to that expressed by SARS-Cov1, MERS-Cov, HCOV-HKU1, HCOV-229E and HCOV-OC43.

FIGS. 9B and 9C/10A and 10B: Selected graphs of dose response ELISA assay comparing reactivity of selected anti-S2 antibodies with Spike expressed by WA01/2020 SARs-CoV2 (WT) to that expressed by SARS-Cov1, MERS-Cov, HCOV-HKU1, HCOV-229E and HCOV-OC43.

FIG. 11: Visualization of critical residues for class I monoclonal antibodies (mAbs) binding to RBD protein. Critical residues (lighter spheres) were visualized on a crystal structure of the receptor binding domain of the Spike protein. Secondary residues (darker spheres) that may contribute to binding are also shown.

FIG. 12: Visualization of critical residues for class III monoclonal antibodies (mAbs) binding to RBD protein. Critical residues (lighter spheres) were visualized on a crystal structure of the receptor binding domain of the Spike protein. Secondary residues (darker spheres) that may contribute to binding are also shown.

FIG. 13: A table summarizing the RBD epitope residues for the antibodies shown in FIGS. 11 and 12.

FIG. 14A-14C: A graph showing the frequency of variable amino acids in SARS-CoV-2 variants (top) and epitope residues for selected antibodies (bottom), indicating that the antibodies are not likely to be impacted by SARS-CoV-2 variants.

FIG. 15A-15B: A) Elisa assay of S2 specific mAbs reacting to different domains of S2 protein. Peptides or short proteins corresponding to FP (aa788-806), HR1 (aa910-988) and HR2 (aa1162-1205). B) schematic representation of fusion between viral spike protein and ACEs receptor in the presence host enzyme TMPRSS2.

FIG. 16A: Representative graph of dose blockade of the ability of RBD specific antibodies to inhibit spike binding to ACE protein. Percent inhibition was calculated based on control wells with no antibody. 6D11F2 was used as a positive control.

FIG. 16B: Table summary IC₅₀ in pM of RBD specific antibodies blocking spike/ACE interaction.

FIG. 17A-17C: Dose response graphs class 1 anti-RBD antibodies ability to inhibit pseudovirus invasion of 293T cells overexpressing ACE and TMPRSS2. Percent inhibition calculated based on no antibody wells as 100%. Pseudovirus inhibition was done with WA01/2020 SARs-CoV2 (WT) (17A), alpha variant (17B) and beta variant (17C).

FIG. 17D: Table summary IC₅₀ in pM of class 1 RBD specific antibodies inhibiting different variants of SARs-CoV2 pseudovirus.

FIG. 18A-18C: Dose response graphs class 3 anti-RBD antibodies ability to inhibit pseudovirus invasion of 293T cells overexpressing ACE and TMPRSS2. Percent inhibition calculated based on no antibody wells as 100%. Pseudovirus inhibition was done with WA01/2020 SARs-CoV2 (WT) (18A), alpha variant (18B) and beta variant (18C).

FIG. 18D: Table summary IC₅₀ in pM of class 3 RBD specific antibodies inhibiting different variants of SARs-CoV2 pseudovirus.

FIG. 19A: Dose response graphs anti-RBD antibodies inhibition of WA01/2020 SARs-CoV2 live virus invasion of Vero E6 cells. AR6959 was used as negative control and NC-2143 was used a negative control. Percent inhibition was calculated based on no antibody control wells as 100% infection.

FIG. 19B: Table summary IC₅₀ in pM of RBD specific antibodies inhibiting of WA01/2020 SARs-CoV2 infection of Vero E6 cells.

FIG. 20A-20B: Dose response graphs of anti-RBD class 1 (A) and class 3 (B) antibodies inhibition of beta variant of SARs-CoV2 live virus invasion of Vero E6 cells. Percent inhibition was calculated based on no antibody control wells as 100% infection.

FIG. 20C: Table summary IC₅₀ in pM of class 1 and 3 RBD specific antibodies inhibiting of Beta variant of SARs-CoV2 infection of vero E6 cells.

FIG. 21: Study schematic for in vivo studies with anti-RBD antibodies: 980, 1589, 4042, and combinations thereof.

FIG. 22A-22B: A) Percent body weight change observed over the 7-day study post challenge with the SARs-CoV-2 virus, isolate WA01/2020. Tested antibodies prevented significant weight loss and reduced viral RNA copies observed in oral swabs compared to IgG controls. B) Percent body weight change observed over the 7-day study post challenge with the SARs-CoV-2 virus, beta variant. Tested antibodies prevented significant weight loss and reduced viral RNA copies observed in oral swabs compared to IgG controls.

FIG. 23: Study schematic for in vivo studies with anti-S2 antibodies: 1872 and 1814.

FIG. 24A-24B: A) Percent body weight change observed over the 7-day study post challenge with the SARs-CoV-2 virus, isolate WA01/2020, and at a dose of 20 mg/kg. Tested antibodies prevented significant weight loss. B) Percent body weight change observed over the 7-day study post challenge with the SARs-CoV-2 virus, isolate WA01/2020. Tested antibodies prevented significant weight loss down to doses of 0.5 mg/kg and showed the expected dose response.

FIG. 25: Percent body weight change observed over the 7-day study post challenge with the SARs-CoV-2 virus, beta variant. Tested antibodies were an anti-RBD binding Ab 980 and an anti-S2 binding Ab 1872. These antibodies given as monotherapy or in combination prevented significant weight loss compared to an IgG control. These data demonstrate the non-competing binding, neutralization, and efficacy of combining an anti-S2 antibody and an anti-RBD antibody.

FIG. 26: Summary table of a subset of RBD-binding antibodies, including their epitope bin (structural class), binding affinity via Biacore and ELISA, ACE2-binding inhibition, efficacy at neutralizing pseudovirus and the WA01/2020 isolate in live virus assays. The table also summarizes each antibody's ability to neutralize variants in pseudo- or live-virus assays (circles) or ability to retain binding affinity to antigens representing SARs-CoV-2 variants (squares).

FIG. 27: Summary table of a subset of S2-binding antibodies, binding affinity via ELISA, efficacy at neutralizing pseudovirus of the SARs-CoV (2003) and the SARs-CoV-2 WA01/2020 isolate. The table also summarizes the ability of the antibodies to neutralize variants in pseudovirus neutralization assays (circles) or ability to retain binding affinity to antigens representing SARs-CoV-2 variants (squares).

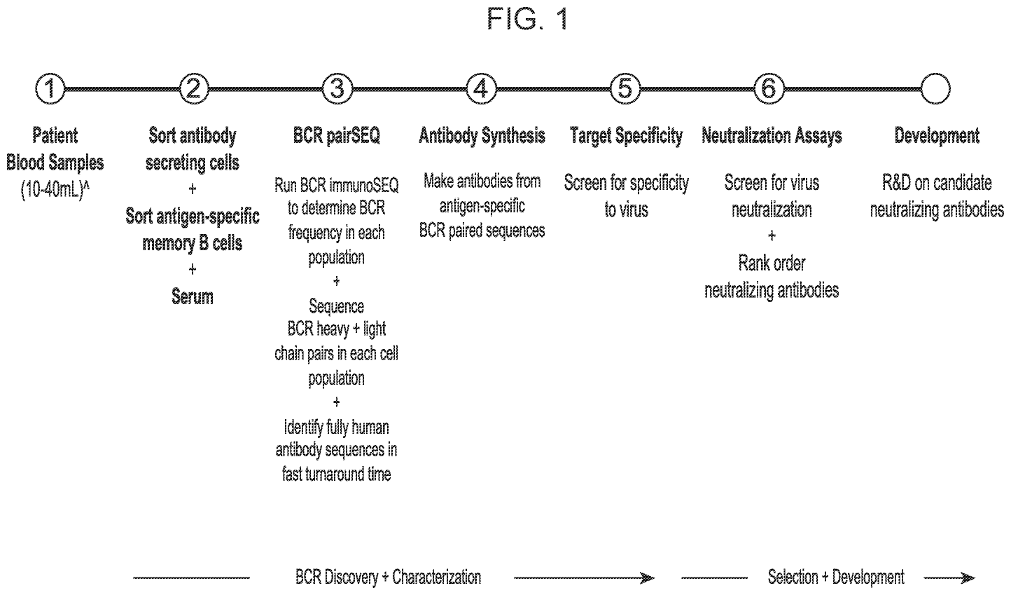

### DETAILED DESCRIPTION

Before the antibodies, compositions and methods of the present disclosure are described in greater detail, it is to be understood that the antibodies, compositions and methods are not limited to particular embodiments described, as such may, of course, vary. It is also to be understood that the terminology used herein is for the purpose of describing particular embodiments only, and is not intended to be limiting, since the scope of the antibodies, compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the antibodies, compositions and methods. The upper and lower limits of these smaller ranges may independently be included in the smaller ranges and are also encompassed within the antibodies, compositions and methods, subject to any specifically excluded limit in the stated range. Where the stated range includes one or both of the limits, ranges excluding either or both of those included limits are also included in the antibodies, compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the antibodies, compositions and methods belong. Although any antibodies, compositions and methods similar or equivalent to those described herein can also be used in the practice or testing of the antibodies, compositions and methods, representative illustrative antibodies, compositions and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present antibodies, compositions and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the antibodies, compositions and methods, which are, for clarity, described in the context of separate embodiments, may also be provided in combination in a single embodiment. Conversely, various features of the antibodies, compositions and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present antibodies, compositions and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present antibodies, compositions and methods. Any recited method can be carried out in the order of events recited or in any other order that is logically possible.

### Antibodies

As summarized above, the present disclosure provides anti-SARS-CoV-2 antigen antibodies. According to some embodiments, an antibody of the present disclosure specifically binds a SARS-CoV-2 antigen such as the S1 subunit of a SARS-CoV-2 spike (S) protein, the receptor-binding domain (RBD) of the S1 subunit of a SARS-CoV-2 spike protein, a SARS-CoV-2 spike (S) protein trimer, the S2 subunit of a SARS-CoV-2 spike protein, a SARS-CoV-2 envelope (E) protein, a SARS-CoV-2 membrane (M) protein, and a SARS-CoV-2 nucleocapsid (N) protein. Details regarding the structure of the SARS-CoV-2 spike protein may be found, e.g., in Lan et al. (2020) Nature 581:215-220; Huang et al. (2020) Acta Pharmacologica Sinica 41:1141-1149; Schaub et al. (2021) Nature Protocols doi.org/10.1038/s41596-021-00623-0; Juraszek et al. (2021) Nature Communications 12, 244; and elsewhere; the disclosures of which are incorporated herein by reference in their entireties for all purposes. In certain embodiments, an anti-SARS-CoV-2 antigen antibody of the present disclosure is a SARS-CoV-2 virus neutralizing antibody. As used herein, a “neutralizing” antibody is an antibody that binds to SARS-CoV-2 virus and interferes with its ability to infect a cell.

In certain embodiments, an antibody of the present disclosure specifically binds a SARS-CoV-2 antigen and competes for binding to the SARS-CoV-2 antigen with an antibody having one, two, three, four, five, or all six complementarity determining regions (CDRs) of one or more of the anti-SARS-CoV-2 antibodies designated herein as antibody 508, 767, 935, 937, 941, 980, 1085, 1213, 1227, 1231, 1238, 1439, 1589, 1671, 1679, 1814, 1815, 1823, 1826, 1851, 1856, 1859, 1864, 1867, 1870, 1871, 1872, 1888, 1915, 1959, 1963, 1969, 1984, 2019, 2020, 2024, 2025, 2050, 2075, 2080, 2432, 2564, 2598, 2606, 2619, 2646, 2706, 2729, 2788, 2793, 2794, 2854, 2866, 2892, 3086, 3091, 3995, 4042, and 4441. In some embodiments, such an antibody comprises one, two, three, four, five, or all six CDRs of an antibody designated herein as antibody 508, 767, 935, 937, 941, 980, 1085, 1213, 1227, 1231, 1238, 1439, 1589, 1671, 1679, 1814, 1815, 1823, 1826, 1851, 1856, 1859, 1864, 1867, 1870, 1871, 1872, 1888, 1915, 1959, 1963, 1969, 1984, 2019, 2020, 2024, 2025, 2050, 2075, 2080, 2432, 2564, 2598, 2606, 2619, 2646, 2706, 2729, 2788, 2793, 2794, 2854, 2866, 2892, 3086, 3091, 3995, 4042, and 4441. In some embodiments, such antibodies comprise a variable heavy chain (V_{H}) polypeptide and/or a variable light chain (V_{L}) polypeptide having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{H} and/or the V_{L} of an antibody designated herein as antibody 508, 767, 935, 937, 941, 980, 1085, 1213, 1227, 1231, 1238, 1439, 1589, 1671, 1679, 1814, 1815, 1823, 1826, 1851, 1856, 1859, 1864, 1867, 1870, 1871, 1872, 1888, 1915, 1959, 1963, 1969, 1984, 2019, 2020, 2024, 2025, 2050, 2075, 2080, 2432, 2564, 2598, 2606, 2619, 2646, 2706, 2729, 2788, 2793, 2794, 2854, 2866, 2892, 3086, 3091, 3995, 4042, and 4441.

Antibodies designated herein as antibody 508, 767, 935, 937, 941, 980, 1085, 1213, 1227, 1231, 1238, 1439, 1589, 1671, 1679, 1814, 1815, 1823, 1826, 1851, 1856, 1859, 1864, 1867, 1870, 1871, 1872, 1888, 1915, 1959, 1963, 1969, 1984, 2019, 2020, 2024, 2025, 2050, 2075, 2080, 2432, 2564, 2598, 2606, 2619, 2646, 2706, 2729, 2788, 2793, 2794, 2854, 2866, 2892, 3086, 3091, 3995, 4042, and 4441 were selected from amongst more than 300,000 identified and more than 3,000 synthesized for desirable manufacturing features such as the lack of free cysteines, non-standard glycosylation sites, site for undesirable post-translational modifications, and biophysical properties affecting stability. Additional selection involved the ability to potently bind to and neutralize SARS-CoV-2 virus, retain neutralization of and/or binding to variants, diversity of epitopes, and the diversity of mechanism of neutralization.

As demonstrated in the Experimental section herein, the epitopes of RBD-binding antibodies were resolved, and these antibodies may be grouped into three classes or epitope bins. These antibodies are non-competing that block ACE2 binding. When administered in combination (see, e.g., the in vivo studies and data in the Experimental section and figures), the antibody combinations demonstrated equivalent efficacy and reduced the impact of viral variants as compared to a single type of antibody administered alone.

The antibodies of the present disclosure that bind to the S2 domain of the spike protein represent a unique binding epitope on the spike protein. These antibodies do not compete with RBD-binding antibodies. The in vivo studies presented herein demonstrate that the combination of an RBD-binding antibody and an S2-binding antibody exhibit equivalent efficacy and as a combination reduce the impact of viral variants.

The amino acid sequence of the S2 domain of the spike protein is far more conserved than the RBD domain [Shah et. All, Fr. Immunology 2021] and therefore represents a novel and attractive epitope that will likely be resistant to viral variants. S2-binding antibodies also neutralized the SARs-CoV (2003) (see Experimental section below) and may neutralize, as evidenced by binding, other coronavirus in the betacoronavirus family.

Moreover, S2-binding antibodies present a unique mechanism of viral neutralization compared RBD-binding antibodies. The S2 binding antibodies do not block the RBD from binding ACE2, but rather neutralize virus by binding to HR1/Fusion Peptide-region within the S2 domain. Effector function was preserved in these antibodies and may contribute to additional mechanisms of neutralization and efficacy.

The amino acid sequences of the V_{H} polypeptides and V_{L} polypeptides of the 508, 767, 935, 937, 941, 980, 1085, 1213, 1227, 1231, 1238, 1439, 1589, 1671, 1679, 1814, 1815, 1823, 1826, 1851, 1856, 1859, 1864, 1867, 1870, 1871, 1872, 1888, 1915, 1959, 1963, 1969, 1984, 2019, 2020, 2024, 2025, 2050, 2075, 2080, 2432, 2564, 2598, 2606, 2619, 2646, 2706, 2729, 2788, 2793, 2794, 2854, 2866, 2892, 3086, 3091, 3995, 4042, and 4441 antibodies are provided in Table 1 below. CDR sequences defined according to IMGT are underlined.

TABLE 1
Amino Acid Sequences of Example Anti-SARS-COV-2 Antibodies

| | |
|---|---|
| 508 V_{H}: SEQ ID NO: 1 | QVQLQESGPRLVKPSETLSLTCTVSGDSISSYYWSWIRQPPGKGLE |
| V_{H} CDR1: SEQ ID NO: 2 | WIGYIYYSGSTNYNPSLKSRVTISVDTSKNQFSLKLSSVTAADTAVY |
| V_{H} CDR2: SEQ ID NO: 3 | YCARKTVAGPAGEFDYWGQGTLVTVSS |
| V_{H} CDR3: SEQ ID NO: 4 | |
| 508 V_{L}: SEQ ID NO: 5 | QSVLTQPPSVSGAPGQRVTISCTGSSSNIGAGYDVHWYQQFPGTA |
| V_{L} CDR1: SEQ ID NO: 6 | PKLLIYGNSNRPSGVPDRFSGSKSGTSASLAITGLQAEDEADYYCQ |
| V_{L} CDR2: SEQ ID NO: 7 | SYDSSLSGPYVFGTGTKVTVL |
| V_{L} CDR3: SEQ ID NO: 8 | |
| 767 V_{H}: SEQ ID NO: 9 | QVQVVQSGAEVKKPGASVKVSCTASGHTFTGYAIHWVRQAPGQR |
| V_{H} CDR1: SEQ ID NO: 10 | LEWMGLINAGGIYRTYSQRFQGRVTITRDTSASTAYMELTGLTSED |
| V_{H} CDR2: SEQ ID NO: 11 | TAVYYCARANYGSGSFSDHWGQGTLVTVSS |
| V_{H} CDR3: SEQ ID NO: 12 | |
| 767 V_{L}: SEQ ID NO: 13 | DIQMTQSPSSLSASVGDRVTITCRASQSISSYLNWYQQKPGKAPKL |
| V_{L} CDR1: SEQ ID NO: 14 | LIYAASSLQSGVPSRFSGSGSGTDFTLTISSLQPEDFATYYCQQSSS |
| V_{L} CDR2: SEQ ID NO: 15 | TPYTFGQGTKLEIK |
| V_{L} CDR3: SEQ ID NO: 16 | |
| 935 V_{H}: SEQ ID NO: 17 | EVQLVESGGGLVQPGGSLRLSCAASGFTVSSNYMSWVRQAPGKG |
| V_{H} CDR1: SEQ ID NO: 18 | LEWVSVIYSGGSTFYADSVKGRFTISRDNSRNTLYLQMNSLRAEDT |
| V_{H} CDR2: SEQ ID NO: 19 | AVYYCARDWGEFFFDFWGQGALVTVSS |
| V_{H} CDR3: SEQ ID NO: 20 | |
| 935 V_{L}: SEQ ID NO: 21 | EIVLTQSPGTLSLSPGERATLSCRASQTISSSYLAWYQQKPGQAPR |
| V_{L} CDR1: SEQ ID NO: 22 | LLISGASSRATGIPDRFSGSGSGTDFTLTISRLEPEDFAVYYCQQYG |
| V_{L} CDR2: SEQ ID NO: 23 | SSPRTFGQGTNVEIK |
| V_{L} CDR3: SEQ ID NO: 24 | |
| 937 V_{H}: SEQ ID NO: 25 | EVQLVESGGGLVQPGRSLRLSCAASGFTFDDYAMHWVRQAPGKG |
| V_{H} CDR1: SEQ ID NO: 26 | LEWVSGISWNSGSIGYADSVKGRFTISRDNAKNSLYLQMNSLKPED |
| V_{H} CDR2: SEQ ID NO: 27 | TALYYCAKSTTSCYNGSQCPTKRPKPLESWGQGTLVIVSA |
| V_{H} CDR3: SEQ ID NO: 28 | |
| 937 V_{L}: SEQ ID NO: 29 | DIQMTQSPSTLSAFVGDRVTITCRASQSINNWLAWYQQKPGEVPKL |
| V_{L} CDR1: SEQ ID NO: 30 | LIYKASSLENGVPSRFSGSGFGTEFTLTISSLQPDDFAIYYCQQYYS |
| V_{L} CDR2: SEQ ID NO: 31 | HSHTFGQGTKLEIK |
| V_{L} CDR3: SEQ ID NO: 32 | |
| 941 V_{H}: SEQ ID NO: 33 | QVQLVQSGAEVKKPGSSVKVSCKASGGTFSNSAINWVRQAPGQG |
| V_{H} CDR1: SEQ ID NO: 34 | LEWMGGIIPLFGAADYAQKFQARVTITADKSTTTSYMELSSLRSEDT |
| V_{H} CDR2: SEQ ID NO: 35 | AVYYCARDTLEHDDVWGNFRLSLPLSFWGQGTLVTVSS |
| V_{H} CDR3: SEQ ID NO: 36 | |
| 941 V_{L}: SEQ ID NO: 37 | EIVMTQSPATLSVSPGERATLSCRASQSVSSKLAWYQQKPGQAPR |
| V_{L} CDR1: SEQ ID NO: 38 | LLFYGASTRATGLPARFSGSGSGTEFTLTISSLQSEDFAVYYCQQY |
| V_{L} CDR2: SEQ ID NO: 39 | NDWPMYTFGPGTRLEIK |
| V_{L} CDR3: SEQ ID NO: 40 | |
| 980 V_{H}: SEQ ID NO: 41 | QVQLVESGGGVVQPGRSLRLSCAASGFTFSRYGMHWVRQAPGK |
| V_{H} CDR1: SEQ ID NO: 42 | GLEWVALISSGGSNKYYADSVKGRFTISRDNSKNTLYLEMNSLRAE |
| V_{H} CDR2: SEQ ID NO: 43 | DTAVYYCAKDAIYDYIWGAYRENWFDPWGQGTLVTVSS |
| V_{H} CDR3: SEQ ID NO: 44 | |
| 980 V_{L}: SEQ ID NO: 45 | DIQMTQSPSSLSASVGDRVTITCRASQSISNYLNWYQQKPGKAPNL |
| V_{L} CDR1: SEQ ID NO: 46 | LIYAASSLQSGVPSRFSGSGSGTDFTLTISSLQPEDFATYYCQQTSS |
| V_{L} CDR2: SEQ ID NO: 47 | PPLTFGQGTKVEIK |
| V_{L} CDR3: SEQ ID NO: 48 | |
| 1085 V_{H}: SEQ ID NO: 49 | QVQLVQSGAEVKKPGSSVKVSCKASGGTFSSYAISWVRQAPGQG |
| V_{H} CDR1: SEQ ID NO: 50 | LEWMGGIIPVFGTTNYAQEFRARVTITADESTNTAYMELRSLSSADT |
| V_{H} CDR2: SEQ ID NO: 51 | GVYYCATGAGEMIEVTIANDVFDIWGQGTRVTVSS |
| V_{H} CDR3: SEQ ID NO: 52 | |
| 1085 V_{L}: SEQ ID NO: 53 | QLVLTQSPSASASLGASVKLTCTLSSGHSSYAIAWHQQQPEKGPR |
| V_{L} CDR1: SEQ ID NO: 54 | YLMKLNSDGSHSKGDGIPDRFSGSSSGAERYLTISSLQSDDEADYY |
| V_{L} CDR2: SEQ ID NO: 55 | CQTWGTAIHVFGTGTKVTVL |
| V_{L} CDR3: SEQ ID NO: 56 | |
| 1213 V_{H}: SEQ ID NO: 57 | QMQLVQSGAEVKKTGSSVKVSCEASENTFTNRYLHWVRQAPGQA |
| V_{H} CDR1: SEQ ID NO: 58 | LEWMGWITPFQGNTHYAQKFQDRVTITRDRSMNTVYMELSSLRSE |
| V_{H} CDR2: SEQ ID NO: 59 | DTAIYYCASGGQYGPGSYYFEFWGQGTLVTVSS |
| V_{H} CDR3: SEQ ID NO: 60 | |
| 1213 V_{L}: SEQ ID NO: 61 | EIVMTQSPATLSVSPGERATLSCRASQSIRNNLAWYRQKPGQAPRL |
| V_{L} CDR1: SEQ ID NO: 62 | LIYGASTRAAGVPARFSGSGSGTEFTLTISSLQSEDFAVYYCQQYIN |
| V_{L} CDR2: SEQ ID NO: 63 | WPPLTFGGGTKVEIK |
| V_{L} CDR3: SEQ ID NO: 64 | |
| 1227 V_{H}: SEQ ID NO: 65 | QMQLVQSGAEVKKTGSSVKVSCEASGYTFTNRYLHWVRQAPGQA |
| V_{H} CDR1: SEQ ID NO: 66 | LEWMGWITPFQGNTNYAQKFQDRVTITRDMSMNTVYMELSSLRSE |
| V_{H} CDR2: SEQ ID NO: 67 | DTAKYYCASGGQYGAGSYYLEDWGQGTLVTVSS |
| V_{H} CDR3: SEQ ID NO: 68 | |
| 1227 V_{L}: SEQ ID NO: 69 | EIVMTQSPATLSVSPGERATLSCRASQSVRSNLAWYRQKPGQAPR |
| V_{L} CDR1: SEQ ID NO: 70 | LLIYGASTRATGVPARFSGSGSGTEFTLTISSLQSEDFAVYYCQQYI |
| V_{L} CDR2: SEQ ID NO: 71 | NWPPLTFGGGTRVEIK |
| V_{L} CDR3: SEQ ID NO: 72 | |
| 1231 V_{H}: SEQ ID NO: 73 | QVQLVQSGSELKKPGASVKVSCTGYGYTFTSYAMNWVRQAPGQG |
| V_{H} CDR1: SEQ ID NO: 74 | LEWMGWINTNTGHPAYAQGFTGRFVFSLDSSVSTAYLQISSLKAED |
| V_{H} CDR2: SEQ ID NO: 75 | TAVYYCATTIRAGGYFDFWGQGTLVTVSS |
| V_{H} CDR3: SEQ ID NO: 76 | |
| 1231 V_{L}: SEQ ID NO: 77 | DIQLTQSPSFLSASVGDRVTITCRASQGISSYLAWFQQKPGKAPKLL |
| V_{L} CDR1: SEQ ID NO: 78 | IYVVSILQSGVPSRFSGSGSGTEFTLTISSLQPEDFATYYCQQLNSY |
| V_{L} CDR2: SEQ ID NO: 79 | PYTFGQGTKLEIK |
| V_{L} CDR3: SEQ ID NO: 80 | |
| 1238 V_{H}: SEQ ID NO: 81 | QVQLVQSGSELKKPGASVKVSCKASGYTFTNYGMNWVRQAPGQG |
| V_{H} CDR1: SEQ ID NO: 82 | LEWMGWINTNTGNPTNAQGFTGRFVFSLDTSVSTAYLQINSLKGE |
| V_{H} CDR2: SEQ ID NO: 83 | DTAVYYCARVADTGEMDVWGQGTTVTVSS |
| V_{H} CDR3: SEQ ID NO: 84 | |
| 1238 V_{L} :SEQ ID NO: 85 | SYELTQPPSVSVSPGQTARITCSGDALPKQYAYWYQQKPGQAPVL |
| V_{L} CDR1: SEQ ID NO: 86 | VIYKDSERPSGIPERFSGSSSGTTVTLTISGVQAEDEADYYCQSADS |
| V_{L} CDR2: SEQ ID NO: 87 | SGSYNWVFGGGTKLTIL |
| V_{L} CDR3: SEQ ID NO: 88 | |
| 1439 V_{H}: SEQ ID NO: 89 | QVQLQESGPGLVKPSETLSLTCTVSGDSISTYYWSWIRQPPGKGLE |
| V_{H} CDR1: SEQ ID NO: 90 | WIGYIHYSGSTNYNPSLKSRVAVSVDTSKNQFSLKLSSVTAADTAV |
| V_{H} CDR2: SEQ ID NO: 91 | YYCARGGGVFGVVINFDYWGQGILVTVSS |
| V_{H} CDR3: SEQ ID NO: 92 | |
| 1439 V_{L}: SEQ ID NO: 93 | NFMLTQPYSVSESPGKTVTISCTRSSGSIASNYVQWYQQRPGSAP |
| V_{L} CDR1: SEQ ID NO: 94 | TTVIYEDNQRPSGVPDRFSGSIDSSSNSASLTISGLKTEDEADYY |
| V_{L} CDR2: SEQ ID NO: 95 | CQSRDSSNPVVFGGGTKLTVL |
| V_{L} CDR3: SEQ ID NO: 96 | |
| 1589 V_{H}: SEQ ID NO: 97 | EVQLVESGGGLIQVGGSLRLSCAASGLTVTSNYMNWVRQGPGKG |
| V_{H} CDR1: SEQ ID NO: 98 | LEWVSLIYSGGTTYYADSVKGRFTISRDDSKNTLYLQMNSLRAEDT |
| V_{H} CDR2: SEQ ID NO: 99 | AVYYCARPIVGARSGMDVWGQGTAVTVSS |
| V_{H} CDR3: SEQ ID NO: 100 | |
| 1589 V_{L}: SEQ ID NO: 101 | DIQMTQSPSSLSASVGDRVTITCQASQDINKYLNWYQQKPGKAPKL |
| V_{L} CDR1: SEQ ID NO: 102 | LIYDASNLETGVPSRFSGSGSGTDFTFTISSLQPEDLATYYCHQFDN |
| V_{L} CDR2: SEQ ID NO: 103 | LPGTFGGGTKVEIK |
| V_{L} CDR3: SEQ ID NO: 104 | |
| 1671 V_{H}: SEQ ID NO: 105 | EVQLVESGGGLVKPGGSLRLSCAASGFTFSTYNMNWVRQAPGKG |
| V_{H} CDR1: SEQ ID NO: 106 | LEWVSSISSNSNYIYYADSMKGRFTISRDNAKNSLYLQMNSLRAED |
| V_{H} CDR2: SEQ ID NO: 107 | TAVYYCARDMDPLPYFDWLLYAFDVWGQGTMVTVSS |
| V_{H} CDR3: SEQ ID NO: 108 | |
| 1671 V_{L}: SEQ ID NO: 109 | EIVLTQSPGTLSLSPGERATLSCRASQSVSGSYLAWYQQKPGQAP |
| V_{L} CDR1: SEQ ID NO: 110 | RLLIYGASSRATGIPDRFSGSGSGTDFTLTISRLEPEDFAVYYCQQY |
| V_{L} CDR2: SEQ ID NO: 111 | GSPIFTFGPGTKVDIK |
| V_{L} CDR3: SEQ ID NO: 112 | |
| 1679 V_{H}: SEQ ID NO: 113 | QITLKESGPTLVKPTQTLTLTCTFSGFSLSTVGVGVAWIRQPPGKAL |
| V_{H} CDR1: SEQ ID NO: 114 | EWLALIYWDDDKRYSPSLKSRLTITKDTSKNHVVLTMTNMDPVDTA |
| V_{H} CDR2: SEQ ID NO: 115 | TYYCAHHIIAALVDVWGKGTTVTVSS |
| V_{H} CDR3: SEQ ID NO: 116 | |
| 1679 V_{L}: SEQ ID NO: 117 | QSALTQPASVSGSPGQSITISCTGTSSDVGGNNYVSWYQHHPGEA |
| V_{L} CDR1: SEQ ID NO: 118 | PKLMIYEVSNRPSGVSNRFSGSKSGNTASLTISGLQAEDEADYYCS |
| V_{L} CDR2: SEQ ID NO: 119 | SYTTSSTLVVFGGGSKLTVL |
| V_{L} CDR3: SEQ ID NO: 120 | |
| 1814 V_{H}: SEQ ID NO: 121 | QVQLQESGPGLVKPSETLSLTCSVSGGSINYYYWSWIRQTPGQGL |
| V_{H} CDR1: SEQ ID NO: 122 | EWIGFIYSSGTTNYNPSLKSRVTMSKDTAKKQFSLKLTSVTAADSAV |
| V_{H} CDR2: SEQ ID NO: 123 | YYCARHSRSCTNGVCQTYYYYALDVWGHGTTVTVSS |
| V_{H} CDR3: SEQ ID NO: 124 | |
| 1814 V_{L}: SEQ ID NO: 125 | QSVLTQPPSVSGAPGQRVTISCTGSGSNIGSGYDVHWYQQLPGRA |
| V_{L} CDR1: SEQ ID NO: 126 | PKLLIYRNRNRPSGVPDRFSGSKSGTSASLAIAGLQSEDEGDYFCQ |
| V_{L} CDR2: SEQ ID NO: 127 | SYDGRLGESAVFGGGTRLTVL |
| V_{L} CDR3: SEQ ID NO: 128 | |
| 1815 V_{H}: SEQ ID NO: 129 | QVQLQESGPGLVKPSETLSLTCSVSGGSINYYYWSWIRKSPGKGL |
| V_{H} CDR1: SEQ ID NO: 130 | EWIGFIYSSGTTNYNPSLKSRVSMSIGTSKRQFSLKLSSVTAADSAV |
| V_{H} CDR2: SEQ ID NO: 131 | YYCARHSRSCTNGVCQTYYYYALDVWGHGTTVTVSS |
| V_{H} CDR3: SEQ ID NO: 132 | |
| 1815 V_{L}: SEQ ID NO: 133 | QSVLTQPPSVSGAPGQRVTISCTGSSSNIGAGYDVHWYQQLPGTA |
| V_{L} CDR1: SEQ ID NO: 134 | PKLLIYANTHRPSGVPDRFSASKSGTSASLAIAGLQAEDEGDYYCQ |
| V_{L} CDR2: SEQ ID NO: 135 | SYDGSLSESAVFGGGTRLTVL |
| V_{L} CDR3: SEQ ID NO: 136 | |
| 1823 V_{H}: SEQ ID NO: 137 | EVQLVESGGGLVKPGGSLRLSCVASGFSFGLYTMNWVRQAPGKG |
| V_{H} CDR1: SEQ ID NO: 138 | LEWVSYISSSTSYKYYADSVKGRVSVSRDNAKNSLYLQLNGLRVED |
| V_{H} CDR2: SEQ ID NO: 139 | TAVYYCARDGYCPNGICTYYGMDVWGQGTTVTVSA |
| V_{H} CDR3: SEQ ID NO: 140 | |
| 1823 V_{L}: SEQ ID NO: 141 | EIVMTQSPATLSVSPGERATLSCRASQSVSSNLAWYQQKPGQAPR |
| V_{L} CDR1: SEQ ID NO: 142 | LLIYGASTRATGIPARFSGSGSGTEFTLTITGLQSEDFAVYYCQQYD |
| V_{L} CDR2: SEQ ID NO: 143 | KWPPAYSFGQGTKVEIK |
| V_{L} CDR3: SEQ ID NO: 144 | |
| 1826 V_{H}: SEQ ID NO: 145 | QVQLQESGPGLVKPSETLSLTCSVTGGSTNYYYWSWIRQPPGKGL |
| V_{H} CDR1: SEQ ID NO: 146 | EWIGYIYYSGTTNYNPSLKDRVTMSVDKSKTQLSLKLNSVTAADTA |
| V_{H} CDR2: SEQ ID NO: 147 | VYYCARHARHCTNGVCQTYYYYGLDVWGLGTTVAVSA |
| V_{H} CDR3: SEQ ID NO: 148 | |
| 1826 V_{L}: SEQ ID NO: 149 | QSVLTQPPSVSAAPGQRVTISCSGSSSNIGAGYDVHWYQHLPGTA |
| V_{L} CDR1: SEQ ID NO: 150 | PKLLIYNNNNRPSGVPDRFSASRSGTSASLAITGVQTEDEADYYCQ |
| V_{L} CDR2: SEQ ID NO: 151 | SFDGRLSESGVFGGGTKLTVL |
| V_{L} CDR3: SEQ ID NO: 152 | |
| 1851 V_{H}: SEQ ID NO: 153 | QVQLQESGPGLVKPSETLSLTCTVSGGSINYYYWSWIRQSPGKGL |
| V_{H} CDR1: SEQ ID NO: 154 | EWIGFIYSSGTTNYNPSLKSRVTMSVDSSKSQFSLKLSSVTAADSA |
| V_{H} CDR2: SEQ ID NO: 155 | VYYCARHSRSCTNGVCQTYYYYALDVWGHGTTVTVSS |
| V_{H} CDR3: SEQ ID NO: 156 | |
| 1851 V_{L}: SEQ ID NO: 157 | QSVLTQPPSVSGAPGQRVTISCTGSSSNIGAGYDVHWYQQLPGTA |
| V_{L} CDR1: SEQ ID NO: 158 | PKLLIYGNSNRPSGVPDRFSASKSGTSASLAIAGLQPEDEGDYYCQ |
| V_{L} CDR2: SEQ ID NO: 159 | SYDGSLSESGVFGGGTRLTVL |
| V_{L} CDR3: SEQ ID NO: 160 | |
| 1856 V_{H}: SEQ ID NO: 161 | EVRLVESGGGLVKPGGSLRLSCAASGFSFSLYTMNWVRQAPGKG |
| V_{H} CDR1: SEQ ID NO: 162 | LEWVSYISSSSSYRYYADSVKGRFSVSRDNAKNTLYLEMNGLRAE |
| V_{H} CDR2: SEQ ID NO: 163 | DTAVYYCARDGYCPRGVCTYYGMDVWGQGTTVTVSA |
| V_{H} CDR3: SEQ ID NO: 164 | |
| 1856 V_{L}: SEQ ID NO: 165 | EIVMTQSPATLSVSPGERATLSCRASQTIGIRLAWYQQKPGQAPRL |
| V_{L} CDR1: SEQ ID NO: 166 | LIYDATIRATGIPARFSGSGSGTDFTLTISGLQSEDFAVYYCQRYNN |
| V_{L} CDR2: SEQ ID NO: 167 | WPPVYTFGQGTKLEMK |
| V_{L} CDR3: SEQ ID NO: 168 | |
| 1859 V_{H}: SEQ ID NO: 169 | EVQLVESGGGLVKPGGSLRLSCAASGFSFNTYTMNWVRQAPGKG |
| V_{H} CDR1: SEQ ID NO: 170 | LEWVSYISSSSSYKYYSDSVKGRFSVSRDNAKKSLYLQMNGLRAE |
| V_{H} CDR2: SEQ ID NO: 171 | DTAVYYCARDGYCPNGVCTYYGMDVWGQGTTVTVSL |
| V_{H} CDR3: SEQ ID NO: 172 | |
| 1859 V_{L}: SEQ ID NO: 173 | EIVMTQSPATLSVSPGERATLSCRASQSVSSNLAWYQQKPGQAPR |
| V_{L} CDR1: SEQ ID NO: 174 | LLIYGASTRATGIPARFSGSGSGTEFTLTISGLQSEDFAVYFCQQYS |
| V_{L} CDR2: SEQ ID NO: 175 | KWPPAYTFGQGTKLEIK |
| V_{L} CDR3: SEQ ID NO: 176 | |
| 1864 V_{H}: SEQ ID NO: 177 | QVQLQESGPGLVKPSETLSLTCTVSGGSINYYYWSWIRQPPGKGL |
| V_{H} CDR1: SEQ ID NO: 178 | EWIGFIYSSGTTNYNPSLKSRVTMSVDSSKSQFSLKLSSVTAADSA |
| V_{H} CDR2: SEQ ID NO: 179 | VYYCARHSRSCTNGVCQTYYYYALDVWGHGTTVTVSS |
| V_{H} CDR3: SEQ ID NO: 180 | |
| 1864 V_{L}: SEQ ID NO: 181 | QSVLTQPPSVSGAPGQRVTISCTGSSSNIGAGYDVHWYQQLPGTA |
| V_{L} CDR1: SEQ ID NO: 182 | PKLLIYGNSNRPSGVPDRFSASKSGTSASLAIAGLQPEDEGDYYCQ |
| V_{L} CDR2: SEQ ID NO: 183 | SYDGSLSESGVFGGGTRLTVL |
| V_{L} CDR3: SEQ ID NO: 184 | |
| 1867 V_{H} :SEQ ID NO: 185 | EVQLVESGGGLVKPGGSLRLSCAASGFSFSTYTMNWVRQAPGKG |
| V_{H} CDR1: SEQ ID NO: 186 | LEWVSYISSSSSYRYYADSVRGRFSVSRDNAKNSLYLQMNGLRVE |
| V_{H} CDR2: SEQ ID NO: 187 | DTAVYYCARDGYCPNGVCTYYGMDVWGQGTTVTVSS |
| V_{H} CDR3: SEQ ID NO: 188 | |
| 1867 V_{L} :SEQ ID NO: 189 | EIVMTQSPATLSVSPGERATLSCRASQSVSSNLAWYQQKPGQAPR |
| V_{L} CDR1: SEQ ID NO: 190 | LLIYGASTRATGIPARFSGSGSGTDFTLTISSLQSEDFAVYYCQQYS |
| V_{L} CDR2: SEQ ID NO: 191 | KWPPAYTFGQGTKLEIK |
| V_{L} CDR3: SEQ ID NO: 192 | |
| 1870 V_{H} :SEQ ID NO: 193 | QVQLQESGPGLVKPSETLSLTCSVSGGSINYYYWSWIRQPPGKGL |
| V_{H} CDR1: SEQ ID NO: 194 | EWIGFIYSSGTTNYNPSLKSRVTMSVDTSKNQFSLKLSSVTAADSA |
| V_{H} CDR2: SEQ ID NO: 195 | VYYCARHSRSCTNGVCQTYYYYALDVWGHGTTVTVSS |
| V_{H} CDR3: SEQ ID NO: 196 | |
| 1870 V_{L} :SEQ ID NO: 197 | QSVLTQPPSVSGAPGQRVTISCTGSSSNIGAGYDVHWYQQLPGTA |
| V_{L} CDR1: SEQ ID NO: 198 | PKLLIYGNSNRPSGVPDRFSASKSGTSASLAIAGLQAEDEGDYYCQ |
| V_{L} CDR2: SEQ ID NO: 199 | SYDGSLSESGVFGGGTRLTVL |
| V_{L} CDR3: SEQ ID NO: 200 | |
| 1871 V_{H} : SEQ ID NO: 201 | EVQLVESGGGLVKPGGSLRLSCVASGFSFSIYSMNWVRQAPGKGL |
| V_{H} CDR1: SEQ ID NO: 202 | EWVSYISSSSSYKYYADSVKGRFSVSRDNAKNSLYLQLNGLRAEDT |
| V_{H} CDR2: SEQ ID NO: 203 | AVYYCARDGYCPKGVCTYYGMDVWGQGTTVTVSA |
| V_{H} CDR3: SEQ ID NO: 204 | |
| 1871 V_{L}: SEQ ID NO: 205 | EIVMTQSPATLSVSPGERVTLSCRASQSVRSRLAWFQQKPGQAPR |
| V_{L} CDR1: SEQ ID NO: 206 | LLIYDASIRATGIPARFSGSGSGTEFTLIISSLQSEDFAVYYCQQYDN |
| V_{L} CDR2: SEQ ID NO: 207 | WPPAYTFGQGTKLEIK |
| V_{L} CDR3: SEQ ID NO: 208 | |
| 1872 V_{H}: SEQ ID NO: 209 | EVQLVESGGGLVKPGGSLRLSCAASGFSFSLYTMNWVRQAPGKG |
| V_{H} CDR1: SEQ ID NO: 210 | LEWVSYISSSSSYRYYADSVKGRFSVSRDNAKNALYLQMNGLRAE |
| V_{H} CDR2: SEQ ID NO: 211 | DTAVYYCARDGYCPRGVCTYYGMDVWGQGTTVTVSA |
| V_{H} CDR3: SEQ ID NO: 212 | |
| 1872 V_{L}: SEQ ID NO: 213 | EIVMTQSPATLSVSPGERATLSCRASQSVGSRLAWYQQKPGQAPR |
| V_{L} CDR1: SEQ ID NO: 214 | LLIYDATIRATGIPARFSGSGSGTDFTLTISGLQSEDFAVYYCQRYNN |
| V_{L} CDR2: SEQ ID NO: 215 | WPPAYTFGQGTKLEIK |
| V_{L} CDR3: SEQ ID NO: 216 | |
| 1888 V_{H}: SEQ ID NO: 217 | EVQLVESGGGLVKPGGSLRLSCAASGFTFSSYSMNWVRQAPGKG |
| V_{H} CDR1: SEQ ID NO: 218 | LEWVSYISSSSSYKYYADSVKGRFSVSRDNAKNSLYLQLNGLRVED |
| V_{H} CDR2: SEQ ID NO: 219 | TAVYYCARDGYCPKGVCTYYGMDVWGQGTTVTVSA |
| V_{H} CDR3: SEQ ID NO: 220 | |
| 1888 V_{L}: SEQ ID NO: 221 | EIVMTQSPATLSVSPGERATLSCRASQSVRSRLAWFQQKPGQPPR |
| V_{L} CDR1: SEQ ID NO: 222 | LLIYDASIRATGIPDRFSGSGSGTEFTLIISGLQSEDFAVYYCQQYDN |
| V_{L} CDR2: SEQ ID NO: 223 | WPPAYTFGQGTKLEIK |
| V_{L} CDR3: SEQ ID NO: 224 | |
| 1915 V_{H}: SEQ ID NO: 225 | QVQLVESGGGVVQPGRSLRLSCAASGFTFSSYAMHWVRQAPGKG |
| V_{H} CDR1: SEQ ID NO: 226 | LEWVAVISYDGSNKYYADSVKGRFTISRDNSKNTLYLQMDSLRAQD |
| V_{H} CDR2: SEQ ID NO: 227 | TAVYYCATPRDMFSSGWSSPFDYWGQGTLVTVSS |
| V_{H} CDR3: SEQ ID NO: 228 | |
| 1915 V_{L}: SEQ ID NO: 229 | DIQMTQSPSSLSASVGDRVTITCQASQDISIYLTWYQQKPGKAPKLL |
| V_{L} CDR1: SEQ ID NO: 230 | IYGASNLEVEVPSRFSGSGSGTEFTLTISSLQPEDFATYFCQHSDDL |
| V_{L} CDR2: SEQ ID NO: 231 | PVTFGGGTKVEVK |
| V_{L} CDR3: SEQ ID NO: 232 | |
| 1959 V_{H}: SEQ ID NO: 233 | QVQLVQSGTEVKKPGSSVRVSCQASGDTFTSHAIIWIRQAPGQGLE |
| V_{H} CDR1: SEQ ID NO: 234 | YLGRIIPVLDITNAAQRFLGRLTLTADKSTTTAYMELSSLRSEDTAVY |
| V_{H} CDR2: SEQ ID NO: 235 | YCARAPFGLIVMYDHWGQGTLVTVTA |
| V_{H} CDR3: SEQ ID NO: 236 | |
| 1959 V_{L}: SEQ ID NO: 237 | EIVLTQSPGTLSLSPGERATLSCRASQSVSGNFLAWYQQKPGQAP |
| V_{L} CDR1: SEQ ID NO: 238 | RLLIHETSKRATGIPDRVSGGGSGTDFTLTISRLEPEDFAVYHCQQY |
| V_{L} CDR2: SEQ ID NO: 239 | GTTAVTFGQGTRLDMK |
| V_{L} CDR3: SEQ ID NO: 240 | |
| 1963 V_{H}: SEQ ID NO: 241 | QVQLVQSGAEVKKPGASVKVSCKASGYTFTSNYIHWLRQAPGQGL |
| V_{H} CDR1: SEQ ID NO: 242 | EWMGIIKPSGTSTISAQKFRGRVAMTRDTSTSTVYMELSSLRFEDT |
| V_{H} CDR2: SEQ ID NO: 243 | AIYYCARDAKRALETWGQGTMVTVSS |
| V_{H} CDR3: SEQ ID NO: 244 | |
| 1963 V_{L}: SEQ ID NO: 245 | QSVLTQPPSVSAAPGQKVTISCSGSSSNIGNNYVSWYQQLPGTAP |
| V_{L} CDR1: SEQ ID NO: 246 | KLLIYDNNKRPSGIPDRFSGSRSGTSATLGITGLQTGDEADYYCGT |
| V_{L} CDR2: SEQ ID NO: 247 | WDSSLSAWVFGGGTKLTVL |
| V_{L} CDR3: SEQ ID NO: 248 | |
| 1969 V_{H}: SEQ ID NO: 249 | QVQLVQSGAEVKKPGASVKVSCKASGYSFTDYYIHWVRQAPGQG |
| V_{H} CDR1: SEQ ID NO: 250 | LEWMGIIKPSAGATVYAQKFRGRVSMTSDTSTGTVYMELSGLKSE |
| V_{H} CDR2: SEQ ID NO: 251 | DTAVYYCARDYNRSFDFWGQGTLVTVSS |
| V_{H} CDR3: SEQ ID NO: 252 | |
| 1969 V_{L}: SEQ ID NO: 253 | QSVLTQPPSVSAAPGQTVTISCAGSTSNIGKNYVSWYQHLPGTAPK |
| V_{L} CDR1: SEQ ID NO: 254 | LLIYDNIKRPSGIPDRFSGSTSGTSATLGITELQTGDEADYYCGTWD |
| V_{L} CDR2: SEQ ID NO: 255 | SSLSAYVFGTGTKVTVL |
| V_{L} CDR3: SEQ ID NO: 256 | |
| 1984 V_{H}: SEQ ID NO: 257 | EVQLVESGGGLVQPGGSLRLSCAASGFTVSSNYMSWVRQAPGKG |
| V_{H} CDR1: SEQ ID NO: 258 | LEWVSVIYSGGGTYYADSVKGRFIISRDNSKNTLYLQMNSLRAEDT |
| V_{H} CDR2: SEQ ID NO: 259 | AVYYCARLSWWGDDNYWGQGTLVTVSS |
| V_{H} CDR3: SEQ ID NO: 260 | |
| 1984 V_{L}: SEQ ID NO: 261 | QAVVTQEPSLTVSPGGTVTLTCGSSTGAVTSGHYPYWFQQKPGQ |
| V_{L} CDR1: SEQ ID NO: 262 | APRTLIYDTSNKHSWTPARFSGSLLGGKAALTLSGAQPEDEADYYC |
| V_{L} CDR2: SEQ ID NO: 263 | LVSYSGARPGVFGGGTKLTVL |
| V_{L} CDR3: SEQ ID NO: 264 | |
| 2019 V_{H}: SEQ ID NO: 265 | QVQLVESGGGVVQPGGSLSLSCAASGLSFSSYGMHWVRQAPGK |
| V_{H} CDR1: SEQ ID NO: 266 | GLEWVAFIRYDVSNKYYADSVKGRFTISRDNSKNTLYLQMNSLRVE |
| V_{H} CDR2: SEQ ID NO: 267 | DTAVYYCATWAPLDDGMDVWGQGTTVTVSS |
| V_{H} CDR3: SEQ ID NO: 268 | |
| 2019 V_{L}: SEQ ID NO: 269 | SYELTQPPSVSVSPGQTARITCSGDALPKQYAYWYQQKPGQAPVL |
| V_{L} CDR1: SEQ ID NO: 270 | VIYKDTERPSGIPERFSGSSSGTTVTLTISGVQAEDEADYYCQSGD |
| V_{L} CDR2: SEQ ID NO: 271 | SSGTFYVVFGGGTKLTVL |
| V_{L} CDR3: SEQ ID NO: 272 | |
| 2020 V_{H}: SEQ ID NO: 273 | QVQLVQSGAEVKKPGASVKVSCKASGYTFTDYFMHWVRQAPGQG |
| V_{H} CDR1: SEQ ID NO: 274 | LEWMGWVNPLSGGTNFAQKFQGRVTMTSDTSITTVYMELSRLRSD |
| V_{H} CDR2: SEQ ID NO: 275 | DTAVYYCARDPPLYSSTYGMDVWGQGTTVTVSS |
| V_{H} CDR3: SEQ ID NO: 276 | |
| 2020 V_{L}: SEQ ID NO: 277 | QSALTQPASVSGSPGQSITISCTGTSSDVGAYNYVSWYQQHPGKA |
| V_{L} CDR1: SEQ ID NO: 278 | PKLMIYDVSDRPSGVSNRFSGSKSGNTASLTISGLQAEDEADYYCS |
| V_{L} CDR2: SEQ ID NO: 279 | SYTISSSVVFGGGTKLTVL |
| V_{L} CDR3: SEQ ID NO: 280 | |
| 2024 V_{H}: SEQ ID NO: 281 | QVQLVQSGAEVKKPGASVKVSCKVSGYTLIELSMHWVRQAPGKGL |
| V_{H} CDR1: SEQ ID NO: 282 | EWMGGFDPEEGETIYAQKFQGRVTMTEDTSTDTAYMELSSLRSED |
| V_{H} CDR2: SEQ ID NO: 283 | TAVYYCTTSPPVGATGAWFDPWGQGTLVTVSA |
| V_{H} CDR3: SEQ ID NO: 284 | |
| 2024 V_{L}: SEQ ID NO: 285 | SYVLTQPPSVSVAPGKTARITCGGNNIGSKSVHWYQQKPGQAPVL |
| V_{L} CDR1: SEQ ID NO: 286 | VVYDDSDRPSGIPERFSGSNSGNTATLTISGLQAEDEADYYCSSYT |
| V_{L} CDR2: SEQ ID NO: 287 | SSSTVVFGGGTKLTVL |
| V_{L} CDR3: SEQ ID NO: 288 | |
| 2025 V_{H}: SEQ ID NO: 289 | QVQLVQSGAEVKKPGASVKVSCKVSGYTLIELSMHWVRQAPGKGL |
| V_{H} CDR1: SEQ ID NO: 290 | EWMGGFDPEDVETIYAQKFQGRVTMTEDTSTDTAYMELSSLRSED |
| V_{H} CDR2: SEQ ID NO: 291 | TAVYYCATAYAVQWRGMIGYWGQGTLVTVSS |
| V_{H} CDR3: SEQ ID NO: 292 | |
| 2025 V_{L}: SEQ ID NO: 293 | QSALTQPASVSGSPGQSITISCTGTSSDVGSYNLVSWYQQHPGKA |
| V_{L} CDR1: SEQ ID NO: 294 | PKLMIYEVSKRPSGVSNRFSGSKSGNTASLTISGLQAEDEADYYCS |
| V_{L} CDR2: SEQ ID NO: 295 | SYAGSSTFSYVFGTGTKVTVL |
| V_{L} CDR3: SEQ ID NO: 296 | |
| 2050 V_{H}: SEQ ID NO: 297 | QVQLVQSGAEVKKPGASVKVPCKVSGYTFTDHFIHWVRQAPGQGL |
| V_{H} CDR1: SEQ ID NO: 298 | EWMGWISPNSGGRNYTQKFQGRVTLTRDTAITTVHMDLSSLTPDD |
| V_{H} CDR2: SEQ ID NO: 299 | TAVYYCARDVRWAQLQGGFDLWGQGTMVTVSS |
| V_{H} CDR3: SEQ ID NO: 300 | |
| 2050 V_{L}: SEQ ID NO: 301 | QSALTQPASVSGSPGQSITISCTGTSSDVGSYNLVSWYQQHPGKA |
| V_{L} CDR1: SEQ ID NO: 302 | PKLMIYEVSKRPSGVSNRFSGSKSGNTASLTISGLQADDEADYYCC |
| V_{L} CDR2: SEQ ID NO: 303 | SYAGDNNFLFGGGTSLTVL |
| V_{L} CDR3: SEQ ID NO: 304 | |
| 2075 V_{H}: SEQ ID NO: 305 | QVQLVQSGAEVKKPGSSVKVSCTASGGTFSIYAFSWVRQAPGQGL |
| V_{H} CDR1: SEQ ID NO: 306 | EWMGGIIPISGTAGYAQKFQGRVTITADESTSTAYMELSSLRSEDTA |
| V_{H} CDR2: SEQ ID NO: 307 | IYYCARKYRYCSGSRCYTYFDYWGQGTLVTVSS |
| V_{H} CDR3: SEQ ID NO: 308 | |
| 2075 V_{L}: SEQ ID NO: 309 | DIQMTQSPSSLSASVGDRVTITCRASQSISSFLNWYQQKPGKAPKL |
| V_{L} CDR1: SEQ ID NO: 310 | LIYAASSLQSGVPSRFSGSGSGTDFTLTISSLQPEDSATYYCQQSY |
| V_{L} CDR2: SEQ ID NO: 311 | STITFGQGTRLEIK |
| V_{L} CDR3: SEQ ID NO: 312 | |
| 2080 V_{H}: SEQ ID NO: 313 | QVQLQQWGAGLLKPSETLSLTCAVYGGSFSGYYWSWIRQPPGKG |
| V_{H} CDR1: SEQ ID NO: 314 | LEWIGRINDIGSTDYNPSLKSRVTISVDTSKNQFSLKLTSVTAADTAV |
| V_{H} CDR2: SEQ ID NO: 315 | YYCAREPFDSEGTFDYWGQGTLVIVSS |
| V_{H} CDR3: SEQ ID NO: 316 | |
| 2080 V_{L}: SEQ ID NO: 317 | DIQMTQPPSTLSASVGDRVTITCRASQSISRWLAWYRQKPGKAPKL |
| V_{L} CDR1: SEQ ID NO: 318 | LIYDASSLQSGVPSRFSGSGSGTEFTLTISSLQPDDLATYYCQQYNT |
| V_{L} CDR2: SEQ ID NO: 319 | YPYTFGQGTKLEIK |
| V_{L} CDR3: SEQ ID NO: 320 | |
| 2432 V_{H}: SEQ ID NO: 321 | QVQLVQSGAEVKKPGSSVKVSCKASGDTFSSYAISWVRQAPGQGL |
| V_{H} CDR1: SEQ ID NO: 322 | EWLGGIIPIFGSADYAQKFQGRVTITADEFTSTAYMELSSLRSEDTA |
| V_{H} CDR2: SEQ ID NO: 323 | VYFCAREKYGDYGEGPLYNFDYWGQGTLVTVSS |
| V_{H} CDR3: SEQ ID NO: 324 | |
| 2432 V_{L}: SEQ ID NO: 325 | DIQMTQSPSSLSASVGDRVTITCRASQSISSYLNWYQQKPGKAPNL |
| V_{L} CDR1: SEQ ID NO: 326 | LIYAASSLQSGVPSRFSGSGSGTDFTLTISSLQPEDFATYYCQQSYS |
| V_{L} CDR2: SEQ ID NO: 327 | TPKTFGQGTKVEIK |
| V_{L} CDR3: SEQ ID NO: 328 | |
| 2564 V_{H}: SEQ ID NO: 329 | QVQLVQSGAEVKKPGSSVRVSCKASGGTFISYTFNWVRQAPGQG |
| V_{H} CDR1: SEQ ID NO: 330 | LEWMGRIIPIFGIVNYAQKFQGRVTIAADKSTSTAYMELSSLRSEDT |
| V_{H} CDR2: SEQ ID NO: 331 | AMYYCATATVDYDSGEEQSSFDPWGQGTLVTVSS |
| V_{H} CDR3: SEQ ID NO: 332 | |
| 2564 V_{L}: SEQ ID NO: 333 | EIVLTQSPGTLSLSPGERATLSCRASQSVSSSYLAWYQQKAGQTP |
| V_{L} CDR1: SEQ ID NO: 334 | RLLIYAASSRATGVPDRFSGSGSGTDFTLTISRLEAEDFAVYYCQQS |
| V_{L} CDR2: SEQ ID NO: 335 | WTFGQGTKVEIK |
| V_{L} CDR3: SEQ ID NO: 336 | |
| 2598 V_{H}: SEQ ID NO: 337 | QVQLQESGPGLVKPSGTLSLTCVVSGGSISSSNWWSWVRQPPGK |
| V_{H} CDR1: SEQ ID NO: 338 | GLEWIGETFHSGSFNYNPSLKSRVTISVDKSKNQFSLKLSSVTAADT |
| V_{H} CDR2: SEQ ID NO: 339 | AIYYCATTRVGYEGHFYYYGMDVWGQGTTVTVSS |
| V_{H} CDR3: SEQ ID NO: 340 | |
| 2598 V_{L}: SEQ ID NO: 341 | DIQMTQSPSSVSASVGDRVTITCRASQGISSWLAWYQQKPGKAPK |
| V_{L} CDR1: SEQ ID NO: 342 | LLIYAASSLQSGVPSRFSGSGSGTDFTLTISSLQPEDFATYYCQQAN |
| V_{L} CDR2: SEQ ID NO: 343 | RFPWTFGQGTKVEIK |
| V_{L} CDR3: SEQ ID NO: 344 | |
| 2606 V_{H}: SEQ ID NO: 345 | EVQLVESGGGLVKPGGSLRLSCAASGFTFSSYSMNWVRQAPGKG |
| V_{H} CDR1: SEQ ID NO: 346 | LEWVSSITSSSGYMYYADSVKGRFTISRDNAKNSLYLQLNSLRAED |
| V_{H} CDR2: SEQ ID NO: 347 | TAVYYCAKDSAFDLWEVRSYYYVMDVWGQGTTVTVSS |
| V_{H} CDR3: SEQ ID NO: 348 | |
| 2606 V_{L}: SEQ ID NO: 349 | EIVMTQSPATLSVSPGERATLSCRASQSVSSNLAWYQQKPGQAPR |
| V_{L} CDR1: SEQ ID NO: 350 | LLIYGASTRATGIPARFSGSGSGTEFTLTISSLQSEDFALYYCQQYN |
| V_{L} CDR2: SEQ ID NO: 351 | NWPRTFGQGTKLEIK |
| V_{L} CDR3: SEQ ID NO: 352 | |
| 2619 V_{H}: SEQ ID NO: 353 | EVQLVESGGGLVQPGRSLRLSCAASGFTFDDYAMHWVRQAPGKG |
| V_{H} CDR1: SEQ ID NO: 354 | LEWVSGISWNSGSIGYADSVKGRFTLSRDNAKNSLFLQMDSLRAE |
| V_{H} CDR2: SEQ ID NO: 355 | DTALYYCAKDVSYRSGSYYRFWGGSGSWGQGTLVTVSS |
| V_{H} CDR3: SEQ ID NO: 356 | |
| 2619 V_{L}: SEQ ID NO: 357 | DIQMTQSPSSLSASVGDRVTITCRASLSIRSYLNWYQQKPGKPPKL |
| V_{L} CDR1: SEQ ID NO: 358 | LIYAASSLQSGVPSRFSGSGSGTDFTLTISSLQPEDFATYYCQQSYS |
| V_{L} CDR2: SEQ ID NO: 359 | TPLTFGGGTKVEIK |
| V_{L} CDR3: SEQ ID NO: 360 | |
| 2646 V_{H}: SEQ ID NO: 361 | QVQLVQSGAEVKKPGASVKVSCKVSGYTLTELSIHWVRQAPGKGL |
| V_{H} CDR1: SEQ ID NO: 362 | EWMGGFDPEDAETIYAQKFQGRVTMTEDTSTDTAYMELSSLRSED |
| V_{H} CDR2: SEQ ID NO: 363 | TAVYYCATATAVAGTVFNYQYHYGLDFWGQGTTVTVSS |
| V_{H} CDR3: SEQ ID NO: 364 | |
| 2646 V_{L}: SEQ ID NO: 365 | DIVMTQTPLSSPVTLGQPASISCRSSQSLVHSDGNTYLSWLQQRPG |
| V_{L} CDR1: SEQ ID NO: 366 | QPPRLLIYKISNRFSGVPDRFSGSGAGTDFTLKISRVEAEDVGVYYC |
| V_{L} CDR2: SEQ ID NO: 367 | MQATQFPHTFGRGTKLEIK |
| V_{L} CDR3: SEQ ID NO: 368 | |
| 2706 V_{H}: SEQ ID NO: 369 | QVQLVQSGAEVKKPGSSVKVSCKASGGTFSTYAISWVRQAPGQGL |
| V_{H} CDR1: SEQ ID NO: 370 | EWMGGIIPLFATANYAQNFQGRVTITADKSTSTAYMELSSLRSEDT |
| V_{H} CDR2: SEQ ID NO: 371 | AVYYCASVRLHLEELSLSHQGDYYYGLDVWGQGTTVTVSS |
| V_{H} CDR3: SEQ ID NO: 372 | |
| 2706 V_{L}: SEQ ID NO: 373 | DIQMTQSPSSLSASVADRVTITCQASQDISHYLNWYQQKPGKAPQL |
| V_{L} CDR1: SEQ ID NO: 374 | LIYDASKLETGAPSRFSGSGSGTDFTFTISSLQPEDIATYYCQQYDH |
| V_{L} CDR2: SEQ ID NO: 375 | LPLTFGGGSKVEIK |
| V_{L} CDR3: SEQ ID NO: 376 | |
| 2729 V_{H}: SEQ ID NO: 377 | QVTLKESGPVLVKPTETLTLTCTVSGFSLSNARMGVSWIRQPPGKA |
| V_{H} CDR1: SEQ ID NO: 378 | LEWLAHIFSNDEKSYSTYLKSRLTISKDSSKSQVVLTMTIMDPVDTA |
| V_{H} CDR2: SEQ ID NO: 379 | TYYCARTAGRYSSRWGHYYYYMDVWGKGTTVTVSS |
| V_{H} CDR3: SEQ ID NO: 380 | |
| 2729 V_{L}: SEQ ID NO: 381 | NFMLTQPHSVSESPGKTVTISCTGSNGSIASNFMQWYQQRPGSAP |
| V_{L} CDR1: SEQ ID NO: 382 | TIVIYEDNQRPSGVPDRFSGSIDGSSNSASLTISGLKTEDEADYYCQ |
| V_{L} CDR2: SEQ ID NO: 383 | SYDSSDSDLGVFGGGTKLTVL |
| V_{L} CDR3: SEQ ID NO: 384 | |
| 2788 V_{H}: SEQ ID NO: 385 | QVQLVQSGAEVKKPGASVKVSCKVSGYTLIELSMHWVRQAPGKGL |
| V_{H} CDR1: SEQ ID NO: 386 | EWMGGFDPEGAETIYAQKFQGRVTMTEDTSTDTAYMELSSLRSDD |
| V_{H} CDR2: SEQ ID NO: 387 | TAVYYCATGPAVFAANWFDPWGQGTLVTVSS |
| V_{H} CDR3: SEQ ID NO: 388 | |
| 2788 V_{L}: SEQ ID NO: 389 | QSALTQPPSASGSPGQSVTISCTGTSSDIGNYNNVSWYQQHPGKA |
| V_{L} CDR1: SEQ ID NO: 390 | PKLMIYDVIKRPSGVPDRFSGSKSGNTASLTVSGLQAEDEADYYCS |
| V_{L} CDR2: SEQ ID NO: 391 | SYAGKSYVFGTGTKVTVL |
| V_{L} CDR3: SEQ ID NO: 392 | |
| 2793 V_{H}: SEQ ID NO: 393 | QVQLVQSGAEVKKPGASVKVSCKVSGFTLPELSIHWVRQAPGKGL |
| V_{H} CDR1: SEQ ID NO: 394 | EWMGGFDPEDAKTIYAQKFQGRVTMTEDTSTDIAYMELNSLRSDD |
| V_{H} CDR2: SEQ ID NO: 395 | TAVYYCATGSPFGVVGNWLDPWGQGTLVTVSS |
| V_{H} CDR3: SEQ ID NO: 396 | |
| 2793 V_{L}: SEQ ID NO: 397 | QSALTQPASVSGSPGQSITISCTGTSSDVGDFSYVSWYQQHPGKA |
| V_{L} CDR1: SEQ ID NO: 398 | PKLMIYEVTKRPSGVSNRFSGSKSGNTASLTISGLQTEDEADYYCT |
| V_{L} CDR2: SEQ ID NO: 399 | SYTSSRLVLFGGGTKLTVL |
| V_{L} CDR3: SEQ ID NO: 400 | |
| 2794 V_{H}: SEQ ID NO: 401 | QVQLVQSGAEVKKPGSSLKVSCKASGGTFNNFAISWVRQAPGQG |
| V_{H} CDR1: SEQ ID NO: 402 | PEWMGRINPILSAAKYAQKFQGRLTITADKSTTTAYMELSSLRSE |
| V_{H} CDR2: SEQ ID NO: 403 | DTAVYYCAPTGTGESWWFDPWGQGTLVTVSS |
| V_{H} CDR3: SEQ ID NO: 404 | |
| 2794 V_{L}: SEQ ID NO: 405 | QSVLTQPPSASGTPGQRVTISCSGSSSNIGTNYVYWYQQLPGTAP |
| V_{L} CDR1: SEQ ID NO: 406 | KVLIYGNNQRPSGVPDRFSGSKSGSSASLAISGLRSEDEADYYCAA |
| V_{L} CDR2: SEQ ID NO: 407 | WDDSLSGPVFGGGTKLTVL |
| V_{L} CDR3: SEQ ID NO: 408 | |
| 2854 V_{H}: SEQ ID NO: 409 | QVQLVQSGAEVKKPGSSVKVSCKASGGTFSSYAISWVRQAPGQG |
| V_{H} CDR1: SEQ ID NO: 410 | LEWMGGIIPIFHTANYAQKFQGRVTITADESTSTTYVELSSLRSE |
| V_{H} CDR2: SEQ ID NO: 411 | DTAMYYCATLPITIFGEEYSFDNWGQGTLVTVSS |
| V_{H} CDR3: SEQ ID NO: 412 | |
| 2854 V_{L}: SEQ ID NO: 413 | DVVMTQSPLSLPVTLGQPASISCRSSQSLVHSDGNTYLSWFQQRP |
| V_{L} CDR1: SEQ ID NO: 414 | GQSPRRLIYKVSDRDSGVPDRFSGSGSGTDFTLKISRVEAEDVGIY |
| V_{L} CDR2: SEQ ID NO: 415 | YCMQGTHWPPLTFGGGTKVEIK |
| V_{L} CDR3: SEQ ID NO: 416 | |
| 2866 V_{H}: SEQ ID NO: 417 | QVQLVQSGAEVKKPGSSVKVSCKASGGTFSSYSISWVRQAPGQG |
| V_{H} CDR1: SEQ ID NO: 418 | LEWMGGLAPIFHTPNYAQKFQGRVTITADESTSTAYMELSSLRSED |
| V_{H} CDR2: SEQ ID NO: 419 | TAVYYCARVAGAGWGVYGAFDYWGQGTLVTVSS |
| V_{H} CDR3: SEQ ID NO: 420 | |
| 2866 V_{L}: SEQ ID NO: 421 | DIVMTQSPDSLAVSLGERATINCKSSQSVLHSSNNKNDLAWYQQK |
| V_{L} CDR1: SEQ ID NO: 422 | PGQPPKLLIYWASTRESGVPDRFSGSGSGTDFTLTISSLQAEDVAV |
| V_{L} CDR2: SEQ ID NO: 423 | YYCQQYYSSPGTFGPGTKVDIK |
| V_{L} CDR3: SEQ ID NO: 424 | |
| 2892 V_{H}: SEQ ID NO: 425 | QVQLVQSGAEVKKPGSSVKVSCKASGDAFISYAISWVRQAPGQGL |
| V_{H} CDR1: SEQ ID NO: 426 | EWMGGIIPIFGTANYAQKFQGRVTISADESTSTAYMELSRLRSED |
| V_{H} CDR2: SEQ ID NO: 427 | TAVYYCARGGPEPYGSGSGYTPRYNWFDPWGQGTLVTVSS |
| V_{H} CDR3: SEQ ID NO: 428 | |
| 2892 V_{L}: SEQ ID NO: 429 | QSALTQPRSVSGSPGQSVTISCTGTSSDVGGYNYVSWYQQHPGK |
| V_{L} CDR1: SEQ ID NO: 430 | APKFMIYDVNRRPSGVPDRFSGSKSGNTASLTISGLQAEDEADY |
| V_{L} CDR2: SEQ ID NO: 431 | YCCSYAGTYTWVFGGGTKLTVL |
| V_{L} CDR3: SEQ ID NO: 432 | |
| 3086 V_{H}: SEQ ID NO: 433 | QVQLQESGPGLVKPSETLSLTCTVSGGSISGHYWSWIRQPPGKGL |
| V_{H} CDR1: SEQ ID NO: 434 | EWIGYIYYSGSTNYNPSLKSRVTISVDTSVNQFSLKLSSVTAADT |
| V_{H} CDR2: SEQ ID NO: 435 | YYCARQEARDAVGNYFFDSWGQGTLVTVSS |
| V_{H} CDR3: SEQ ID NO: 436 | AV |
| 3086 V_{L}: SEQ ID NO: 437 | QSALTQPASVSGSPGQSITISCSGTSSDIGSYDLVSWYQQHPGKAP |
| V_{L} CDR1: SEQ ID NO: 438 | KLLIYDVTKRPSGVSHRFSGSKSGNTASLTISGLQGEDEADYYCCS |
| V_{L} CDR2: SEQ ID NO: 439 | YVHFSTWVFGGGTKLTVL |
| V_{L} CDR3: SEQ ID NO: 440 | |
| 3091 V_{H}: SEQ ID NO: 441 | QMQLVQSGPEVKKPGTSVKVSCKASGFTFSSSAVQWVRQARGQG |
| V_{H} CDR1: SEQ ID NO: 442 | LEWIGWIVVGSGNANYAQKLQERVSITRDMSTSTAYMELSSLRPED |
| V_{H} CDR2: SEQ ID NO: 443 | TAVYYCAAPHCSRTICHDGFDMWGQGTMVTVSS |
| V_{H} CDR3: SEQ ID NO: 444 | |
| 3091 V_{L}: SEQ ID NO: 445 | EIVLTQSPGTLSLSPGERATLSCRASQSVRSSYLAWYQQKPGQAP |
| V_{L} CDR1: SEQ ID NO: 446 | RLLMFVASSRATGIPDRFSGSGSGTDFTLTISRLEPEDFAVYYCQ |
| V_{L} CDR2: SEQ ID NO: 447 | QYDTSPWTFGQGTKVEIK |
| V_{L} CDR3: SEQ ID NO: 448 | |
| 3995 V_{H}: SEQ ID NO: 449 | EVQLVQSGAEVKKPGSSVKVSCKASGGTFSMHTIRWVRQAPGQGLE |
| V_{H} CDR1: SEQ ID NO: 450 | WMGRIIPMLGIVNYAQKFQGRVTISADKSTSTAYMELSSLTSEDT |
| V_{H} CDR2: SEQ ID NO: 451 | AMYYCAKGSHDVFDIWGQGTMVTVSS |
| V_{H} CDR3: SEQ ID NO: 452 | |
| 3995 V_{L}: SEQ ID NO: 453 | DIQMTQSPSTLSASVGDRVTITCRASQSISSWLAWYQQKPGKAPKL |
| V_{L} CDR1: SEQ ID NO: 454 | LIYDASSLESGVPSRFSGSGSGTEFTLTISSLQPDDFATYYCQQYN |
| V_{L} CDR2: SEQ ID NO: 455 | SYSPITFGQGTRLEIK |
| V_{L} CDR3: SEQ ID NO: 456 | |
| 4042 V_{H}: SEQ ID NO: 457 | QITLKESGPTLVKPTQTLTLTCTFSGFSLSSGGVGVGWIRQPPGKA |
| V_{H} CDR1: SEQ ID NO: 458 | LEWLALIYWDDDKRYRPSLKSRLTITRDTSTNQVVLTMTNMDPVDT |
| V_{H} CDR2: SEQ ID NO: 459 | ATYFCARHQIATVFDHWGQGTLVTVSS |
| V_{H} CDR3: SEQ ID NO: 460 | |
| 4042 V_{L}: SEQ ID NO: 461 | QSALTQPASVSGSPGQSITISCTGTSSDVGGYNYVSWYQQHPGKA |
| V_{L} CDR1: SEQ ID NO: 462 | PKLMIYEVSNRPSGVSSRFSGSKSGNTASLTISGLQAEDEADYYCS |
| V_{L} CDR2: SEQ ID NO: 463 | SYTRSSPLVAFGGGTKVTVL |
| V_{L} CDR3: SEQ ID NO: 464 | |
| 4441 V_{H}: SEQ ID NO: 465 | EVQLVESGGGLVQPGRSLRLSCAASGLTFEDYAMHWVRQPPGKG |
| V_{H} CDR1: SEQ ID NO: 466 | LEWVSGVSWNSGTIGYADSVKGRFTISRDNAKNSLYLHMRSLGAE |
| V_{H} CDR2: SEQ ID NO: 467 | DTAMYYCAKDMGGRFSFFSLENDAFDIWGQGTMVIVSS |
| V_{H} CDR3: SEQ ID NO: 468 | |
| 4441 V_{L}: SEQ ID NO: 469 | SYELTQPPSVSVSPGQTARITCSGDALPKQSTYWYQQKPGQAPVL |
| V_{L} CDR1: SEQ ID NO: 470 | VIYKDIERPSGIPERFSGSSSGTTVTLTISGVQAEDEADYYCQSA |
| V_{L} CDR2: SEQ ID NO: 471 | DSSDTYVFGTGTKVTVL |
| V_{L} CDR3: SEQ ID NO: 472 | |

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 508. CDR sequences may be defined according to IMGT. In certain embodiments, the SARS-CoV-2 antigen is the receptor-binding domain (RBD) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 508; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 508; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 508.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 767. CDR sequences may be defined according to IMGT. In certain embodiments, the SARS-CoV-2 antigen is the receptor-binding domain (RBD) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 767; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 767; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 767.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 935. CDR sequences may be defined according to IMGT. In certain embodiments, the SARS-CoV-2 antigen is the receptor-binding domain (RBD) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 935; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 935; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 935.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 937. CDR sequences may be defined according to IMGT. In certain embodiments, the SARS-CoV-2 antigen is the receptor-binding domain (RBD) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 937; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 937; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 937.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 941. CDR sequences may be defined according to IMGT. In certain embodiments, the SARS-CoV-2 antigen is the receptor-binding domain (RBD) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 941; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 941; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 941.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 980. CDR sequences may be defined according to IMGT. In certain embodiments, the SARS-CoV-2 antigen is the receptor-binding domain (RBD, e.g., class III) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 980; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 980; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 980.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1085. CDR sequences may be defined according to IMGT. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1085; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1085; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1085.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1213. CDR sequences may be defined according to IMGT. In certain embodiments, the SARS-CoV-2 antigen is the receptor-binding domain (RBD) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1213; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1213; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1213.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1227. CDR sequences may be defined according to IMGT. In certain embodiments, the SARS-CoV-2 antigen is the receptor-binding domain (RBD) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1227; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1227; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1227.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1231. CDR sequences may be defined according to IMGT. In certain embodiments, the SARS-CoV-2 antigen is the receptor-binding domain (RBD) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1231; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1231; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1231.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1238. CDR sequences may be defined according to IMGT. In certain embodiments, the SARS-CoV-2 antigen is the S1 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1238; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1238; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1238.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1439. CDR sequences may be defined according to IMGT. In certain embodiments, the SARS-CoV-2 antigen is the receptor-binding domain (RBD) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1439; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1439; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1439.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1589. CDR sequences may be defined according to IMGT. In certain embodiments, the SARS-CoV-2 antigen is the receptor-binding domain (RBD, e.g., class I) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1589; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1589; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1589.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1671. CDR sequences may be defined according to IMGT. In certain embodiments, the SARS-CoV-2 antigen is the S1 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1671; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1671; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1671.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1679. CDR sequences may be defined according to IMGT. In certain embodiments, the SARS-CoV-2 antigen is the receptor-binding domain (RBD) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1679; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1679; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1679.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1814. CDR sequences may be defined according to IMGT. In certain embodiments, the SARS-CoV-2 antigen is the S2 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1814; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1814; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1814.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1815. CDR sequences may be defined according to IMGT. In certain embodiments, the SARS-CoV-2 antigen is the S2 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1815; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1815; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1815.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1823. CDR sequences may be defined according to IMGT. In certain embodiments, the SARS-CoV-2 antigen is the S2 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1823; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1823; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1823.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1826. CDR sequences may be defined according to IMGT. In certain embodiments, the SARS-CoV-2 antigen is the S2 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1826; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1826; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1826.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1851. CDR sequences may be defined according to IMGT. In certain embodiments, the SARS-CoV-2 antigen is the S2 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1851; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1851; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1851.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1856. CDR sequences may be defined according to IMGT. In certain embodiments, the SARS-CoV-2 antigen is the S2 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1856; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1856; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1856.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1859. CDR sequences may be defined according to IMGT. In certain embodiments, the SARS-CoV-2 antigen is the S2 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1859; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1859; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1859.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1864. CDR sequences may be defined according to IMGT. In certain embodiments, the SARS-CoV-2 antigen is the S2 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1864; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1864; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1864.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1867. CDR sequences may be defined according to IMGT. In certain embodiments, the SARS-CoV-2 antigen is the S2 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1867; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1867; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1867.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1870. CDR sequences may be defined according to IMGT. In certain embodiments, the SARS-CoV-2 antigen is the S2 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1870; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1870; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1870.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1871. CDR sequences may be defined according to IMGT. In certain embodiments, the SARS-CoV-2 antigen is the S2 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1871; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1871; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1871.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1872. CDR sequences may be defined according to IMGT. In certain embodiments, the SARS-CoV-2 antigen is the S2 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1872; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1872; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1872.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1888. CDR sequences may be defined according to IMGT. In certain embodiments, the SARS-CoV-2 antigen is the S2 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1888; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1888; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1888.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1915. CDR sequences may be defined according to IMGT. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1915; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1915; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1915.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1959. CDR sequences may be defined according to IMGT. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1959; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1959; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1959.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1963. CDR sequences may be defined according to IMGT. In certain embodiments, the SARS-CoV-2 antigen is the S2 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1963; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1963; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1963.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1969. CDR sequences may be defined according to IMGT. In certain embodiments, the SARS-CoV-2 antigen is the S2 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 1969; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1969; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1969.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 1984. CDR sequences may be defined according to IMGT. In certain embodiments, the SARS-CoV-2 antigen is the receptor-binding domain (RBD, e.g., class I) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, designated herein as antibody 1984; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 1984; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 1984.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2019. CDR sequences may be defined according to IMGT. In certain embodiments, the SARS-CoV-2 antigen is a SARS-CoV-2 spike (S) protein trimer. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 2019; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2019; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2019.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2020. CDR sequences may be defined according to IMGT. In certain embodiments, the SARS-CoV-2 antigen is the receptor-binding domain (RBD, e.g., class II) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, designated herein as antibody 2020; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2020; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2020.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2024. CDR sequences may be defined according to IMGT. In certain embodiments, the SARS-CoV-2 antigen is a SARS-CoV-2 spike (S) protein trimer. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 2024; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2024; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2024.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2025. CDR sequences may be defined according to IMGT. In certain embodiments, the SARS-CoV-2 antigen is the receptor-binding domain (RBD, e.g., class II) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, designated herein as antibody 2025; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2025; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2025.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2050. CDR sequences may be defined according to IMGT. In certain embodiments, the SARS-CoV-2 antigen is the receptor-binding domain (RBD, e.g., class II) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 2050; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2050; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2050.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2075. CDR sequences may be defined according to IMGT. In certain embodiments, the SARS-CoV-2 antigen is the receptor-binding domain (RBD, e.g., class II) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, designated herein as antibody 2075; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2075; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2075.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2080. CDR sequences may be defined according to IMGT. In certain embodiments, the SARS-CoV-2 antigen is the S1 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 2080; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2080; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2080.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2432. CDR sequences may be defined according to IMGT. In certain embodiments, the SARS-CoV-2 antigen is a SARS-CoV-2 spike (S) protein trimer. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 2432; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2432; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2432.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2564. CDR sequences may be defined according to IMGT. In certain embodiments, the SARS-CoV-2 antigen is the receptor-binding domain (RBD, e.g., class 1) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 2564; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2564; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2564.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2598. CDR sequences may be defined according to IMGT. In certain embodiments, the SARS-CoV-2 antigen is the S1 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 2598; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2598; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2598.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2606. CDR sequences may be defined according to IMGT. In certain embodiments, the SARS-CoV-2 antigen is a SARS-CoV-2 spike (S) protein trimer. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 2606; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2606; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2606.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2619. CDR sequences may be defined according to IMGT. In certain embodiments, the SARS-CoV-2 antigen is the receptor-binding domain (RBD, e.g., class III) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, designated herein as antibody 2619; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2619; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2619.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2646. CDR sequences may be defined according to IMGT. In certain embodiments, the SARS-CoV-2 antigen is the S1 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 2646; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2646; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2646.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2706. CDR sequences may be defined according to IMGT. In certain embodiments, the SARS-CoV-2 antigen is the receptor-binding domain (RBD, e.g., class III) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, designated herein as antibody 2706; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2706; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2706.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2729. CDR sequences may be defined according to IMGT. In certain embodiments, the SARS-CoV-2 antigen is the receptor-binding domain (RBD, e.g., class III) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 2729; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2729; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2729.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2788. CDR sequences may be defined according to IMGT. In certain embodiments, the SARS-CoV-2 antigen is the S1 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 2788; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2788; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2788.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2793. CDR sequences may be defined according to IMGT. In certain embodiments, the SARS-CoV-2 antigen is the S1 subunit of a SARS-CoV-2 spike (S) protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 2793; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2793; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2793.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2794. CDR sequences may be defined according to IMGT. In certain embodiments, the SARS-CoV-2 antigen is the receptor-binding domain (RBD, e.g., class I) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, designated herein as antibody 2794; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2794; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2794.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2854. CDR sequences may be defined according to IMGT. In certain embodiments, the SARS-CoV-2 antigen is a SARS-CoV-2 spike (S) protein trimer. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 2854; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2854; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2854.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2866. CDR sequences may be defined according to IMGT. In certain embodiments, the SARS-CoV-2 antigen is a SARS-CoV-2 spike (S) protein trimer. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 2866; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2866; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2866.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 2892. CDR sequences may be defined according to IMGT. In certain embodiments, the SARS-CoV-2 antigen is the receptor-binding domain (RBD, e.g., class I) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 2892; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 2892; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 2892.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 3086. CDR sequences may be defined according to IMGT. In certain embodiments, the SARS-CoV-2 antigen is the receptor-binding domain (RBD, e.g., class I) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, designated herein as antibody 3086; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 3086; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 3086.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 3091. CDR sequences may be defined according to IMGT. In certain embodiments, the SARS-CoV-2 antigen is the receptor-binding domain (RBD, e.g., class I) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 3091; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 3091; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 3091.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 3995. CDR sequences may be defined according to IMGT. In certain embodiments, the SARS-CoV-2 antigen is the receptor-binding domain (RBD, e.g., class III) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, designated herein as antibody 3995; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 3995; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 3995.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 4042. CDR sequences may be defined according to IMGT. In certain embodiments, the SARS-CoV-2 antigen is the receptor-binding domain (RBD, e.g., class III) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{H} polypeptide of the antibody designated herein as antibody 4042; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 4042; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 4042.

According to some embodiments, an antibody of the present disclosure specifically binds a SARS-CoV-2 antigen and comprises—or competes for binding to the SARS-CoV-2 antigen with an antibody comprising—one, two, three, four, five, or all six CDRs of the antibody designated herein as antibody 4441. CDR sequences may be defined according to IMGT. In certain embodiments, the SARS-CoV-2 antigen is the receptor-binding domain (RBD, e.g., class III) of the S1 subunit of a SARS-CoV-2 spike protein. In certain embodiments, such an antibody comprises: a V_{H} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, designated herein as antibody 4441; a V_{L} polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the V_{L} polypeptide of the antibody designated herein as antibody 4441; or both. According to some embodiments, such an antibody comprises one or more amino acid substitutions (e.g., one or more conservative amino acid substitutions) in one or more framework regions of the V_{H} polypeptide, the V_{L} polypeptide, or both, as compared to the corresponding one or more framework regions of the V_{H} polypeptide, the V_{L} polypeptide, or both, of the antibody designated herein as antibody 4441.

In certain embodiments, the CDRs are defined according to the IMGT numbering system. According to some embodiments, the CDRs are defined according to the Kabat numbering system.

In certain embodiments, antibody variants having one or more amino acid substitutions relative to a V_{H} and/or V_{L} amino acid sequence set forth in Table 1 are provided. Sites of interest for substitutional mutagenesis include one or more CDRs and/or one or more framework regions (FRs). Conservative substitutions are shown in the following table under the heading of “preferred substitutions.” More substantial changes are provided in the following table under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, improved developability, improved manufacturability, and/or the like.

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, lie;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

Any suitable approach for determining whether a first antibody competes with a second antibody for binding to a SARS-CoV-2 antigen may be employed. Non-limiting examples of such approaches include competition ELISA, competitive SARS-CoV-2 antigen binding assays, and the like.

Methods are available for measuring the affinity of an anti-SARS-CoV-2 antigen antibody for a SARS-CoV-2 antigen using direct binding or competition binding assays. In a direct binding assay, the equilibrium binding constant (K_{D}) may be measured using a candidate anti-SARS-CoV-2 antigen antibody conjugated to a fluorophore or radioisotope, or a candidate anti-SARS-CoV-2 antigen antibody that contains an N- or C-terminal epitope tag for detection by a labeled antibody. If labels or tags are not feasible or desired, a competition binding assay can be used to determine the half-maximal inhibitory concentration (ICSO), the amount of unlabeled candidate anti-SARS-CoV-2 antigen antibody at which 50% of the maximal signal of the labeled competitor is detectable. A K_{D} value can then be calculated from the measured IC₅₀ value. Ligand depletion will be more pronounced when measuring high-affinity interactions over a lower concentration range, and can be avoided or minimized by decreasing the SARS-CoV-2 (or antigen thereof) added in the experiment or by increasing the binding reaction volumes.

The amino acid sequences of SARS-CoV-2 antigens that may be used to determine whether an antibody of the present disclosure competes for binding to a SARS-CoV-2 antigen with a second antibody are provided in Table 2 below.

TABLE 2
SARS-COV-2 Antigen Amino Acid Sequences

| | |
|---|---|
| Receptor- | RVQPTESIVRFPNITNLCPFGEVFN |
| binding domain | ATRFASVYAWNRKRISNCVADYSVL |
| (RBD)- | YNSASFSTFKCYGVSPTKLNDLCFT |
| WA01/2020 strain | NVYADSFVIRGDEVRQIAPGQTGKI |
| (SEQ ID NO: 498) | ADYNYKLPDDFTGCVIAWNSNNLDS |
| | KVGGNYNYLYRLFRKSNLKPFERDI |
| | STEIYQAGSTPCNGVEGFNCYFPLQ |
| | SYGFQPTNGVGYQPYRVVVLSFELL |
| | HAPATVCGPKKSTNLVKNKCVNF |
| S1 subunit- | SQCVNLTTRTQLPPAYTNSFTRGVY |
| WA01/2020 | YPDKVFRSSVLHSTQDLFLPFFSNV |
| strain | TWFHAIHVSGTNGTKRFDNPVLPFN |
| (SEQ ID NO: 499) | DGVYFASTEKSNIIRGWIFGTTLDS |
| | KTQSLLIVNNATNVVIKVCEFQFCN |
| | DPFLGVYYHKNNKSWMESEFRVYSS |
| | ANNCTFEYVSQPFLMDLEGKQGNFK |
| | NLREFVFKNIDGYFKIYSKHTPINL |
| | VRDLPQGFSALEPLVDLPIGINITR |
| | FQTLLALHRSYLTPGDSSSGWTAGA |
| | AAYYVGYLQPRTFLLKYNENGTITD |
| | AVDCALDPLSETKCTLKSFTVEKGI |
| | YQTSNFRVQPTESIVRFPNITNLCP |
| | FGEVFNATRFASVYAWNRKRISNCV |
| | ADYSVLYNSASFSTFKCYGVSPTKL |
| | NDLCFTNVYADSFVIRGDEVRQIAP |
| | GQTGKIADYNYKLPDDFTGCVIAWN |
| | SNNLDSKVGGNYNYLYRLFRKSNLK |
| | PFERDISTEIYQAGSTPCNGVEGFN |
| | CYFPLQSYGFQPTNGVGYQPYRVVV |
| | LSFELLHAPATVCGPKKSTNLVKNK |
| | CVNFNFNGLTGTGVLTESNKKFLPF |
| | QQFGRDIADTTDAVRDPQTLEILDI |
| | TPCSFGGVSVITPGTNTSNQVAVLY |
| | QDVNCTEVPVAIHADQLTPTWRVYS |
| | TGSNVFQTRAGCLIGAEHVNNSYEC |
| | DIPIGAGICASYQTQTNSPRRAR |
| S2 subunit- | SVASQSIIAYTMSLGAENSVAYSNN |
| WA01/2020 | SIAIPTNFTISVTTEILPVSMTKTS |
| strain | VDCTMYICGDSTECSNLLLQYGSFC |
| (SEQ ID NO: 500) | TQLNRALTGIAVEQDKNTQEVFAQV |
| | KQIYKTPPIKDFGGFNFSQILPDPS |
| | KPSKRSFIEDLLFNKVTLADAGFIK |
| | QYGDCLGDIAARDLICAQKFNGLTV |
| | LPPLLTDEMIAQYTSALLAGTITSG |
| | WTFGAGAALQIPFAMQMAYRFNGIG |
| | VTQNVLYENQKLIANQFNSAIGKIQ |
| | DSLSSTASALGKLQDVVNQNAQALN |
| | TLVKQLSSNFGAISSVLNDILSRLD |
| | KVEAEVQIDRLITGRLQSLQTYVTQ |
| | QLIRAAEIRASANLAATKMSECVLG |
| | QSKRVDFCGKGYHLMSFPQSAPHGV |
| | VFLHVTYVPAQEKNFTTAPAICHDG |
| | KAHFPREGVFVSNGTHWFVTQRNFY |
| | EPQIITTDNTFVSGNCDVVIGIVNN |
| | TVYDPLQPELDSFKEELDKYFKNHT |
| | SPDVDLGDISGINASVVNIQKEIDR |
| | LNEVAKNLNESLIDLQELGKYEQYI |
| | KWPWYIWLGFIAGLIAIVMVTIMLC |
| | CMTSCCSCLKGCCSCGSCCKFDEDD |
| | SEPVLKGVKLHYT |
| Trimer- | MFVFLVLLPLVSSQCVNLTTRTQLP |
| WA01/2020 strain | PAYTNSFTRGVYYPDKVFRSSVLHS |
| (SEQ ID NO: 501) | TQDLFLPFFSNVTWFHAIHVSGTNG |
| | TKRFDNPVLPFNDGVYFASTEKSNI |
| | IRGWIFGTTLDSKTQSLLIVNNATN |
| | VVIKVCEFQFCNDPFLGVYYHKNNK |
| | SWMESEFRVYSSANNCTFEYVSQPF |
| | LMDLEGKQGNFKNLREFVFKNIDGY |
| | FKIYSKHTPINLVRDLPQGFSALEP |
| | LVDLPIGINITRFQTLLALHRSYLT |
| | PGDSSSGWTAGAAAYYVGYLQPRTF |
| | LLKYNENGTITDAVDCALDPLSETK |
| | CTLKSFTVEKGIYQTSNFRVQPTES |
| | IVRFPNITNLCPFGEVFNATRFASV |
| | YAWNRKRISNCVADYSVLYNSASFS |
| | TFKCYGVSPTKLNDLCFTNVYADSF |
| | VIRGDEVRQIAPGQTGKIADYNYKL |
| | PDDFTGCVIAWNSNNLDSKVGGNYN |
| | YLYRLFRKSNLKPFERDISTEIYQA |
| | GSTPCNGVEGFNCYFPLQSYGFQPT |
| | NGVGYQPYRVVVLSFELLHAPATVC |
| | GPKKSTNLVKNKCVNFNFNGLTGTG |
| | VLTESNKKFLPFQQFGRDIADTTDA |
| | VRDPQTLEILDITPCSFGGVSVITP |
| | GTNTSNQVAVLYQDVNCTEVPVAIH |
| | ADQLTPTWRVYSTGSNVFQTRAGCL |
| | IGAEHVNNSYECDIPIGAGICASYQ |
| | TQTNSPRRARSVASQSIIAYTMSLG |
| | AENSVAYSNNSIAIPTNFTISVTTE |
| | ILPVSMTKTSVDCTMYICGDSTECS |
| | NLLLQYGSFCTQLNRALTGIAVEQD |
| | KNTQEVFAQVKQIYKTPPIKDFGGF |
| | NFSQILPDPSKPSKRSFIEDLLFNK |
| | VTLADAGFIKQYGDCLGDIAARDLI |
| | CAQKFNGLTVLPPLLTDEMIAQYTS |
| | ALLAGTITSGWTFGAGAALQIPFAM |
| | QMAYRFNGIGVTQNVLYENQKLIAN |
| | QFNSAIGKIQDSLSSTASALGKLQD |
| | VVNQNAQALNTLVKQLSSNFGAISS |
| | VLNDILSRLDKVEAEVQIDRLITGR |
| | LQSLQTYVTQQLIRAAEIRASANLA |
| | ATKMSECVLGQSKRVDFCGKGYHLM |
| | SFPQSAPHGVVFLHVTYVPAQEKNF |
| | TTAPAICHDGKAHFPREGVFVSNGT |
| | HWFVTQRNFYEPQIITTDNTFVSGN |
| | CDVVIGIVNNTVYDPLQPELDSFKE |
| | ELDKYFKNHTSPDVDLGDISGINAS |
| | VVNIQKEIDRLNEVAKNLNESLIDL |
| | QELGKYEQYIKWPWYIWLGFIAGLI |
| | AIVMVTIMLCCMTSCCSCLKGCCSC |
| | GSCCKFDEDDSEPVLKGVKLHYT |

The term “antibody” may include an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the cell surface molecule of the target cell, including, but not limited to single chain Fv (scFv), Fab, (Fab′)₂, (scFv′)₂, and diabodies; chimeric antibodies; monoclonal antibodies, human antibodies, humanized antibodies (e.g., humanized whole antibodies, humanized half antibodies, or humanized antibody fragments, e.g., humanized scFv); and fusion proteins comprising an antigen-binding portion of an antibody and a non-antibody protein. In some embodiments, the antibody is selected from an IgG, Fv, single chain antibody, scFv, Fab, F(ab′)2, or Fab′. The antibodies may be detectably labeled, e.g., with an in vivo imaging agent, a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like.

An immunoglobulin light or heavy chain variable region is composed of a “framework” region (FR) interrupted by three hypervariable regions, also called “complementarity determining regions” or “CDRs”. The extent of the framework region and CDRs can be defined based on databases known in the art. See, for example, “Sequences of Proteins of Immunological Interest,” E. Kabat et al., Sequences of proteins of immunological interest, 4th ed. U.S. Dept. Health and Human Services, Public Health Services, Bethesda, MD (1987), Lefranc et al. IMGT, the international ImMunoGeneTics Information System®. Nucl. Acids Res., 2005, 33:D593-D597 (www.imgt.org/textes/IMGTScientificChart/), and/or V Base at vbase.mrc-cpe.cam.ac.uk/). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

Any anti-SARS-CoV-2 antigen antibody of the present disclosure may be a monoclonal antibody. As used herein, the term “monoclonal antibody” refers to an antibody composition having a homogeneous antibody population. The term is not limited by the manner in which it is made. The term encompasses whole immunoglobulin molecules, as well as Fab molecules, F(ab′)₂ fragments, Fv fragments, single chain fragment variable (scFv), fusion proteins comprising an antigen-binding portion of an antibody and a non-antibody protein, and other molecules that exhibit immunological binding properties of the parent monoclonal antibody molecule. Methods of making monoclonal antibodies are known in the art and described more fully below.

Any anti-SARS-CoV-2 antigen antibody of the present disclosure may be a recombinant or modified antibody, e.g., a chimeric, deimmunized and/or an in vitro generated antibody. The term “recombinant” or “modified” antibody as used herein is intended to include all antibodies that are prepared, expressed, created, or isolated by recombinant means, such as (i) antibodies expressed from one or more recombinant expression vectors transfected into a host cell; (ii) antibodies isolated from a recombinant, combinatorial antibody library; (iii) antibodies isolated from an animal (e.g., a mouse) that is transgenic for human immunoglobulin genes; or (iv) antibodies prepared, expressed, created, or isolated by any other means that involves splicing of human immunoglobulin gene sequences to other DNA sequences. Such recombinant antibodies include, e.g., chimeric, deimmunized, and/or in vitro generated antibodies.

Any anti-SARS-CoV-2 antigen antibody of the present disclosure may be isolated. By “isolated” is meant that the antibody is separated from all or some of the components that accompany it in nature. “Isolated” also refers to the state of an antibody separated from all or some of the components that accompany it during manufacture, e.g., chemical synthesis, recombinant expression, culture medium, and/or the like.

Any anti-SARS-CoV-2 antigen antibody of the present disclosure (e.g., a human anti-SARS-CoV-2 antigen antibody) may comprise an extent and/or pattern of glycosylation which is different from the extent and/or pattern of glycosylation of an antibody produced in nature, e.g., produced in an animal (e.g., produced in a human). For example, an anti-SARS-CoV-2 antigen antibody of the present disclosure may be a recombinant antibody (e.g., a monoclonal antibody) expressed from one or more recombinant expression vectors transfected into a host cell, where the expressed recombinant anti-SARS-CoV-2 antigen antibody comprises a different extent of glycosylation, a different glycosylation pattern, or both, as compared to the extent of glycosylation and/or glycosylation pattern of the antibody when produced in nature, e.g., when produced in an animal in response to a SARS-CoV-2 virus infection (e.g., when produced in a human in response to a SARS-CoV-2 virus infection).

In some embodiments, an anti-SARS-CoV-2 antigen antibody of the present disclosure comprises a heavy chain comprising an Fc region, and the Fc region is heterologous to the V_{H} of the antibody—that is, the Fc region comprises an amino acid sequence (e.g., one or more amino acid substitutions, deletions and/or insertions), one or more post-translational modifications, and/or the like, such that an antibody comprising the combination of the Fc region and the V_{H} does not occur in nature, e.g., is different from an anti-SARS-CoV-2 antigen antibody produced in an animal in response to a SARS-CoV-2 virus infection (e.g., different from an anti-SARS-CoV-2 antigen antibody produced in a human in response to a SARS-CoV-2 virus infection).

In certain embodiments, one or more amino acid modifications may be introduced into the Fc region of an antibody provided herein, thereby generating an Fc region variant. The Fc region variant may comprise a murine Fc region sequence (e.g.: IgG1, IgG2a or IgG2b) comprising an amino acid modification (e.g., substitution) at one or more amino acid positions. The Fc region variant may comprise a human Fc region sequence (e.g., a human IgG1, IgG2, IgG3 or IgG4 Fc region) comprising an amino acid modification (e.g., substitution) at one or more amino acid positions (e.g., an IgG4 isotype including the S228P mutation).

In certain embodiments, the Fc region is mutated to increase its affinity to FcRn at pH 6.0 and consequently extend the antibody half-life. Antibodies with enhanced affinity to FcRn include those with substitution of one or more of Fc region residues 252, 253, 254, 256, 428, 434, including the so called YTE mutation with substitution M252Y/S254T/T256E (Dall' Acqua et al, J Immunol. 169:5171-5180 (2002)) or LS mutation M428L/N434S (Zalevsky et al, Nat Biotechnol. 28(2): 157-159 (2010)).

In certain embodiments, the invention contemplates an antibody variant that possesses some but not all effector functions, which make it a desirable candidate for applications in which the half life of the antibody in vivo is important yet certain effector functions (such as complement activation and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes and microglia express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Natl Acad. Sci. USA 82:1499-1502 (1985); 5,821,337 (see Bruggemann, M. et al., J. Exp. Med. 166:1351-1361 (1987)).

Antibodies with reduced effector function include those with substitution of one or more of Fc region residues 234, 235, 238, 265, 269, 270, 297, 327 and 329 (U.S. Pat. No. 6,737,056). Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001)). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581) or the so-called “DANG” FC mutant with substitution of residues 265 to alanine and 297 to Glycine. Alternatively, antibodies with reduced effector function include those with substitution of one or more of Fc region residues 234, 235 and 329, so-called “PG-LALA” Fc mutant with substitution of residues 234 and 235 to alanine and 329 to glycine (Lo, M. et al., Journal of Biochemistry, 292, 3900-3908). Other known mutations at position 234, 235 and 321, the so called TM mutant containing mutations L234F/L235E/P331S in the CH2 domain, can be used (Oganesyan et al. Acta Cryst. D64, 700-704. (2008)). Antibodies from the human IgG4 isotype include mutations S228P/L235E to stabilize the hinge and to reduce FgR binding (Schlothauer et al, PEDS, 29 (10):457-466).

Other Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821.

The phrases “specifically binds”, “specific for”, “immunoreactive” and “immunoreactivity”, and “antigen binding specificity”, when referring to an antibody, refer to a binding reaction with an antigen which is highly preferential to the antigen or a fragment thereof, so as to be determinative of the presence of the antigen in the presence of a heterogeneous population of antigens (e.g., proteins and other biologics, e.g., in a sample). Thus, under designated immunoassay conditions, the specified antibodies bind to a particular SARS-CoV-2 antigen and do not bind in a significant amount to other antigens present in the sample. Specific binding to an antigen under such conditions may require an antibody that is selected for its specificity for a particular antigen. For example, an anti-SARS-CoV-2 antigen antibody can specifically bind to a SARS-CoV-2 antigen, and does not exhibit comparable binding (e.g., does not exhibit detectable binding) to other proteins present in a sample.

In some embodiments, an antibody of the present disclosure “specifically binds” a SARS-CoV-2 antigen if it binds to or associates with the SARS-CoV-2 antigen (e.g., the S1 subunit of a SARS-CoV-2 spike (S) protein, the receptor-binding domain (RBD) of the S1 subunit of a SARS-CoV-2 spike protein, the S2 subunit of a SARS-CoV-2 spike protein, a SARS-CoV-2 envelope (E) protein, a SARS-CoV-2 membrane (M) protein, or a SARS-CoV-2 nucleocapsid (N) protein) with an affinity or Kₐ (that is, an equilibrium association constant of a particular binding interaction with units of 1/M) of, for example, greater than or equal to about 105 M⁻¹. In certain embodiments, the antibody binds to SARS-CoV-2 with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Kₐ of at least 107 M⁻¹, at least 10⁸ M⁻¹, at least 109 M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{D}) of a particular binding interaction with units of M (e.g., 10⁻⁵ M to 10⁻¹³ M, or less). In some embodiments, specific binding means the antibody binds to SARS-CoV-2 with a K_{D} of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the antibody for the SARS-CoV-2 antigen can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; or the like.

An “epitope” is a site on an antigen (e.g., a site on a SARS-CoV-2 antigen such as the S1 subunit of a SARS-CoV-2 spike (S) protein, the receptor-binding domain (RBD) of the S1 subunit of a SARS-CoV-2 spike protein, the S2 subunit of a SARS-CoV-2 spike protein, a SARS-CoV-2 envelope (E) protein, a SARS-CoV-2 membrane (M) protein, and a SARS-CoV-2 nucleocapsid (N) protein) to which an antibody binds. Epitopes can be formed both from contiguous amino acids or noncontiguous amino acids juxtaposed by folding (e.g., tertiary folding) of a protein. Epitopes formed from contiguous amino acids are typically retained on exposure to denaturing solvents whereas epitopes formed by folding are typically lost on treatment with denaturing solvents. An epitope typically includes at least 3, and more usually, at least 5 or 8-10 amino acids in a linear or spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996). Several commercial laboratories offer epitope mapping services. Epitopes bound by an antibody immunoreactive with a SARS-CoV-2 antigen can reside, e.g., on the surface of SARS-CoV-2 or an antigen thereof, so that such epitopes are considered SARS-CoV-2-surface accessible, solvent accessible, and/or SARS-CoV-2-surface exposed.

According to some embodiments, an antibody of the present disclosure is an IgG antibody. In certain embodiments, such an antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{H} encoded by the polynucleotide set forth in SEQ ID NO:473, and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{L} encoded by the polynucleotide set forth in SEQ ID NO:474,
wherein the antibody comprises one or more, two or more, three or more, four or more, five or six of the complementarity determining regions (CDRs) of the antibody encoded by the polynucleotides set forth in SEQ ID NO:473 and SEQ ID NO:474;
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{H} encoded by the polynucleotide set forth in SEQ ID NO:475, and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{L} encoded by the polynucleotide set forth in SEQ ID NO:476,
wherein the antibody comprises one or more, two or more, three or more, four or more, five or six of the complementarity determining regions (CDRs) of the antibody encoded by the polynucleotides set forth in SEQ ID NO:475 and SEQ ID NO:476;
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{H} encoded by the polynucleotide set forth in SEQ ID NO:477, and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{L} encoded by the polynucleotide set forth in SEQ ID NO:478,
wherein the antibody comprises one or more, two or more, three or more, four or more, five or six of the complementarity determining regions (CDRs) of the antibody encoded by the polynucleotides set forth in SEQ ID NO:477 and SEQ ID NO:478;
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{H} encoded by the polynucleotide set forth in SEQ ID NO:479, and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{L} encoded by the polynucleotide set forth in SEQ ID NO:480,
wherein the antibody comprises one or more, two or more, three or more, four or more, five or six of the complementarity determining regions (CDRs) of the antibody encoded by the polynucleotides set forth in SEQ ID NO:479 and SEQ ID NO:480;
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{H} encoded by the polynucleotide set forth in SEQ ID NO:481, and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{L} encoded by the polynucleotide set forth in SEQ ID NO:482,
wherein the antibody comprises one or more, two or more, three or more, four or more, five or six of the complementarity determining regions (CDRs) of the antibody encoded by the polynucleotides set forth in SEQ ID NO:481 and SEQ ID NO:482;
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{H} encoded by the polynucleotide set forth in SEQ ID NO:483, and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{L} encoded by the polynucleotide set forth in SEQ ID NO:484,
wherein the antibody comprises one or more, two or more, three or more, four or more, five or six of the complementarity determining regions (CDRs) of the antibody encoded by the polynucleotides set forth in SEQ ID NO:483 and SEQ ID NO:484;
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{H} encoded by the polynucleotide set forth in SEQ ID NO:485, and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{L} encoded by the polynucleotide set forth in SEQ ID NO:486,
wherein the antibody comprises one or more, two or more, three or more, four or more, five or six of the complementarity determining regions (CDRs) of the antibody encoded by the polynucleotides set forth in SEQ ID NO:485 and SEQ ID NO:486;
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{H} encoded by the polynucleotide set forth in SEQ ID NO:487, and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{L} encoded by the polynucleotide set forth in SEQ ID NO:488,
wherein the antibody comprises one or more, two or more, three or more, four or more, five or six of the complementarity determining regions (CDRs) of the antibody encoded by the polynucleotides set forth in SEQ ID NO:487 and SEQ ID NO:488;
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{H} encoded by the polynucleotide set forth in SEQ ID NO:489, and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{L} encoded by the polynucleotide set forth in SEQ ID NO:490,
wherein the antibody comprises one or more, two or more, three or more, four or more, five or six of the complementarity determining regions (CDRs) of the antibody encoded by the polynucleotides set forth in SEQ ID NO:489 and SEQ ID NO:490;
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{H} encoded by the polynucleotide set forth in SEQ ID NO:491, and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{L} encoded by the polynucleotide set forth in SEQ ID NO:492,
wherein the antibody comprises one or more, two or more, three or more, four or more, five or six of the complementarity determining regions (CDRs) of the antibody encoded by the polynucleotides set forth in SEQ ID NO:491 and SEQ ID NO:492;
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{H} encoded by the polynucleotide set forth in SEQ ID NO:493, and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{L} encoded by the polynucleotide set forth in SEQ ID NO:494,
wherein the antibody comprises one or more, two or more, three or more, four or more, five or six of the complementarity determining regions (CDRs) of the antibody encoded by the polynucleotides set forth in SEQ ID NO:493 and SEQ ID NO:494; or a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{H} encoded by the polynucleotide set forth in SEQ ID NO:495, and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{L} encoded by the polynucleotide set forth in SEQ ID NO:496,
wherein the antibody comprises one or more, two or more, three or more, four or more, five or six of the complementarity determining regions (CDRs) of the antibody encoded by the polynucleotides set forth in SEQ ID NO:495 and SEQ ID NO:496.

In certain embodiments, provided is an antibody encoded by the polynucleotides set forth in: SEQ ID NO:473 and SEQ ID NO:474; SEQ ID NO:475 and SEQ ID NO:476; SEQ ID NO:477 and SEQ ID NO:478; SEQ ID NO:479 and SEQ ID NO:480; SEQ ID NO:481 and SEQ ID NO:482; SEQ ID NO:483 and SEQ ID NO:484; SEQ ID NO:485 and SEQ ID NO:486; SEQ ID NO:487 and SEQ ID NO:488; SEQ ID NO:489 and SEQ ID NO:490; SEQ ID NO:491 and SEQ ID NO:492; SEQ ID NO:493 and SEQ ID NO:494; or SEQ ID NO:495 and SEQ ID NO:496.

Fusion Proteins

Aspects of the present disclosure further include fusion proteins. The fusion proteins comprise a variable heavy chain (V_{H}) polypeptide, a variable light chain (V_{L}) polypeptide, or both, of an antibody of the present disclosure, fused directly or indirectly to a heterologous amino acid sequence. “Heterologous” as used in the context of a nucleic acid or polypeptide generally means that the nucleic acid or polypeptide is from a different origin (e.g., molecule of different sequence, different species origin, and the like) than that with which the nucleic acid or polypeptide is associated or joined, such that the nucleic acid or polypeptide is one that is not found in nature. For example, in a fusion protein, a light chain polypeptide and a reporter polypeptide (e.g., GFP, luciferase, etc.) are said to be “heterologous” to one another. Similarly, a CDR from a non-human antibody and a constant region from a human antibody are said to be “heterologous” to one another.

In certain embodiments, a fusion protein of the present disclosure comprises the heterologous sequence of amino acids fused to the C-terminus of the chain of the antibody. According to some embodiments, the antibody is a single chain antibody as described elsewhere herein, e.g., an scFv.

In certain embodiments, a fusion protein of the present disclosure is a chimeric antigen receptor (CAR) comprising a single chain antibody of the present disclosure, a transmembrane domain, and an intracellular signaling domain.

A CAR of the present disclosure may include one or more linker sequences between the various domains. A “variable region linking sequence” is an amino acid sequence that connects a heavy chain variable region to a light chain variable region and provides a spacer function compatible with interaction of the two sub-binding domains so that the resulting polypeptide retains a specific binding affinity to the same target molecule as an antibody that includes the same light and heavy chain variable regions. A non-limiting example of a variable region linking sequence is a serine-glycine linker, such as a serine-glycine linker that includes the amino acid sequence GGGGSGGGGSGGGGS (G₄S)₃ (SEQ ID NO:497). In certain aspects, a linker separates one or more heavy or light chain variable domains, hinge domains, transmembrane domains, co-stimulatory domains, and/or primary signaling domains. In particular embodiments, the CAR includes one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids in length.

In some embodiments, the antigen binding domain of the CAR is followed by one or more spacer domains that moves the antigen binding domain away from the effector cell surface (e.g., the surface of a T cell expressing the CAR) to enable proper cell/cell contact, antigen binding and/or activation. The spacer domain (and any other spacer domains, linkers, and/or the like described herein) may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain may include the amino acid sequence of a naturally occurring immunoglobulin hinge region or an altered immunoglobulin hinge region. In one embodiment, the spacer domain includes the CH2 and/or CH3 of IgG1, IgG4, or IgD. Illustrative spacer domains suitable for use in the CARs described herein include the hinge region derived from the extracellular regions of type 1 membrane proteins such as CD8a and CD4, which may be wild-type hinge regions from these molecules or variants thereof. In certain aspects, the hinge domain includes a CD8a hinge region. In some embodiments, the hinge is a PD-1 hinge or CD152 hinge.

The “transmembrane domain” (Tm domain) is the portion of the CAR that fuses the extracellular binding portion and intracellular signaling domain and anchors the CAR to the plasma membrane of the cell (e.g., immune effector cell). The Tm domain may be derived either from a natural, synthetic, semi-synthetic, or recombinant source. In some embodiments, the Tm domain is derived from (e.g., includes at least the transmembrane region(s) or a functional portion thereof) of the alpha or beta chain of the T-cell receptor, CD35, CD3ζ, CD3γ, CD3δ, CD4, CD5, CD8α, CD9, CD16, CD22, CD27, CD28, CD33, CD37, CD45, CD64, CD80, CD86, CD134, CD137, CD152, CD154, or PD-1.

In one embodiment, a CAR includes a Tm domain derived from CD8α. In certain aspects, a CAR includes a Tm domain derived from CD8a and a short oligo- or polypeptide linker, e.g., between 1, 2, 3, 4, 5, 6, 7, 8, 9, or 10 amino acids in length, that links the Tm domain and the intracellular signaling domain of the CAR. A glycine-serine linker may be employed as such a linker, for example.

The “intracellular signaling” domain of a CAR refers to the part of a CAR that participates in transducing the signal from CAR binding to a target molecule/antigen into the interior of the immune effector cell to elicit effector cell function, e.g., activation, cytokine production, proliferation and/or cytotoxic activity, including the release of cytotoxic factors to the CAR-bound target cell, or other cellular responses elicited with target molecule/antigen binding to the extracellular CAR domain. Accordingly, the term “intracellular signaling domain” refers to the portion of a protein which transduces the effector function signal and that directs the cell to perform a specialized function. To the extent that a truncated portion of an intracellular signaling domain is used, such truncated portion may be used in place of a full-length intracellular signaling domain as long as it transduces the effector function signal. The term intracellular signaling domain is meant to include any truncated portion of an intracellular signaling domain sufficient for transducing effector function signal.

Signals generated through the T cell receptor (TCR) alone are insufficient for full activation of the T cell, and a secondary or costimulatory signal is also required. Thus, T cell activation is mediated by two distinct classes of intracellular signaling domains: primary signaling domains that initiate antigen-dependent primary activation through the TCR (e.g., a TCR/CD3 complex) and costimulatory signaling domains that act in an antigen-independent manner to provide a secondary or costimulatory signal. As such, a CAR of the present disclosure may include an intracellular signaling domain that includes one or more “costimulatory signaling domains” and a “primary signaling domain.” Primary signaling domains regulate primary activation of the TCR complex either in a stimulatory manner, or in an inhibitory manner. Primary signaling domains that act in a stimulatory manner may contain signaling motifs which are known as immunoreceptor tyrosine-based activation motifs (or “ITAMs”). Non-limiting examples of ITAM-containing primary signaling domains suitable for use in a CAR of the present disclosure include those derived from FcRγ, FcRβ, CD3γ, CD3δ, CD3ε, CD3ζ, CD22, CD79α, CD79β, and CD66δ. In certain embodiments, a CAR includes a CD3 primary signaling domain and one or more costimulatory signaling domains. The intracellular primary signaling and costimulatory signaling domains are operably linked to the carboxyl terminus of the transmembrane domain.

In some embodiments, the CAR includes one or more costimulatory signaling domains to enhance the efficacy and expansion of immune effector cells (e.g., T cells) expressing the CAR. As used herein, the term “costimulatory signaling domain” or “costimulatory domain” refers to an intracellular signaling domain of a costimulatory molecule or an active fragment thereof. Example costimulatory molecules suitable for use in CARs contemplated in particular embodiments include TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, K_{D}2C, SLP76, TRIM, and ZAP70. In some embodiments, the CAR includes one or more costimulatory signaling domains selected from the group consisting of 4-1BB (CD137), CD28, and CD134, and a CD3 primary signaling domain.

A CAR of the present disclosure may include any variety of suitable domains including but not limited to a leader sequence; hinge, spacer and/or linker domain(s); transmembrane domain(s); costimulatory domain(s); signaling domain(s) (e.g., CD3C domain(s)); ribosomal skip element(s); restriction enzyme sequence(s); reporter protein domains; and/or the like.

In certain aspects, a CAR of the present disclosure includes a single chain antibody (e.g., any of the scFvs of the present disclosure) that binds to the SARS-CoV-2 antigen; a transmembrane domain from a polypeptide selected from the group consisting of: CD4, CD8α, CD154, and PD-1; one or more intracellular costimulatory signaling domains from a polypeptide selected from the group consisting of: 4-1BB (CD137), CD28, and CD134; and an intracellular signaling domain from a polypeptide selected from the group consisting of: FcRγ, FcRβ, CD3γ, CD3δ, CD3ε, CD3, CD22, CD79α, CD79β, and CD66δ. Such a CAR may further include a spacer domain between the antigen-binding portion and the transmembrane domain, e.g., a CD8 alpha hinge.

According to some embodiments, provided are CARs that comprise—from N-terminus to C-terminus—a variable heavy chain (V_{H}) polypeptide of an antibody described herein, a linker, the variable light chain (V_{L}) of the antibody, a CD8 hinge region (which in some embodiments is an extended CD8 hinge region), a CD8 transmembrane domain, a 4-1BB costimulatory domain, and a CD3C signaling domain. According to certain embodiments, provided are CARs that comprise—from N-terminus to C-terminus—a variable light chain (V_{L}) polypeptide of an antibody described herein, a linker, the variable heavy chain (V_{H}) of the antibody, a CD8 hinge region (which in some embodiments is an extended CD8 hinge region), a CD8 transmembrane domain, a 4-1BB costimulatory domain, and a CD3C signaling domain. In certain embodiments, provided are CARs that comprise—from N-terminus to C-terminus—a variable heavy chain (V_{H}) polypeptide of an antibody described herein, a linker, the variable light chain (V_{L}) of the antibody, a CD28 hinge region, a CD28 transmembrane domain, a 4-1BB costimulatory domain, and a CD3C signaling domain. According to some embodiments, provided are CARs that comprise—from N-terminus to C-terminus—a variable light chain (V_{L}) polypeptide of an antibody described herein, a linker, the variable heavy chain (V_{H}) of the antibody, a CD28 hinge region, a CD28 transmembrane domain, a 4-1BB costimulatory domain, and a CD3C signaling domain. Any of the CARs of the present disclosure may include a domain N-terminal to the V_{H} polypeptide. For example, a leader sequence (e.g., a GM-CSFR leader sequence) may be present at the N-terminus of a CAR of the present disclosure.

Conjugates

Also provided are conjugates. The conjugates include an anti-SARS-CoV-2 antigen antibody of the present disclosure or a fusion protein comprising such an antibody, and an agent conjugated to the antibody or fusion protein. The term “conjugated” generally refers to a chemical linkage, either covalent or non-covalent, usually covalent, that proximally associates one molecule of interest with a second molecule of interest. In some embodiments, the agent is selected from a half-life extending moiety, a labeling agent, and a therapeutic agent. For half-life extension, for example, the antibodies of the present disclosure can optionally be modified to provide for improved pharmacokinetic profile (e.g., by PEGylation, hyperglycosylation, and the like). Modifications that can enhance serum half-life are of interest. A subject antibody may be “PEGylated”, as containing one or more poly(ethylene glycol) (PEG) moieties. Methods and reagents suitable for PEGylation of a protein are well known in the art and may be found in U.S. Pat. No. 5,849,860. PEG suitable for conjugation to a protein is generally soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons. The PEG conjugated to the subject protein can be linear. The PEG conjugated to the subject protein may also be branched. Branched PEG derivatives such as those described in U.S. Pat. No. 5,643,575, “star-PEGs” and multi-armed PEGs. Star PEGs are described in the art including, e.g., in U.S. Pat. No. 6,046,305.

Where the subject antibody is to be isolated from a source, the subject antibody can be conjugated to moieties the facilitate purification, such as members of specific binding pairs, e.g., biotin (member of biotin-avidin specific binding pair), a lectin, and the like. The antibody can also be bound to (e.g., immobilized onto) a solid support, including, but not limited to, polystyrene plates or beads, magnetic beads, test strips, membranes, and the like.

Where the antibodies are to be detected in an assay, the antibodies may contain a detectable label, e.g., a radioisotope (e.g., ¹²⁵I; ³⁵S, and the like), an enzyme which generates a detectable product (e.g., luciferase, β-galactosidase, horse radish peroxidase, alkaline phosphatase, and the like), a fluorescent protein, a chromogenic protein, dye (e.g., fluorescein isothiocyanate, rhodamine, phycoerythrin, and the like); fluorescence emitting metals, e.g., ¹⁵²Eu, or others of the lanthanide series, attached to the protein through metal chelating groups such as EDTA; chemiluminescent compounds, e.g., luminol, isoluminol, acridinium salts, and the like; bioluminescent compounds, e.g., luciferin; fluorescent proteins; and the like. Indirect labels include antibodies specific for a subject protein, wherein the antibody may be detected via a secondary antibody; and members of specific binding pairs, e.g., biotin-avidin, and the like.

According to some embodiments, the agent is a labeling agent. By “labeling agent” (or “detectable label”) is meant the agent detectably labels the antibody or fusion protein, such that the antibody or fusion protein may be detected in an application of interest (e.g., in vitro and/or in vivo research and/or clinical applications). Detectable labels of interest include radioisotopes (e.g., gamma or positron emitters), enzymes that generate a detectable product (e.g., horseradish peroxidase, alkaline phosphatase, luciferase, etc.), fluorescent proteins, paramagnetic atoms, and the like. In certain aspects, the antibody or fusion protein is conjugated to a specific binding partner of detectable label, e.g., conjugated to biotin such that detection may occur via a detectable label that includes avidin/streptavidin.

In certain embodiments, the agent is a labeling agent that finds use in in vivo imaging, such as near-infrared (NIR) optical imaging, single-photon emission computed tomography (SPECT)±CT imaging, positron emission tomography (PET)±CT imaging, nuclear magnetic resonance (NMR) spectroscopy, or the like. Labeling agents that find use in such applications include, but are not limited to, fluorescent labels, radioisotopes, and the like. In certain aspects, the labeling agent is a multi-modal in vivo imaging agent that permits in vivo imaging using two or more imaging approaches (e.g., see Thorp-Greenwood and Coogan (2011) Dalton Trans. 40:6129-6143).

In certain embodiments, the labeling agent is an in vivo imaging agent that finds use in near-infrared (NIR) imaging applications. Such agents include, but are not limited to, a Kodak X-SIGHT dye, Pz 247, DyLight 750 and 800 Fluors, Cy 5.5 and 7 Fluors, Alexa Fluor 680 and 750 Dyes, IRDye 680 and 800CW Fluors. According to some embodiments, the labeling agent is an in vivo imaging agent that finds use in SPECT imaging applications, non-limiting examples of which include ^{99m}Tc, ¹¹¹In, ¹²³I, ²⁰¹Tl, and ¹³³Xe. In certain embodiments, the labeling agent is an in vivo imaging agent that finds use in PET imaging applications, e.g., ¹¹C, ¹³N, ¹⁵O, ¹⁸F, ⁶⁴Cu, ⁶²Cu, ¹²⁴I, ⁷⁶Br, ⁸²Rb, ⁶⁸Ga, or the like.

Any of the above agents that are used to modify the subject antibody or fusion protein may be linked to the antibody via a linker, e.g., a flexible linker. If present, the linker molecules are generally of sufficient length to permit the antibody or fusion protein and a linked carrier to allow some flexible movement between the antibody or fusion protein and the carrier. The linker molecules are generally about 6-50 atoms long. The linker molecules may also be, for example, aryl acetylene, ethylene glycol oligomers containing 2-10 monomer units, diamines, diacids, amino acids, or combinations thereof.

Where the linkers are peptide, the linkers can be of any of a suitable of different lengths, such as from 1 amino acid (e.g., Gly) to 20 or more amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Flexible linkers include glycine polymers (G)ₙ, glycine-serine polymers, glycine-alanine polymers, alanine-serine polymers, and other flexible linkers known in the art. Glycine and glycine-serine polymers may be used where relatively unstructured amino acids are of interest, and may serve as a neutral tether between components. The ordinarily skilled artisan will recognize that design of a peptide conjugated to any elements described above can include linkers that are all or partially flexible, such that the linker can include a flexible linker as well as one or more portions that confer less flexible structure.

According to some embodiments, the antibody is conjugated to the agent via a non-cleavable linker. Non-cleavable linkers of interest include, but are not limited to, thioether linkers. An example of a thioether linker that may be employed includes a succinimidyl 4-(N-maleimidomethyl)cyclohexane-1-carboxylate (SMCC) linker.

In certain embodiments, the antibody is conjugated to the agent via a cleavable linker. According to some embodiments, the linker is a chemically-labile linker, such as an acid-cleavable linker that is stable at neutral pH (bloodstream pH 7.3-7.5) but undergoes hydrolysis upon internalization into the mildly acidic endosomes (pH 5.0-6.5) and lysosomes (pH 4.5-5.0) of a target cell. Chemically-labile linkers include, but are not limited to, hydrazone-based linkers, oxime-based linkers, carbonate-based linkers, ester-based linkers, etc. In certain embodiments, the linker is an enzyme-labile linker, such as an enzyme-labile linker that is stable in the bloodstream but undergoes enzymatic cleavage upon internalization into a target cell, e.g., by a lysosomal protease (such as cathepsin or plasmin) in a lysosome of the target cell. Enzyme-labile linkers include, but are not limited to, linkers that include peptidic bonds, e.g., dipeptide-based linkers such as valine-citrulline (VC) linkers, such as a maleimidocaproyl-valine-citruline-p-aminobenzyl (MC-vc-PAB) linker, a valyl-alanyl-para-aminobenzyloxy (Val-Ala-PAB) linker, and the like. Chemically-labile linkers, enzyme-labile, and non-cleavable linkers are known and described in detail, e.g., in Ducry & Stump (2010) Bioconjugate Chem. 21:5-13; Nolting, B. (2013) Methods Mol Biol. 1045:71-100; Tsuchikama and An (2018) Protein & Cell 9(1):33-46; and elsewhere.

Numerous strategies are available for linking agents to an antibody or fusion protein directly, or indirectly via a linker. For example, the agent may be derivatized by covalently attaching a linker to the agent, where the linker has a functional group capable of reacting with a “chemical handle” on the antibody or fusion protein. The functional group on the linker may vary and may be selected based on compatibility with the chemical handle on the antibody or fusion protein. According to one embodiment, the chemical handle on the antibody or fusion protein is provided by incorporation of an unnatural amino acid having the chemical handle into the antibody or fusion protein. Unnatural amino acids which find use for preparing the conjugates of the present disclosure include those having a functional group selected from an azide, alkyne, alkene, amino-oxy, hydrazine, aldehyde (e.g., formylglycine, e.g., SMARTag™ technology from Catalent Pharma Solutions), nitrone, nitrile oxide, cyclopropene, norbornene, iso-cyanide, aryl halide, and boronic acid functional group. Unnatural amino acids which may be incorporated into an antibody of a conjugate of the present disclosure, which unnatural amino acid may be selected to provide a functional group of interest, are known and described in, e.g., Maza et al. (2015) Bioconjug. Chem. 26(9):1884-9; Patterson et al. (2014) ACS Chem. Biol. 9:592-605; Adumeau et al. (2016) Mol. Imaging Biol. (2):153-65; and elsewhere. An unnatural amino acid may be incorporated into an antibody via chemical synthesis or recombinant approaches (e.g., using a suitable orthogonal amino acyl tRNA synthetase-tRNA pair for incorporation of the unnatural amino acid during translation of the antibody in a host cell).

The functional group of an unnatural amino acid present in the antibody may be an azide, alkyne, alkene, amino-oxy, hydrazine, aldehyde, asaldehyde, nitrone, nitrile oxide, cyclopropene, norbornene, iso-cyanide, aryl halide, boronic acid, diazo, tetrazine, tetrazole, quadrocyclane, iodobenzene, or other suitable functional group, and the functional group on the linker is selected to react with the functional group of the unnatural amino acid (or vice versa). As just one example, an azide-bearing unnatural amino acid (e.g., 5-azido-L-norvaline, or the like) may be incorporated into the antibody and the linker portion of a linker-sialic acid modulator moiety may include an alkyne functional group, such that the antibody and linker-sialic acid modulator moiety are covalently conjugated via azide-alkyne cycloaddition. Conjugation may be carried out using, e.g., a copper-catalyzed azide-alkyne cycloaddition reaction.

In some embodiments, the chemical handle on the antibody does not involve an unnatural amino acid. An antibody containing no unnatural amino acids may be conjugated to the agent by utilizing, e.g., nucleophilic functional groups of the antibody (such as the N-terminal amine or the primary amine of lysine, or any other nucleophilic amino acid residue) as a nucleophile in a substitution reaction with a moiety bearing a reactive leaving group or other electrophilic group. An example would be to prepare a sialic acid modulator-linker or drug-linker moiety bearing an N-hydroxysuccinimidyl (NHS) ester and allow it to react with the antibody under aqueous conditions at elevated pH (˜10) or in polar organic solvents such as DMSO with an added non-nucleophilic base, such as N,N-diisopropylethylamine.

It will be appreciated that the particular approach for attaching a linker, agent and/or antibody or fusion protein to each other may vary depending upon the particular linker, agent and/or antibody or fusion protein and functional groups selected and employed for conjugating the various components to each other.

Bispecific Antibodies

Also provided are bispecific antibodies. According to some embodiments, a bispecific antibody of the present disclosure includes a first antigen-binding domain (e.g., a Fab arm, scFv, or the like) that specifically binds a SARS-CoV-2 antigen, where the first antigen binding domain includes a V_{H} polypeptide and a V_{L} polypeptide of an antibody of the present disclosure. In certain embodiments, the bispecific antibody includes a second antigen-binding domain (e.g., a Fab arm, scFv, or the like) that specifically binds a SARS-CoV-2 antigen, e.g., the same or different SARS-CoV-2 antigen bound by the first antigen-binding domain. According to some embodiments, the bispecific antibody includes a second antigen-binding domain (e.g., a Fab arm, scFv, or the like) that specifically binds an antigen other than a SARS-CoV-2 antigen. Examples of antigens other than SARS-CoV-2 to which the second antigen-binding domain may specifically bind include, but are not limited to, a cell surface antigen expressed on the surface of a cell physically associated with SARS-CoV-2 particles, e.g., a cell being infected by SARS-CoV-2 particles. In certain embodiments, the second antigen-binding domain may specifically bind an immune cell surface antigen. By way of example, the immune cell surface antigen may be a T cell surface antigen.

Bispecific antibodies of the present disclosure include antibodies having a full length antibody structure, and bispecific antibody fragments. “Full length” as used herein refers to an antibody having two full length antibody heavy chains and two lull length antibody light chains. A full-length antibody heavy chain (HC) consists of well-known heavy chain variable and constant domains VH, CH1, CH2, and CH3. A full-length antibody light chain (LC) consists of well-known light chain variable and constant domains VL and CL. The full-length antibody may be lacking the C-terminal lysine in either one or both heavy chains. The term “Fab arm” refers to one heavy chain-light chain pair that specifically binds an antigen.

Full length bispecific antibodies may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each bind a distinct epitope.

The “knob-in-hole” strategy (see, e.g., PCT Intl. Publ. No. WO 2006/028936) may be used to generate full length bispecific antibodies. Briefly, selected amino acids forming the interface of the CHS domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y7F405A, T366W/F405W, F405W/Y407A, T394W/Y407T, T3945/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface may be used, as described in US Pat. Publ. No. US2010/0015133; US Pat. Publ. No. US2009/0182127; US Pat. Publ. No. U82010/028637 or US Pat. Publ. No. US2011/0123532. In other strategies. heterodimerization may be promoted by following substitutions (expressed as modified position in the first CH3 domain of the first heavy chain/modified position in the second CH3 domain of the second heavy chain): L351 Y_F405A_Y407V T394W, T3661_K392M_T394W/F405A_Y407V, T366L_K392M_T394W/F405A_Y407V, L351 Y_Y407A′T366A_K409F, L351Y_Y407A/T366V_K409F, Y407A/T366A_K409F, or T350V_L351Y_F405A_Y407V/T350V_T366L_K392L_T394W as described in U.S. Pat. Pubi. No. US2012/0149876 or U.S. Pat. Publ. No. US2013/0195849.

Also provided are single chain bispecific antibodies. In some embodiments, a single chain bispecific antibody of the present disclosure is a bispecific scFv. Details regarding bispecific scFvs may be found, e.g., in Zhou et al. (2017) J Cancer 8(18):3689-3696.

Methods of Producing Antibodies and Fusion Proteins

Using the information provided herein, the anti-SARS-CoV-2 antigen antibodies and fusion proteins of the present disclosure may be prepared using techniques known to those of skill in the art. For example, a nucleic acid sequence encoding the amino acid sequence of an antibody of the present disclosure can be used to express the antibodies. The polypeptide sequences provided herein (see, e.g., Table 1) can be used to determine appropriate nucleic acid sequences encoding the antibodies and the nucleic acids sequences then used to express one or more antibodies specific for SARS-CoV-2. The nucleic acid sequence(s) can be optimized to reflect particular codon “preferences” for various expression systems according to methods known to those of skill in the art.

Using the amino acid and/or polynucleotide sequence information provided herein, the nucleic acids may be synthesized according to a number of standard methods known to those of skill in the art. Oligonucleotide synthesis, is preferably carried out on commercially available solid phase oligonucleotide synthesis machines or manually synthesized using, for example, a solid phase phosphoramidite triester method.

Once a nucleic acid encoding a subject antibody or fusion protein is synthesized, it can be amplified and/or cloned according to standard methods. Molecular cloning techniques to achieve these ends are known in the art. A wide variety of cloning and in vitro amplification methods suitable for the construction of recombinant nucleic acids are known to persons of skill in the art and are the subjects of numerous textbooks and laboratory manuals.

Expression of natural or synthetic nucleic acids encoding the antibodies of the present disclosure can be achieved by operably linking a nucleic acid encoding the antibody to a promoter (which is either constitutive or inducible), and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the antibody. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain high levels of expression of a cloned nucleic acid it is common to construct expression plasmids which typically contain a strong promoter to direct transcription, a ribosome binding site for translational initiation, and a transcription/translation terminator, each in functional orientation to each other and to the protein-encoding sequence. Examples of regulatory regions suitable for this purpose in E. coli are the promoter and operator region of the E. coli tryptophan biosynthetic pathway, the leftward promoter of phage lambda (P_{L}), and the L-arabinose (araBAD) operon. The inclusion of selection markers in DNA vectors transformed in E. coli is also useful. Examples of such markers include genes specifying resistance to ampicillin, tetracycline, or chloramphenicol. Expression systems for expressing antibodies are available using, for example, E. coli, Bacillus sp. and Salmonella. E. coli systems may also be used.

The antibody gene(s) may also be subcloned into an expression vector that allows for the addition of a tag (e.g., FLAG, hexahistidine, and the like) at the C-terminal end or the N-terminal end of the antibody (e.g., IgG, Fab, scFv, etc.) to facilitate purification. Methods of transfecting and expressing genes in mammalian cells are known in the art. Transducing cells with nucleic acids can involve, for example, incubating lipidic microparticles containing nucleic acids with cells or incubating viral vectors containing nucleic acids with cells within the host range of the vector. The culture of cells used in the present disclosure, including cell lines and cultured cells from tissue (e.g., tumor) or blood samples is well known in the art.

Once the nucleic acid encoding a subject antibody is isolated and cloned, one can express the nucleic acid in a variety of recombinantly engineered cells known to those of skill in the art. Examples of such cells include bacteria, yeast, filamentous fungi, insect (e.g. those employing baculoviral vectors), and mammalian cells.

Isolation and purification of a subject antibody can be accomplished according to methods known in the art. For example, a protein can be isolated from a lysate of cells genetically modified to express the protein constitutively and/or upon induction, or from a synthetic reaction mixture, by immunoaffinity purification (or precipitation using Protein L or A), washing to remove non-specifically bound material, and eluting the specifically bound antibody. The isolated antibody can be further purified by dialysis and other methods normally employed in protein purification methods. In one embodiment, the antibody may be isolated using metal chelate chromatography methods. Antibodies of the present disclosure may contain modifications to facilitate isolation, as discussed above.

The subject antibodies may be prepared in substantially pure or isolated form (e.g., free from other polypeptides). The protein can be present in a composition that is enriched for the polypeptide relative to other components that may be present (e.g., other polypeptides or other host cell components). Purified antibodies may be provided such that the antibody is present in a composition that is substantially free of other expressed proteins, e.g., less than 90%, usually less than 60% and more usually less than 50% of the composition is made up of other expressed proteins.

The antibodies produced by prokaryotic cells may require exposure to chaotropic agents for proper folding. During purification from E. coli, for example, the expressed protein can be optionally denatured and then renatured. This can be accomplished, e.g., by solubilizing the bacterially produced antibodies in a chaotropic agent such as guanidine HCl. The antibody is then renatured, either by slow dialysis or by gel filtration. Alternatively, nucleic acid encoding the antibodies may be operably linked to a secretion signal sequence such as pelB so that the antibodies are secreted into the periplasm in correctly-folded form.

The present disclosure also provides cells that produce the antibodies of the present disclosure, where suitable cells include eukaryotic cells, e.g., mammalian cells. The cells can be a hybrid cell or “hybridoma” that is capable of reproducing antibodies in vitro (e.g. monoclonal antibodies, such as IgG). For example, the present disclosure provides a recombinant host cell (also referred to herein as a “genetically modified host cell”) that is genetically modified with one or more nucleic acids comprising a nucleotide sequence encoding a heavy and/or light chain of an antibody of the present disclosure.

Techniques for creating recombinant DNA versions of the antigen-binding regions of antibody molecules which bypass the generation of hybridomas are also contemplated herein. DNA is cloned into a bacterial (e.g., bacteriophage), yeast (e.g. Saccharomyces or Pichia) insect or mammalian expression system, for example. One example of a suitable technique uses a bacteriophage lambda vector system having a leader sequence that causes the expressed antibody (e.g., Fab or scFv) to migrate to the periplasmic space (between the bacterial cell membrane and the cell wall) or to be secreted. One can rapidly generate great numbers of functional fragments (e.g., Fab or scFv) for those which bind the tumor associated antigen.

Antibodies that specifically bind SARS-CoV-2 can be prepared using a wide variety of techniques known in the art including the use of hybridoma, recombinant, phage display technologies, or a combination thereof. For example, an antibody may be made and isolated using methods of phage display. Phage display is used for the high-throughput screening of protein interactions. Phages may be utilized to display antigen-binding domains expressed from a repertoire or combinatorial antibody library (e.g., human or murine). Phage expressing an antigen binding domain that binds SARS-CoV-2 can be selected or identified with SARS-CoV-2, e.g., using labeled SARS-CoV-2 bound or captured to a solid surface or bead. Phage used in these methods are typically filamentous phage including fd and M13 binding domains expressed from phage with Fab, Fv (individual Fv region from light or heavy chains) or disulfide stabilized Fv antibody domains recombinantly fused to either the phage gene III or gene VIII protein. Exemplary methods are set forth, for example, in U.S. Pat. No. 5,969,108, Hoogenboom, H. R. and Chames, Immunol. Today 2000, 21:371; Nagy et al. Nat. Med. 2002, 8:801; Huie et al., Proc. Natl. Acad. Sci. USA 2001, 98:2682; Lui et al., J. Mol. Biol. 2002, 315:1063, each of which is incorporated herein by reference. Several publications (e.g., Marks et al., Bio/Technology 1992, 10:779-783) have described the production of high affinity human antibodies by chain shuffling, as well as combinatorial infection and in vivo recombination as a strategy for constructing large phage libraries. In another embodiment, ribosomal display can be used to replace bacteriophage as the display platform (see, e.g., Hanes et al., Nat. Biotechnol. 2000, 18:1287; Wilson et al., Proc. Natl. Acad. Sci. USA 2001, 98:3750; or Irving et al., J. Immunol. Methods 2001, 248:31). Cell surface libraries may be screened for antibodies (Boder et al., Proc. Natl. Acad. Sci. USA 2000, 97:10701; Daugherty et al., J. Immunol. Methods 2000, 243:211). Such procedures provide alternatives to traditional hybridoma techniques for the isolation and subsequent cloning of monoclonal antibodies.

After phage selection, the antibody coding regions from the phage can be isolated and used to generate whole antibodies, including human antibodies, or any desired antigen binding fragment, and expressed in any desired host, including mammalian cells, insect cells, plant cells, yeast, and bacteria. For example, techniques to recombinantly produce Fv, scFv, Fab, F(ab′)₂, and Fab′ fragments may be employed using methods known in the art.

In certain embodiments, an antibody of the present disclosure is identified by the following steps: sort memory B cells obtained from blood samples of individuals having a SARS-CoV-2 infection or individuals who previously had a SARS-CoV-2 infection (e.g., individuals having or who previously had COVID-19); sequence the B-cell receptors (BCRs) and pair the heavy and light chains (e.g., using pairSEQ™ by Adaptive Biotechnologies®); analyze BCR paired clonal lineages and/or SHM variants within those lineages to select for high likelihood of SARS-CoV-2 specific features; and select the top paired BCR candidates for antibody synthesis and functional characterization. Aspects of the present disclosure include methods of identifying an anti-SARS-CoV-2 antigen antibody by implementing such steps.

Nucleic Acids, Expression Vectors and Cells

In view of the section above regarding methods of producing the antibodies of the present disclosure, it will be appreciated that the present disclosure also provides nucleic acids, expression vectors and cells.

In certain embodiments, provided is a nucleic acid encoding a variable heavy chain (V_{H}) polypeptide, a variable light chain (V_{L}) polypeptide, or both, of an antibody of the present disclosure. According to some embodiments, provided is a nucleic acid encoding a V_{H} polypeptide, a V_{L} polypeptide, or both, of an antibody of the present disclosure, e.g., an antibody comprising a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{H} of antibody 508, 767, 935, 937, 941, 980, 1085, 1213, 1227, 1231, 1238, 1439, 1589, 1671, 1679, 1814, 1815, 1823, 1826, 1851, 1856, 1859, 1864, 1867, 1870, 1871, 1872, 1888, 1915, 1959, 1963, 1969, 1984, 2019, 2020, 2024, 2025, 2050, 2075, 2080, 2432, 2564, 2598, 2606, 2619, 2646, 2706, 2729, 2788, 2793, 2794, 2854, 2866, 2892, 3086, 3091, 3995, 4042, or 4441; and a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{L} of antibody 508, 767, 935, 937, 941, 980, 1085, 1213, 1227, 1231, 1238, 1439, 1589, 1671, 1679, 1814, 1815, 1823, 1826, 1851, 1856, 1859, 1864, 1867, 1870, 1871, 1872, 1888, 1915, 1959, 1963, 1969, 1984, 2019, 2020, 2024, 2025, 2050, 2075, 2080, 2432, 2564, 2598, 2606, 2619, 2646, 2706, 2729, 2788, 2793, 2794, 2854, 2866, 2892, 3086, 3091, 3995, 4042, or 4441, respectively; wherein the antibody comprises one or more, two or more, three or more, four or more, five or six of the complementarity determining regions (CDRs) of antibody 508, 767, 935, 937, 941, 980, 1085, 1213, 1227, 1231, 1238, 1439, 1589, 1671, 1679, 1814, 1815, 1823, 1826, 1851, 1856, 1859, 1864, 1867, 1870, 1871, 1872, 1888, 1915, 1959, 1963, 1969, 1984, 2019, 2020, 2024, 2025, 2050, 2075, 2080, 2432, 2564, 2598, 2606, 2619, 2646, 2706, 2729, 2788, 2793, 2794, 2854, 2866, 2892, 3086, 3091, 3995, 4042, or 4441, respectively. Non-limiting examples of such nucleic acids are provided in Example 2 hereinbelow. In certain embodiments, a nucleic acid of the present disclosure comprises the V_{H}- or V_{L}-encoding region of the polynucleotide sequence set forth in any one of SEQ ID NOs:473-496. In certain embodiments, a nucleic acid of the present disclosure comprises the heavy chain- or light chain-encoding region of the polynucleotide sequence set forth in any one of SEQ ID NOs:473-496. According to some embodiments, a nucleic acid of the present disclosure comprises the polynucleotide sequence set forth in any one of SEQ ID NOs:473-496, or a polynucleotide sequence comprising 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% nucleotide sequence identity with the polynucleotide sequence set forth in any one of SEQ ID NOs:473-496.

In certain embodiments, provided is a nucleic acid encoding a variable heavy chain (V_{H}) polypeptide, a variable light chain (V_{L}) polypeptide, or both, of an antibody of the present disclosure, wherein the antibody is a single chain antibody (e.g., an scFv), and the nucleic acid encodes the single chain antibody.

Any of the nucleic acids of the present disclosure may be operably linked to a heterologous expression control sequence, e.g., a heterologous promoter.

Also provided are expression vectors comprising any of the nucleic acids of the present disclosure.

Cells that comprise any of the nucleic acids and/or expression vectors of the present disclosure are also provided. According to some embodiments, a cell of the present disclosure includes a nucleic acid that encodes the V_{H} polypeptide of the antibody and the V_{L} polypeptide of the antibody. In certain such embodiments, the antibody is a single chain antibody (e.g., an scFv), and the nucleic acid encodes the single chain antibody. According to some embodiments, provided is a cell comprising a first nucleic acid encoding a variable heavy chain (V_{H}) polypeptide of an antibody of the present disclosure, and a second nucleic acid encoding a variable light chain (V_{L}) polypeptide of the antibody. In certain embodiments, such a cell comprises a first expression vector comprising the first nucleic acid, and a second expression vector comprising the second nucleic acid.

According to some embodiments, provided is a cell comprising a nucleic acid encoding a V_{H} polypeptide, a V_{L} polypeptide, or both, of an antibody comprising a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{H} of antibody 508, 767, 935, 937, 941, 980, 1085, 1213, 1227, 1231, 1238, 1439, 1589, 1671, 1679, 1814, 1815, 1823, 1826, 1851, 1856, 1859, 1864, 1867, 1870, 1871, 1872, 1888, 1915, 1959, 1963, 1969, 1984, 2019, 2020, 2024, 2025, 2050, 2075, 2080, 2432, 2564, 2598, 2606, 2619, 2646, 2706, 2729, 2788, 2793, 2794, 2854, 2866, 2892, 3086, 3091, 3995, 4042, or 4441; and a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence of the V_{L} of antibody 508, 767, 935, 937, 941, 980, 1085, 1213, 1227, 1231, 1238, 1439, 1589, 1671, 1679, 1814, 1815, 1823, 1826, 1851, 1856, 1859, 1864, 1867, 1870, 1871, 1872, 1888, 1915, 1959, 1963, 1969, 1984, 2019, 2020, 2024, 2025, 2050, 2075, 2080, 2432, 2564, 2598, 2606, 2619, 2646, 2706, 2729, 2788, 2793, 2794, 2854, 2866, 2892, 3086, 3091, 3995, 4042, or 4441, respectively; wherein the antibody comprises one or more, two or more, three or more, four or more, five or six of the complementarity determining regions (CDRs) of antibody 508, 767, 935, 937, 941, 980, 1085, 1213, 1227, 1231, 1238, 1439, 1589, 1671, 1679, 1814, 1815, 1823, 1826, 1851, 1856, 1859, 1864, 1867, 1870, 1871, 1872, 1888, 1915, 1959, 1963, 1969, 1984, 2019, 2020, 2024, 2025, 2050, 2075, 2080, 2432, 2564, 2598, 2606, 2619, 2646, 2706, 2729, 2788, 2793, 2794, 2854, 2866, 2892, 3086, 3091, 3995, 4042, or 4441, respectively. Non-limiting examples of such nucleic acids are provided in Example 2 hereinbelow. In some embodiments, a cell of the present disclosure comprises a nucleic acid comprising the V_{H}- or V_{L}-encoding region of the polynucleotide sequence set forth in any one of SEQ ID NOs:473-496. In certain embodiments, a cell of the present disclosure comprises a nucleic acid comprising the heavy chain- or light chain-encoding region of the polynucleotide sequence set forth in any one of SEQ ID NOs:473-496. According to some embodiments, a cell of the present disclosure comprises a nucleic acid comprising the polynucleotide sequence set forth in any one of SEQ ID NOs:473-496, or a polynucleotide sequence comprising 70% or greater, 75% or greater, 80% or greater, 85% or greater, 90% or greater, 95% or greater, or 99% or greater nucleotide sequence identity with the polynucleotide sequence set forth in any one of SEQ ID NOs:473-496.

In certain embodiments, provided are cells comprising the polynucleotides set forth in: SEQ ID NO:473 and SEQ ID NO:474; SEQ ID NO:475 and SEQ ID NO:476; SEQ ID NO:477 and SEQ ID NO:478; SEQ ID NO:479 and SEQ ID NO:480; SEQ ID NO:481 and SEQ ID NO:482; SEQ ID NO:483 and SEQ ID NO:484; SEQ ID NO:485 and SEQ ID NO:486; SEQ ID NO:487 and SEQ ID NO:488; SEQ ID NO:489 and SEQ ID NO:490; SEQ ID NO:491 and SEQ ID NO:492; SEQ ID NO:493 and SEQ ID NO:494; or SEQ ID NO:495 and SEQ ID NO:496.

Any of the cells of the present disclosure may comprise the nucleic acid(s) operably linked to a heterologous expression control sequence, e.g., a heterologous promoter.

Also provided are methods of making an antibody of the present disclosure, the methods including culturing a cell of the present disclosure under conditions suitable for the cell to express the antibody, wherein the antibody is produced. The conditions suitable for the cell to express the antibody may vary. Non-limiting examples of such conditions include culturing the cell in a suitable container (e.g., a cell culture plate or well thereof), in suitable medium (e.g., cell culture medium, such as DMEM, RPMI, MEM, IMDM, DMEM/F-12, or the like) at a suitable temperature (e.g., 32° C.-42° C., such as 37° C.) and pH (e.g., pH 7.0-7.7, such as pH 7.4) in an environment having a suitable percentage of CO₂, e.g., from 3% to 10%, such as 5%.

### Compositions

The present disclosure also provides compositions. In certain embodiments, the compositions find use, e.g., in practicing the methods of the present disclosure.

According to some embodiments, a composition of the present disclosure includes an antibody of the present disclosure. For example, the antibody may be any of the antibodies described in the Antibodies section hereinabove, which is incorporated but not reiterated herein for purposes of brevity. According to some embodiments, a composition of the present disclosure includes a conjugate of the present disclosure. In some embodiments, a composition of the present disclosure includes a fusion protein of the present disclosure.

In certain aspects, a composition of the present disclosure includes the antibody present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

As summarized above, aspects of the present disclosure include pharmaceutical compositions. In some embodiments, a pharmaceutical composition of the present disclosure includes an anti-SARS-CoV-2 antigen antibody of the present disclosure (or conjugate or fusion protein comprising same), and a pharmaceutically acceptable carrier.

As will be appreciated, the pharmaceutical compositions of the present disclosure may include any of the agents and features described herein in the Antibodies and Methods sections, which are incorporated but not reiterated in detail herein for purposes of brevity.

In certain embodiments, a pharmaceutical composition of the present disclosure comprises a therapeutically effective amount of an antibody that competes for binding to SARS-CoV-2 with an antibody comprising the six CDRs of the antibody designated herein as antibody 941; or an antibody that comprises the six CDRs of the antibody designated herein as antibody 941; or an antibody that comprises the V_{H} and V_{L} of the antibody designated herein as antibody 941. The antibody may be part of a fusion protein or conjugate of the present disclosure.

According to some embodiments, a pharmaceutical composition of the present disclosure comprises a therapeutically effective amount of an antibody that competes for binding to SARS-CoV-2 with an antibody comprising the six CDRs of the antibody designated herein as antibody 980; or an antibody that comprises the six CDRs of the antibody designated herein as antibody 980; or an antibody that comprises the V_{H} and V_{L} of the antibody designated herein as antibody 980. The antibody may be part of a fusion protein or conjugate of the present disclosure.

In certain embodiments, a pharmaceutical composition of the present disclosure comprises a therapeutically effective amount of an antibody that competes for binding to SARS-CoV-2 with an antibody comprising the six CDRs of the antibody designated herein as antibody 1589; or an antibody that comprises the six CDRs of the antibody designated herein as antibody 1589; or an antibody that comprises the V_{H} and V_{L} of the antibody designated herein as antibody 1589. The antibody may be part of a fusion protein or conjugate of the present disclosure.

Any of the pharmaceutical composition of the present disclosure may comprise a “cocktail” of two or more different antibodies, where at least one of the antibodies is an antibody of the present disclosure. In certain embodiments, a pharmaceutical composition of the present disclosure comprises a therapeutically effective amount of a cocktail of two or more different antibodies, where at least two of the two or more different antibodies are antibodies of the present disclosure. According to some embodiments, a pharmaceutical composition of the present disclosure comprises a cocktail of two or more, three or more, four or more, or five or more, of the antibodies of the present disclosure, e.g., antibodies that compete for binding to a SARS-CoV-2 antigen(s) with antibodies comprising the six CDRs of the antibodies designated herein as antibodies 508, 767, 935, 937, 941, 980, 1085, 1213, 1227, 1231, 1238, 1439, 1589, 1671, 1679, 1814, 1815, 1823, 1826, 1851, 1856, 1859, 1864, 1867, 1870, 1871, 1872, 1888, 1915, 1959, 1963, 1969, 1984, 2019, 2020, 2024, 2025, 2050, 2075, 2080, 2432, 2564, 2598, 2606, 2619, 2646, 2706, 2729, 2788, 2793, 2794, 2854, 2866, 2892, 3086, 3091, 3995, 4042, and/or 4441; or antibodies that comprise the six CDRs of the antibodies designated herein as antibodies 508, 767, 935, 937, 941, 980, 1085, 1213, 1227, 1231, 1238, 1439, 1589, 1671, 1679, 1814, 1815, 1823, 1826, 1851, 1856, 1859, 1864, 1867, 1870, 1871, 1872, 1888, 1915, 1959, 1963, 1969, 1984, 2019, 2020, 2024, 2025, 2050, 2075, 2080, 2432, 2564, 2598, 2606, 2619, 2646, 2706, 2729, 2788, 2793, 2794, 2854, 2866, 2892, 3086, 3091, 3995, 4042, and/or 4441; or antibodies that comprise the V_{H} and V_{L} of the antibodies designated herein as antibodies 508, 767, 935, 937, 941, 980, 1085, 1213, 1227, 1231, 1238, 1439, 1589, 1671, 1679, 1814, 1815, 1823, 1826, 1851, 1856, 1859, 1864, 1867, 1870, 1871, 1872, 1888, 1915, 1959, 1963, 1969, 1984, 2019, 2020, 2024, 2025, 2050, 2075, 2080, 2432, 2564, 2598, 2606, 2619, 2646, 2706, 2729, 2788, 2793, 2794, 2854, 2866, 2892, 3086, 3091, 3995, 4042, or4441. The antibodies may be present as fusion proteins or conjugates of the present disclosure.

In certain embodiments, a pharmaceutical composition of the present disclosure comprises a therapeutically effective amount of a cocktail comprising antibodies that compete for binding to SARS-CoV-2 antigen(s) with antibodies comprising the six CDRs of the antibodies designated herein as antibodies 941 and 980; or antibodies that comprise the six CDRs of the antibodies designated herein as antibodies 941 and 980; or antibodies that comprise the V_{H} and V_{L} of the antibodies designated herein as antibodies 941 and 980. The antibodies may be present as fusion proteins or conjugates of the present disclosure.

According to some embodiments, a pharmaceutical composition of the present disclosure comprises a therapeutically effective amount of a cocktail comprising antibodies that compete for binding to SARS-CoV-2 with antibodies comprising the six CDRs of the antibodies designated herein as antibodies 941 and 1589; or antibodies that comprise the six CDRs of the antibodies designated herein as antibodies 941 and 1589; or antibodies that comprise the V_{H} and V_{L} of the antibodies designated herein as antibodies 941 and 1589. The antibodies may be present as fusion proteins or conjugates of the present disclosure.

In certain embodiments, a pharmaceutical composition of the present disclosure comprises a therapeutically effective amount of a cocktail comprising antibodies that compete for binding to SARS-CoV-2 with antibodies comprising the six CDRs of the antibodies designated herein as antibodies 980 and 1589; or antibodies that comprise the six CDRs of the antibodies designated herein as antibodies 980 and 1589; or antibodies that comprise the V_{H} and V_{L} of the antibodies designated herein as antibodies 980 and 1589. The antibodies may be present as fusion proteins or conjugates of the present disclosure.

The one or more antibodies (including any of the conjugates or fusion proteins of the present disclosure) can be incorporated into a variety of formulations for therapeutic administration. More particularly, the anti-SARS-CoV-2 antigen antibody can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the agents for administration to the individual (e.g., suitable for human administration) are generally sterile and may further be free of detectable pyrogens or other contaminants contraindicated for administration to a patient according to a selected route of administration.

In pharmaceutical dosage forms, the agent(s) can be administered in the form of their pharmaceutically acceptable salts, or they may also be used alone or in appropriate association, as well as in combination, with other pharmaceutically active compounds. The following methods and carriers/excipients are merely examples and are in no way limiting.

For oral preparations, the agent(s) can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agent(s) can be formulated for parenteral (e.g., intravenous, intra-arterial, intraosseous, intramuscular, intracerebral, intracerebroventricular, intrathecal, subcutaneous, etc.) administration. In certain aspects, the agent(s) are formulated for injection by dissolving, suspending or emulsifying the agent(s) in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the agent(s) may be prepared by mixing the agent(s) having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the agent(s) may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the antibody and/or T cell activator against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, the pharmaceutical composition includes the antibody and/or T cell activator, and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Methods

Aspects of the present disclosure include methods comprising administering an anti-SARS-CoV-2 antigen antibody of the present disclosure (or conjugate or fusion protein comprising same) to an individual in need thereof. In certain embodiments, provided are methods of treating an individual having or suspected of having a SARS-CoV-2 infection, the method comprising administering to the individual a therapeutically effective amount of any of the antibodies, fusion proteins, or conjugates of the present disclosure. In certain embodiments, provided are methods of treating an individual who is not suspected of having a SARS-CoV-2 infection, the method comprising administering to the individual a therapeutically effective amount of any of the antibodies, fusion proteins, or conjugates of the present disclosure, where the therapeutically effective amount of the antibody, fusion protein, or conjugate is administered prophylactically, e.g., to prevent the individual from experiencing one or more symptoms of a SARS-CoV-2 infection (e.g., one or more symptoms of COVID-19) in the event that the individual is exposed to SARS-CoV-2 virus. In some embodiments, the individual who is not suspected of having a SARS-CoV-2 infection is an immunocompromised individual. Non-limiting examples of immunocompromised individuals include those with HIV/AIDS, cancer, patients taking one or more immunosuppressive drugs (e.g., transplant patients), those with inherited diseases that affect the immune system (e.g., congenital agammaglobulinemia, congenital IgA deficiency, etc.), and/or the like.

The anti-SARS-CoV-2 antibodies, fusion proteins, or conjugates may be administered to any of a variety of subjects. In certain aspects, the individual is a “mammal” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the individual is a human. In certain aspects, the individual is an animal model (e.g., a mouse model, a primate model, or the like) of a SARS-CoV-2 infection, e.g., an animal model of COVID-19.

The anti-SARS-CoV-2 antibodies, fusion proteins or conjugates are administered in a therapeutically effective amount. By “therapeutically effective amount” is meant a dosage sufficient to produce a desired result, e.g., an amount sufficient to effect beneficial or desired therapeutic (including prophylactic) results, such as the prevention or a reduction in a symptom of a SARS-CoV-2 infection (e.g., a symptom of COVID-19), as compared to a control. In some embodiments, the therapeutically effective amount is sufficient to slow the progression of, or reduce, one or more symptoms of a SARS-CoV-2 infection (e.g., one or more COVID-19 symptoms) selected from viral load, hypoxia (e.g., oxygen saturation levels below 95%, e.g., as measured by pulse oximetry), pneumonia, acute respiratory distress syndrome, thrombosis in the pulmonary microcirculation, and/or the like. According to some embodiments, the therapeutically effective amount slows the progression of, or reduces, one or more of such symptoms by 10% or more, 20% or more, 30% or more, 40% or more, 50% or more, 60% or more, 70% or more, 80% or more, 90% or more, or 100% or more, as compared to the one or more symptoms in the absence of the administration of the anti-SARS-CoV-2 antibodies, fusion proteins, or conjugates. An effective amount can be administered in one or more administrations.

When the methods include administering a combination of the anti-SARS-CoV-2 antigen antibody (or fusion protein or conjugate) and a second agent (e.g., a second anti-SARS-CoV-2 antigen antibody (or fusion protein or conjugate) of the present disclosure; or a second agent approved for treatment of a SARS-CoV-2 infection, e.g., COVID-19, a non-limiting example of which is a SARS-CoV-2 polymerase inhibitor, e.g., remdesivir), the anti-SARS-CoV-2 antigen antibody and the second agent may be administered concurrently (e.g., in the same or separate formulations), sequentially, or both. For example, according to certain embodiments, the second agent is administered to the individual prior to administration of the anti-SARS-CoV-2 antigen antibody, concurrently with administration of the anti-SARS-CoV-2 antigen antibody, or both. In some embodiments, the anti-SARS-CoV-2 antigen antibody is administered to the individual prior to administration of the second agent, concurrently with administration of the second agent, or both.

In certain aspects, the one or more agents are administered according to a dosing regimen approved for individual use. In some embodiments, the administration of the anti-SARS-CoV-2 antigen antibody permits the second agent to be administered according to a dosing regimen that involves one or more lower and/or less frequent doses, and/or a reduced number of cycles as compared with that utilized when the second agent is administered without administration of the anti-SARS-CoV-2 antigen antibody. In some embodiments, the administration of the second agent permits the anti-SARS-CoV-2 antigen antibody to be administered according to a dosing regimen that involves one or more lower and/or less frequent doses, and/or a reduced number of cycles as compared with that utilized when the anti-SARS-CoV-2 antigen antibody is administered without administration of the second agent.

As noted above, in certain embodiments, one or more doses of the anti-SARS-CoV-2 antigen antibody and second agent are administered at the same time; in some such embodiments, such agents may be administered present in the same pharmaceutical composition. In some embodiments, however, the anti-SARS-CoV-2 antigen antibody and second agent are administered to the individual in different compositions and/or at different times. For example, the anti-SARS-CoV-2 antigen antibody may be administered prior to administration of the second agent (e.g., in a particular cycle). Alternatively, the second agent may be administered prior to administration of the anti-SARS-CoV-2 antigen antibody (e.g., in a particular cycle). The second agent to be administered may be administered a period of time that starts at least 1 hour, 3 hours, 6 hours, 12 hours, 24 hours, 48 hours, 72 hours, or up to 5 days or more after the administration of the first agent.

In some embodiments, administration of one agent is specifically timed relative to administration of another agent. For example, in some embodiments, a first agent is administered so that a particular effect is observed (or expected to be observed, for example based on population studies showing a correlation between a given dosing regimen and the particular effect of interest).

In certain aspects, desired relative dosing regimens for agents administered in combination may be assessed or determined empirically, for example using ex vivo, in vivo and/or in vitro models; in some embodiments, such assessment or empirical determination is made in vivo, in a patient population (e.g., so that a correlation is established), or alternatively in a particular subject of interest.

In some embodiments, the anti-SARS-CoV-2 antigen antibody and second agent are administered according to an intermittent dosing regimen including at least two cycles. Where two or more agents are administered in combination, and each by such an intermittent, cycling, regimen, individual doses of different agents may be interdigitated with one another. In certain aspects, one or more doses of the second agent is administered a period of time after a dose of the first agent. In some embodiments, each dose of the second agent is administered a period of time after a dose of the first agent. In certain aspects, each dose of the first agent is followed after a period of time by a dose of the second agent. In some embodiments, two or more doses of the first agent are administered between at least one pair of doses of the second agent; in certain aspects, two or more doses of the second agent are administered between al least one pair of doses of the first agent. In some embodiments, different doses of the same agent are separated by a common interval of time; in some embodiments, the interval of time between different doses of the same agent varies. In certain aspects, different doses of the different agents are separated from one another by a common interval of time; in some embodiments, different doses of the different agents are separated from one another by different intervals of time.

One exemplary protocol for interdigitating two intermittent, cycled dosing regimens (e.g., for potentiating the effect of the anti-SARS-CoV-2 antigen antibody), may include: (a) a first dosing period during which a therapeutically effective amount a first agent is administered to an individual; (b) a first resting period; (c) a second dosing period during which a therapeutically effective amount of a second agent and, optionally, a third agent, is administered to the individual; and (d) a second resting period.

In some embodiments, the first resting period and second resting period may correspond to an identical number of hours or days. Alternatively, in some embodiments, the first resting period and second resting period are different, with either the first resting period being longer than the second one or, vice versa. In some embodiments, each of the resting periods corresponds to 120 hours, 96 hours, 72 hours, 48 hours, 24 hours, 12 hours, 6 hours, 30 hours, 1 hour, or less. In some embodiments, if the second resting period is longer than the first resting period, it can be defined as a number of days or weeks rather than hours (for instance 1 day, 3 days, 5 days, 1 week, 2, weeks, 4 weeks or more).

If the first resting period's length is determined by existence or development of a particular biological or therapeutic event, then the second resting period's length may be determined on the basis of different factors, separately or in combination. Exemplary such factors may include the identity and/or properties (e.g., pharmacokinetic properties) of the first agent, and/or one or more features of the patient's response to therapy with the first agent. In some embodiments, length of one or both resting periods may be adjusted in light of pharmacokinetic properties (e.g., as assessed via plasma concentration levels) of one or the other (or both) of the administered agents. For example, a relevant resting period might be deemed to be completed when plasma concentration of the relevant agent is below about 1 μg/ml, 0.1 μg/ml, 0.01 μg/ml or 0.001 μg/ml, optionally upon evaluation or other consideration of one or more features of the individual's response.

In certain aspects, the number of cycles for which a particular agent is administered may be determined empirically. Also, in some embodiments, the precise regimen followed (e.g., number of doses, spacing of doses (e.g., relative to each other or to another event such as administration of another therapy), amount of doses, etc.) may be different for one or more cycles as compared with one or more other cycles.

The anti-SARS-CoV-2 antigen antibody, and if also administered, a second agent, may be administered via a route of administration independently selected from oral, parenteral (e.g., by intravenous, intra-arterial, subcutaneous, intramuscular, or epidural injection), topical, or nasal administration. According to certain embodiments, the anti-SARS-CoV-2 antigen antibody is administered parenterally.

As described above, aspects of the present disclosure include methods for treating an individual having or suspected of having a SARS-CoV-2 infection, e.g., COVID-19. By treatment is meant at least the prevention or an amelioration of one or more symptoms associated with the SARS-CoV-2 infection (e.g., COVID-19) of the individual, where amelioration is used in a broad sense to refer to at least a reduction in the magnitude of a parameter, e.g., symptom, associated with the SARS-CoV-2 infection. Non-limiting examples of such symptoms include one or more of viral load, hypoxia (e.g., oxygen saturation levels below 95%, e.g., as measured by pulse oximetry), pneumonia, acute respiratory distress syndrome, thrombosis in the pulmonary microcirculation, and/or the like. As such, treatment also includes situations where the SARS-CoV-2 infection, or at least one or more symptoms associated therewith, are completely inhibited, e.g., prevented from happening, or stopped, e.g., terminated, such that the individual no longer suffers from the SARS-CoV-2 infection, or at least the symptoms that characterize the SARS-CoV-2 infection.

In certain embodiments, the treatment methods comprise administering a therapeutically effective amount of an antibody that competes for binding to SARS-CoV-2 with an antibody comprising the six CDRs of the antibody designated herein as antibody 941; or an antibody that comprises the six CDRs of the antibody designated herein as antibody 941; or an antibody that comprises the V_{H} and V_{L} of the antibody designated herein as antibody 941. The antibody may be present as a fusion protein or conjugate of the present disclosure.

According to some embodiments, the treatment methods comprise administering a therapeutically effective amount of an antibody that competes for binding to SARS-CoV-2 with an antibody comprising the six CDRs of the antibody designated herein as antibody 980; or an antibody that comprises the six CDRs of the antibody designated herein as antibody 980; or an antibody that comprises the V_{H} and V_{L} of the antibody designated herein as antibody 980. The antibody may be present as a fusion protein or conjugate of the present disclosure.

In certain embodiments, the treatment methods comprise administering a therapeutically effective amount of an antibody that competes for binding to SARS-CoV-2 with an antibody comprising the six CDRs of the antibody designated herein as antibody 1589; or an antibody that comprises the six CDRs of the antibody designated herein as antibody 1589; or an antibody that comprises the V_{H} and V_{L} of the antibody designated herein as antibody 1589. The antibody may be present as a fusion protein or conjugate of the present disclosure.

Any of the treatment methods of the present disclosure may comprise administering a therapeutically effective amount of a “cocktail” of two or more antibodies to the individual, where at least one of the antibodies is an antibody of the present disclosure. In certain embodiments, the treatment methods comprise administering a therapeutically effective amount of a cocktail of two or more antibodies to the individual, where at least two of the antibodies are antibodies of the present disclosure. According to some embodiments, the treatment methods comprise administering a therapeutically effective amount of a cocktail of two or more, three or more, four or more, five or more, six or more, seven or more, eight or more, nine or more, ten or more, eleven, or each of the antibodies of the present disclosure, e.g., antibodies that compete for binding to SARS-CoV-2 with antibodies comprising the six CDRs of the antibodies designated herein as antibodies 508, 767, 935, 937, 941, 980, 1085, 1213, 1227, 1231, 1238, 1439, 1589, 1671, 1679, 1814, 1815, 1823, 1826, 1851, 1856, 1859, 1864, 1867, 1870, 1871, 1872, 1888, 1915, 1959, 1963, 1969, 1984, 2019, 2020, 2024, 2025, 2050, 2075, 2080, 2432, 2564, 2598, 2606, 2619, 2646, 2706, 2729, 2788, 2793, 2794, 2854, 2866, 2892, 3086, 3091, 3995, 4042, and/or 4441; or antibodies that comprise the six CDRs of the antibodies designated herein as antibodies 508, 767, 935, 937, 941, 980, 1085, 1213, 1227, 1231, 1238, 1439, 1589, 1671, 1679, 1814, 1815, 1823, 1826, 1851, 1856, 1859, 1864, 1867, 1870, 1871, 1872, 1888, 1915, 1959, 1963, 1969, 1984, 2019, 2020, 2024, 2025, 2050, 2075, 2080, 2432, 2564, 2598, 2606, 2619, 2646, 2706, 2729, 2788, 2793, 2794, 2854, 2866, 2892, 3086, 3091, 3995, 4042, and/or 4441; or antibodies that comprise the V_{H} and V_{L} of the antibodies designated herein as antibodies 508, 767, 935, 937, 941, 980, 1085, 1213, 1227, 1231, 1238, 1439, 1589, 1671, 1679, 1814, 1815, 1823, 1826, 1851, 1856, 1859, 1864, 1867, 1870, 1871, 1872, 1888, 1915, 1959, 1963, 1969, 1984, 2019, 2020, 2024, 2025, 2050, 2075, 2080, 2432, 2564, 2598, 2606, 2619, 2646, 2706, 2729, 2788, 2793, 2794, 2854, 2866, 2892, 3086, 3091, 3995, 4042, and/or 4441. The antibodies may be present as fusion proteins or conjugates of the present disclosure.

In certain embodiments, the treatment methods comprise administering a therapeutically effective amount of a cocktail comprising antibodies that compete for binding to SARS-CoV-2 with antibodies comprising the six CDRs of the antibodies designated herein as antibodies 941 and 980; or antibodies that comprise the six CDRs of the antibodies designated herein as antibodies 941 and 980; or antibodies that comprise the V_{H} and V_{L} of the antibodies designated herein as antibodies 941 and 980. The antibodies may be present as fusion proteins or conjugates of the present disclosure.

According to some embodiments, the treatment methods comprise administering a therapeutically effective amount of a cocktail comprising antibodies that compete for binding to SARS-CoV-2 with antibodies comprising the six CDRs of the antibodies designated herein as antibodies 941 and 1589; or antibodies that comprise the six CDRs of the antibodies designated herein as antibodies 941 and 1589; or antibodies that comprise the V_{H} and V_{L} of the antibodies designated herein as antibodies 941 and 1589. The antibodies may be present as fusion proteins or conjugates of the present disclosure.

In certain embodiments, the treatment methods comprise administering a therapeutically effective amount of a cocktail comprising antibodies that compete for binding to SARS-CoV-2 with antibodies comprising the six CDRs of the antibodies designated herein as antibodies 980 and 1589; or antibodies that comprise the six CDRs of the antibodies designated herein as antibodies 980 and 1589; or antibodies that comprise the V_{H} and V_{L} of the antibodies designated herein as antibodies 980 and 1589. The antibodies may be present as fusion proteins or conjugates of the present disclosure.

When a cocktail of antibodies is administered, the antibodies may be present in separate pharmaceutical compositions or may be administered in a single pharmaceutical composition.

### Kits

As summarized above, the present disclosure provides kits. The kits find use, e.g., in practicing the methods of the present disclosure. In some embodiments, a subject kit includes a composition (e.g., a pharmaceutical composition) that includes any of the anti-SARS-CoV-2 antibodies, fusion proteins, or conjugates of the present disclosure (e.g., any of the anti-SARS-CoV-2 antibodies, fusion proteins or conjugates described elsewhere herein—including any desired combination thereof). In some embodiments, provided are kits that include any of the pharmaceutical compositions described herein, including any of the pharmaceutical compositions described above in the section relating to the compositions of the present disclosure. Kits of the present disclosure may include instructions for administering the pharmaceutical composition to an individual in need thereof, including but not limited to, an individual having or suspected of having a SARS-CoV-2 infection, e.g., COVID-19.

The subject kits may include a quantity of the compositions, present in unit dosages, e.g., ampoules, or a multi-dosage format. As such, in certain embodiments, the kits may include one or more (e.g., two or more) unit dosages (e.g., ampoules) of a composition that includes any of the anti-SARS-CoV-2 antibodies, fusion proteins, conjugates, or cells of the present disclosure (e.g., any of the anti-SARS-CoV-2 antibodies, fusion proteins, conjugates, or cells described elsewhere herein). The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular anti-SARS-CoV-2 antigen antibody employed, the effect to be achieved, and the pharmacodynamics associated with the anti-SARS-CoV-2 antigen antibody, in the individual. In yet other embodiments, the kits may include a single multi dosage amount of the composition.

As will be appreciated, the kits of the present disclosure may include any of the agents and features described above in the sections relating to the subject antibodies, methods and compositions, which are not reiterated in detail herein for purposes of brevity.

Components of the kits may be present in separate containers, or multiple components may be present in a single container. A suitable container includes a single tube (e.g., vial), ampoule, one or more wells of a plate (e.g., a 96-well plate, a 384-well plate, etc.), or the like.

The instructions (e.g., instructions for use (IFU)) included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

Notwithstanding the appended claims, the present disclosure is also defined by the following embodiments:
1. An antibody that specifically binds a SARS-CoV-2 antigen and competes for binding to the SARS-CoV-2 antigen with an antibody comprising:
   a variable heavy chain (V_{H}) polypeptide comprising
      the V_{H} CDR1, V_{H} CDR2 and V_{H} CDR3 of the V_{H} set forth in SEQ ID NO:1, and
   a variable light chain (V_{L}) polypeptide comprising
      the V_{L} CDR1, V_{L} CDR2 and V_{L} CDR3 of the V_{L} set forth in SEQ ID NO:5;
   a variable heavy chain (V_{H}) polypeptide comprising
      the V_{H} CDR1, V_{H} CDR2 and V_{H} CDR3 of the V_{H} set forth in SEQ ID NO:9, and
   a variable light chain (V_{L}) polypeptide comprising
      the V_{L} CDR1, V_{L} CDR2 and V_{L} CDR3 of the V_{L} set forth in SEQ ID NO:13;
   a variable heavy chain (V_{H}) polypeptide comprising
      the V_{H} CDR1, V_{H} CDR2 and V_{H} CDR3 of the V_{H} set forth in SEQ ID NO:17, and
   a variable light chain (V_{L}) polypeptide comprising
      the V_{L} CDR1, V_{L} CDR2 and V_{L} CDR3 of the V_{L} set forth in SEQ ID NO:21;
   a variable heavy chain (V_{H}) polypeptide comprising
      the V_{H} CDR1, V_{H} CDR2 and V_{H} CDR3 of the V_{H} set forth in SEQ ID NO:25, and
   a variable light chain (V_{L}) polypeptide comprising
      the V_{L} CDR1, V_{L} CDR2 and V_{L} CDR3 of the V_{L} set forth in SEQ ID NO:29;
   a variable heavy chain (V_{H}) polypeptide comprising
      the V_{H} CDR1, V_{H} CDR2 and V_{H} CDR3 of the V_{H} set forth in SEQ ID NO:33, and
   a variable light chain (V_{L}) polypeptide comprising
      the V_{L} CDR1, V_{L} CDR2 and V_{L} CDR3 of the V_{L} set forth in SEQ ID NO:37;
   a variable heavy chain (V_{H}) polypeptide comprising
      the V_{H} CDR1, V_{H} CDR2 and V_{H} CDR3 of the V_{H} set forth in SEQ ID NO:41, and
   a variable light chain (V_{L}) polypeptide comprising
      the V_{L} CDR1, V_{L} CDR2 and V_{L} CDR3 of the V_{L} set forth in SEQ ID NO:45;
   a variable heavy chain (V_{H}) polypeptide comprising
      the V_{H} CDR1, V_{H} CDR2 and V_{H} CDR3 of the V_{H} set forth in SEQ ID NO:49, and
   a variable light chain (V_{L}) polypeptide comprising
      the V_{L} CDR1, V_{L} CDR2 and V_{L} CDR3 of the V_{L} set forth in SEQ ID NO:53;
   a variable heavy chain (V_{H}) polypeptide comprising
      the V_{H} CDR1, V_{H} CDR2 and V_{H} CDR3 of the V_{H} set forth in SEQ ID NO:57, and
   a variable light chain (V_{L}) polypeptide comprising
      the V_{L} CDR1, V_{L} CDR2 and V_{L} CDR3 of the V_{L} set forth in SEQ ID NO:61;
   a variable heavy chain (V_{H}) polypeptide comprising
      the V_{H} CDR1, V_{H} CDR2 and V_{H} CDR3 of the V_{H} set forth in SEQ ID NO:65, and
   a variable light chain (V_{L}) polypeptide comprising
      the V_{L} CDR1, V_{L} CDR2 and V_{L} CDR3 of the V_{L} set forth in SEQ ID NO:69;
   a variable heavy chain (V_{H}) polypeptide comprising
      the V_{H} CDR1, V_{H} CDR2 and V_{H} CDR3 of the V_{H} set forth in SEQ ID NO:73, and
   a variable light chain (V_{L}) polypeptide comprising
      the V_{L} CDR1, V_{L} CDR2 and V_{L} CDR3 of the V_{L} set forth in SEQ ID NO:77;
   a variable heavy chain (V_{H}) polypeptide comprising
      the V_{H} CDR1, V_{H} CDR2 and V_{H} CDR3 of the V_{H} set forth in SEQ ID NO:81, and
   a variable light chain (V_{L}) polypeptide comprising
      the V_{L} CDR1, V_{L} CDR2 and V_{L} CDR3 of the V_{L} set forth in SEQ ID NO:85; or
   a variable heavy chain (V_{H}) polypeptide comprising
      the V_{H} CDR1, V_{H} CDR2 and V_{H} CDR3 of the V_{H} set forth in SEQ ID NO:89, and
   a variable light chain (V_{L}) polypeptide comprising
      the V_{L} CDR1, V_{L} CDR2 and V_{L} CDR3 of the V_{L} set forth in SEQ ID NO:93;
   a variable heavy chain (V_{H}) polypeptide comprising
      the V_{H} CDR1, V_{H} CDR2 and V_{H} CDR3 of the V_{H} set forth in SEQ ID NO:97, and
   a variable light chain (V_{L}) polypeptide comprising
      the V_{L} CDR1, V_{L} CDR2 and V_{L} CDR3 of the V_{L} set forth in SEQ ID NO:101;
   a variable heavy chain (V_{H}) polypeptide comprising
      the V_{H} CDR1, V_{H} CDR2 and V_{H} CDR3 of the V_{H} set forth in SEQ ID NO:105, and
   a variable light chain (V_{L}) polypeptide comprising
      the V_{L} CDR1, V_{L} CDR2 and V_{L} CDR3 of the V_{L} set forth in SEQ ID NO:109;
   a variable heavy chain (V_{H}) polypeptide comprising
      the V_{H} CDR1, V_{H} CDR2 and V_{H} CDR3 of the V_{H} set forth in SEQ ID NO: 113, and
   a variable light chain (V_{L}) polypeptide comprising
      the V_{L} CDR1, V_{L} CDR2 and V_{L} CDR3 of the V_{L} set forth in SEQ ID NO:117;
   a variable heavy chain (V_{H}) polypeptide comprising
      the V_{H} CDR1, V_{H} CDR2 and V_{H} CDR3 of the V_{H} set forth in SEQ ID NO:121, and
   a variable light chain (V_{L}) polypeptide comprising
      the V_{L} CDR1, V_{L} CDR2 and V_{L} CDR3 of the V_{L} set forth in SEQ ID NO:125;
   a variable heavy chain (V_{H}) polypeptide comprising
      the V_{H} CDR1, V_{H} CDR2 and V_{H} CDR3 of the V_{H} set forth in SEQ ID NO:129, and
   a variable light chain (V_{L}) polypeptide comprising
      the V_{L} CDR1, V_{L} CDR2 and V_{L} CDR3 of the V_{L} set forth in SEQ ID NO:133;
   a variable heavy chain (V_{H}) polypeptide comprising
      the V_{H} CDR1, V_{H} CDR2 and V_{H} CDR3 of the V_{H} set forth in SEQ ID NO:137, and
   a variable light chain (V_{L}) polypeptide comprising
      the V_{L} CDR1, V_{L} CDR2 and V_{L} CDR3 of the V_{L} set forth in SEQ ID NO:141;
   a variable heavy chain (V_{H}) polypeptide comprising
      the V_{H} CDR1, V_{H} CDR2 and V_{H} CDR3 of the V_{H} set forth in SEQ ID NO:145, and
   a variable light chain (V_{L}) polypeptide comprising
      the V_{L} CDR1, V_{L} CDR2 and V_{L} CDR3 of the V_{L} set forth in SEQ ID NO:149;
   a variable heavy chain (V_{H}) polypeptide comprising
      the V_{H} CDR1, V_{H} CDR2 and V_{H} CDR3 of the V_{H} set forth in SEQ ID NO:153, and
   a variable light chain (V_{L}) polypeptide comprising
      the V_{L} CDR1, V_{L} CDR2 and V_{L} CDR3 of the V_{L} set forth in SEQ ID NO:157;
   a variable heavy chain (V_{H}) polypeptide comprising
      the V_{H} CDR1, V_{H} CDR2 and V_{H} CDR3 of the V_{H} set forth in SEQ ID NO:161, and
   a variable light chain (V_{L}) polypeptide comprising
      the V_{L} CDR1, V_{L} CDR2 and V_{L} CDR3 of the V_{L} set forth in SEQ ID NO:165;
   a variable heavy chain (V_{H}) polypeptide comprising
      the V_{H} CDR1, V_{H} CDR2 and V_{H} CDR3 of the V_{H} set forth in SEQ ID NO:169, and
   a variable light chain (V_{L}) polypeptide comprising
      the V_{L} CDR1, V_{L} CDR2 and V_{L} CDR3 of the V_{L} set forth in SEQ ID NO:173;
   a variable heavy chain (V_{H}) polypeptide comprising
      the V_{H} CDR1, V_{H} CDR2 and V_{H} CDR3 of the V_{H} set forth in SEQ ID NO:177, and
   a variable light chain (V_{L}) polypeptide comprising
      the V_{L} CDR1, V_{L} CDR2 and V_{L} CDR3 of the V_{L} set forth in SEQ ID NO:181;
   a variable heavy chain (V_{H}) polypeptide comprising
      the V_{H} CDR1, V_{H} CDR2 and V_{H} CDR3 of the V_{H} set forth in SEQ ID NO:185, and
   a variable light chain (V_{L}) polypeptide comprising
      the V_{L} CDR1, V_{L} CDR2 and V_{L} CDR3 of the V_{L} set forth in SEQ ID NO:189;
   a variable heavy chain (V_{H}) polypeptide comprising
      the V_{H} CDR1, V_{H} CDR2 and V_{H} CDR3 of the V_{H} set forth in SEQ ID NO:193, and
   a variable light chain (V_{L}) polypeptide comprising
      the V_{L} CDR1, V_{L} CDR2 and V_{L} CDR3 of the V_{L} set forth in SEQ ID NO:197;
   a variable heavy chain (V_{H}) polypeptide comprising
      the V_{H} CDR1, V_{H} CDR2 and V_{H} CDR3 of the V_{H} set forth in SEQ ID NO:201, and
   a variable light chain (V_{L}) polypeptide comprising
      the V_{L} CDR1, V_{L} CDR2 and V_{L} CDR3 of the V_{L} set forth in SEQ ID NO:205;
   a variable heavy chain (V_{H}) polypeptide comprising
      the V_{H} CDR1, V_{H} CDR2 and V_{H} CDR3 of the V_{H} set forth in SEQ ID NO:209, and
   a variable light chain (V_{L}) polypeptide comprising
      the V_{L} CDR1, V_{L} CDR2 and V_{L} CDR3 of the V_{L} set forth in SEQ ID NO:213;
   a variable heavy chain (V_{H}) polypeptide comprising
      the V_{H} CDR1, V_{H} CDR2 and V_{H} CDR3 of the V_{H} set forth in SEQ ID NO:217, and
   a variable light chain (V_{L}) polypeptide comprising
      the V_{L} CDR1, V_{L} CDR2 and V_{L} CDR3 of the V_{L} set forth in SEQ ID NO:221;
   a variable heavy chain (V_{H}) polypeptide comprising
      the V_{H} CDR1, V_{H} CDR2 and V_{H} CDR3 of the V_{H} set forth in SEQ ID NO:225, and
   a variable light chain (V_{L}) polypeptide comprising
      the V_{L} CDR1, V_{L} CDR2 and V_{L} CDR3 of the V_{L} set forth in SEQ ID NO:229;
   a variable heavy chain (V_{H}) polypeptide comprising
      the V_{H} CDR1, V_{H} CDR2 and V_{H} CDR3 of the V_{H} set forth in SEQ ID NO:233, and
   a variable light chain (V_{L}) polypeptide comprising
      the V_{L} CDR1, V_{L} CDR2 and V_{L} CDR3 of the V_{L} set forth in SEQ ID NO:237;
   a variable heavy chain (V_{H}) polypeptide comprising
      the V_{H} CDR1, V_{H} CDR2 and V_{H} CDR3 of the V_{H} set forth in SEQ ID NO:241, and
   a variable light chain (V_{L}) polypeptide comprising
      the V_{L} CDR1, V_{L} CDR2 and V_{L} CDR3 of the V_{L} set forth in SEQ ID NO:245;
   a variable heavy chain (V_{H}) polypeptide comprising
      the V_{H} CDR1, V_{H} CDR2 and V_{H} CDR3 of the V_{H} set forth in SEQ ID NO:249, and
   a variable light chain (V_{L}) polypeptide comprising
      the V_{L} CDR1, V_{L} CDR2 and V_{L} CDR3 of the V_{L} set forth in SEQ ID NO:253;
   a variable heavy chain (V_{H}) polypeptide comprising
      the V_{H} CDR1, V_{H} CDR2 and V_{H} CDR3 of the V_{H} set forth in SEQ ID NO:257, and
   a variable light chain (V_{L}) polypeptide comprising
      the V_{L} CDR1, V_{L} CDR2 and V_{L} CDR3 of the V_{L} set forth in SEQ ID NO:261;
   a variable heavy chain (V_{H}) polypeptide comprising
      the V_{H} CDR1, V_{H} CDR2 and V_{H} CDR3 of the V_{H} set forth in SEQ ID NO:265, and
   a variable light chain (V_{L}) polypeptide comprising
      the V_{L} CDR1, V_{L} CDR2 and V_{L} CDR3 of the V_{L} set forth in SEQ ID NO:269;
   a variable heavy chain (V_{H}) polypeptide comprising
      the V_{H} CDR1, V_{H} CDR2 and V_{H} CDR3 of the V_{H} set forth in SEQ ID NO:273, and
   a variable light chain (V_{L}) polypeptide comprising
      the V_{L} CDR1, V_{L} CDR2 and V_{L} CDR3 of the V_{L} set forth in SEQ ID NO:277;
   a variable heavy chain (V_{H}) polypeptide comprising
      the V_{H} CDR1, V_{H} CDR2 and V_{H} CDR3 of the V_{H} set forth in SEQ ID NO:281, and
   a variable light chain (V_{L}) polypeptide comprising
      the V_{L} CDR1, V_{L} CDR2 and V_{L} CDR3 of the V_{L} set forth in SEQ ID NO:285;
   a variable heavy chain (V_{H}) polypeptide comprising
      the V_{H} CDR1, V_{H} CDR2 and V_{H} CDR3 of the V_{H} set forth in SEQ ID NO:289, and
   a variable light chain (V_{L}) polypeptide comprising
      the V_{L} CDR1, V_{L} CDR2 and V_{L} CDR3 of the V_{L} set forth in SEQ ID NO:293;
   a variable heavy chain (V_{H}) polypeptide comprising
      the V_{H} CDR1, V_{H} CDR2 and V_{H} CDR3 of the V_{H} set forth in SEQ ID NO:297, and
   a variable light chain (V_{L}) polypeptide comprising
      the V_{L} CDR1, V_{L} CDR2 and V_{L} CDR3 of the V_{L} set forth in SEQ ID NO:301;
   a variable heavy chain (V_{H}) polypeptide comprising
      the V_{H} CDR1, V_{H} CDR2 and V_{H} CDR3 of the V_{H} set forth in SEQ ID NO:305, and
   a variable light chain (V_{L}) polypeptide comprising
      the V_{L} CDR1, V_{L} CDR2 and V_{L} CDR3 of the V_{L} set forth in SEQ ID NO:309;
   a variable heavy chain (V_{H}) polypeptide comprising
      the V_{H} CDR1, V_{H} CDR2 and V_{H} CDR3 of the V_{H} set forth in SEQ ID NO:313, and
   a variable light chain (V_{L}) polypeptide comprising
      the V_{L} CDR1, V_{L} CDR2 and V_{L} CDR3 of the V_{L} set forth in SEQ ID NO:317;
   a variable heavy chain (V_{H}) polypeptide comprising
      the V_{H} CDR1, V_{H} CDR2 and V_{H} CDR3 of the V_{H} set forth in SEQ ID NO:321, and
   a variable light chain (V_{L}) polypeptide comprising
      the V_{L} CDR1, V_{L} CDR2 and V_{L} CDR3 of the V_{L} set forth in SEQ ID NO:325;
   a variable heavy chain (V_{H}) polypeptide comprising
      the V_{H} CDR1, V_{H} CDR2 and V_{H} CDR3 of the V_{H} set forth in SEQ ID NO:329, and
   a variable light chain (V_{L}) polypeptide comprising
      the V_{L} CDR1, V_{L} CDR2 and V_{L} CDR3 of the V_{L} set forth in SEQ ID NO:333;
   a variable heavy chain (V_{H}) polypeptide comprising
      the V_{H} CDR1, V_{H} CDR2 and V_{H} CDR3 of the V_{H} set forth in SEQ ID NO:337, and
   a variable light chain (V_{L}) polypeptide comprising
      the V_{L} CDR1, V_{L} CDR2 and V_{L} CDR3 of the V_{L} set forth in SEQ ID NO:341;
   a variable heavy chain (V_{H}) polypeptide comprising
      the V_{H} CDR1, V_{H} CDR2 and V_{H} CDR3 of the V_{H} set forth in SEQ ID NO:345, and
   a variable light chain (V_{L}) polypeptide comprising
      the V_{L} CDR1, V_{L} CDR2 and V_{L} CDR3 of the V_{L} set forth in SEQ ID NO:349;
   a variable heavy chain (V_{H}) polypeptide comprising
      the V_{H} CDR1, V_{H} CDR2 and V_{H} CDR3 of the V_{H} set forth in SEQ ID NO:353, and
   a variable light chain (V_{L}) polypeptide comprising
      the V_{L} CDR1, V_{L} CDR2 and V_{L} CDR3 of the V_{L} set forth in SEQ ID NO:357;
   a variable heavy chain (V_{H}) polypeptide comprising
      the V_{H} CDR1, V_{H} CDR2 and V_{H} CDR3 of the V_{H} set forth in SEQ ID NO:361, and
   a variable light chain (V_{L}) polypeptide comprising
      the V_{L} CDR1, V_{L} CDR2 and V_{L} CDR3 of the V_{L} set forth in SEQ ID NO:365;
   a variable heavy chain (V_{H}) polypeptide comprising
      the V_{H} CDR1, V_{H} CDR2 and V_{H} CDR3 of the V_{H} set forth in SEQ ID NO:369, and
   a variable light chain (V_{L}) polypeptide comprising
      the V_{L} CDR1, V_{L} CDR2 and V_{L} CDR3 of the V_{L} set forth in SEQ ID NO:373;
   a variable heavy chain (V_{H}) polypeptide comprising
      the V_{H} CDR1, V_{H} CDR2 and V_{H} CDR3 of the V_{H} set forth in SEQ ID NO:377, and
   a variable light chain (V_{L}) polypeptide comprising
      the V_{L} CDR1, V_{L} CDR2 and V_{L} CDR3 of the V_{L} set forth in SEQ ID NO:381;
   a variable heavy chain (V_{H}) polypeptide comprising
      the V_{H} CDR1, V_{H} CDR2 and V_{H} CDR3 of the V_{H} set forth in SEQ ID NO:385, and
   a variable light chain (V_{L}) polypeptide comprising
      the V_{L} CDR1, V_{L} CDR2 and V_{L} CDR3 of the V_{L} set forth in SEQ ID NO:389;
   a variable heavy chain (V_{H}) polypeptide comprising
      the V_{H} CDR1, V_{H} CDR2 and V_{H} CDR3 of the V_{H} set forth in SEQ ID NO:393, and
   a variable light chain (V_{L}) polypeptide comprising
      the V_{L} CDR1, V_{L} CDR2 and V_{L} CDR3 of the V_{L} set forth in SEQ ID NO:397;
   a variable heavy chain (V_{H}) polypeptide comprising
      the V_{H} CDR1, V_{H} CDR2 and V_{H} CDR3 of the V_{H} set forth in SEQ ID NO:401, and
   a variable light chain (V_{L}) polypeptide comprising
      the V_{L} CDR1, V_{L} CDR2 and V_{L} CDR3 of the V_{L} set forth in SEQ ID NO:405;
   a variable heavy chain (V_{H}) polypeptide comprising
      the V_{H} CDR1, V_{H} CDR2 and V_{H} CDR3 of the V_{H} set forth in SEQ ID NO:409, and
   a variable light chain (V_{L}) polypeptide comprising
      the V_{L} CDR1, V_{L} CDR2 and V_{L} CDR3 of the V_{L} set forth in SEQ ID NO:413;
   a variable heavy chain (V_{H}) polypeptide comprising
      the V_{H} CDR1, V_{H} CDR2 and V_{H} CDR3 of the V_{H} set forth in SEQ ID NO:417, and
   a variable light chain (V_{L}) polypeptide comprising
      the V_{L} CDR1, V_{L} CDR2 and V_{L} CDR3 of the V_{L} set forth in SEQ ID NO:421;
   a variable heavy chain (V_{H}) polypeptide comprising
      the V_{H} CDR1, V_{H} CDR2 and V_{H} CDR3 of the V_{H} set forth in SEQ ID NO:425, and
   a variable light chain (V_{L}) polypeptide comprising
      the V_{L} CDR1, V_{L} CDR2 and V_{L} CDR3 of the V_{L} set forth in SEQ ID NO:429;
   a variable heavy chain (V_{H}) polypeptide comprising
      the V_{H} CDR1, V_{H} CDR2 and V_{H} CDR3 of the V_{H} set forth in SEQ ID NO:433, and
   a variable light chain (V_{L}) polypeptide comprising
      the V_{L} CDR1, V_{L} CDR2 and V_{L} CDR3 of the V_{L} set forth in SEQ ID NO:437;
   a variable heavy chain (V_{H}) polypeptide comprising
      the V_{H} CDR1, V_{H} CDR2 and V_{H} CDR3 of the V_{H} set forth in SEQ ID NO:441, and
   a variable light chain (V_{L}) polypeptide comprising
      the V_{L} CDR1, V_{L} CDR2 and V_{L} CDR3 of the V_{L} set forth in SEQ ID NO:445;
   a variable heavy chain (V_{H}) polypeptide comprising
      the V_{H} CDR1, V_{H} CDR2 and V_{H} CDR3 of the V_{H} set forth in SEQ ID NO:449, and
   a variable light chain (V_{L}) polypeptide comprising
      the V_{L} CDR1, V_{L} CDR2 and V_{L} CDR3 of the V_{L} set forth in SEQ ID NO:453;
   a variable heavy chain (V_{H}) polypeptide comprising
      the V_{H} CDR1, V_{H} CDR2 and V_{H} CDR3 of the V_{H} set forth in SEQ ID NO:457, and
   a variable light chain (V_{L}) polypeptide comprising
      the V_{L} CDR1, V_{L} CDR2 and V_{L} CDR3 of the V_{L} set forth in SEQ ID NO:461; or
   a variable heavy chain (V_{H}) polypeptide comprising
      the V_{H} CDR1, V_{H} CDR2 and V_{H} CDR3 of the V_{H} set forth in SEQ ID NO:465, and
   a variable light chain (V_{L}) polypeptide comprising
      the V_{L} CDR1, V_{L} CDR2 and V_{L} CDR3 of the V_{L} set forth in SEQ ID NO:469.
2. The antibody of embodiment 1, wherein the antibody comprises:
   a variable heavy chain (V_{H}) polypeptide comprising
      the V_{H} CDR1, V_{H} CDR2 and V_{H} CDR3 of the V_{H} set forth in SEQ ID NO:1, and
   a variable light chain (V_{L}) polypeptide comprising
      the V_{L} CDR1, V_{L} CDR2 and V_{L} CDR3 of the V_{L} set forth in SEQ ID NO:5;
   a variable heavy chain (V_{H}) polypeptide comprising
      the V_{H} CDR1, V_{H} CDR2 and V_{H} CDR3 of the V_{H} set forth in SEQ ID NO:9, and
   a variable light chain (V_{L}) polypeptide comprising
      the V_{L} CDR1, V_{L} CDR2 and V_{L} CDR3 of the V_{L} set forth in SEQ ID NO:13;
   a variable heavy chain (V_{H}) polypeptide comprising
      the V_{H} CDR1, V_{H} CDR2 and V_{H} CDR3 of the V_{H} set forth in SEQ ID NO:17, and
   a variable light chain (V_{L}) polypeptide comprising
      the V_{L} CDR1, V_{L} CDR2 and V_{L} CDR3 of the V_{L} set forth in SEQ ID NO:21;
   a variable heavy chain (V_{H}) polypeptide comprising
      the V_{H} CDR1, V_{H} CDR2 and V_{H} CDR3 of the V_{H} set forth in SEQ ID NO:25, and
   a variable light chain (V_{L}) polypeptide comprising
      the V_{L} CDR1, V_{L} CDR2 and V_{L} CDR3 of the V_{L} set forth in SEQ ID NO:29;
   a variable heavy chain (V_{H}) polypeptide comprising
      the V_{H} CDR1, V_{H} CDR2 and V_{H} CDR3 of the V_{H} set forth in SEQ ID NO:33, and
   a variable light chain (V_{L}) polypeptide comprising
      the V_{L} CDR1, V_{L} CDR2 and V_{L} CDR3 of the V_{L} set forth in SEQ ID NO:37;
   a variable heavy chain (V_{H}) polypeptide comprising
      the V_{H} CDR1, V_{H} CDR2 and V_{H} CDR3 of the V_{H} set forth in SEQ ID NO:41, and
   a variable light chain (V_{L}) polypeptide comprising
      the V_{L} CDR1, V_{L} CDR2 and V_{L} CDR3 of the V_{L} set forth in SEQ ID NO:45;
   a variable heavy chain (V_{H}) polypeptide comprising
      the V_{H} CDR1, V_{H} CDR2 and V_{H} CDR3 of the V_{H} set forth in SEQ ID NO:49, and
   a variable light chain (V_{L}) polypeptide comprising
      the V_{L} CDR1, V_{L} CDR2 and V_{L} CDR3 of the V_{L} set forth in SEQ ID NO:53;
   a variable heavy chain (V_{H}) polypeptide comprising
      the V_{H} CDR1, V_{H} CDR2 and V_{H} CDR3 of the V_{H} set forth in SEQ ID NO:57, and
   a variable light chain (V_{L}) polypeptide comprising
      the V_{L} CDR1, V_{L} CDR2 and V_{L} CDR3 of the V_{L} set forth in SEQ ID NO:61;
   a variable heavy chain (V_{H}) polypeptide comprising
      the V_{H} CDR1, V_{H} CDR2 and V_{H} CDR3 of the V_{H} set forth in SEQ ID NO:65, and
   a variable light chain (V_{L}) polypeptide comprising
      the V_{L} CDR1, V_{L} CDR2 and V_{L} CDR3 of the V_{L} set forth in SEQ ID NO:69;
   a variable heavy chain (V_{H}) polypeptide comprising
      the V_{H} CDR1, V_{H} CDR2 and V_{H} CDR3 of the V_{H} set forth in SEQ ID NO:73, and
   a variable light chain (V_{L}) polypeptide comprising
      the V_{L} CDR1, V_{L} CDR2 and V_{L} CDR3 of the V_{L} set forth in SEQ ID NO:77;
   a variable heavy chain (V_{H}) polypeptide comprising
      the V_{H} CDR1, V_{H} CDR2 and V_{H} CDR3 of the V_{H} set forth in SEQ ID NO:81, and
   a variable light chain (V_{L}) polypeptide comprising
      the V_{L} CDR1, V_{L} CDR2 and V_{L} CDR3 of the V_{L} set forth in SEQ ID NO:85; or
   a variable heavy chain (V_{H}) polypeptide comprising
      the V_{H} CDR1, V_{H} CDR2 and V_{H} CDR3 of the V_{H} set forth in SEQ ID NO:89, and
   a variable light chain (V_{L}) polypeptide comprising
      the V_{L} CDR1, V_{L} CDR2 and V_{L} CDR3 of the V_{L} set forth in SEQ ID NO:93;
   a variable heavy chain (V_{H}) polypeptide comprising
      the V_{H} CDR1, V_{H} CDR2 and V_{H} CDR3 of the V_{H} set forth in SEQ ID NO:97, and
   a variable light chain (V_{L}) polypeptide comprising
      the V_{L} CDR1, V_{L} CDR2 and V_{L} CDR3 of the V_{L} set forth in SEQ ID NO:101;
   a variable heavy chain (V_{H}) polypeptide comprising
      the V_{H} CDR1, V_{H} CDR2 and V_{H} CDR3 of the V_{H} set forth in SEQ ID NO:105, and
   a variable light chain (V_{L}) polypeptide comprising
      the V_{L} CDR1, V_{L} CDR2 and V_{L} CDR3 of the V_{L} set forth in SEQ ID NO:109;
   a variable heavy chain (V_{H}) polypeptide comprising
      the V_{H} CDR1, V_{H} CDR2 and V_{H} CDR3 of the V_{H} set forth in SEQ ID NO: 113, and
   a variable light chain (V_{L}) polypeptide comprising
      the V_{L} CDR1, V_{L} CDR2 and V_{L} CDR3 of the V_{L} set forth in SEQ ID NO:117;
   a variable heavy chain (V_{H}) polypeptide comprising
      the V_{H} CDR1, V_{H} CDR2 and V_{H} CDR3 of the V_{H} set forth in SEQ ID NO:121, and
   a variable light chain (V_{L}) polypeptide comprising
      the V_{L} CDR1, V_{L} CDR2 and V_{L} CDR3 of the V_{L} set forth in SEQ ID NO:125;
   a variable heavy chain (V_{H}) polypeptide comprising
      the V_{H} CDR1, V_{H} CDR2 and V_{H} CDR3 of the V_{H} set forth in SEQ ID NO:129, and
   a variable light chain (V_{L}) polypeptide comprising
      the V_{L} CDR1, V_{L} CDR2 and V_{L} CDR3 of the V_{L} set forth in SEQ ID NO:133;
   a variable heavy chain (V_{H}) polypeptide comprising
      the V_{H} CDR1, V_{H} CDR2 and V_{H} CDR3 of the V_{H} set forth in SEQ ID NO:137, and
   a variable light chain (V_{L}) polypeptide comprising
      the V_{L} CDR1, V_{L} CDR2 and V_{L} CDR3 of the V_{L} set forth in SEQ ID NO:141;
   a variable heavy chain (V_{H}) polypeptide comprising
      the V_{H} CDR1, V_{H} CDR2 and V_{H} CDR3 of the V_{H} set forth in SEQ ID NO:145, and
   a variable light chain (V_{L}) polypeptide comprising
      the V_{L} CDR1, V_{L} CDR2 and V_{L} CDR3 of the V_{L} set forth in SEQ ID NO:149;
   a variable heavy chain (V_{H}) polypeptide comprising
      the V_{H} CDR1, V_{H} CDR2 and V_{H} CDR3 of the V_{H} set forth in SEQ ID NO:153, and
   a variable light chain (V_{L}) polypeptide comprising
      the V_{L} CDR1, V_{L} CDR2 and V_{L} CDR3 of the V_{L} set forth in SEQ ID NO:157;
   a variable heavy chain (V_{H}) polypeptide comprising
      the V_{H} CDR1, V_{H} CDR2 and V_{H} CDR3 of the V_{H} set forth in SEQ ID NO:161, and
   a variable light chain (V_{L}) polypeptide comprising
      the V_{L} CDR1, V_{L} CDR2 and V_{L} CDR3 of the V_{L} set forth in SEQ ID NO:165;
   a variable heavy chain (V_{H}) polypeptide comprising
      the V_{H} CDR1, V_{H} CDR2 and V_{H} CDR3 of the V_{H} set forth in SEQ ID NO:169, and
   a variable light chain (V_{L}) polypeptide comprising
      the V_{L} CDR1, V_{L} CDR2 and V_{L} CDR3 of the V_{L} set forth in SEQ ID NO:173;
   a variable heavy chain (V_{H}) polypeptide comprising
      the V_{H} CDR1, V_{H} CDR2 and V_{H} CDR3 of the V_{H} set forth in SEQ ID NO:177, and
   a variable light chain (V_{L}) polypeptide comprising
      the V_{L} CDR1, V_{L} CDR2 and V_{L} CDR3 of the V_{L} set forth in SEQ ID NO:181;
   a variable heavy chain (V_{H}) polypeptide comprising
      the V_{H} CDR1, V_{H} CDR2 and V_{H} CDR3 of the V_{H} set forth in SEQ ID NO:185, and
   a variable light chain (V_{L}) polypeptide comprising
      the V_{L} CDR1, V_{L} CDR2 and V_{L} CDR3 of the V_{L} set forth in SEQ ID NO:189;
   a variable heavy chain (V_{H}) polypeptide comprising
      the V_{H} CDR1, V_{H} CDR2 and V_{H} CDR3 of the V_{H} set forth in SEQ ID NO:193, and
   a variable light chain (V_{L}) polypeptide comprising
      the V_{L} CDR1, V_{L} CDR2 and V_{L} CDR3 of the V_{L} set forth in SEQ ID NO:197;
   a variable heavy chain (V_{H}) polypeptide comprising
      the V_{H} CDR1, V_{H} CDR2 and V_{H} CDR3 of the V_{H} set forth in SEQ ID NO:201, and
   a variable light chain (V_{L}) polypeptide comprising
      the V_{L} CDR1, V_{L} CDR2 and V_{L} CDR3 of the V_{L} set forth in SEQ ID NO:205;
   a variable heavy chain (V_{H}) polypeptide comprising
      the V_{H} CDR1, V_{H} CDR2 and V_{H} CDR3 of the V_{H} set forth in SEQ ID NO:209, and
   a variable light chain (V_{L}) polypeptide comprising
      the V_{L} CDR1, V_{L} CDR2 and V_{L} CDR3 of the V_{L} set forth in SEQ ID NO:213;
   a variable heavy chain (V_{H}) polypeptide comprising
      the V_{H} CDR1, V_{H} CDR2 and V_{H} CDR3 of the V_{H} set forth in SEQ ID NO:217, and
   a variable light chain (V_{L}) polypeptide comprising
      the V_{L} CDR1, V_{L} CDR2 and V_{L} CDR3 of the V_{L} set forth in SEQ ID NO:221;
   a variable heavy chain (V_{H}) polypeptide comprising
      the V_{H} CDR1, V_{H} CDR2 and V_{H} CDR3 of the V_{H} set forth in SEQ ID NO:225, and
   a variable light chain (V_{L}) polypeptide comprising
      the V_{L} CDR1, V_{L} CDR2 and V_{L} CDR3 of the V_{L} set forth in SEQ ID NO:229;
   a variable heavy chain (V_{H}) polypeptide comprising
      the V_{H} CDR1, V_{H} CDR2 and V_{H} CDR3 of the V_{H} set forth in SEQ ID NO:233, and
   a variable light chain (V_{L}) polypeptide comprising
      the V_{L} CDR1, V_{L} CDR2 and V_{L} CDR3 of the V_{L} set forth in SEQ ID NO:237;
   a variable heavy chain (V_{H}) polypeptide comprising
      the V_{H} CDR1, V_{H} CDR2 and V_{H} CDR3 of the V_{H} set forth in SEQ ID NO:241, and
   a variable light chain (V_{L}) polypeptide comprising
      the V_{L} CDR1, V_{L} CDR2 and V_{L} CDR3 of the V_{L} set forth in SEQ ID NO:245;
   a variable heavy chain (V_{H}) polypeptide comprising
      the V_{H} CDR1, V_{H} CDR2 and V_{H} CDR3 of the V_{H} set forth in SEQ ID NO:249, and
   a variable light chain (V_{L}) polypeptide comprising
      the V_{L} CDR1, V_{L} CDR2 and V_{L} CDR3 of the V_{L} set forth in SEQ ID NO:253;
   a variable heavy chain (V_{H}) polypeptide comprising
      the V_{H} CDR1, V_{H} CDR2 and V_{H} CDR3 of the V_{H} set forth in SEQ ID NO:257, and
   a variable light chain (V_{L}) polypeptide comprising
      the V_{L} CDR1, V_{L} CDR2 and V_{L} CDR3 of the V_{L} set forth in SEQ ID NO:261;
   a variable heavy chain (V_{H}) polypeptide comprising
      the V_{H} CDR1, V_{H} CDR2 and V_{H} CDR3 of the V_{H} set forth in SEQ ID NO:265, and
   a variable light chain (V_{L}) polypeptide comprising
      the V_{L} CDR1, V_{L} CDR2 and V_{L} CDR3 of the V_{L} set forth in SEQ ID NO:269;
   a variable heavy chain (V_{H}) polypeptide comprising
      the V_{H} CDR1, V_{H} CDR2 and V_{H} CDR3 of the V_{H} set forth in SEQ ID NO:273, and
   a variable light chain (V_{L}) polypeptide comprising
      the V_{L} CDR1, V_{L} CDR2 and V_{L} CDR3 of the V_{L} set forth in SEQ ID NO:277;
   a variable heavy chain (V_{H}) polypeptide comprising
      the V_{H} CDR1, V_{H} CDR2 and V_{H} CDR3 of the V_{H} set forth in SEQ ID NO:281, and
   a variable light chain (V_{L}) polypeptide comprising
      the V_{L} CDR1, V_{L} CDR2 and V_{L} CDR3 of the V_{L} set forth in SEQ ID NO:285;
   a variable heavy chain (V_{H}) polypeptide comprising
      the V_{H} CDR1, V_{H} CDR2 and V_{H} CDR3 of the V_{H} set forth in SEQ ID NO:289, and
   a variable light chain (V_{L}) polypeptide comprising
      the V_{L} CDR1, V_{L} CDR2 and V_{L} CDR3 of the V_{L} set forth in SEQ ID NO:293;
   a variable heavy chain (V_{H}) polypeptide comprising
      the V_{H} CDR1, V_{H} CDR2 and V_{H} CDR3 of the V_{H} set forth in SEQ ID NO:297, and
   a variable light chain (V_{L}) polypeptide comprising
      the V_{L} CDR1, V_{L} CDR2 and V_{L} CDR3 of the V_{L} set forth in SEQ ID NO:301;
   a variable heavy chain (V_{H}) polypeptide comprising
      the V_{H} CDR1, V_{H} CDR2 and V_{H} CDR3 of the V_{H} set forth in SEQ ID NO:305, and
   a variable light chain (V_{L}) polypeptide comprising
      the V_{L} CDR1, V_{L} CDR2 and V_{L} CDR3 of the V_{L} set forth in SEQ ID NO:309;
   a variable heavy chain (V_{H}) polypeptide comprising
      the V_{H} CDR1, V_{H} CDR2 and V_{H} CDR3 of the V_{H} set forth in SEQ ID NO:313, and
   a variable light chain (V_{L}) polypeptide comprising
      the V_{L} CDR1, V_{L} CDR2 and V_{L} CDR3 of the V_{L} set forth in SEQ ID NO:317;
   a variable heavy chain (V_{H}) polypeptide comprising
      the V_{H} CDR1, V_{H} CDR2 and V_{H} CDR3 of the V_{H} set forth in SEQ ID NO:321, and
   a variable light chain (V_{L}) polypeptide comprising
      the V_{L} CDR1, V_{L} CDR2 and V_{L} CDR3 of the V_{L} set forth in SEQ ID NO:325;
   a variable heavy chain (V_{H}) polypeptide comprising
      the V_{H} CDR1, V_{H} CDR2 and V_{H} CDR3 of the V_{H} set forth in SEQ ID NO:329, and
   a variable light chain (V_{L}) polypeptide comprising
      the V_{L} CDR1, V_{L} CDR2 and V_{L} CDR3 of the V_{L} set forth in SEQ ID NO:333;
   a variable heavy chain (V_{H}) polypeptide comprising
      the V_{H} CDR1, V_{H} CDR2 and V_{H} CDR3 of the V_{H} set forth in SEQ ID NO:337, and
   a variable light chain (V_{L}) polypeptide comprising
      the V_{L} CDR1, V_{L} CDR2 and V_{L} CDR3 of the V_{L} set forth in SEQ ID NO:341;
   a variable heavy chain (V_{H}) polypeptide comprising
      the V_{H} CDR1, V_{H} CDR2 and V_{H} CDR3 of the V_{H} set forth in SEQ ID NO:345, and
   a variable light chain (V_{L}) polypeptide comprising
      the V_{L} CDR1, V_{L} CDR2 and V_{L} CDR3 of the V_{L} set forth in SEQ ID NO:349;
   a variable heavy chain (V_{H}) polypeptide comprising
      the V_{H} CDR1, V_{H} CDR2 and V_{H} CDR3 of the V_{H} set forth in SEQ ID NO:353, and
   a variable light chain (V_{L}) polypeptide comprising
      the V_{L} CDR1, V_{L} CDR2 and V_{L} CDR3 of the V_{L} set forth in SEQ ID NO:357;
   a variable heavy chain (V_{H}) polypeptide comprising
      the V_{H} CDR1, V_{H} CDR2 and V_{H} CDR3 of the V_{H} set forth in SEQ ID NO:361, and
   a variable light chain (V_{L}) polypeptide comprising
      the V_{L} CDR1, V_{L} CDR2 and V_{L} CDR3 of the V_{L} set forth in SEQ ID NO:365;
   a variable heavy chain (V_{H}) polypeptide comprising
      the V_{H} CDR1, V_{H} CDR2 and V_{H} CDR3 of the V_{H} set forth in SEQ ID NO:369, and
   a variable light chain (V_{L}) polypeptide comprising
      the V_{L} CDR1, V_{L} CDR2 and V_{L} CDR3 of the V_{L} set forth in SEQ ID NO:373;
   a variable heavy chain (V_{H}) polypeptide comprising
      the V_{H} CDR1, V_{H} CDR2 and V_{H} CDR3 of the V_{H} set forth in SEQ ID NO:377, and
   a variable light chain (V_{L}) polypeptide comprising
      the V_{L} CDR1, V_{L} CDR2 and V_{L} CDR3 of the V_{L} set forth in SEQ ID NO:381;
   a variable heavy chain (V_{H}) polypeptide comprising
      the V_{H} CDR1, V_{H} CDR2 and V_{H} CDR3 of the V_{H} set forth in SEQ ID NO:385, and
   a variable light chain (V_{L}) polypeptide comprising
      the V_{L} CDR1, V_{L} CDR2 and V_{L} CDR3 of the V_{L} set forth in SEQ ID NO:389;
   a variable heavy chain (V_{H}) polypeptide comprising
      the V_{H} CDR1, V_{H} CDR2 and V_{H} CDR3 of the V_{H} set forth in SEQ ID NO:393, and
   a variable light chain (V_{L}) polypeptide comprising
      the V_{L} CDR1, V_{L} CDR2 and V_{L} CDR3 of the V_{L} set forth in SEQ ID NO:397;
   a variable heavy chain (V_{H}) polypeptide comprising
      the V_{H} CDR1, V_{H} CDR2 and V_{H} CDR3 of the V_{H} set forth in SEQ ID NO:401, and
   a variable light chain (V_{L}) polypeptide comprising
      the V_{L} CDR1, V_{L} CDR2 and V_{L} CDR3 of the V_{L} set forth in SEQ ID NO:405;
   a variable heavy chain (V_{H}) polypeptide comprising
      the V_{H} CDR1, V_{H} CDR2 and V_{H} CDR3 of the V_{H} set forth in SEQ ID NO:409, and
   a variable light chain (V_{L}) polypeptide comprising
      the V_{L} CDR1, V_{L} CDR2 and V_{L} CDR3 of the V_{L} set forth in SEQ ID NO:413;
   a variable heavy chain (V_{H}) polypeptide comprising
      the V_{H} CDR1, V_{H} CDR2 and V_{H} CDR3 of the V_{H} set forth in SEQ ID NO:417, and
   a variable light chain (V_{L}) polypeptide comprising
      the V_{L} CDR1, V_{L} CDR2 and V_{L} CDR3 of the V_{L} set forth in SEQ ID NO:421;
   a variable heavy chain (V_{H}) polypeptide comprising
      the V_{H} CDR1, V_{H} CDR2 and V_{H} CDR3 of the V_{H} set forth in SEQ ID NO:425, and
   a variable light chain (V_{L}) polypeptide comprising
      the V_{L} CDR1, V_{L} CDR2 and V_{L} CDR3 of the V_{L} set forth in SEQ ID NO:429;
   a variable heavy chain (V_{H}) polypeptide comprising
      the V_{H} CDR1, V_{H} CDR2 and V_{H} CDR3 of the V_{H} set forth in SEQ ID NO:433, and
   a variable light chain (V_{L}) polypeptide comprising
      the V_{L} CDR1, V_{L} CDR2 and V_{L} CDR3 of the V_{L} set forth in SEQ ID NO:437;
   a variable heavy chain (V_{H}) polypeptide comprising
      the V_{H} CDR1, V_{H} CDR2 and V_{H} CDR3 of the V_{H} set forth in SEQ ID NO:441, and
   a variable light chain (V_{L}) polypeptide comprising
      the V_{L} CDR1, V_{L} CDR2 and V_{L} CDR3 of the V_{L} set forth in SEQ ID NO:445;
   a variable heavy chain (V_{H}) polypeptide comprising
      the V_{H} CDR1, V_{H} CDR2 and V_{H} CDR3 of the V_{H} set forth in SEQ ID NO:449, and
   a variable light chain (V_{L}) polypeptide comprising
      the V_{L} CDR1, V_{L} CDR2 and V_{L} CDR3 of the V_{L} set forth in SEQ ID NO:453;
   a variable heavy chain (V_{H}) polypeptide comprising
      the V_{H} CDR1, V_{H} CDR2 and V_{H} CDR3 of the V_{H} set forth in SEQ ID NO:457, and
   a variable light chain (V_{L}) polypeptide comprising
      the V_{L} CDR1, V_{L} CDR2 and V_{L} CDR3 of the V_{L} set forth in SEQ ID NO:461; or
   a variable heavy chain (V_{H}) polypeptide comprising
      the V_{H} CDR1, V_{H} CDR2 and V_{H} CDR3 of the V_{H} set forth in SEQ ID NO:465, and
   a variable light chain (V_{L}) polypeptide comprising
      the V_{L} CDR1, V_{L} CDR2 and V_{L} CDR3 of the V_{L} set forth in SEQ ID NO:469.
3. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:1; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:5.
4. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:9; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:13.
5. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:17; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:21.
6. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:25; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:29.
7. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:33; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:37.
8. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:41; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:45.
9. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:49; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:53.
10. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:57; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:61.
11. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:65; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:69.
12. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:73; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:77.
13. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:81; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:85.
14. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:89; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:93.
15. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:97; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:101.
16. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:105; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:109.
17. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO: 113; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO: 117.
18. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:121; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:125.
19. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:129; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:133.
20. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:137; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:141.
21. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:145; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:149.
22. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:153; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:157.
23. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:161; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:165.
24. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:169; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:173.
25. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:177; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:181.
26. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:185; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:189.
27. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:193; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:197.
28. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:201; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:205.
29. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:209; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:213.
30. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:217; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:221.
31. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:225; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:229.
32. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:233; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:237.
33. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:241; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:245.
34. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:249; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:253.
35. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:257; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:261.
36. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:265; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:269.
37. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:273; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:277.
38. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:281; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:285.
39. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:289; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:293.
40. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:297; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:301.
41. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:305; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:309.
42. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:313; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:317.
43. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:321; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:325.
44. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:329; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:333.
45. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:337; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:341.
46. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:345; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:349.
47. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:353; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:357.
48. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:361; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:365.
49. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:369; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:373.
50. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:377; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:381.
51. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:385; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:389.
52. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:393; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:397.
53. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:401; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:405.
54. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:409; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:413.
55. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:417; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:421.
56. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:425; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:429.
57. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:433; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:437.
58. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:441; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:445.
59. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:449; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:453.
60. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:457; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:461.
61. The antibody of embodiment 1 or embodiment 2, wherein the antibody comprises:
   a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:465; and
   a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:469.
62. The antibody of any one of embodiments 1 to 61, wherein the antibody is selected from the group consisting of: an IgG, Fv, single chain antibody, scFv, Fab, F(ab′)2, or Fab′.
63. The antibody of any one of embodiments 1 to 61, wherein the antibody is an IgG.
64. The antibody of embodiment 63, wherein the antibody is an IgG1.
65. The antibody of any one of embodiments 1 to 64, wherein the antibody comprises an Fc region, and the Fc region is heterologous to the V_{H} of the antibody.
66. The antibody of embodiment 65, wherein the Fc region is a variant Fc region.
67. The antibody of embodiment 66, wherein the variant Fc region comprises one or more amino acid substitutions, one or more amino acid insertions, one or more amino acid deletions, or any combination thereof, relative to a wild-type Fc region.
68. The antibody of any one of embodiments 1 to 61, wherein the antibody is a Fab.
69. The antibody of any one of embodiments 1 to 61, wherein the antibody is a single chain antibody.
70. The antibody of embodiment 69, wherein the antibody is an scFv.
71. The antibody of any one of embodiments 1 to 67, wherein the antibody is a recombinant antibody.
72. The antibody of any one of embodiments 1 to 67, wherein the antibody is a monoclonal antibody.
73. The antibody of any one of embodiments 1 to 72, wherein the antibody comprises an extent of glycosylation, a glycosylation pattern, or both, which is different from the extent of glycosylation, the glycosylation pattern, or both, of a naturally occurring antibody.
74. The antibody of any one of embodiments 1 to 73, wherein the antibody specifically binds a SARS-CoV-2 antigen selected from the group consisting of: the S1 subunit of a SARS-CoV-2 spike (S) protein, the receptor-binding domain (RBD) of the S1 subunit of a SARS-CoV-2 spike protein, the S2 subunit of a SARS-CoV-2 spike protein, a SARS-CoV-2 spike (S) protein trimer, a SARS-CoV-2 envelope (E) protein, a SARS-CoV-2 membrane (M) protein, and a SARS-CoV-2 nucleocapsid (N) protein.
75. The antibody of any one of embodiments 1 to 74, wherein the antibody is a bispecific antibody comprising a first antigen-binding domain that specifically binds SARS-CoV-2, and wherein the first antigen binding domain comprises a V_{H} polypeptide-V_{L} polypeptide pair as defined in any one of embodiments 1 to 61.
76. The antibody of embodiment 75, wherein the bispecific antibody comprises a second antigen-binding domain that specifically binds a SARS-CoV-2 antigen.
77. The antibody of embodiment 75, wherein the bispecific antibody comprises a second antigen-binding domain that specifically binds an antigen other than a SARS-CoV-2 antigen.
78. A fusion protein, comprising:
   a chain of an antibody of any one of embodiments 1 to 61 fused to a heterologous sequence of amino acids.
79. The fusion protein of embodiment 78, wherein the heterologous sequence of amino acids is fused to the C-terminus of the chain of the antibody.
80. The fusion protein of embodiment 78 or embodiment 79, wherein the antibody is the single chain antibody of embodiment 69 or 70.
81. A conjugate, comprising:
   an antibody of any one of embodiments 1 to 61 or a fusion protein of any one of embodiments 78 to 80; and an agent conjugated to the antibody or fusion protein.
82. The conjugate of embodiment 81, wherein the agent is a detectable label or a half-life extending moiety.
83. The conjugate of embodiment 82, wherein the detectable label is a radiolabel.
84. The conjugate of embodiment 82, wherein the detectable label is an in vivo imaging agent.
85. The conjugate of any one of embodiments 81 to 84, wherein the agent is conjugated to the antibody or fusion protein via a non-cleavable linker.
86. The conjugate of any one of embodiments 81 to 84, wherein the agent is conjugated to the antibody or fusion protein via a cleavable linker.
87. A nucleic acid encoding a variable heavy chain (V_{H}) polypeptide, a variable light chain (V_{L}) polypeptide, or both, of the antibody of any one of embodiments 1 to 70.
88. The nucleic acid of embodiment 87, wherein the antibody is a single chain antibody, and wherein the nucleic acid encodes the single chain antibody.
89. The nucleic acid of embodiment 88, wherein the single chain antibody is an scFv.
90. An expression vector comprising the nucleic acid of any one of embodiments 87 to 89.
91. A cell comprising the nucleic acid of any one of embodiments 87 to 89 or the expression vector of embodiment 90.
92. The cell of embodiment 91, wherein the nucleic acid encodes the V_{H} polypeptide of the antibody and the V_{L} polypeptide of the antibody.
93. The cell of embodiment 92, wherein the antibody is a single chain antibody, and wherein the nucleic acid encodes the single chain antibody.
94. The cell of embodiment 93, wherein the single chain antibody is an scFv.
95. A cell comprising:
   a first nucleic acid encoding a variable heavy chain (V_{H}) polypeptide of the antibody of any one of embodiments 1 to 68; and
   a second nucleic acid encoding a variable light chain (V_{L}) polypeptide of the antibody.
96. The cell of embodiment 95, comprising:
   a first expression vector comprising the first nucleic acid; and
   a second expression vector comprising the second nucleic acid.
97. A method of making the antibody of any one of embodiments 1 to 77, comprising culturing the cell of any one of embodiments 91 to 96 under conditions suitable for the cell to express the antibody, wherein the antibody is produced.
98. The method according to embodiment 97, further comprising, prior to the culturing, transfecting the cell or an ancestor thereof with an expression vector encoding the V_{H}, the V_{L}, or both, of the antibody.
99. A pharmaceutical composition, comprising:
   the antibody of any one of embodiments 1 to 77; and
   a pharmaceutically acceptable carrier.
100. A pharmaceutical composition, comprising:
   two or more different antibodies each having a V_{H} and V_{L} pair as defined in any one of embodiments 1 to 61; and
   a pharmaceutically acceptable carrier.
101. The pharmaceutical composition of embodiment 100, wherein the two or more different antibodies comprise:
   a first antibody that specifically binds the receptor-binding domain (RBD) of the S1 subunit of a SARS-CoV-2 spike (S) protein; and
   a second antibody that specifically binds the S2 subunit of the SARS-CoV-2 spike (S) protein.
102. The pharmaceutical composition of embodiment 100, wherein the two or more different antibodies comprise:
   a first antibody which is a class I RBD-binding antibody; and
   a second antibody which is a class III RBD-binding antibody.
103. A pharmaceutical composition, comprising:
   the fusion protein of any one of embodiments 78 to 80; and
   a pharmaceutically acceptable carrier.
104. A pharmaceutical composition, comprising:
   the conjugate of any one of embodiments 81 to 86; and
   a pharmaceutically acceptable carrier.
105. The pharmaceutical composition of any one of embodiments 99 to 104, comprising:
   a first antibody comprising:
      a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:25, and
      a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:29,
      wherein the antibody comprises one or more, two or more, three or more, four or more, five or six of the complementarity determining regions (CDRs) of an antibody comprising the V_{H} and V_{L} set forth in SEQ ID NO:25 and SEQ ID NO:29, respectively; and
   a second antibody comprising:
      a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:33, and
      a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:37,
      wherein the antibody comprises one or more, two or more, three or more, four or more, five or six of the complementarity determining regions (CDRs) of an antibody comprising the V_{H} and V_{L} set forth in SEQ ID NO:33 and SEQ ID NO:37, respectively.
106. The pharmaceutical composition of any one of embodiments 99 to 104, comprising:
   a first antibody comprising:
      a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:25, and
      a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:29,
      wherein the antibody comprises one or more, two or more, three or more, four or more, five or six of the complementarity determining regions (CDRs) of an antibody comprising the V_{H} and V_{L} set forth in SEQ ID NO:25 and SEQ ID NO:29, respectively; and
   a second antibody comprising:
      a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:81, and
      a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:85,
      wherein the antibody comprises one or more, two or more, three or more, four or more, five or six of the complementarity determining regions (CDRs) of an antibody comprising the V_{H} and V_{L} set forth in SEQ ID NO:81 and SEQ ID NO:85, respectively.
107. The pharmaceutical composition of any one of embodiments 99 to 104, comprising:
   a first antibody comprising:
      a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:33, and
      a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:37,
      wherein the antibody comprises one or more, two or more, three or more, four or more, five or six of the complementarity determining regions (CDRs) of an antibody comprising the V_{H} and V_{L} set forth in SEQ ID NO:33 and SEQ ID NO:37, respectively; and
   a second antibody comprising:
      a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:81, and
      a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:85,
      wherein the antibody comprises one or more, two or more, three or more, four or more, five or six of the complementarity determining regions (CDRs) of an antibody comprising the V_{H} and V_{L} set forth in SEQ ID NO:81 and SEQ ID NO:85, respectively.
108. The pharmaceutical composition of any one of embodiments 99 to 107, wherein the pharmaceutical composition is formulated for parenteral administration.
109. The pharmaceutical composition of embodiment 108, wherein the pharmaceutical composition is formulated for intravenous, intramuscular, or subcutaneous administration.
110. The pharmaceutical composition of any one of embodiments 99 to 107, wherein the pharmaceutical composition is formulated for inhalational administration.
111. The pharmaceutical composition of any one of embodiments 99 to 107, wherein the pharmaceutical composition is formulated for intranasal administration.
112. The pharmaceutical composition of any one of embodiments 99 to 108, wherein the composition provides a unit dosage effective to neutralize a SARS-CoV-2 virus infection.
113. A kit, comprising:
the pharmaceutical composition of any one of embodiments 99 to 112; and instructions for administering an effective amount of the pharmaceutical composition to an individual in need thereof.
114. The kit of embodiment 113, wherein the pharmaceutical composition is present in one or more unit dosages.
115. The kit of embodiment 113, wherein the pharmaceutical composition is present in two or more unit dosages.
116. The kit of any one of embodiments 113 to 115, wherein the individual in need thereof has or is suspected of having a SARS-CoV-2 infection.
117. The kit of any one of embodiments 113 to 115, wherein the individual in need thereof is known to be immunocompromised and is not suspected of having a SARS-CoV-2 infection.
118. A method comprising administering to an individual in need thereof a therapeutically effective amount of the antibody of any one of embodiments 1 to 77, the fusion protein of any one of embodiments 78 to 80, or the conjugate of any one of embodiments 81 to 86.
119. The method according to embodiment 118, wherein the individual has a SARS-CoV-2 infection, and wherein the method is effective in neutralizing the SARS-CoV-2 infection in the individual.
120. The method according to embodiment 118, wherein the antibody, fusion protein or conjugate is administered prophylactically to an individual who does not have a SARS-CoV-2 infection.
121. The method according to embodiment 120, wherein the individual who does not have a SARS-CoV-2 infection is immunocompromised.
122. The method according to embodiment 121, wherein the individual is immunocompromised by virtue of having cancer, taking one or more immunosuppressive drugs, being a transplant recipient, having HIV/AIDS, having an inherited disease that affects the immune system, having congenital agammaglobulinemia, having congenital IgA deficiency, being elderly, or any combination thereof.
123. A method of treating an individual having a SARS-CoV-2 infection, the method comprising:
   administering to the individual a therapeutically effective amount of the antibody of any one of embodiments 1 to 77, the fusion protein of any one of embodiments 78 to 80, or the conjugate of any one of embodiments 81 to 86.
124. The method according to any one of embodiments 118 to 123, the method comprising administering to the individual a therapeutically effective amount of a combination of two or more different antibodies each having a V_{H} and V_{L} pair as defined in any one of embodiments 1 to 61.
125. The method according to embodiment 124, wherein the two or more different antibodies comprise:
   a first antibody that specifically binds the receptor-binding domain (RBD) of the S1 subunit of a SARS-CoV-2 spike (S) protein; and
   a second antibody that specifically binds the S2 subunit of the SARS-CoV-2 spike (S) protein.
126. The method according to embodiment 124, wherein the two or more different antibodies comprise:
   a first antibody which is a class I RBD-binding antibody; and
   a second antibody which is a class III RBD-binding antibody.
127. The method according to embodiment 123, the method comprising administering to the individual a therapeutically effective amount of a combination of two or more different antibodies, or fusion proteins or conjugates comprising same, the two or more different antibodies comprising:
   a first antibody comprising:
      a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:25, and
      a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:29,
      wherein the antibody comprises one or more, two or more, three or more, four or more, five or six of the complementarity determining regions (CDRs) of an antibody comprising the V_{H} and V_{L} set forth in SEQ ID NO:25 and SEQ ID NO:29, respectively; and
   a second antibody comprising:
      a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:33, and
      a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:37,
      wherein the antibody comprises one or more, two or more, three or more, four or more, five or six of the complementarity determining regions (CDRs) of an antibody comprising the V_{H} and V_{L} set forth in SEQ ID NO:33 and SEQ ID NO:37, respectively.
128. The method according to embodiment 123, the method comprising administering to the individual a therapeutically effective amount of a combination of two or more different antibodies, or fusion proteins or conjugates comprising same, the two or more different antibodies comprising:
   a first antibody comprising:
      a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:25, and
      a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:29,
      wherein the antibody comprises one or more, two or more, three or more, four or more, five or six of the complementarity determining regions (CDRs) of an antibody comprising the V_{H} and V_{L} set forth in SEQ ID NO:25 and SEQ ID NO:29, respectively; and
   a second antibody comprising:
      a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:81, and
      a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:85,
      wherein the antibody comprises one or more, two or more, three or more, four or more, five or six of the complementarity determining regions (CDRs) of an antibody comprising the V_{H} and V_{L} set forth in SEQ ID NO:81 and SEQ ID NO:85, respectively.
129. The method according to embodiment 123, the method comprising administering to the individual a therapeutically effective amount of a combination of two or more different antibodies, or fusion proteins or conjugates comprising same, the two or more different antibodies comprising:
   a first antibody comprising:
      a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:33, and
      a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:37,
      wherein the antibody comprises one or more, two or more, three or more, four or more, five or six of the complementarity determining regions (CDRs) of an antibody comprising the V_{H} and V_{L} set forth in SEQ ID NO:33 and SEQ ID NO:37, respectively; and
   a second antibody comprising:
      a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:81, and
      a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:85,
      wherein the antibody comprises one or more, two or more, three or more, four or more, five or six of the complementarity determining regions (CDRs) of an antibody comprising the V_{H} and V_{L} set forth in SEQ ID NO:81 and SEQ ID NO:85, respectively.
130. The method according to any one of embodiments 118 to 129, wherein the administering is by parenteral administration.
131. The method according to embodiment 130, wherein the administering is by intravenous, intramuscular, or subcutaneous administration.
132. The method according to any one of embodiments 118 to 129, wherein the administering is by inhalational administration.
133. The method according to any one of embodiments 118 to 129, wherein the administering is by intranasal administration.

The following examples are offered by way of illustration and not by way of limitation.

### EXPERIMENTAL

### Example 1—Identification of Paired B-Cell Receptor (BCR) Sequences

The high-throughput immunoSEQ® Assay (Adaptive Biotechnologies®) and pairSEQ® assay (Adaptive Biotechnologies®) were applied to identify and pair BCRs from blood of COVID-19 patients.

10-40 mL of blood was obtained from COVID-19 patients who were either in the late stage of an acute infection or had clinically cleared and recovered from the SARS-CoV-2 virus. Blood samples were sorted to select antibody secreting B cells (ASCs) using relevant markers (e.g., CD3−/CD19+/CD27hi/CD38hi) or antigen-specific memory B cells with protein comprised of his-tagged SARS-CoV-2 spike trimer and tetramerized biotinylated RBD protein.

Genomic DNA (gDNA) was extracted from unsorted cells (PBMCs or Whole Blood) and run on the immunoSEQ® Assay to identify the frequency of BCRs in the unsorted B cell population. pairSEQ was run on a fixed number of ASCs and antigen-specific memory B cells. In the pairSEQ assay, ASCs and Memory B cells were allocated to each well in 96-well plates, where memory B cells were be allocated on different plates from ASCs. mRNA was extracted, converted to cDNA and amplified by BCR-specific primers. Well-specific barcodes were attached to the sequences, and the BCR molecules pooled for sequencing. Computational demultiplexing followed to map each BCR sequence back to the wells in which it originated. Because of the massive immune repertoire diversity, the probability that two or more BCR clones occupy exactly the same sets of wells is miniscule. So, a pair of BCR heavy and light chain sequences that uniquely share a set of wells is an accurate BCR Paired Sequence. More than 300,000 paired clonal lineages and SHM variants within those lineages were identified in this manner.

BCR Paired Sequences were further evaluated and characterized per the methods below to identify the ones most effective in neutralizing the SARS-CoV-2 virus.

### Example 2—Antibody Synthesis

Paired BCR sequences were selected via an initial in-silico design analysis. The goal of this step was to conduct an initial screening of the CDR sequences to identify antibodies derived from patients at the optimal timepoint and structural features (e.g., free cysteines, degradation hotspots, susceptible deamidation and oxidation sites in the CDR regions) of the antibodies that may cause downstream antibody development vulnerabilities. More than 3,300 antibodies were synthesized onto an IgG₁ backbone based on these selection criteria.

Antibodies in IgG format were expressed from plasmids. In this example, the V_{H} and V_{L} of each antibody were expressed from separate plasmids. Antibody 508 was expressed using a 508 V_{H}-encoding plasmid (SEQ ID NO:473) and a 508 V_{L}-encoding plasmid (SEQ ID NO:474). Antibody 767 was expressed using a 767 V_{H}-encoding plasmid (SEQ ID NO:475) and a 767 V_{L}-encoding plasmid (SEQ ID NO:476). Antibody 935 was expressed using a 935 V_{H}-encoding plasmid (SEQ ID NO:477) and a 935 V_{L}-encoding plasmid (SEQ ID NO:478). Antibody 941 was expressed using a 941 V_{H}-encoding plasmid (SEQ ID NO:479) and a 941 V_{L}-encoding plasmid (SEQ ID NO:480). Antibody 980 was expressed using a 980 V_{H}-encoding plasmid (SEQ ID NO:481) and a 980 V_{L}-encoding plasmid (SEQ ID NO:482). Antibody 1085 was expressed using a 1085 V_{H}-encoding plasmid (SEQ ID NO:483) and a 1085 V_{L}-encoding plasmid (SEQ ID NO:484). Antibody 1213 was expressed using a 1213 V_{H}-encoding plasmid (SEQ ID NO:485) and a 1213 V_{L}-encoding plasmid (SEQ ID NO:486). Antibody 1227 was expressed using a 1227 V_{H}-encoding plasmid (SEQ ID NO:487) and a 1227 V_{L}-encoding plasmid (SEQ ID NO:488). Antibody 1231 was expressed using a 1231 V_{H}-encoding plasmid (SEQ ID NO:489) and a 1231 V_{L}-encoding plasmid (SEQ ID NO:490). Antibody 1439 was expressed using a 1439 V_{H}-encoding plasmid (SEQ ID NO:491) and a 1439 V_{L}-encoding plasmid (SEQ ID NO:492). Antibody 1589 was expressed using a 1589 V_{H}-encoding plasmid (SEQ ID NO:493) and a 1589 V_{L}-encoding plasmid (SEQ ID NO:494). Antibody 1679 was expressed using a 1679 V_{H}-encoding plasmid (SEQ ID NO:495) and a 1679 V_{L}-encoding plasmid (SEQ ID NO:496).

### Example 3—Target Specificity and Affinity

Antigen specificity for synthesized antibodies was initially determined using an ELISA assay targeting the spike protein of SARS-CoV-2. The RBD, S1 domain, S2 domain, nucleocapsid and the trimeric form of the spike protein were used as targets proteins. In addition to the WA01/2020 strain RBD sequence, sensitivity to variance were tested using RBD with mutations found in beta and delta variants. Reactivity of S2 antibodies to other corona viruses was tested by immobilization of spike proteins form SARS-Cov1, MERS-Cov, HCOV-HKU1, HCOV-229E and HCOV-OC43.

EC₅₀ of functional antibodies (identified via ACE2 blockade combined with live or pseudovirus neutralization) was determined by performing a response ELISA with concentration starting from 1 μg/ml and three-fold dilution. mAbs were tested with 7-10-dose data point. Human IgG were included as negative control and Anti-S antibody, Clone 6D11F2, was used as a positive control.

To determine whether RBD specific antibodies of interest bound to similar epitopes, competitive ELISA was used. His tagged RBD protein was used as antigen. Unlabeled and biotin-labeled antibodies was simultaneously added to RBD coated plates. Amount of antibody bound was detected using streptavidin HRP. Level of inhibition was calculated based on sample with no unlabeled antibody.

Confirmation of affinity of selected mAbs was determined by Biacore 8K per the manufacturer's instructions. Briefly RBD, S1 or Trimer were immobilized under 25 degrees Celsius while HBS-EP was used as the running buffer. The sensor chip surface of flow cells 1 and 2 were activated. Antigens were diluted in NaAc and injected into the flow cell 2 to achieve conjugation and while flow cell 1 was set as blank. Antibodies were injected over the surface as association phase, followed by injecting running buffer as dissociation phase. All the data were processed using the Biacore 8K Evaluation software version 1.1. Flow cell 1 and blank injection of buffer in each cycle were used as double reference for Response Units subtraction.

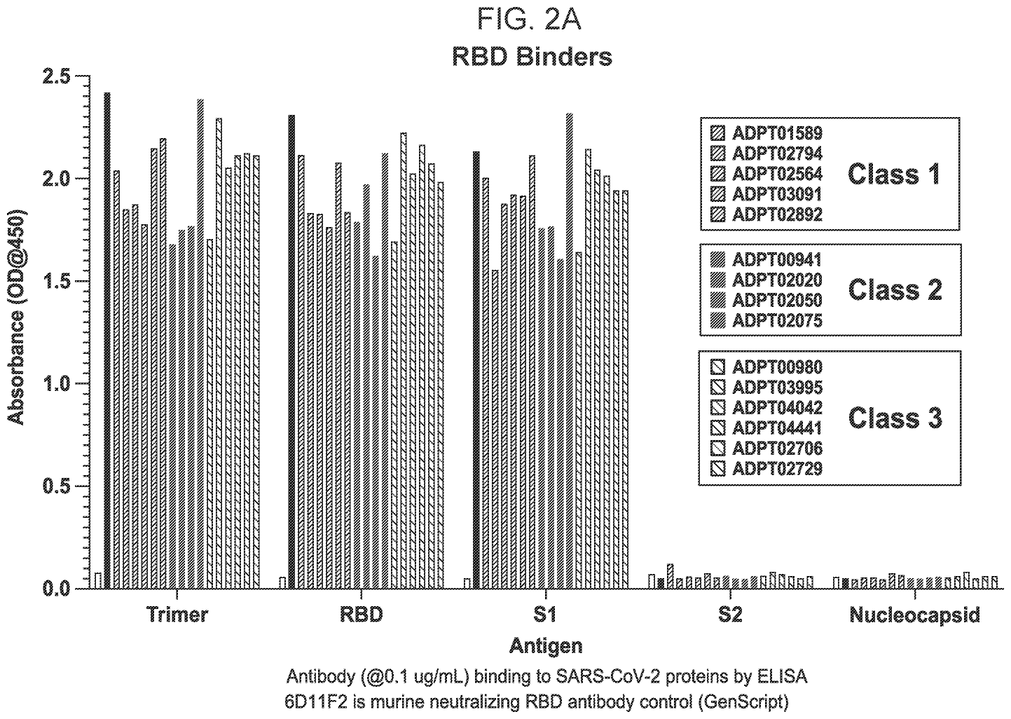

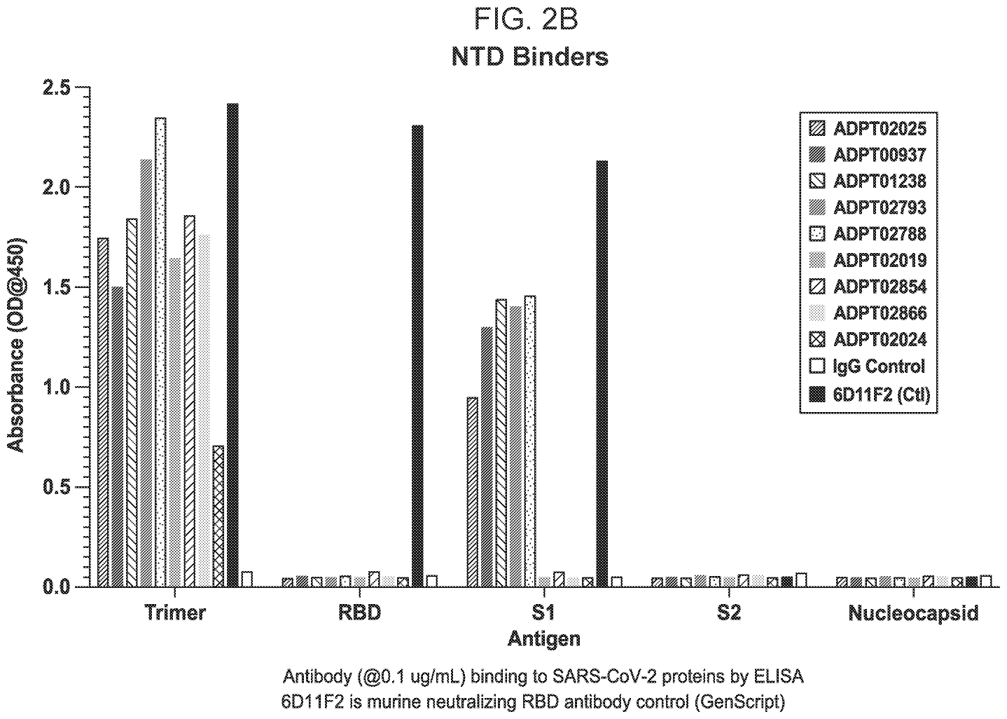

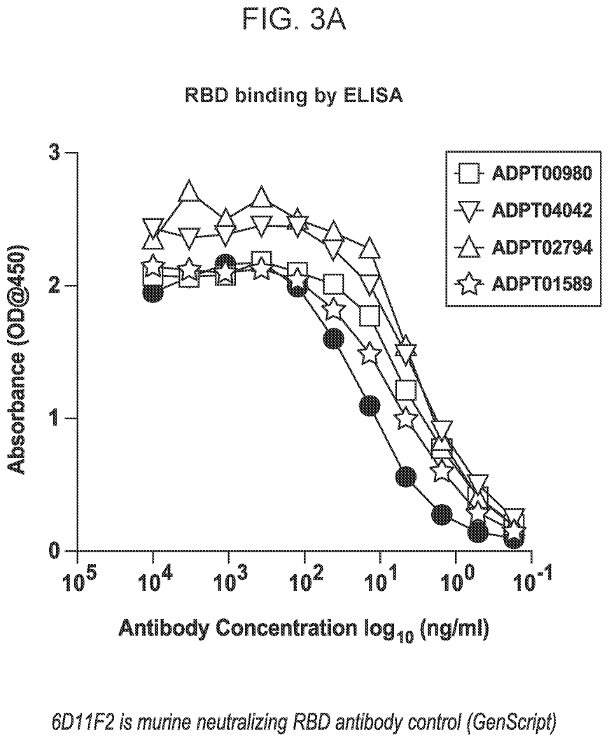

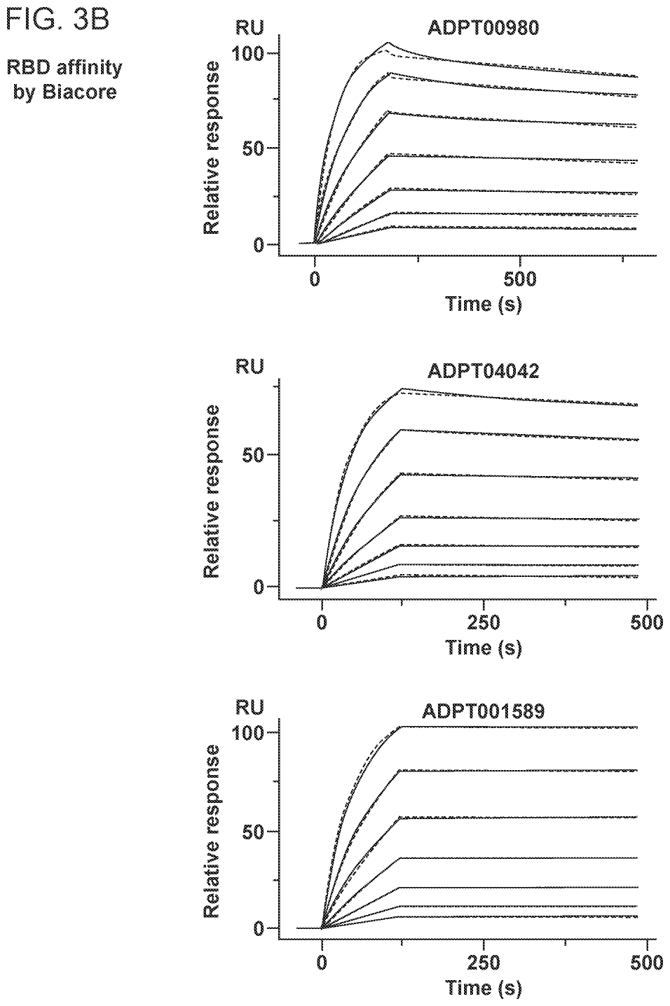

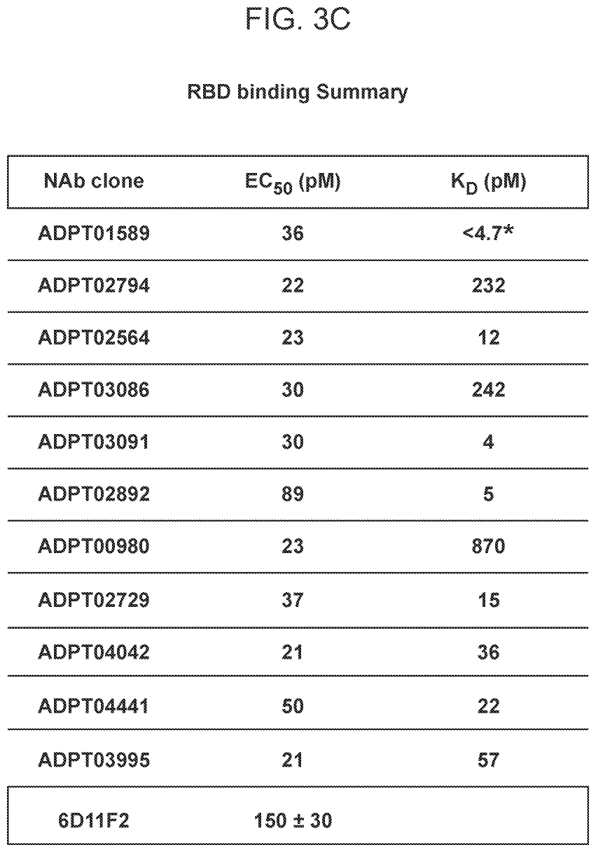

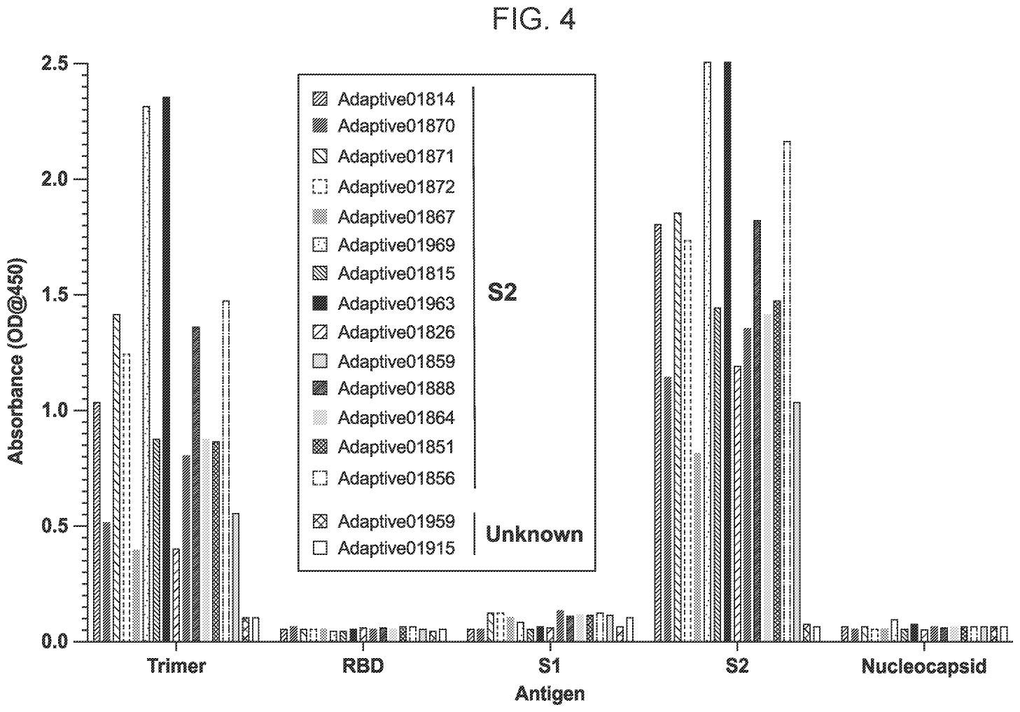

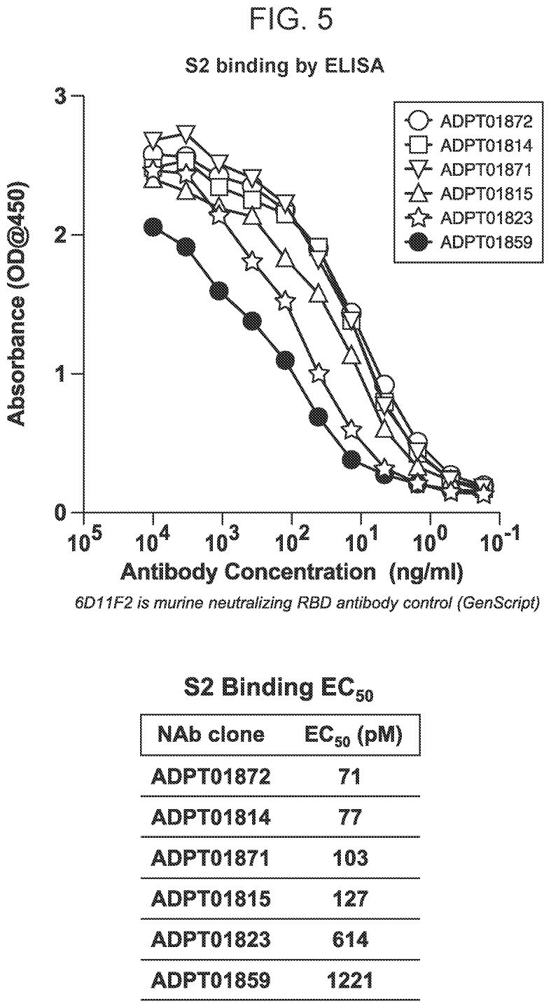

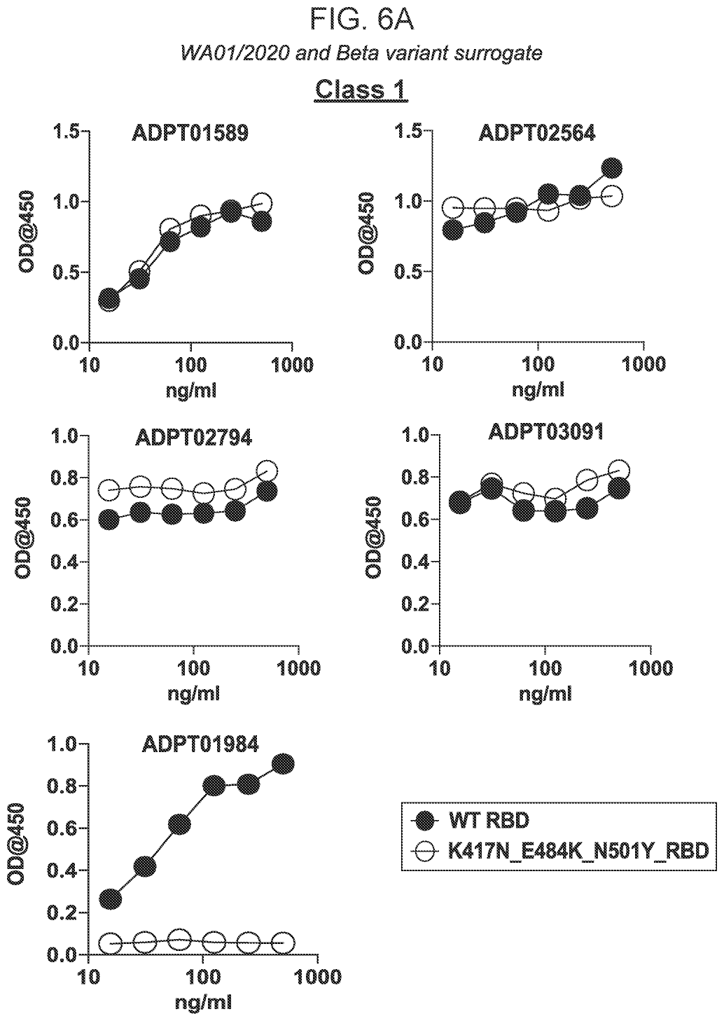

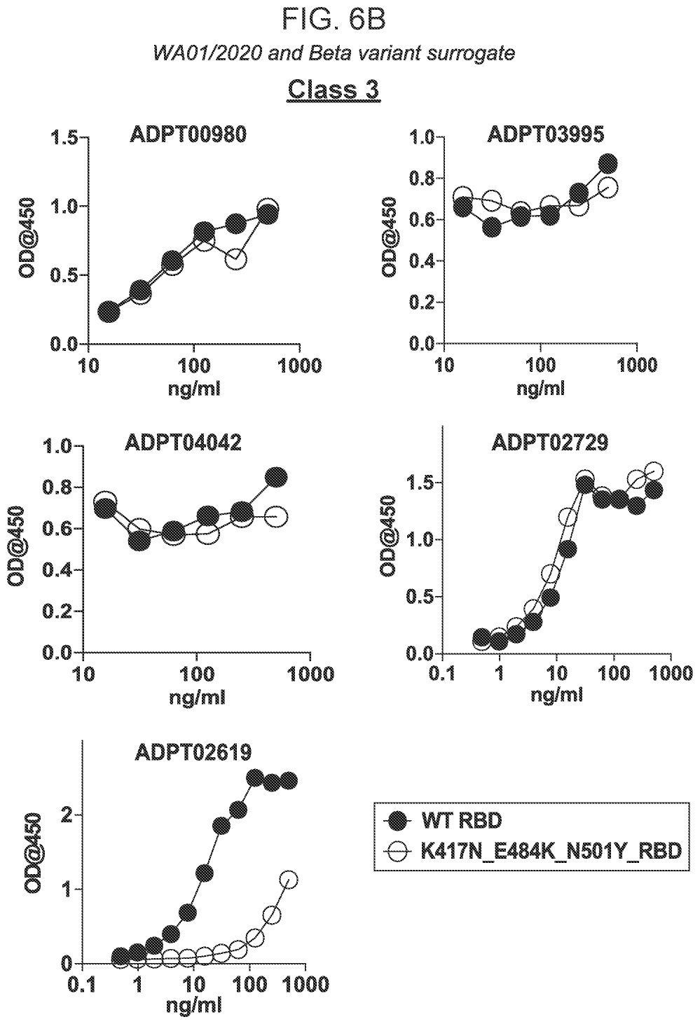

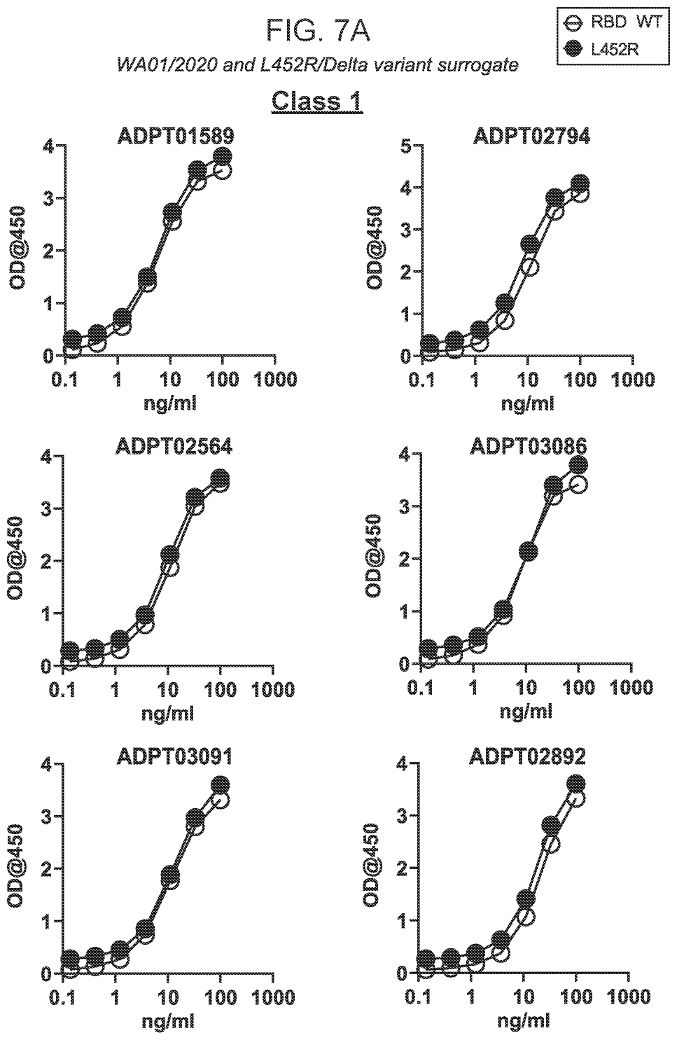

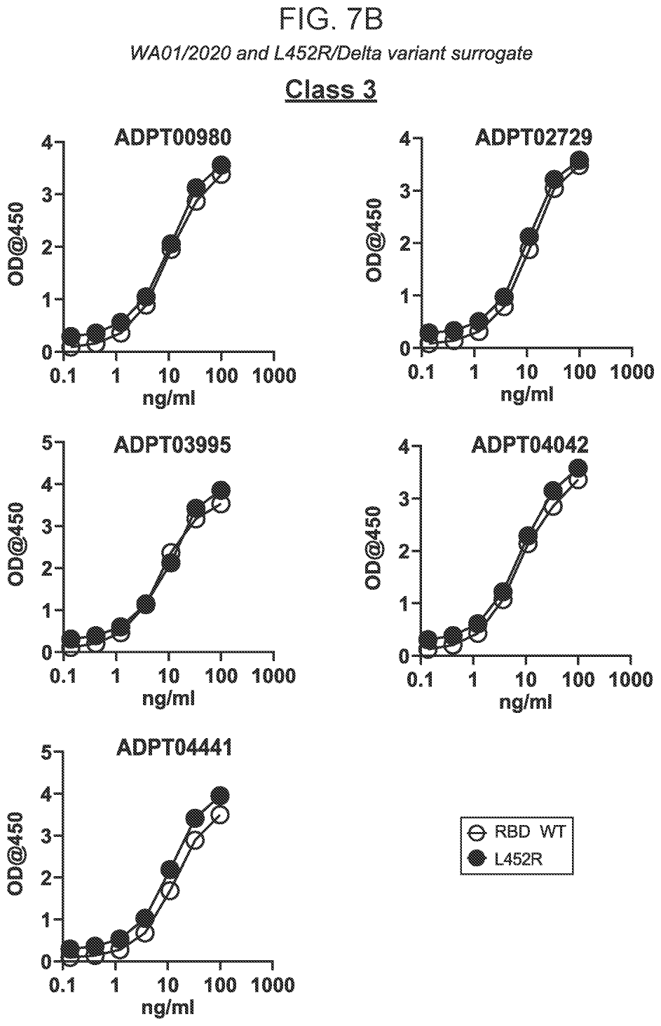

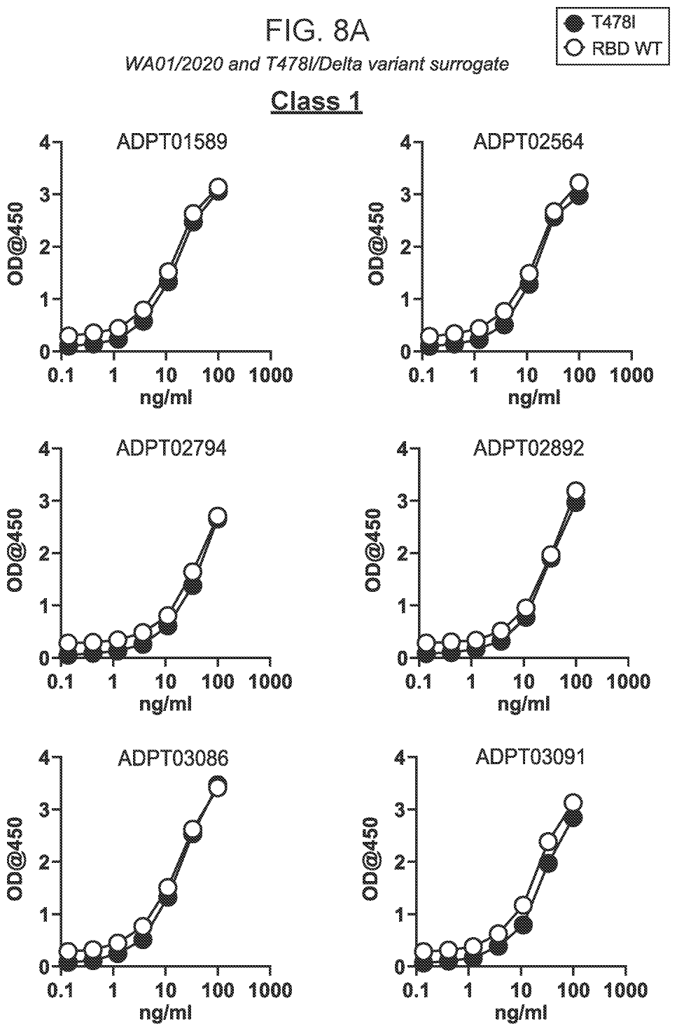

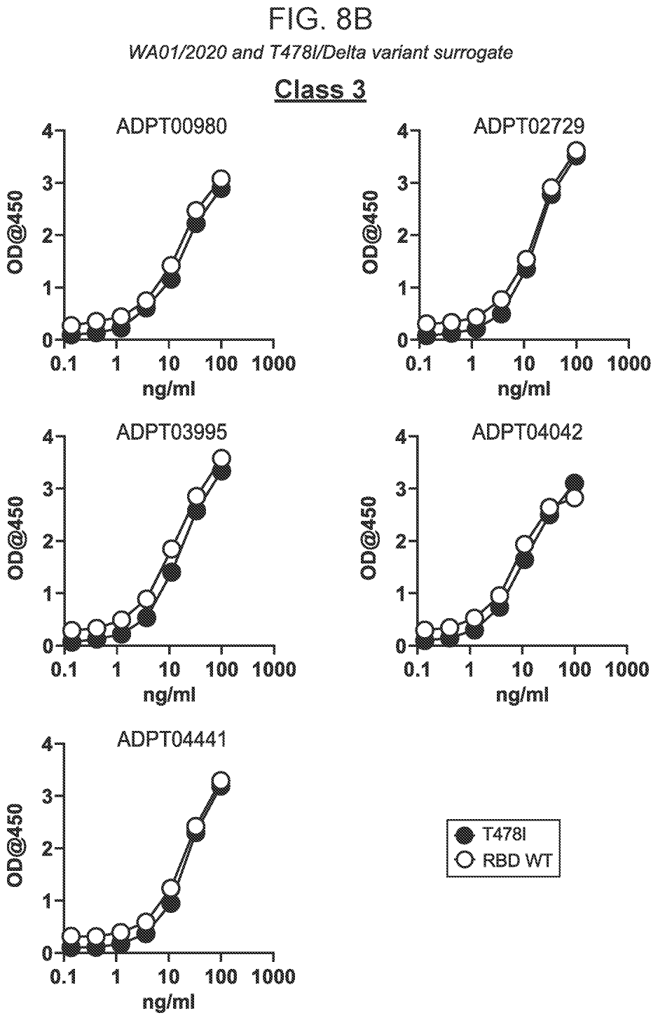

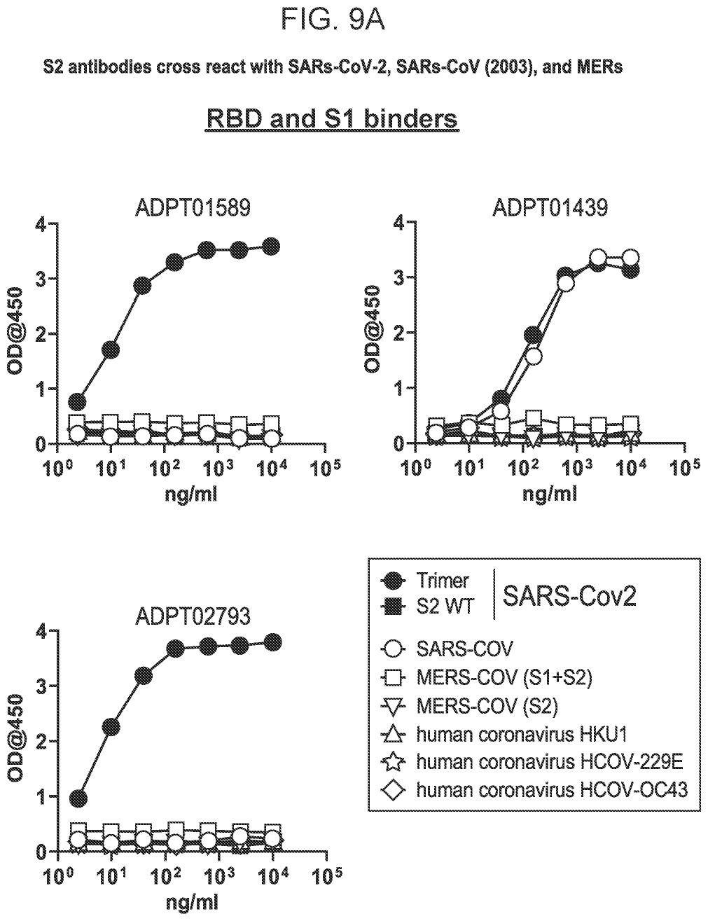

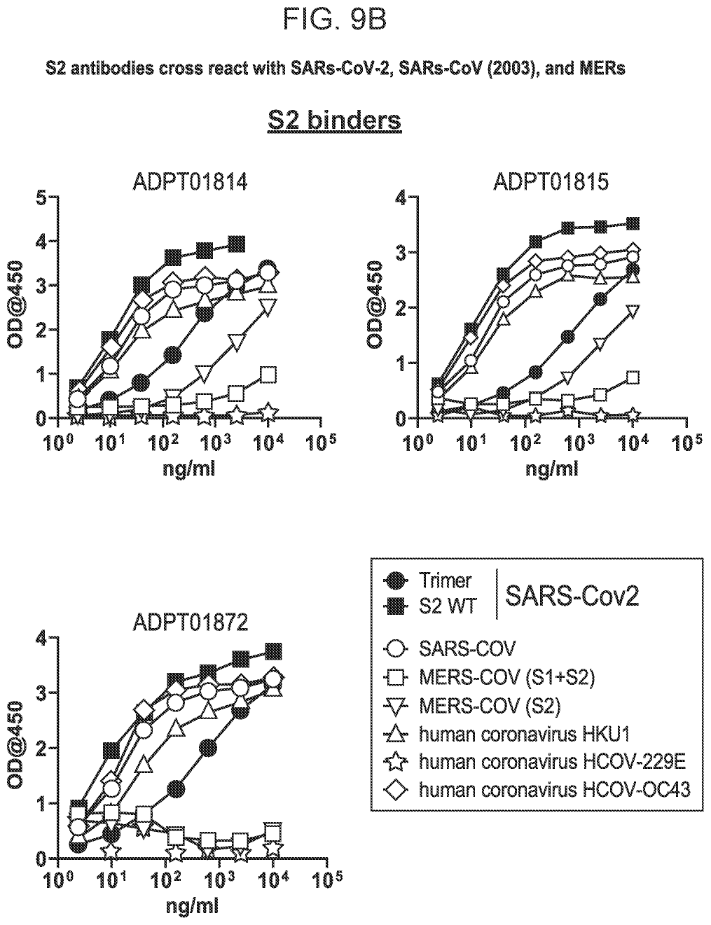

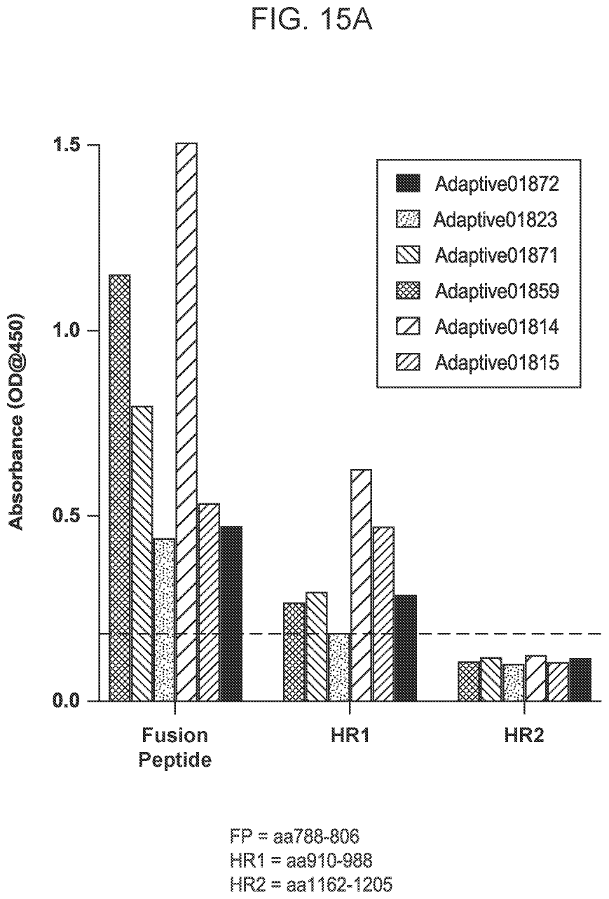

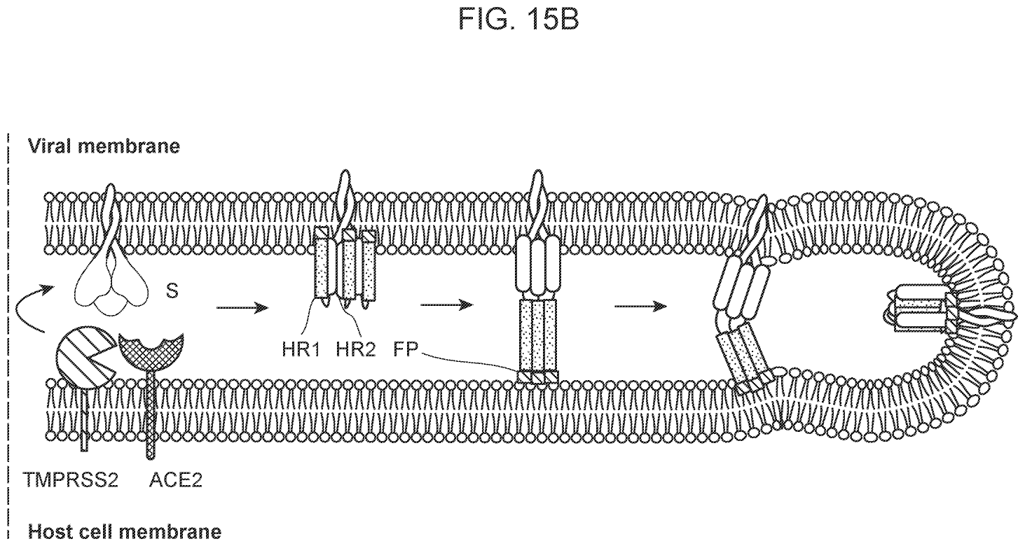

Results are shown in FIGS. 2-10 and 15. FIG. 2A: Antibodies react to RBD domain of the spike protein but do not bind to S2 domain or the nucleocapsid. Black bars in both graphs represent positive control. FIG. 2B: ELISA data of non-RBD/non-S2 antibodies. The antibodies bound to either Trimer alone or Trimer and S1 but not RBD, S2 or nucleocapsid suggesting they are specific to N-terminal domain. Black bars in both graphs represent positive control. FIG. 3A: Anti-RBD antibody candidates display pM affinity by ELISA. Representative graphs of a dose response ELISA with RBD specific antibodies. FIG. 3B: Representative sensorgrams of anti RBD antibodies confirming high affinity to RBD protein. FIG. 3C: Summary table with pM binding affinities of RBD antibodies by ELISA and Biocore. FIG. 4: ELISA screening data of antibodies isolated from a patient during acute phase of immune response. The majority of the antibodies reacted to trimer and S2 but not RBD, S1 or nucleocapsid. Two antibodies did not react spike or nucleocapsid. FIG. 5: Selected anti-S2 antibodies show high affinity binding by ELISA. The table shows a summary of EC₅₀ in pM. FIG. 6A: Dose response ELISA assay comparing reactivity of class 1 antibodies to RBD protein expressed by WA01/2020 SARs-CoV2 (WT) compared to those in the Beta variant (K417N, E484K and N501Y). FIG. 6B: Dose response ELISA assay comparing reactivity of class 3 antibodies to RBD protein expressed by WA01/2020 SARs-CoV2 (WT) compared to those in the Beta variant (K417N, E484K and N501Y). FIG. 7A: Dose response ELISA assay comparing reactivity of class 1 antibodies to RBD protein expressed by WA01/2020 SARs-CoV2 (WT) compared to that in the delta variant (L452R). FIG. 7: Dose response ELISA assay comparing reactivity of class 3 antibodies to RBD protein expressed by WA1/2020 SARs-CoV2 (WT) compared to that in the delta variant (1L452R). FIG. 8A: Dose response ELISA assay comparing reactivity of class 1 antibodies to RBD protein expressed by WA01/2020 SARs-CoV2 (WT) compared to that in the delta variant (T4781). FIG. 8B: Dose response ELISA assay comparing reactivity of class 3 antibodies to RBD protein expressed by WA01/2020 SARs-CoV2 (WT) compared to that in the delta variant (T4781). FIG. 9A: Dose response ELISA assay comparing reactivity of selected anti-RBD antibodies of Spike expressed by WA01/2020 SARs-CoV2 (WT) to that expressed by SARS-Cov1, MERS-Cov, HCOV-HKU1, HCOV-229E and HCOV-OC43. FIG. 9B and 9C/10A and 10B: Selected graphs of dose response ELISA assay comparing reactivity of selected anti-S2 antibodies with Spike expressed by WA1/2020 SARs-CoV2 (WT) to that expressed by SARS-Cov1, MERS-Cov, HCOV-HKU1, HCOV-229E and HCOV-OC43. FIG. 15A-15B: A) Elisa assay of S2 specific mAbs reacting to different domains of S2 protein. Peptides or short proteins corresponding to FP (aa788-806), HR1 (aa910-988) and HR2 (aa1162-1205). B) schematic representation of fusion between viral spike protein and ACEs receptor in the presence host enzyme TMPRSS2.

Additional data for this example is provided in the following table:

| | | | Heavy | | | Antigen |
|---|---|---|---|---|---|---|
| | | Light | chain | Structural | Biacore, Kd | Binding, EC50 |
| Antibody | Isotype | chain | germline | data | (pM) | (pM) |
| ADPT00508 | IgG3 | LV1-40 | V4-59 | Spike | 351 | 35 |
| | | | | RBD | | |
| ADPT00767 | IgG1 | KV1-39, | V1-3 | Spike | 24300 | 3470 |
| | | KV1D-39 | | RBD | | |
| ADPT00935 | IgG1 | KV3-20 | V3-53 | Spike | 406 | 24 |
| | | | | RBD | | |
| ADPT00937 | IgG1 | KV1-5 | V3-9 | Spike | 3190 | 217 |
| | | | | RBD | | |
| ADPT00941 | IgG1 | KV3-15 | V1-69 | Spike | 62 | 24 |
| | | | | RBD | | |
| ADPT00980 | IgG1 | KV1-39, | V3-30, | Spike | 870 | 23 |
| | | KV1D-39 | V3-30-5 | RBD, | | |
| | | | | Class III | | |
| ADPT01085 | IgG1 | LV4-69 | V1-69, | Unknown | NA | NA |
| | | | V1-69D | | | |
| ADPT01213 | IgG1 | KV3-15 | V1-45 | Spike | 1720 | 51 |
| | | | | RBD | | |
| ADPT01227 | IgG1 | KV3-15 | V1-45 | Spike | 2740 | 107 |
| | | | | RBD | | |
| ADPT01231 | IgG1 | KV1-9 | V7-4-1 | Spike | 2540 | 18 |
| | | | | RBD | | |
| ADPT01238 | IgG1 | LV3-25 | V7-4-1 | S1 | 27 | S1: 28; |
| | | | | | | Trimer: 69 |
| ADPT01439 | IgG1 | LV6-57 | V4-59 | Spike | 17.6 | 9.9 |
| | | | | RBD | | |
| ADPT01589 | IgG1 | KV1-33, | V3-53 | Spike | <4.7* | 36 |
| | | KV1D-33 | | RBD, | | |
| | | | | Class I | | |
| ADPT01671 | IgG1 | KV3-20 | V3-21 | S1 | 50.6 | TBD |
| ADPT01679 | IgG1 | LV2-14 | V2-5 | RBD | 1010 | 3 |
| ADPT01814 | IgG1 | LV1-40 | V4-59 | S2 | TBD | 77 |
| ADPT01815 | IgG1 | LV1-40 | V4-59 | S2 | TBD | 127 |
| ADPT01823 | IgA2 | KV3-15 | V3-21 | S2 | TBD | 614 |
| ADPT01826 | IgG1 | LV1-40 | V4-59 | S2 | TBD | TBD |
| ADPT01851 | IgG1 | LV1-40 | V4-59 | S2 | TBD | TBD |
| ADPT01856 | IgG3 | KV3-15, | V3-21 | S2 | TBD | TBD |
| | | KV3D-15 | | | | |
| ADPT01859 | IgG3 | KV3-15 | V3-21 | S2 | TBD | 1221 |
| ADPT01864 | IgG1 | LV1-40 | V4-59 | S2 | TBD | TBD |
| ADPT01867 | IgA2 | KV3-15 | V3-21 | S2 | TBD | 1094 |
| ADPT01870 | IgG1 | LV1-40 | V4-59 | S2 | TBD | TBD |
| ADPT01871 | IgG3 | KV3-15 | V3-21 | S2 | TBD | 103 |
| ADPT01872 | IgG3 | KV3-15 | V3-21 | S2 | TBD | 71 |
| ADPT01888 | IgG1 | KV3-15 | V3-21 | S2 | TBD | TBD |
| ADPT01915 | IgA2 | KV1-33, | V3-30, | Unknown | TBD | NA |
| | | KV1D-33 | V3-30-3 | | | |
| ADPT01959 | IgG1 | KV3-20 | V1-69 | Unknown | TBD | NA |
| ADPT01963 | IgG1 | LV1-51 | V1-46 | S2 | TBD | 41 |
| ADPT01969 | IgG1 | LV1-51 | V1-46 | S2 | TBD | 35 |
| ADPT01984 | IgA2 | LV7-46 | V3-66 | Spike | 74 | 25 |
| | | | | RBD, | | |
| | | | | Class I | | |
| ADPT02019 | IgG1 | LV3-25 | V3-30, | Trimer | 57 | S1: no binding; |
| | | | V3-30-5 | | | Trimer: 24 |
| ADPT02020 | IgG1 | LV2-14 | V1-2 | Spike | 1.1 | 88 |
| | | | | RBD, | | |
| | | | | Class II | | |
| ADPT02024 | IgG1 | LV3-21 | V1-24 | Trimer | 15600 | 1154 |
| ADPT02025 | IgA2 | LV2-23 | V1-24 | Spike | 7130 | 113 |
| | | | | RBD, | | |
| | | | | Class II | | |
| ADPT02050 | IgG1 | LV2-23 | V1-2 | Spike | 115 | 25 |
| | | | | RBD, | | |
| | | | | Class II | | |
| ADPT02075 | IgG1 | KV1-39, | V1-69, | Spike | <0.7 | 73 |
| | | KV1D-39 | V1-69D | RBD, | | |
| | | | | Class II | | |
| ADPT02080 | IgG1 | KV1-5 | V4-34 | S1 | 690 | 25 |
| ADPT02432 | IgG1 | KV1-39, | V1-69, | Trimer | 243 | S1: no binding; |
| | | KV1D-39 | V1-69D | | | Trimer: 24 |
| ADPT02564 | IgG1 | KV3-20 | V1-69 | Spike | 12 | 23 |
| | | | | RBD, | | |
| | | | | Class I | | |
| ADPT02598 | IgG1 | KV1-12, | V4-4 | S1 | 8.23 | S1: 84; |
| | | KV1D-12 | | | | Trimer: 43 |
| ADPT02606 | IgG1 | KV3-15 | V3-21 | Trimer | 7.87 | S1: no binding; |
| | | | | | | Trimer: 57 |
| ADPT02619 | IgG1 | KV1-39, | V3-9 | Spike | 105 | 37 |
| | | KV1D-39 | | RBD, | | |
| | | | | Class III | | |
| ADPT02646 | IgG1 | KV2-24 | V1-24 | S1 | 2.96 | S1: 33; |
| | | | | | | Trimer: 24 |
| ADPT02788 | IgG1 | LV2-8 | V1-24 | S1 | TBD | S1: 58; |
| | | | | | | Trimer: 21 |
| ADPT02793 | IgG1 | LV2-14 | V1-24 | S1 | 398 | S1: 113; |
| | | | | | | Trimer: 68 |
| ADPT02794 | IgG1 | LV1-47 | V1-69 | Spike | 232 | 22 |
| | | | | RBD, | | |
| | | | | Class I | | |
| ADPT02854 | IgG1 | KV2-30 | V1-69, | Trimer | 112 | S1: no binding; |
| | | | V1-69D | | | Trimer: 152 |
| ADPT02866 | IgG1 | KV4-1 | V1-69, | Trimer | 154 | S1: no binding; |
| | | | V1-69D | | | Trimer: 182 |
| ADPT03091 | IgG1 | KV3-20 | V1-58 | Spike | 4 | 30 |
| | | | | RBD, | | |
| | | | | Class I | | |
| ADPT03995 | IgG1 | KV1-5 | V1-69 | Spike | 57 | 21 |
| | | | | RBD, | | |
| | | | | Class III | | |
| ADPT04042 | IgG1 | LV2-14 | V2-5 | Spike | 36 | 21 |
| | | | | RBD, | | |
| | | | | Class III | | |
| ADPT04441 | IgG1 | LV3-25 | V3-9 | Spike | 22 | 50 |
| | | | | RBD, | | |
| | | | | Class III | | |
| ADPT02892 | IGG1 | LV2-11 | V1-69, | Spike | 5 | 89 |
| | | | V1-69D | RBD, | | |
| | | | | Class I | | |
| ADPT03086 | IGG1 | LV2-23 | V4-59 | Spike | 242 | 30 |
| | | | | RBD, | | |
| | | | | Class 1 | | |
| ADPT02729 | IGG1 | LV6-57 | V2-26 | Spike | 15 | 37 |
| | | | | RBD, | | |
| | | | | Class III | | |
| ADPT02706 | IGG1 | KV1-33, | V1-69 | Spike | 29 | 17 |
| | | KV1D-33 | | RBD, | | |
| | | | | Class III | | |

### Example 4—Epitope Mapping and Specificity Testing

The specificity of the antibodies to SARS-CoV-2 was evaluated using a protein membrane array consisting of a library of over 4000 cell surface proteins. Epitope mapping was performed using shotgun mutagenesis consisting of a library of cells engineered to express SARS-CoV-2 containing single amino acid substitutions of alanine at each position. Critical amino acids for antibody binding to SARS-CoV-2 were identified using flow cytometry.

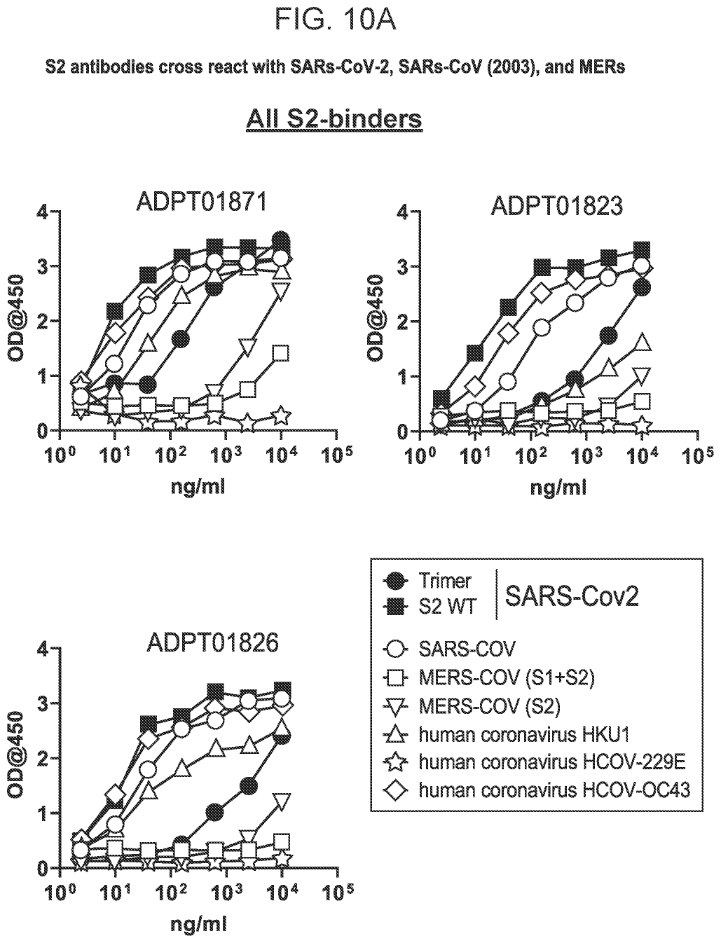

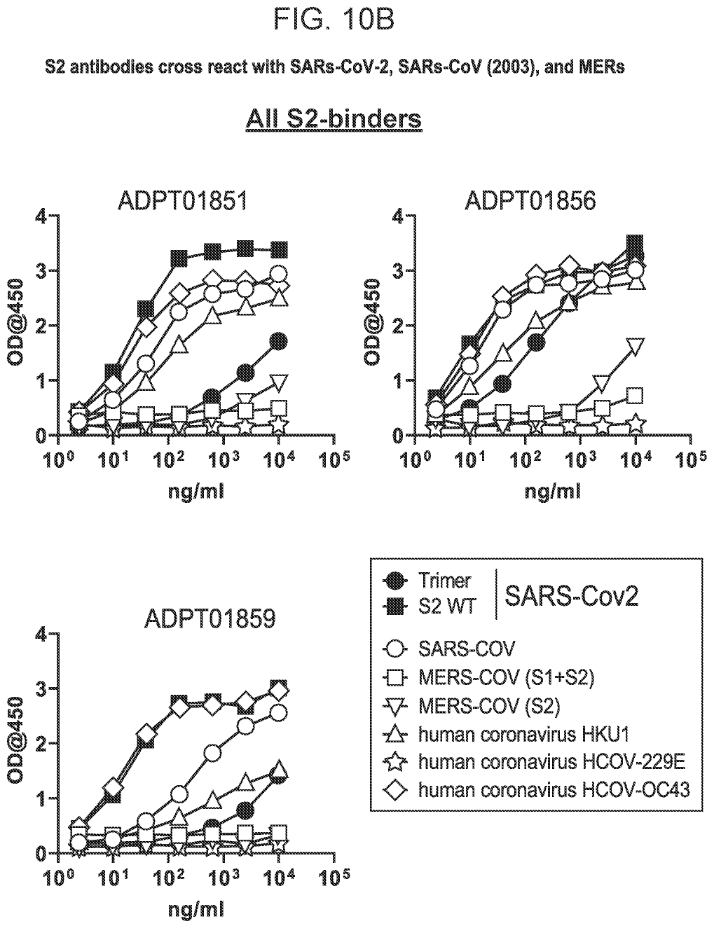

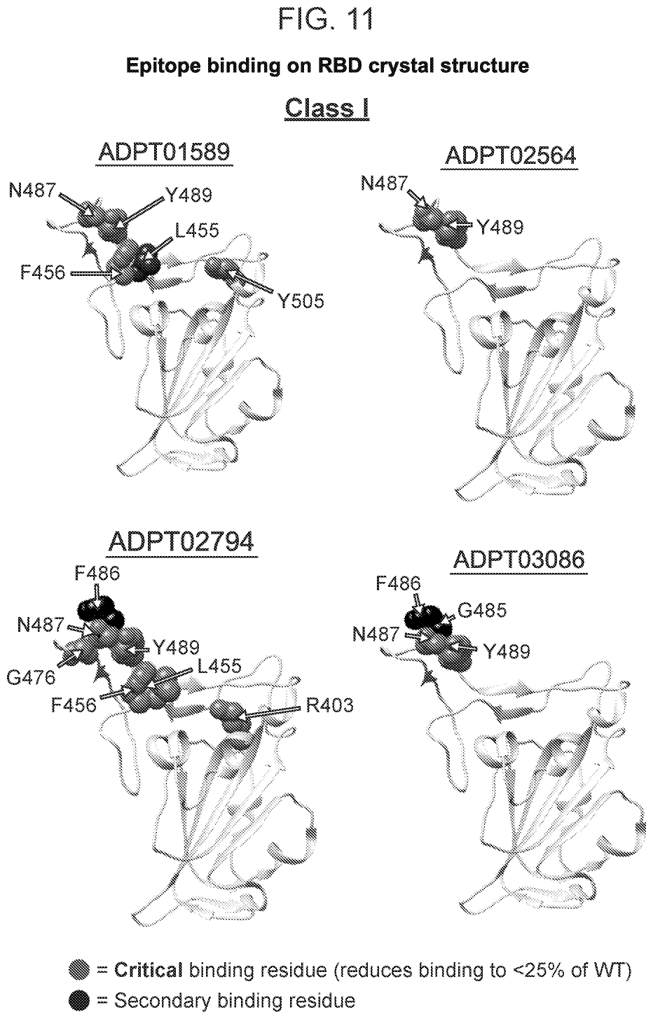

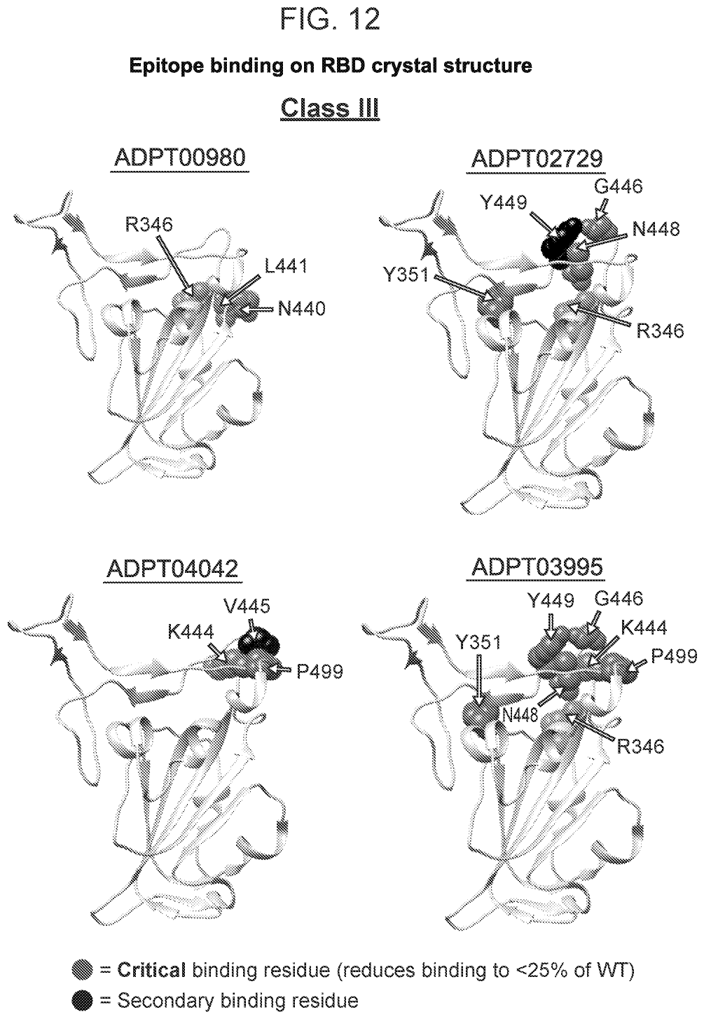

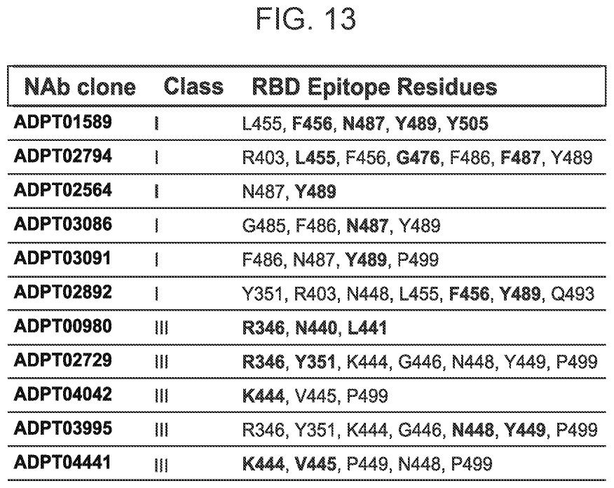

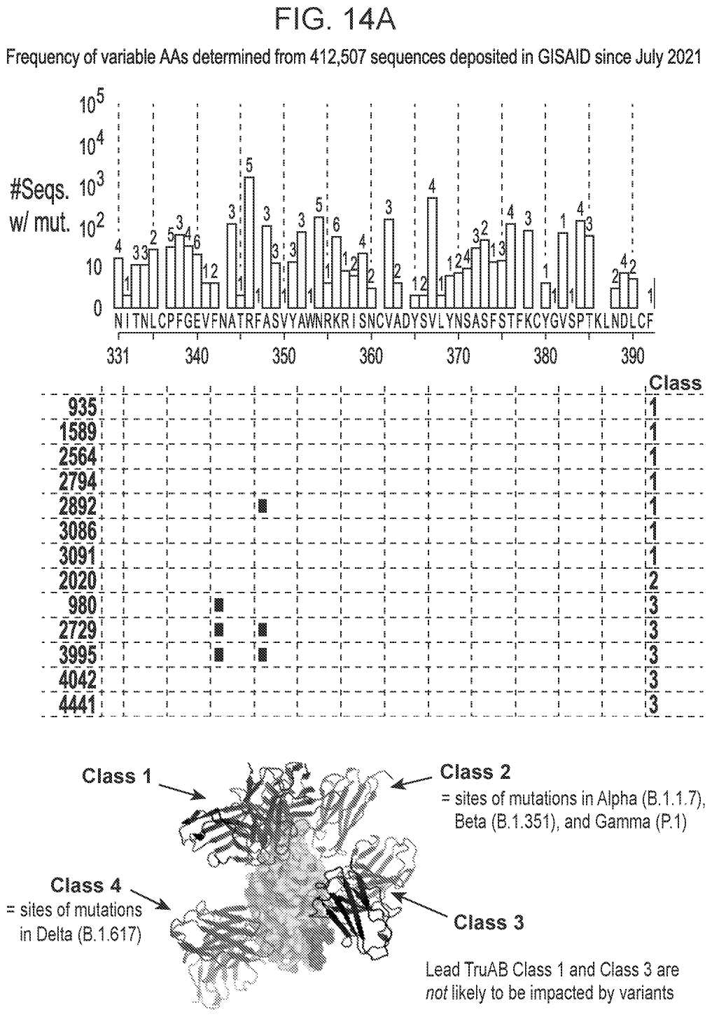

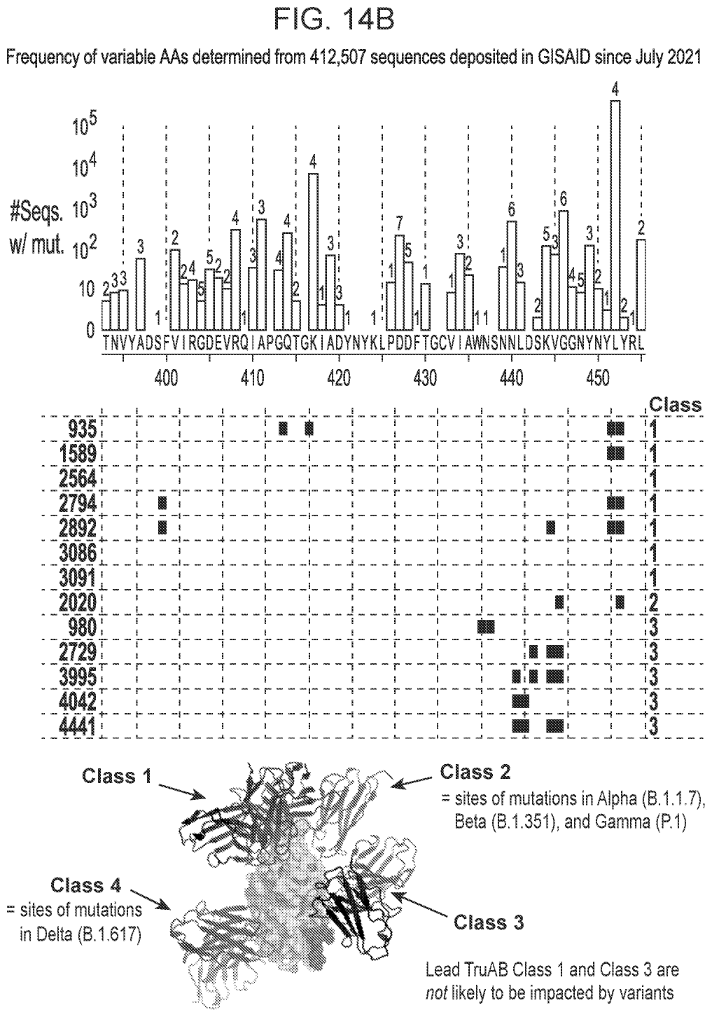

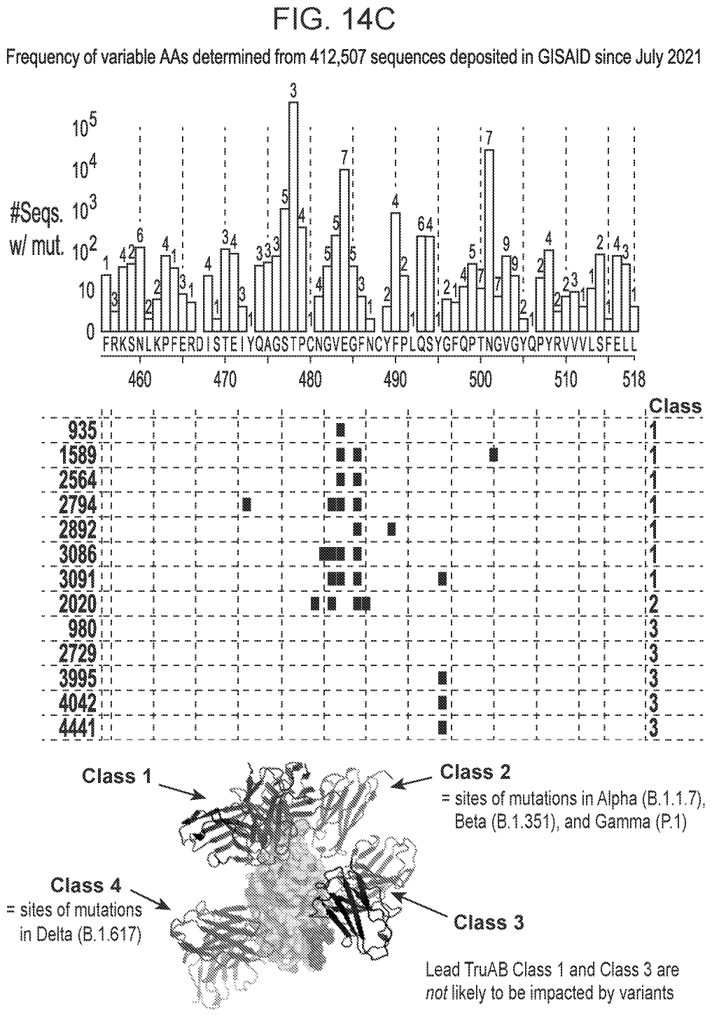

Results are shown in FIGS. 11-14. FIG. 11: Visualization of critical residues for class I monoclonal antibodies (mAbs) binding to RBD protein. Critical residues (lighter spheres) were visualized on a crystal structure of the receptor binding domain of the Spike protein. Secondary residues (darker spheres) that may contribute to binding are also shown. FIG. 12: Visualization of critical residues for class III monoclonal antibodies (mAbs) binding to RBD protein. Critical residues (lighter spheres) were visualized on a crystal structure of the receptor binding domain of the Spike protein. Secondary residues (darker spheres) that may contribute to binding are also shown. FIG. 13: A table summarizing the RBD epitope residues for the antibodies shown in FIGS. 11 and 12. FIG. 14A-14C: A graph showing the frequency of variable amino acids in SARS-CoV-2 variants (top) and epitope residues for selected antibodies (bottom), indicating that the antibodies are not likely to be impacted by SARS-CoV-2 variants.

Additional epitope mapping data is provided in the following table:

| Antibody | Critical Residues |
|---|---|
| ADPT02019 | K147, E156, R246, L249, G257 |
| ADPT01238 | K182, G184, N185, F186, K187, N211, V213, D215 |
| ADPT02080 | K97, K187, V213, R214 |
| ADPT02854 | L18, T19, A67, I68, F79, N81, D138, L249 |
| ADPT01671 | Y38, K41, D228 |
| ADPT02866 | L18, F79 |
| ADPT02793 | Y145, K147, W152, R246, Y248, P251 |
| ADPT02025 | Y145, K147, W152, R246, Y248 |
| ADPT00935 | K417, D420, L455, F456, N487 |
| ADPT02075 | F456, E484, G485, Y489, F490 |
| ADPT00980 | R346, N440, L441 |
| ADPT02020 | Y449, F456, E484, F486, Y489, F490 |
| ADPT00941 | S349, E484, F490 |
| ADPT01984 | F456, E484, G485, F486, Y489 |
| ADPT01589 | L455, F456, N487, Y489, Y505 |
| ADPT02050 | E484, G485, F486 |
| ADPT04042 | K444, V445, P499 |
| ADPT03995 | R346, Y351, K444, G446, N448, Y449, P499 |
| ADPT02729 | R346, Y351, G446, N448, Y449 |
| ADPT04441 | K444, V445, P449, N448, P499 |
| ADPT02892 | Y351, R403, N448, L455, F456, Y489, Q493 |
| ADPT02564 | N487, Y489 |
| ADPT02794 | R403, L455, F456, G476, F486, N487, Y489 |
| ADPT03091 | F486, N487, Y489, P499 |
| ADPT03086 | G485, F486, N487, Y489 |

### Example 5—ACE2 Blockade and Pseudovirus Neutralization Assay

Antibodies with confirmed antigen specificity to one or more proteins of SARS-CoV-2 were assessed for blockade of ACE2 via ELISA and pseudovirus neutralization assay. The ability of candidate antibodies to block the interaction between viral RBD protein and human ACE surface receptor was measured by ELISA. IC50 was calculated based performing inhibition assay with serial dilution of mAbs starting from 30 μg/ml and three-fold dilution for a total of 10 points in duplicate. Anti-S antibody, Clone 6D11F2 & human IgG were included as positive and negative controls respectively. Non RBD antibodies (S1, S2, trimer alone or nucleocapsid) that did not block ACE were advanced to both live or Pseudovirus because this antibodies could use other mechanisms to block the virus.

Pseudovirus neutralization assay: A lentiviral pseudotype bearing spike viral protein expressing a firefly luciferase read-out was used to screen for functional antibody responses against SARS-CoV-2 under BSL2 laboratory conditions. ACE2/TMPRSS2 expressing cell line was used as target cells. IC50 of selected mAbs was determined by performing a serial dilution starting from 0.1-10ug/ml and three-fold dilution for a total of 10 dilutions in triplicate. Anti-S antibody, Clone 6D11F2 & human IgG will be included as positive/negative control.

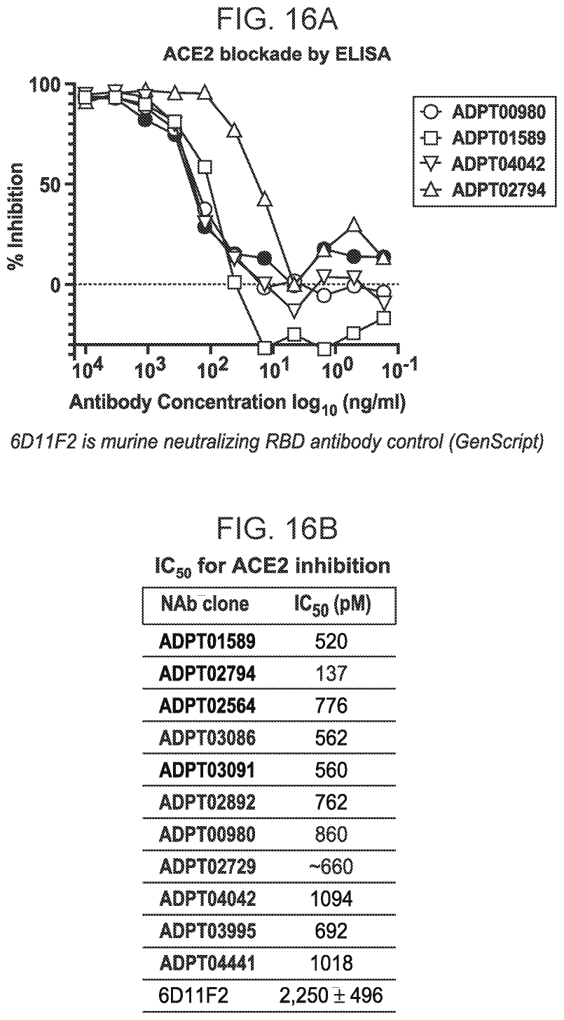

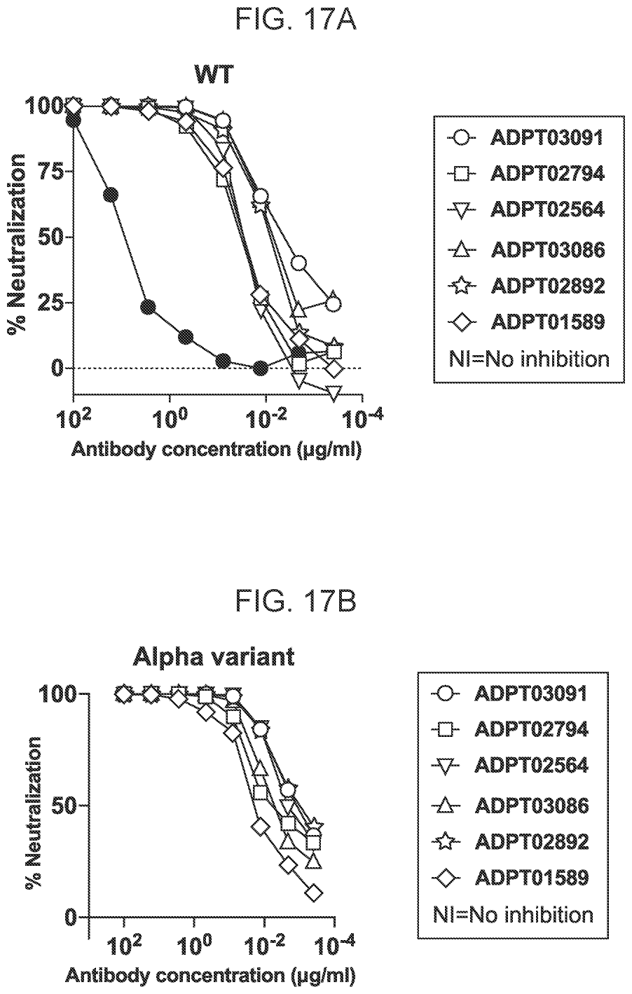

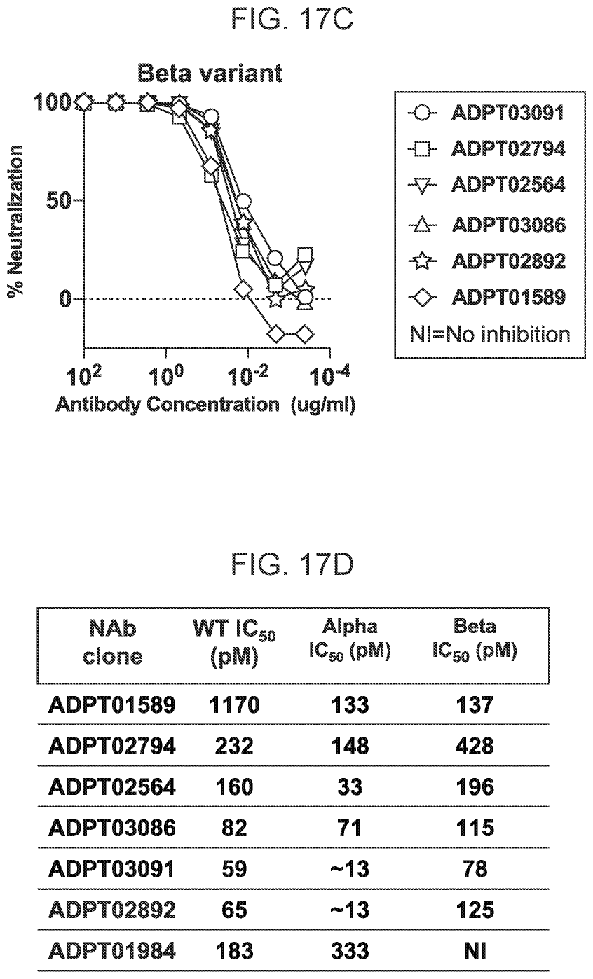

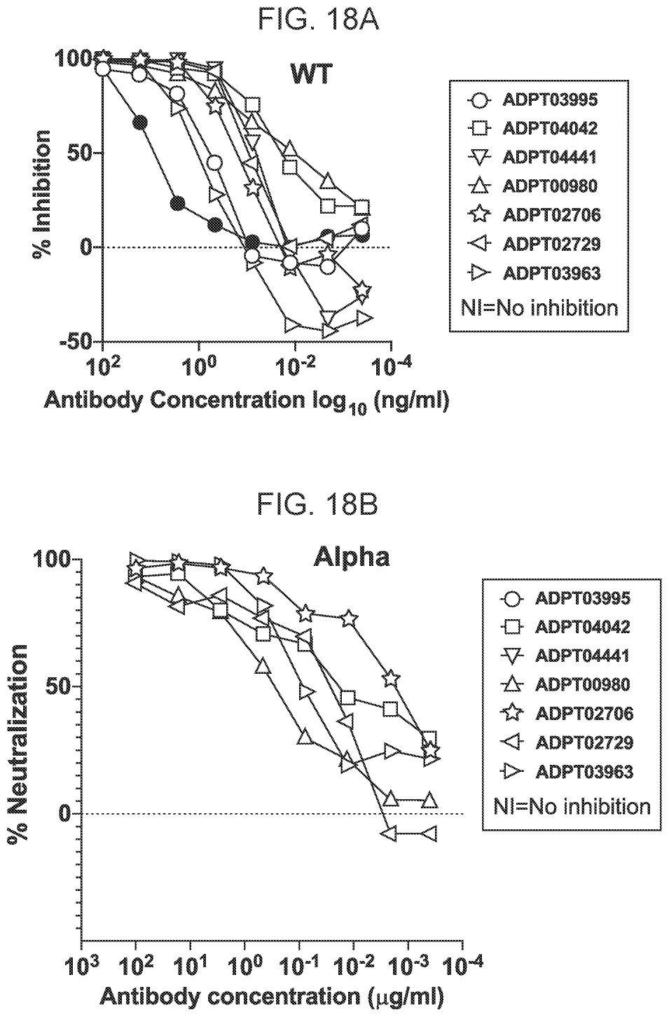

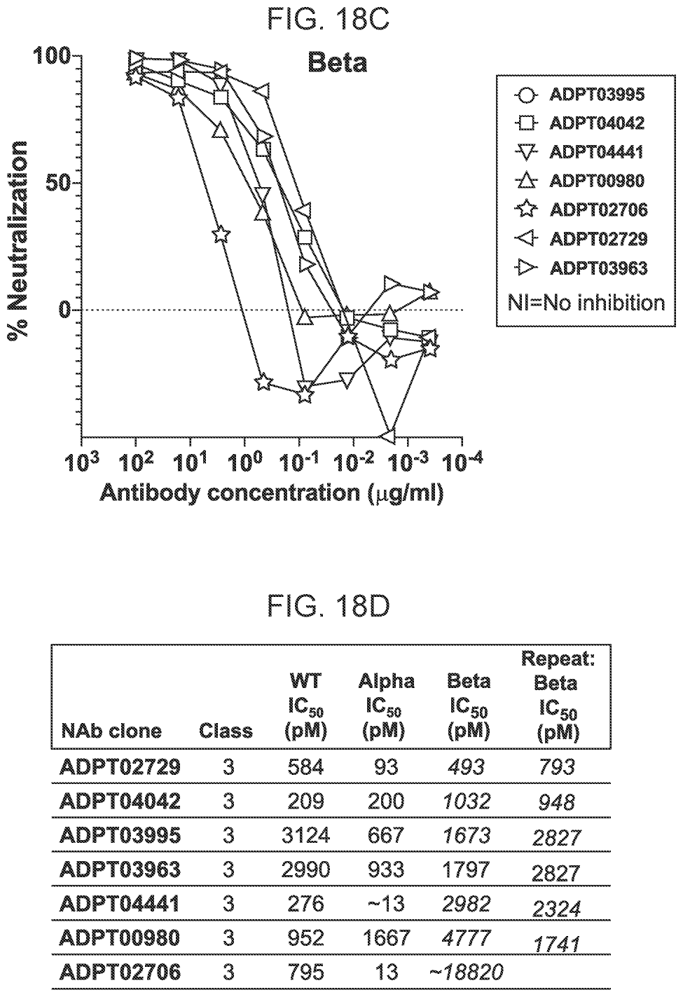

Results are shown in FIGS. 16-18. FIG. 16A: Representative graph of dose blockade of the ability of RBD specific antibodies to inhibit spike binding to ACE protein. Percent inhibition was calculated based on control wells with no antibody. 6D11F2 was used as a positive control. FIG. 161B: Table summary IC₅₀ in pM of RBD specific antibodies blocking spike/ACE interaction. FIG. 17A-17C: Dose response graphs class 1 anti-RBD antibodies ability to inhibit pseudovirus invasion of 293T cells overexpressing ACE and TMPRSS2. Percent inhibition calculated based on no antibody wells as 100%. Pseudovirus inhibition was done with WA01/2020 SARs-CoV2 (WT) (17A), alpha variant (17B) and beta variant (17C). FIG. 17D: Table summary IC₅₀ in pM of class 1 RBD specific antibodies inhibiting different variants of SARs-CoV2 pseudovirus. FIG. 18A-18C: Dose response graphs class 3 anti-RBD antibodies ability to inhibit pseudovirus invasion of 293T cells overexpressing ACE and TMPRSS2. Percent inhibition calculated based on no antibody wells as 100%. Pseudovirus inhibition was done with WA01/2020 SARs-CoV2 (WT) (18A), alpha variant (18B) and beta variant (18C). FIG. 180: Table summary IC₅₀ in pM of class 3 RBD specific antibodies inhibiting different variants of SARs-CoV2 pseudovirus.

Additional blockade and pseudovirus neutralization data is provided in the following table. NI=No Inhibition by the antibody of binding or the virus. NA or empty=not tested.

Neut,

| | ACE2 | Pseudo Neut, | Pseudo Neut, | Pseudo Neut, | Pseudo Neut, |
|---|---|---|---|---|---|
| | Blockade, | SARS-CoV | WA1/2020 | B1.1.7 IC50 | B1.351 IC50 |
| Antibody | IC50 (pM) | 2003 IC50 (nM) | IC50 (pM) | (pM) | (pM) |
| ADPT00508 | 971 | NI | 11046 | TBD | TBD |
| ADPT00767 | 20900 | TBD | TBD | TBD | TBD |
| ADPT00935 | 830 | NI | 420 | 1167 | NI |
| ADPT00937 | TBD | TBD | NA | NA | NA |
| ADPT00941 | 960 | NI | 610 | 533 | NI |
| ADPT00980 | 860 | NI | 952 | 1,667 | 1,741 |
| ADPT01085 | NA | NA | NA | NA | NA |
| ADPT01213 | 7740 | NA | 90600 | TBD | TBD |
| ADPT01227 | 14500 | NA | 115933 | TBD | TBD |
| ADPT01231 | 3690 | NA | 95000 | TBD | TBD |
| ADPT01238 | NA | NA | NA | NA | NA |
| ADPT01439 | 117 | 29 | 30237 | NA | NA |
| ADPT01589 | 520 | NI | 1,170 | 133 | 137 |
| ADPT01671 | NA | NA | NA | NI | NI |
| ADPT01679 | 70 | NA | >163 | NA | NA |
| ADPT01814 | NA | 6.5 | 170.8 | 237 | 32 |
| ADPT01815 | NA | 26 | 10.2 | NI | 345 |
| ADPT01823 | NA | 210 | NI | 850 | 133 |
| ADPT01826 | NA | TBD | TBD | TBD | TBD |
| ADPT01851 | NA | NI | NI | TBD | TBD |
| ADPT01856 | NA | >111 | TBD | TBD | TBD |
| ADPT01859 | NA | 115 | 889 | 230 | 73 |
| ADPT01864 | NA | NA | NA | TBD | TBD |
| ADPT01867 | NA | 135 | NI | TBD | TBD |
| ADPT01870 | NA | TBD | NA | TBD | TBD |
| ADPT01871 | NA | 30 | 440 | 110 | 208 |
| ADPT01872 | NA | 27 | 133 | 130 | 75 |
| ADPT01888 | NA | >111 | >111 | TBD | TBD |
| ADPT01915 | NA | NI | NI | TBD | TBD |
| ADPT01959 | NA | NI | NI | TBD | TBD |
| ADPT01963 | NA | 69 | NI | TBD | TBD |
| ADPT01969 | NA | 97 | NI | TBD | TBD |
| ADPT01984 | 512 | NI | 183 | 333 | NA |
| ADPT02019 | NA | NA | NA | NA | NA |
| ADPT02020 | 1408 | NI | 214 | 13 | NA |
| ADPT02024 | NA | NA | NA | NA | NA |
| ADPT02025 | TBD | NA | NA | NA | NA |
| ADPT02050 | 727 | NI | 427 | 200 | NA |
| ADPT02075 | 930 | NI | 783 | ~7 | NA |
| ADPT02080 | NA | NA | NA | NA | NA |
| ADPT02432 | NA | NA | NA | NA | NA |
| ADPT02564 | 776 | NI | 160 | 33 | 196 |
| ADPT02598 | NA | NA | NA | NA | NA |
| ADPT02606 | NA | NA | NA | NA | NA |
| ADPT02619 | 479 | NI | 213 | ~7 | NA |
| ADPT02646 | NA | NA | NA | NA | NA |
| ADPT02788 | NA | NA | NA | NA | NA |
| ADPT02793 | NA | NA | NA | NA | NA |
| ADPT02794 | 137 | NI | 232 | 148 | 428 |
| ADPT02854 | NA | NA | NA | NA | NA |
| ADPT02866 | NA | NA | NA | NA | NA |
| ADPT03091 | 560 | NI | 59 | ~13 | 78 |
| ADPT03995 | 692 | NI | 3,124 | 667 | 2,827 |
| ADPT04042 | 1094 | NI | 209 | 200 | 948 |
| ADPT04441 | 1018 | NI | 276 | ~13 | 2,324 |
| ADPT02892 | 762 | NI | 65 | ~13 | 125 |
| ADPT03086 | 562 | NI | 82 | 71 | 115 |
| ADPT02729 | ~660 | NI | 584 | 93 | 793 |
| ADPT02706 | 510 | NI | 795 | 13 | >18,000 |

### Example 6—Live Virus Neutralization Assay

Neutralization of SARS-CoV-2 was determined using a microneutralization assay. A SARS-CoV-2 viral stock generated from in vitro passaging of strain USA-WA1/2020 (BEI Resources Lot No. 70035360 or equivalent) from a qualified lot was be used in the MN assay. The candidate antibodies were analyzed with seven-point four-fold serial dilution from a defined starting concentration. The primary assay endpoint was IC₅₀, which is the antibody concentration that neutralizes 50% of the input virus. This assay was performed following a third party, Battelle Standard Operating Procedures. Compared to the “no virus” control and “virus only” controls within the assay, the viral infectivity post antibody neutralization was quantified using an in situ Enzyme-Linked Immunosorbent Assay (ELISA) readout performed by following Battelle SOP. A neutralizing monoclonal antibody (mAb) specifically targeting the SARS-CoV-2 spike protein was be used as PC and a non-neutralizing antibody was used as NC in the MN assay. Selected antibodies that inhibited pseudovirus and not live virus was still selected because it suggested that mechanism of action might be present in the specific cell type used replicate the virus.

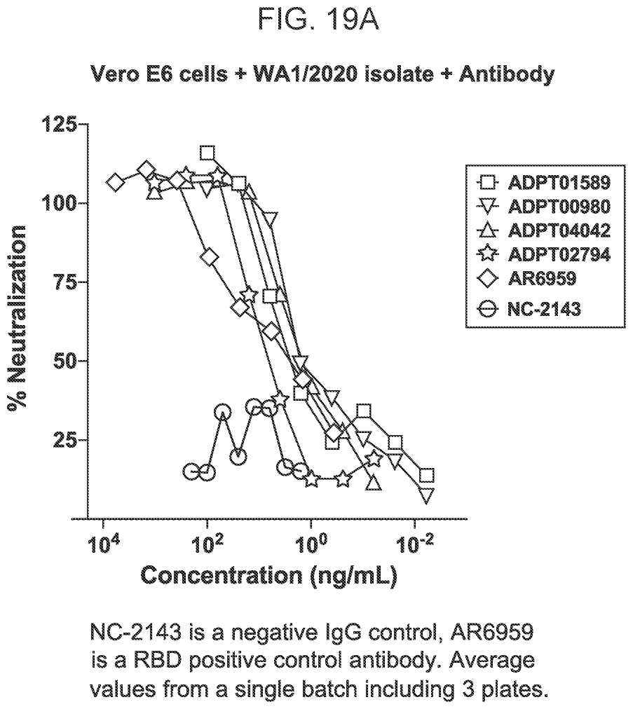

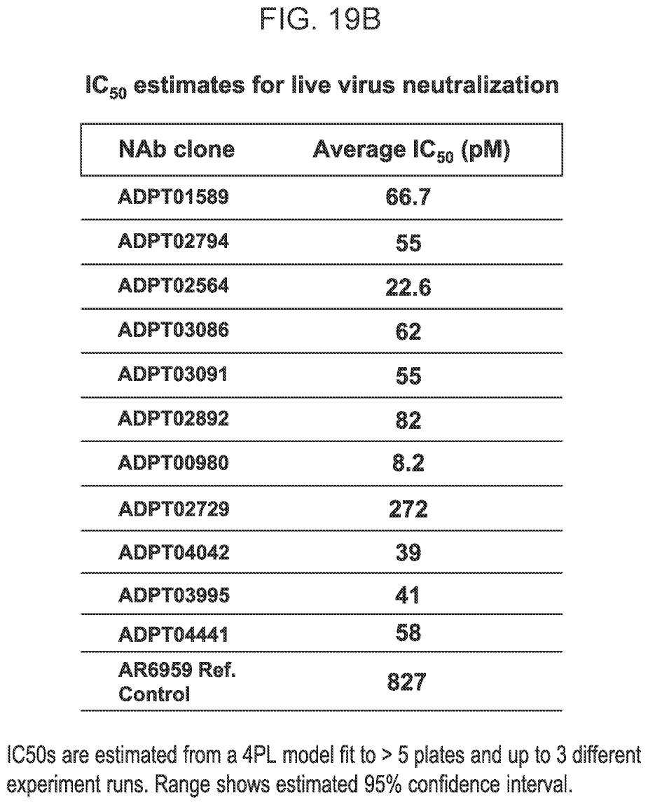

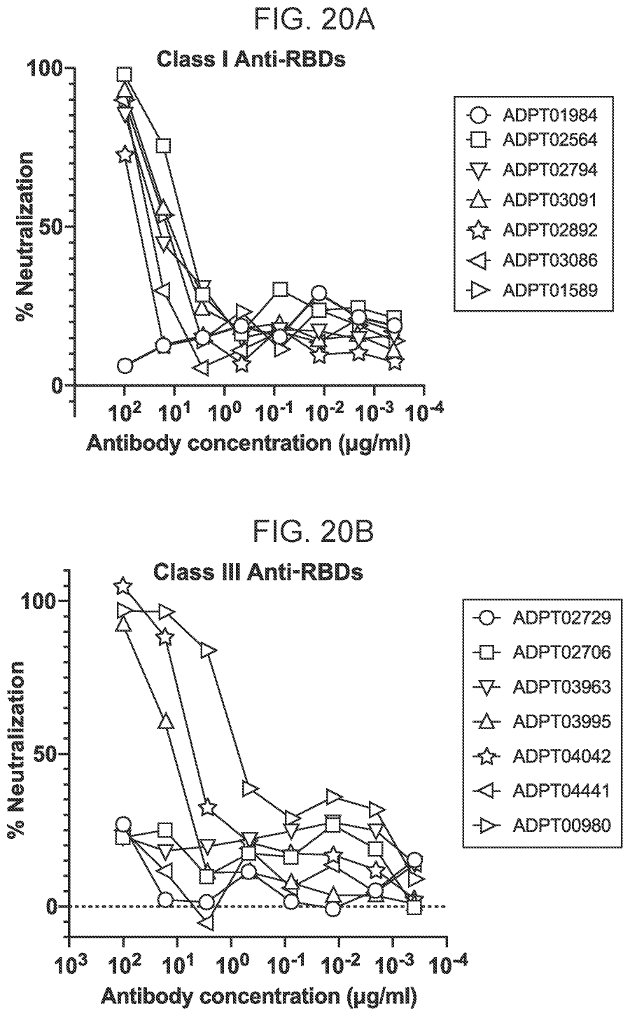

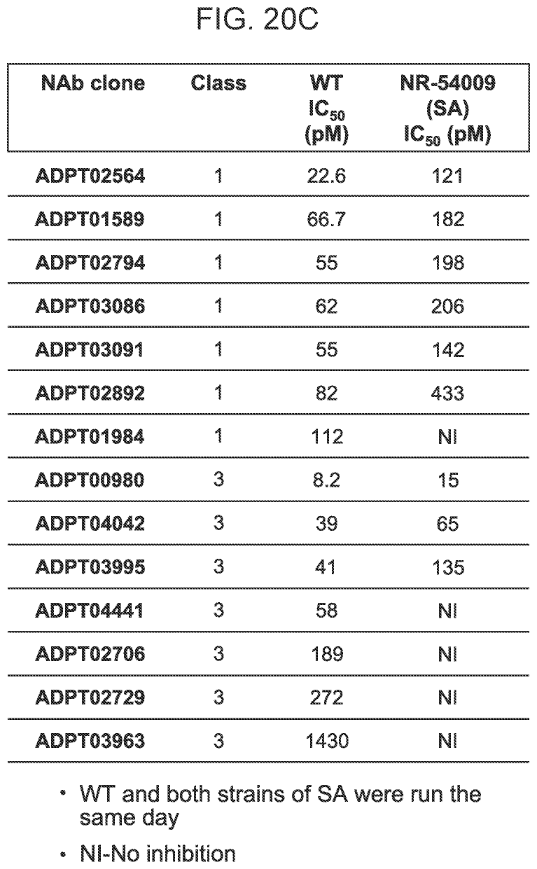

Results are shown in FIGS. 19-20. FIG. 19A: Dose response graphs anti-RBD antibodies inhibition of WA01/2020 SARs-CoV2 live virus invasion of Vero E6 cells. AR6959 was used as negative control and NC-2143 was used a negative control. Percent inhibition was calculated based on no antibody control wells as 100% infection. FIG. 19B: Table summary IC₅₀ in pM of RBD specific antibodies inhibiting of WA01/2020 SARs-CoV2 infection of Vero E6 cells. FIG. 20A-20B: Dose response graphs of anti-RBD class 1 (A) and class 3 (B) antibodies inhibition of beta variant of SARs-CoV2 live virus invasion of Vero E6 cells. Percent inhibition was calculated based on no antibody control wells as 100% infection. FIG. 20C: Table summary IC₅₀ in pM of class 1 and 3 RBD specific antibodies inhibiting of Beta variant of SARs-CoV2 infection of Vero E6 cells.

Additional live virus neutralization data is provided in the following table. NI=No Inhibition by the antibody of binding or the virus.

| | Live Neut, | Live Neut, |
|---|---|---|
| | WA1/2020 | B1.351 |
| Antibody name | IC50 (pM) | IC50 (pM) |
| ADPT00508 | 6130 | TBD |
| ADPT00767 | 1730 | TBD |
| ADPT00935 | 64 | TBD |
| ADPT00937 | 140 | TBD |
| ADPT00941 | 24 | NI |
| ADPT00980 | 25 | 83 |
| ADPT01085 | TBD | TBD |
| ADPT01213 | 2590 | TBD |
| ADPT01227 | 1490 | TBD |
| ADPT01231 | 9510 | TBD |
| ADPT01238 | 400 | TBD |
| ADPT01439 | TBD | TBD |
| ADPT01589 | 43 | 167 |
| ADPT01671 | 9 | TBD |
| ADPT01679 | 19 | TBD |
| ADPT01814 | NI | TBD |
| ADPT01815 | NI | TBD |
| ADPT01823 | NI | TBD |
| ADPT01826 | NI | TBD |
| ADPT01851 | NI | TBD |
| ADPT01856 | NI | TBD |
| ADPT01859 | NI | TBD |
| ADPT01864 | NI | TBD |
| ADPT01867 | NI | TBD |
| ADPT01870 | NI | TBD |
| ADPT01871 | NI | TBD |
| ADPT01872 | NI | TBD |
| ADPT01888 | NI | TBD |
| ADPT01915 | NI | TBD |
| ADPT01959 | NI | TBD |
| ADPT01963 | NI | TBD |
| ADPT01969 | NI | TBD |
| ADPT01984 | 150 | NI |
| ADPT02019 | 107 | TBD |
| ADPT02020 | 14 | NI |
| ADPT02024 | 95 | TBD |
| ADPT02025 | 118 | TBD |
| ADPT02050 | 36 | TBD |
| ADPT02075 | 12 | TBD |
| ADPT02080 | 169 | TBD |
| ADPT02432 | 26 | TBD |
| ADPT02564 | 7 | 121 |
| ADPT02598 | 586 | TBD |
| ADPT02606 | 474 | TBD |
| ADPT02619 | 118 | TBD |
| ADPT02646 | 19 | TBD |
| ADPT02788 | 117 | TBD |
| ADPT02793 | 77 | TBD |
| ADPT02794 | 17 | TBD |
| ADPT02854 | 164 | TBD |
| ADPT02866 | 649 | TBD |
| ADPT03091 | 8 | 172 |
| ADPT03995 | 51 | 135 |
| ADPT04042 | 15 | 140 |
| ADPT04441 | 85 | NI |
| ADPT02892 | 10 | 403 |
| ADPT03086 | 7 | 210 |
| ADPT02729 | 111 | TBD |
| ADPT02706 | 171 | NI |

### Example 7—In Vivo Studies

In vivo studies were performed with Golden Syrian Hamsters. Hamsters were acclimated for 10 days upon arrival and divided into 5 animals per group. Animals were treated intraperitoneally with antibodies on day −2 (SD −2)—48 hours before challenge. Throughout the study, animals were weighed daily and clinically observed prior to challenge, and twice daily post-challenge. COVID-19-scoring will take place during morning observation sessions.

Oral swabs were taken at 3 timepoints beginning on day 2 (SD 2) and continuing days 4 and 7 (SD 4 and 7). Genomic and sub-genomic PCR assays were conducted on the swabs.

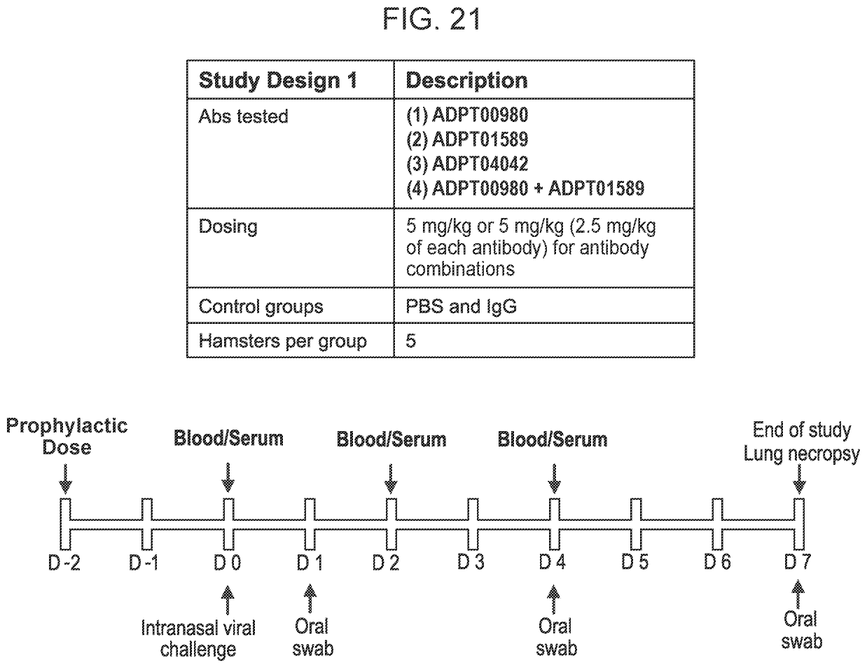

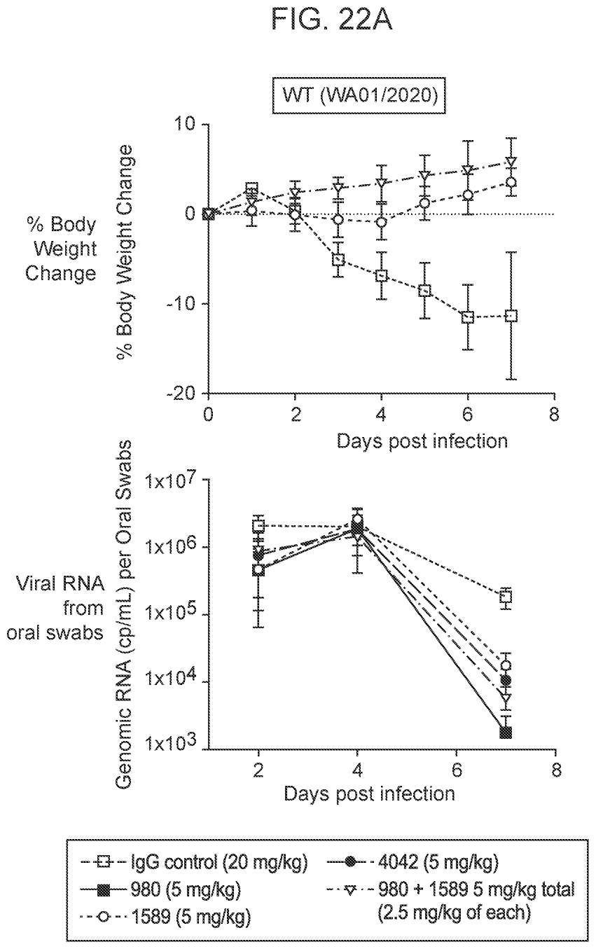

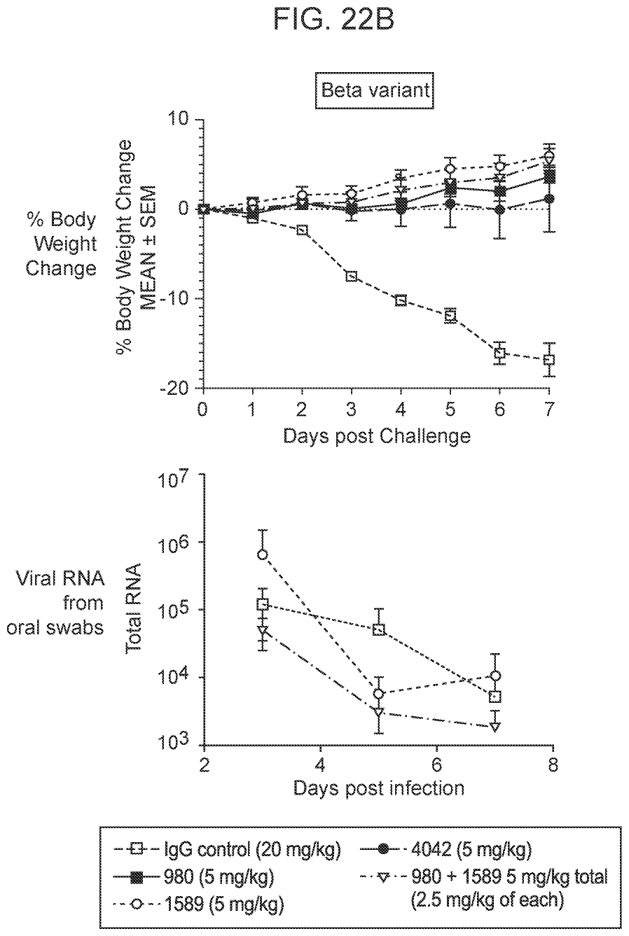

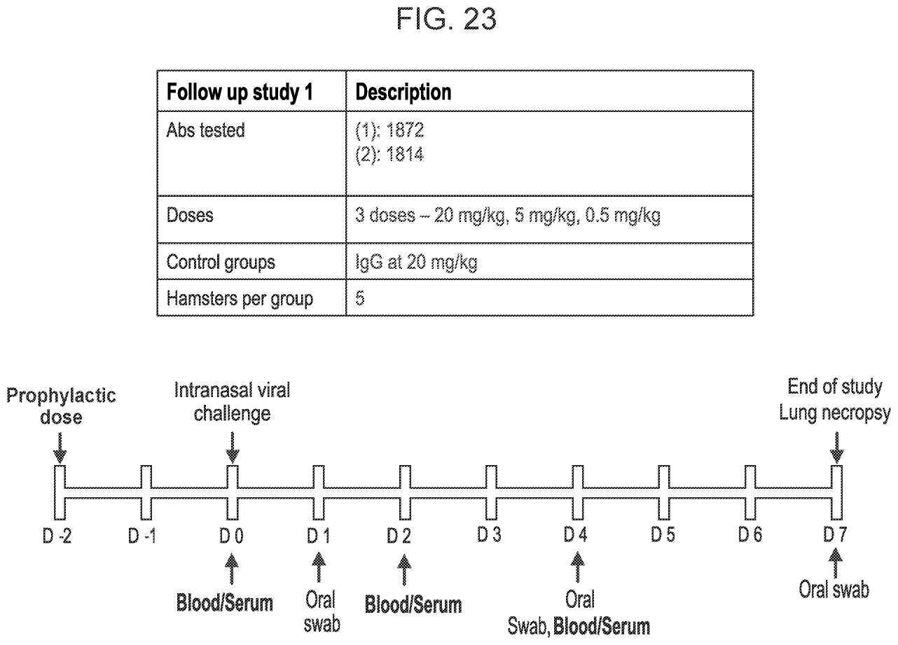

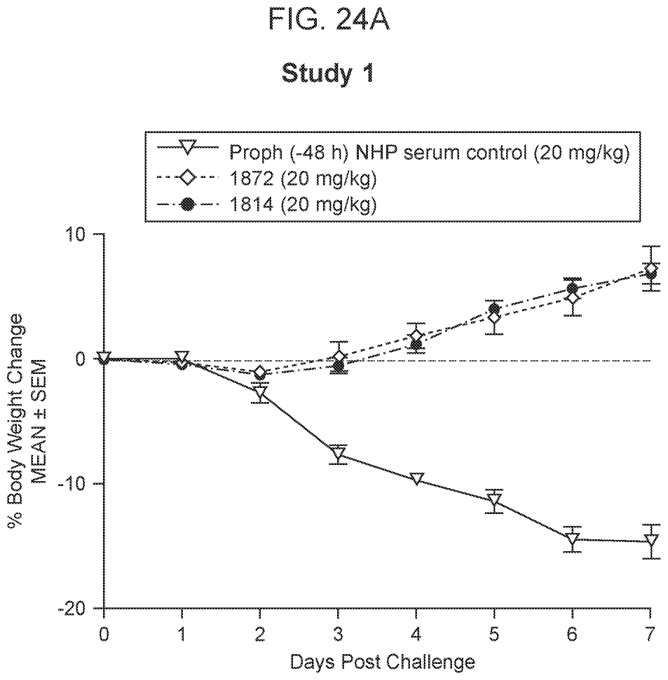

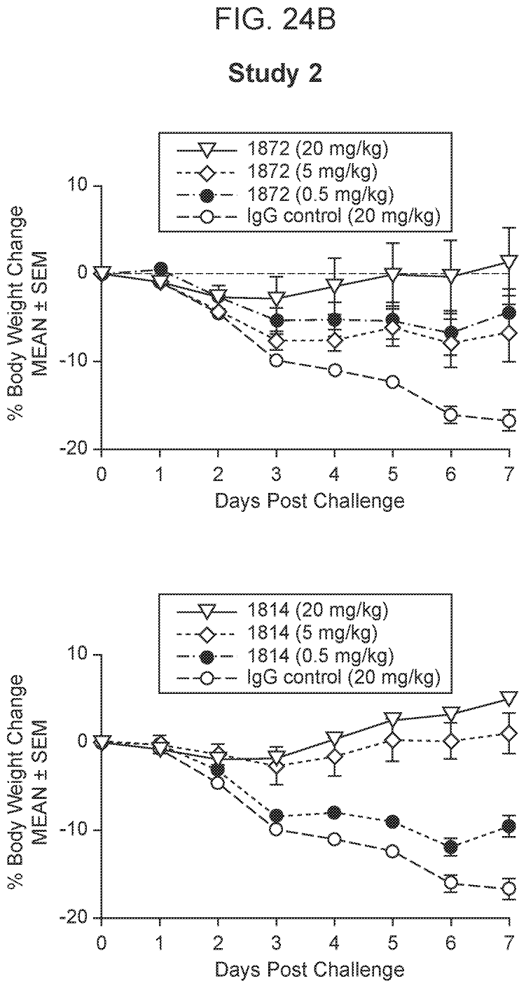

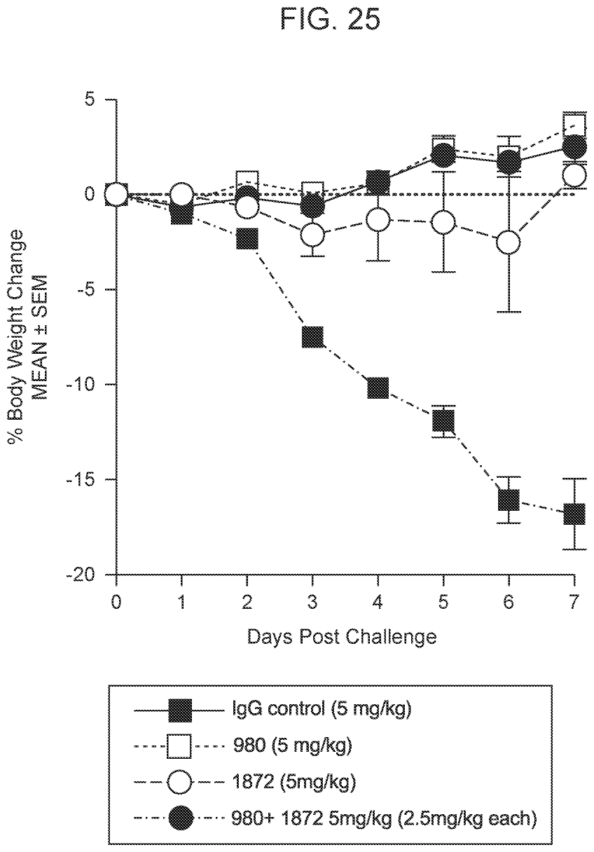

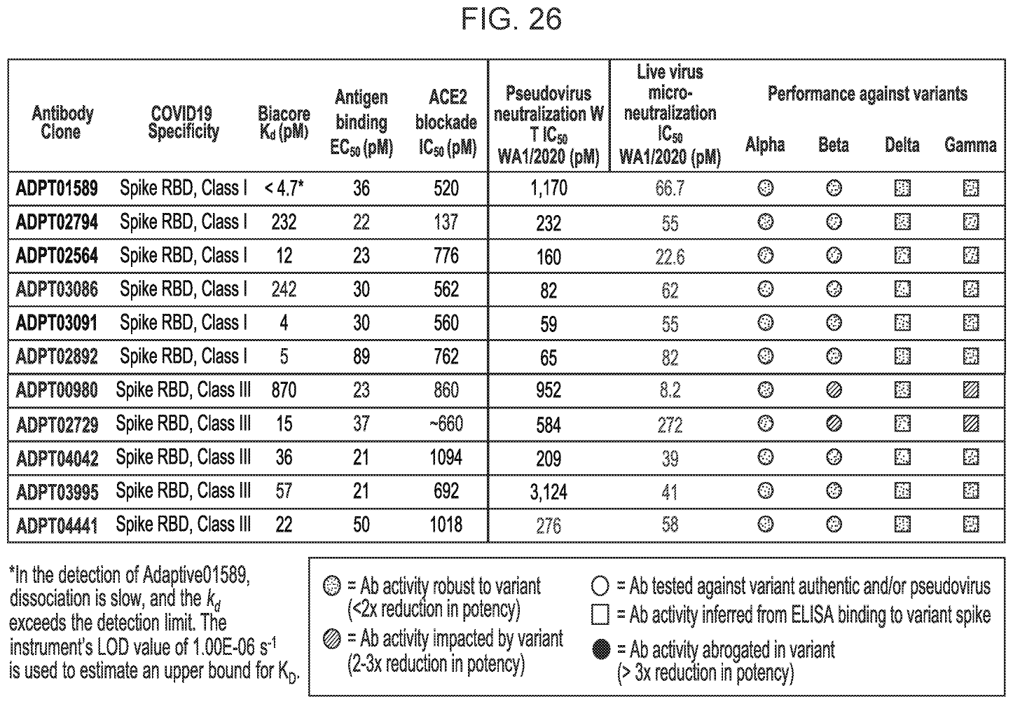

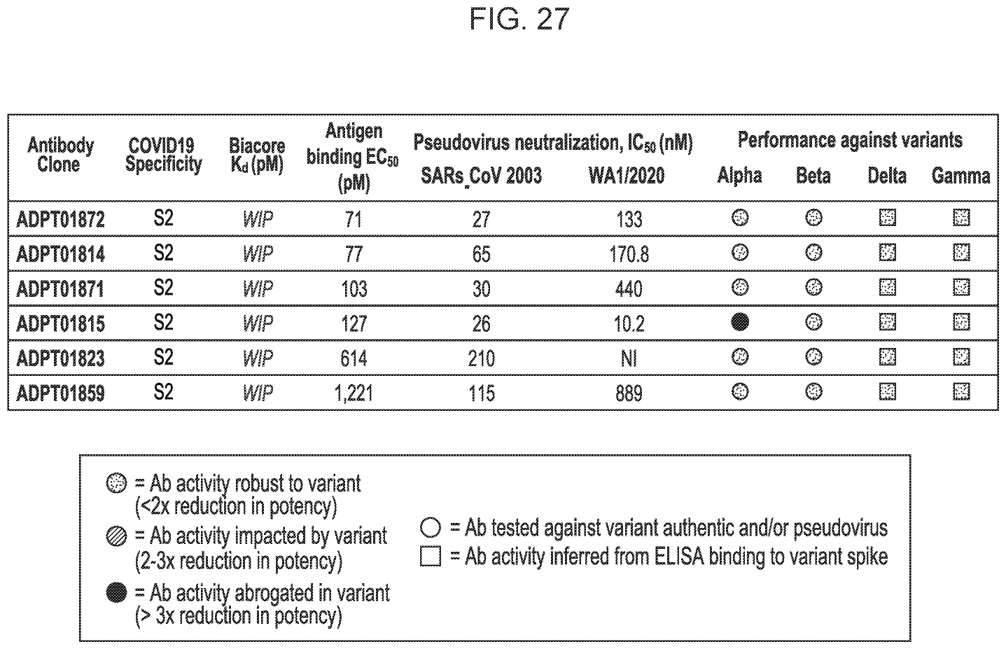

Results are shown in FIGS. 21-27. FIG. 21: Study schematic for in vivo studies with anti-RBD antibodies: 980, 1589, 4042, and combinations thereof. FIG. 22A-22B: A) Percent body weight change observed over the 7-day study post challenge with the SARs-CoV-2 virus, isolate WA01/2020. Tested antibodies prevented significant weight loss and reduced viral RNA copies observed in oral swabs compared to IgG controls. B) Percent body weight change observed over the 7-day study post challenge with the SARs-CoV-2 virus, beta variant. Tested antibodies prevented significant weight loss and reduced viral RNA copies observed in oral swabs compared to IgG controls. FIG. 23: Study schematic for in vivo studies with anti-S2 antibodies: 1872 and 1814. FIG. 24A-24B: A) Percent body weight change observed over the 7-day study post challenge with the SARs-CoV-2 virus, isolate WA01/2020, and at a dose of 20 mg/kg. Tested antibodies prevented significant weight loss. B) Percent body weight change observed over the 7-day study post challenge with the SARs-CoV-2 virus, isolate WA01/2020. Tested antibodies prevented significant weight loss down to doses of 0.5 mg/kg and showed the expected dose response. FIG. 25: Percent body weight change observed over the 7-day study post challenge with the SARs-CoV-2 virus, beta variant. Tested antibodies were an anti-RBD binding Ab 980 and an anti-S2 binding Ab 1872. These antibodies given as monotherapy or in combination prevented significant weight loss compared to an IgG control. These data demonstrate the non-competing binding, neutralization, and efficacy of combining an anti-S2 antibody and an anti-RBD antibody. FIG. 26: Summary of a subset of RBD-binding antibodies, including their epitope bin (structural class), binding affinity via Biacore and ELISA, ACE2-binding inhibition, efficacy at neutralizing pseudovirus and the WA01/2020 isolate in live virus assays. The table also summarizes each antibody's ability to neutralize variants in pseudo- or live-virus assays (circles) or ability to retain binding affinity to antigens representing SARs-CoV-2 variants (squares). FIG. 27: Summary of a subset of S2-binding antibodies, binding affinity via ELISA, efficacy at neutralizing pseudovirus of the SARs-CoV (2003) and the SARs-CoV-2 WA01/2020 isolate. The table also summarizes the ability of the antibodies to neutralize variants in pseudovirus neutralization assays (circles) or ability to retain binding affinity to antigens representing SARs-CoV-2 variants (squares).

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. An antibody that specifically binds a SARS-CoV-2 antigen and competes for binding to the SARS-CoV-2 antigen with an antibody comprising:
a variable heavy chain (V_{H}) polypeptide comprising
the V_{H} CDR1, V_{H} CDR2 and V_{H} CDR3 of the V_{H} set forth in SEQ ID NO:1, and
a variable light chain (V_{L}) polypeptide comprising
the V_{L} CDR1, V_{L} CDR2 and V_{L} CDR3 of the V_{L} set forth in SEQ ID NO:5;
a variable heavy chain (V_{H}) polypeptide comprising
the V_{H} CDR1, V_{H} CDR2 and V_{H} CDR3 of the V_{H} set forth in SEQ ID NO:9, and
a variable light chain (V_{L}) polypeptide comprising
the V_{L} CDR1, V_{L} CDR2 and V_{L} CDR3 of the V_{L} set forth in SEQ ID NO:13;
a variable heavy chain (V_{H}) polypeptide comprising
the V_{H} CDR1, V_{H} CDR2 and V_{H} CDR3 of the V_{H} set forth in SEQ ID NO:17, and
a variable light chain (V_{L}) polypeptide comprising
the V_{L} CDR1, V_{L} CDR2 and V_{L} CDR3 of the V_{L} set forth in SEQ ID NO:21;
a variable heavy chain (V_{H}) polypeptide comprising
the V_{H} CDR1, V_{H} CDR2 and V_{H} CDR3 of the V_{H} set forth in SEQ ID NO:25, and
a variable light chain (V_{L}) polypeptide comprising
the V_{L} CDR1, V_{L} CDR2 and V_{L} CDR3 of the V_{L} set forth in SEQ ID NO:29;
a variable heavy chain (V_{H}) polypeptide comprising
the V_{H} CDR1, V_{H} CDR2 and V_{H} CDR3 of the V_{H} set forth in SEQ ID NO:33, and
a variable light chain (V_{L}) polypeptide comprising
the V_{L} CDR1, V_{L} CDR2 and V_{L} CDR3 of the V_{L} set forth in SEQ ID NO:37;
a variable heavy chain (V_{H}) polypeptide comprising
the V_{H} CDR1, V_{H} CDR2 and V_{H} CDR3 of the V_{H} set forth in SEQ ID NO:41, and
a variable light chain (V_{L}) polypeptide comprising
the V_{L} CDR1, V_{L} CDR2 and V_{L} CDR3 of the V_{L} set forth in SEQ ID NO:45;
a variable heavy chain (V_{H}) polypeptide comprising
the V_{H} CDR1, V_{H} CDR2 and V_{H} CDR3 of the V_{H} set forth in SEQ ID NO:49, and
a variable light chain (V_{L}) polypeptide comprising
the V_{L} CDR1, V_{L} CDR2 and V_{L} CDR3 of the V_{L} set forth in SEQ ID NO:53;
a variable heavy chain (V_{H}) polypeptide comprising
the V_{H} CDR1, V_{H} CDR2 and V_{H} CDR3 of the V_{H} set forth in SEQ ID NO:57, and
a variable light chain (V_{L}) polypeptide comprising
the V_{L} CDR1, V_{L} CDR2 and V_{L} CDR3 of the V_{L} set forth in SEQ ID NO:61;
a variable heavy chain (V_{H}) polypeptide comprising
the V_{H} CDR1, V_{H} CDR2 and V_{H} CDR3 of the V_{H} set forth in SEQ ID NO:65, and
a variable light chain (V_{L}) polypeptide comprising
the V_{L} CDR1, V_{L} CDR2 and V_{L} CDR3 of the V_{L} set forth in SEQ ID NO:69;
a variable heavy chain (V_{H}) polypeptide comprising
the V_{H} CDR1, V_{H} CDR2 and V_{H} CDR3 of the V_{H} set forth in SEQ ID NO:73, and
a variable light chain (V_{L}) polypeptide comprising
the V_{L} CDR1, V_{L} CDR2 and V_{L} CDR3 of the V_{L} set forth in SEQ ID NO:77;
a variable heavy chain (V_{H}) polypeptide comprising
the V_{H} CDR1, V_{H} CDR2 and V_{H} CDR3 of the V_{H} set forth in SEQ ID NO:81, and
a variable light chain (V_{L}) polypeptide comprising
the V_{L} CDR1, V_{L} CDR2 and V_{L} CDR3 of the V_{L} set forth in SEQ ID NO:85; or
a variable heavy chain (V_{H}) polypeptide comprising
the V_{H} CDR1, V_{H} CDR2 and V_{H} CDR3 of the V_{H} set forth in SEQ ID NO:89, and
a variable light chain (V_{L}) polypeptide comprising
the V_{L} CDR1, V_{L} CDR2 and V_{L} CDR3 of the V_{L} set forth in SEQ ID NO:93;
a variable heavy chain (V_{H}) polypeptide comprising
the V_{H} CDR1, V_{H} CDR2 and V_{H} CDR3 of the V_{H} set forth in SEQ ID NO:97, and
a variable light chain (V_{L}) polypeptide comprising
the V_{L} CDR1, V_{L} CDR2 and V_{L} CDR3 of the V_{L} set forth in SEQ ID NO:101;
a variable heavy chain (V_{H}) polypeptide comprising
the V_{H} CDR1, V_{H} CDR2 and V_{H} CDR3 of the V_{H} set forth in SEQ ID NO:105, and
a variable light chain (V_{L}) polypeptide comprising
the V_{L} CDR1, V_{L} CDR2 and V_{L} CDR3 of the V_{L} set forth in SEQ ID NO:109;
a variable heavy chain (V_{H}) polypeptide comprising
the V_{H} CDR1, V_{H} CDR2 and V_{H} CDR3 of the V_{H} set forth in SEQ ID NO: 113, and
a variable light chain (V_{L}) polypeptide comprising
the V_{L} CDR1, V_{L} CDR2 and V_{L} CDR3 of the V_{L} set forth in SEQ ID NO:117;
a variable heavy chain (V_{H}) polypeptide comprising
the V_{H} CDR1, V_{H} CDR2 and V_{H} CDR3 of the V_{H} set forth in SEQ ID NO:121, and
a variable light chain (V_{L}) polypeptide comprising
the V_{L} CDR1, V_{L} CDR2 and V_{L} CDR3 of the V_{L} set forth in SEQ ID NO:125;
a variable heavy chain (V_{H}) polypeptide comprising
the V_{H} CDR1, V_{H} CDR2 and V_{H} CDR3 of the V_{H} set forth in SEQ ID NO:129, and
a variable light chain (V_{L}) polypeptide comprising
the V_{L} CDR1, V_{L} CDR2 and V_{L} CDR3 of the V_{L} set forth in SEQ ID NO:133;
a variable heavy chain (V_{H}) polypeptide comprising
the V_{H} CDR1, V_{H} CDR2 and V_{H} CDR3 of the V_{H} set forth in SEQ ID NO:137, and
a variable light chain (V_{L}) polypeptide comprising
the V_{L} CDR1, V_{L} CDR2 and V_{L} CDR3 of the V_{L} set forth in SEQ ID NO:141;
a variable heavy chain (V_{H}) polypeptide comprising
the V_{H} CDR1, V_{H} CDR2 and V_{H} CDR3 of the V_{H} set forth in SEQ ID NO:145, and
a variable light chain (V_{L}) polypeptide comprising
the V_{L} CDR1, V_{L} CDR2 and V_{L} CDR3 of the V_{L} set forth in SEQ ID NO:149;
a variable heavy chain (V_{H}) polypeptide comprising
the V_{H} CDR1, V_{H} CDR2 and V_{H} CDR3 of the V_{H} set forth in SEQ ID NO:153, and
a variable light chain (V_{L}) polypeptide comprising
the V_{L} CDR1, V_{L} CDR2 and V_{L} CDR3 of the V_{L} set forth in SEQ ID NO:157;
a variable heavy chain (V_{H}) polypeptide comprising
the V_{H} CDR1, V_{H} CDR2 and V_{H} CDR3 of the V_{H} set forth in SEQ ID NO:161, and
a variable light chain (V_{L}) polypeptide comprising
the V_{L} CDR1, V_{L} CDR2 and V_{L} CDR3 of the V_{L} set forth in SEQ ID NO:165;
a variable heavy chain (V_{H}) polypeptide comprising
the V_{H} CDR1, V_{H} CDR2 and V_{H} CDR3 of the V_{H} set forth in SEQ ID NO:169, and
a variable light chain (V_{L}) polypeptide comprising
the V_{L} CDR1, V_{L} CDR2 and V_{L} CDR3 of the V_{L} set forth in SEQ ID NO:173;
a variable heavy chain (V_{H}) polypeptide comprising
the V_{H} CDR1, V_{H} CDR2 and V_{H} CDR3 of the V_{H} set forth in SEQ ID NO:177, and
a variable light chain (V_{L}) polypeptide comprising
the V_{L} CDR1, V_{L} CDR2 and V_{L} CDR3 of the V_{L} set forth in SEQ ID NO:181;
a variable heavy chain (V_{H}) polypeptide comprising
the V_{H} CDR1, V_{H} CDR2 and V_{H} CDR3 of the V_{H} set forth in SEQ ID NO:185, and
a variable light chain (V_{L}) polypeptide comprising
the V_{L} CDR1, V_{L} CDR2 and V_{L} CDR3 of the V_{L} set forth in SEQ ID NO:189;
a variable heavy chain (V_{H}) polypeptide comprising
the V_{H} CDR1, V_{H} CDR2 and V_{H} CDR3 of the V_{H} set forth in SEQ ID NO:193, and
a variable light chain (V_{L}) polypeptide comprising
the V_{L} CDR1, V_{L} CDR2 and V_{L} CDR3 of the V_{L} set forth in SEQ ID NO:197;
a variable heavy chain (V_{H}) polypeptide comprising
the V_{H} CDR1, V_{H} CDR2 and V_{H} CDR3 of the V_{H} set forth in SEQ ID NO:201, and
a variable light chain (V_{L}) polypeptide comprising
the V_{L} CDR1, V_{L} CDR2 and V_{L} CDR3 of the V_{L} set forth in SEQ ID NO:205;
a variable heavy chain (V_{H}) polypeptide comprising
the V_{H} CDR1, V_{H} CDR2 and V_{H} CDR3 of the V_{H} set forth in SEQ ID NO:209, and
a variable light chain (V_{L}) polypeptide comprising
the V_{L} CDR1, V_{L} CDR2 and V_{L} CDR3 of the V_{L} set forth in SEQ ID NO:213;
a variable heavy chain (V_{H}) polypeptide comprising
the V_{H} CDR1, V_{H} CDR2 and V_{H} CDR3 of the V_{H} set forth in SEQ ID NO:217, and
a variable light chain (V_{L}) polypeptide comprising
the V_{L} CDR1, V_{L} CDR2 and V_{L} CDR3 of the V_{L} set forth in SEQ ID NO:221;
a variable heavy chain (V_{H}) polypeptide comprising
the V_{H} CDR1, V_{H} CDR2 and V_{H} CDR3 of the V_{H} set forth in SEQ ID NO:225, and
a variable light chain (V_{L}) polypeptide comprising
the V_{L} CDR1, V_{L} CDR2 and V_{L} CDR3 of the V_{L} set forth in SEQ ID NO:229;
a variable heavy chain (V_{H}) polypeptide comprising
the V_{H} CDR1, V_{H} CDR2 and V_{H} CDR3 of the V_{H} set forth in SEQ ID NO:233, and
a variable light chain (V_{L}) polypeptide comprising
the V_{L} CDR1, V_{L} CDR2 and V_{L} CDR3 of the V_{L} set forth in SEQ ID NO:237;
a variable heavy chain (V_{H}) polypeptide comprising
the V_{H} CDR1, V_{H} CDR2 and V_{H} CDR3 of the V_{H} set forth in SEQ ID NO:241, and
a variable light chain (V_{L}) polypeptide comprising
the V_{L} CDR1, V_{L} CDR2 and V_{L} CDR3 of the V_{L} set forth in SEQ ID NO:245;
a variable heavy chain (V_{H}) polypeptide comprising
the V_{H} CDR1, V_{H} CDR2 and V_{H} CDR3 of the V_{H} set forth in SEQ ID NO:249, and
a variable light chain (V_{L}) polypeptide comprising
the V_{L} CDR1, V_{L} CDR2 and V_{L} CDR3 of the V_{L} set forth in SEQ ID NO:253;
a variable heavy chain (V_{H}) polypeptide comprising
the V_{H} CDR1, V_{H} CDR2 and V_{H} CDR3 of the V_{H} set forth in SEQ ID NO:257, and
a variable light chain (V_{L}) polypeptide comprising
the V_{L} CDR1, V_{L} CDR2 and V_{L} CDR3 of the V_{L} set forth in SEQ ID NO:261;
a variable heavy chain (V_{H}) polypeptide comprising
the V_{H} CDR1, V_{H} CDR2 and V_{H} CDR3 of the V_{H} set forth in SEQ ID NO:265, and
a variable light chain (V_{L}) polypeptide comprising
the V_{L} CDR1, V_{L} CDR2 and V_{L} CDR3 of the V_{L} set forth in SEQ ID NO:269;
a variable heavy chain (V_{H}) polypeptide comprising
the V_{H} CDR1, V_{H} CDR2 and V_{H} CDR3 of the V_{H} set forth in SEQ ID NO:273, and
a variable light chain (V_{L}) polypeptide comprising
the V_{L} CDR1, V_{L} CDR2 and V_{L} CDR3 of the V_{L} set forth in SEQ ID NO:277;
a variable heavy chain (V_{H}) polypeptide comprising
the V_{H} CDR1, V_{H} CDR2 and V_{H} CDR3 of the V_{H} set forth in SEQ ID NO:281, and
a variable light chain (V_{L}) polypeptide comprising
the V_{L} CDR1, V_{L} CDR2 and V_{L} CDR3 of the V_{L} set forth in SEQ ID NO:285;
a variable heavy chain (V_{H}) polypeptide comprising
the V_{H} CDR1, V_{H} CDR2 and V_{H} CDR3 of the V_{H} set forth in SEQ ID NO:289, and
a variable light chain (V_{L}) polypeptide comprising
the V_{L} CDR1, V_{L} CDR2 and V_{L} CDR3 of the V_{L} set forth in SEQ ID NO:293;
a variable heavy chain (V_{H}) polypeptide comprising
the V_{H} CDR1, V_{H} CDR2 and V_{H} CDR3 of the V_{H} set forth in SEQ ID NO:297, and
a variable light chain (V_{L}) polypeptide comprising
the V_{L} CDR1, V_{L} CDR2 and V_{L} CDR3 of the V_{L} set forth in SEQ ID NO:301;
a variable heavy chain (V_{H}) polypeptide comprising
the V_{H} CDR1, V_{H} CDR2 and V_{H} CDR3 of the V_{H} set forth in SEQ ID NO:305, and
a variable light chain (V_{L}) polypeptide comprising
the V_{L} CDR1, V_{L} CDR2 and V_{L} CDR3 of the V_{L} set forth in SEQ ID NO:309;
a variable heavy chain (V_{H}) polypeptide comprising
the V_{H} CDR1, V_{H} CDR2 and V_{H} CDR3 of the V_{H} set forth in SEQ ID NO:313, and
a variable light chain (V_{L}) polypeptide comprising
the V_{L} CDR1, V_{L} CDR2 and V_{L} CDR3 of the V_{L} set forth in SEQ ID NO:317;
a variable heavy chain (V_{H}) polypeptide comprising
the V_{H} CDR1, V_{H} CDR2 and V_{H} CDR3 of the V_{H} set forth in SEQ ID NO:321, and
a variable light chain (V_{L}) polypeptide comprising
the V_{L} CDR1, V_{L} CDR2 and V_{L} CDR3 of the V_{L} set forth in SEQ ID NO:325;
a variable heavy chain (V_{H}) polypeptide comprising
the V_{H} CDR1, V_{H} CDR2 and V_{H} CDR3 of the V_{H} set forth in SEQ ID NO:329, and
a variable light chain (V_{L}) polypeptide comprising
the V_{L} CDR1, V_{L} CDR2 and V_{L} CDR3 of the V_{L} set forth in SEQ ID NO:333;
a variable heavy chain (V_{H}) polypeptide comprising
the V_{H} CDR1, V_{H} CDR2 and V_{H} CDR3 of the V_{H} set forth in SEQ ID NO:337, and
a variable light chain (V_{L}) polypeptide comprising
the V_{L} CDR1, V_{L} CDR2 and V_{L} CDR3 of the V_{L} set forth in SEQ ID NO:341;
a variable heavy chain (V_{H}) polypeptide comprising
the V_{H} CDR1, V_{H} CDR2 and V_{H} CDR3 of the V_{H} set forth in SEQ ID NO:345, and
a variable light chain (V_{L}) polypeptide comprising
the V_{L} CDR1, V_{L} CDR2 and V_{L} CDR3 of the V_{L} set forth in SEQ ID NO:349;
a variable heavy chain (V_{H}) polypeptide comprising
the V_{H} CDR1, V_{H} CDR2 and V_{H} CDR3 of the V_{H} set forth in SEQ ID NO:353, and
a variable light chain (V_{L}) polypeptide comprising
the V_{L} CDR1, V_{L} CDR2 and V_{L} CDR3 of the V_{L} set forth in SEQ ID NO:357;
a variable heavy chain (V_{H}) polypeptide comprising
the V_{H} CDR1, V_{H} CDR2 and V_{H} CDR3 of the V_{H} set forth in SEQ ID NO:361, and
a variable light chain (V_{L}) polypeptide comprising
the V_{L} CDR1, V_{L} CDR2 and V_{L} CDR3 of the V_{L} set forth in SEQ ID NO:365;
a variable heavy chain (V_{H}) polypeptide comprising
the V_{H} CDR1, V_{H} CDR2 and V_{H} CDR3 of the V_{H} set forth in SEQ ID NO:369, and
a variable light chain (V_{L}) polypeptide comprising
the V_{L} CDR1, V_{L} CDR2 and V_{L} CDR3 of the V_{L} set forth in SEQ ID NO:373;
a variable heavy chain (V_{H}) polypeptide comprising
the V_{H} CDR1, V_{H} CDR2 and V_{H} CDR3 of the V_{H} set forth in SEQ ID NO:377, and
a variable light chain (V_{L}) polypeptide comprising
the V_{L} CDR1, V_{L} CDR2 and V_{L} CDR3 of the V_{L} set forth in SEQ ID NO:381;
a variable heavy chain (V_{H}) polypeptide comprising
the V_{H} CDR1, V_{H} CDR2 and V_{H} CDR3 of the V_{H} set forth in SEQ ID NO:385, and
a variable light chain (V_{L}) polypeptide comprising
the V_{L} CDR1, V_{L} CDR2 and V_{L} CDR3 of the V_{L} set forth in SEQ ID NO:389;
a variable heavy chain (V_{H}) polypeptide comprising
the V_{H} CDR1, V_{H} CDR2 and V_{H} CDR3 of the V_{H} set forth in SEQ ID NO:393, and
a variable light chain (V_{L}) polypeptide comprising
the V_{L} CDR1, V_{L} CDR2 and V_{L} CDR3 of the V_{L} set forth in SEQ ID NO:397;
a variable heavy chain (V_{H}) polypeptide comprising
the V_{H} CDR1, V_{H} CDR2 and V_{H} CDR3 of the V_{H} set forth in SEQ ID NO:401, and
a variable light chain (V_{L}) polypeptide comprising
the V_{L} CDR1, V_{L} CDR2 and V_{L} CDR3 of the V_{L} set forth in SEQ ID NO:405;
a variable heavy chain (V_{H}) polypeptide comprising
the V_{H} CDR1, V_{H} CDR2 and V_{H} CDR3 of the V_{H} set forth in SEQ ID NO:409, and
a variable light chain (V_{L}) polypeptide comprising
the V_{L} CDR1, V_{L} CDR2 and V_{L} CDR3 of the V_{L} set forth in SEQ ID NO:413;
a variable heavy chain (V_{H}) polypeptide comprising
the V_{H} CDR1, V_{H} CDR2 and V_{H} CDR3 of the V_{H} set forth in SEQ ID NO:417, and
a variable light chain (V_{L}) polypeptide comprising
the V_{L} CDR1, V_{L} CDR2 and V_{L} CDR3 of the V_{L} set forth in SEQ ID NO:421;
a variable heavy chain (V_{H}) polypeptide comprising
the V_{H} CDR1, V_{H} CDR2 and V_{H} CDR3 of the V_{H} set forth in SEQ ID NO:425, and
a variable light chain (V_{L}) polypeptide comprising
the V_{L} CDR1, V_{L} CDR2 and V_{L} CDR3 of the V_{L} set forth in SEQ ID NO:429;
a variable heavy chain (V_{H}) polypeptide comprising
the V_{H} CDR1, V_{H} CDR2 and V_{H} CDR3 of the V_{H} set forth in SEQ ID NO:433, and
a variable light chain (V_{L}) polypeptide comprising
the V_{L} CDR1, V_{L} CDR2 and V_{L} CDR3 of the V_{L} set forth in SEQ ID NO:437;
a variable heavy chain (V_{H}) polypeptide comprising
the V_{H} CDR1, V_{H} CDR2 and V_{H} CDR3 of the V_{H} set forth in SEQ ID NO:441, and
a variable light chain (V_{L}) polypeptide comprising
the V_{L} CDR1, V_{L} CDR2 and V_{L} CDR3 of the V_{L} set forth in SEQ ID NO:445;
a variable heavy chain (V_{H}) polypeptide comprising
the V_{H} CDR1, V_{H} CDR2 and V_{H} CDR3 of the V_{H} set forth in SEQ ID NO:449, and
a variable light chain (V_{L}) polypeptide comprising
the V_{L} CDR1, V_{L} CDR2 and V_{L} CDR3 of the V_{L} set forth in SEQ ID NO:453;
a variable heavy chain (V_{H}) polypeptide comprising
the V_{H} CDR1, V_{H} CDR2 and V_{H} CDR3 of the V_{H} set forth in SEQ ID NO:457, and
a variable light chain (V_{L}) polypeptide comprising
the V_{L} CDR1, V_{L} CDR2 and V_{L} CDR3 of the V_{L} set forth in SEQ ID NO:461; or
a variable heavy chain (V_{H}) polypeptide comprising
the V_{H} CDR1, V_{H} CDR2 and V_{H} CDR3 of the V_{H} set forth in SEQ ID NO:465, and
a variable light chain (V_{L}) polypeptide comprising
the V_{L} CDR1, V_{L} CDR2 and V_{L} CDR3 of the V_{L} set forth in SEQ ID NO:469.

2. The antibody of claim 1, wherein the antibody comprises:
a variable heavy chain (V_{H}) polypeptide comprising
the V_{H} CDR1, V_{H} CDR2 and V_{H} CDR3 of the V_{H} set forth in SEQ ID NO:1, and
a variable light chain (V_{L}) polypeptide comprising
the V_{L} CDR1, V_{L} CDR2 and V_{L} CDR3 of the V_{L} set forth in SEQ ID NO:5;
a variable heavy chain (V_{H}) polypeptide comprising
the V_{H} CDR1, V_{H} CDR2 and V_{H} CDR3 of the V_{H} set forth in SEQ ID NO:9, and
a variable light chain (V_{L}) polypeptide comprising
the V_{L} CDR1, V_{L} CDR2 and V_{L} CDR3 of the V_{L} set forth in SEQ ID NO:13;
a variable heavy chain (V_{H}) polypeptide comprising
the V_{H} CDR1, V_{H} CDR2 and V_{H} CDR3 of the V_{H} set forth in SEQ ID NO:17, and
a variable light chain (V_{L}) polypeptide comprising
the V_{L} CDR1, V_{L} CDR2 and V_{L} CDR3 of the V_{L} set forth in SEQ ID NO:21;
a variable heavy chain (V_{H}) polypeptide comprising
the V_{H} CDR1, V_{H} CDR2 and V_{H} CDR3 of the V_{H} set forth in SEQ ID NO:25, and
a variable light chain (V_{L}) polypeptide comprising
the V_{L} CDR1, V_{L} CDR2 and V_{L} CDR3 of the V_{L} set forth in SEQ ID NO:29;
a variable heavy chain (V_{H}) polypeptide comprising
the V_{H} CDR1, V_{H} CDR2 and V_{H} CDR3 of the V_{H} set forth in SEQ ID NO:33, and
a variable light chain (V_{L}) polypeptide comprising
the V_{L} CDR1, V_{L} CDR2 and V_{L} CDR3 of the V_{L} set forth in SEQ ID NO:37;
a variable heavy chain (V_{H}) polypeptide comprising
the V_{H} CDR1, V_{H} CDR2 and V_{H} CDR3 of the V_{H} set forth in SEQ ID NO:41, and
a variable light chain (V_{L}) polypeptide comprising
the V_{L} CDR1, V_{L} CDR2 and V_{L} CDR3 of the V_{L} set forth in SEQ ID NO:45;
a variable heavy chain (V_{H}) polypeptide comprising
the V_{H} CDR1, V_{H} CDR2 and V_{H} CDR3 of the V_{H} set forth in SEQ ID NO:49, and
a variable light chain (V_{L}) polypeptide comprising
the V_{L} CDR1, V_{L} CDR2 and V_{L} CDR3 of the V_{L} set forth in SEQ ID NO:53;
a variable heavy chain (V_{H}) polypeptide comprising
the V_{H} CDR1, V_{H} CDR2 and V_{H} CDR3 of the V_{H} set forth in SEQ ID NO:57, and
a variable light chain (V_{L}) polypeptide comprising
the V_{L} CDR1, V_{L} CDR2 and V_{L} CDR3 of the V_{L} set forth in SEQ ID NO:61;
a variable heavy chain (V_{H}) polypeptide comprising
the V_{H} CDR1, V_{H} CDR2 and V_{H} CDR3 of the V_{H} set forth in SEQ ID NO:65, and
a variable light chain (V_{L}) polypeptide comprising
the V_{L} CDR1, V_{L} CDR2 and V_{L} CDR3 of the V_{L} set forth in SEQ ID NO:69;
a variable heavy chain (V_{H}) polypeptide comprising
the V_{H} CDR1, V_{H} CDR2 and V_{H} CDR3 of the V_{H} set forth in SEQ ID NO:73, and
a variable light chain (V_{L}) polypeptide comprising
the V_{L} CDR1, V_{L} CDR2 and V_{L} CDR3 of the V_{L} set forth in SEQ ID NO:77;
a variable heavy chain (V_{H}) polypeptide comprising
the V_{H} CDR1, V_{H} CDR2 and V_{H} CDR3 of the V_{H} set forth in SEQ ID NO:81, and
a variable light chain (V_{L}) polypeptide comprising
the V_{L} CDR1, V_{L} CDR2 and V_{L} CDR3 of the V_{L} set forth in SEQ ID NO:85; or
a variable heavy chain (V_{H}) polypeptide comprising
the V_{H} CDR1, V_{H} CDR2 and V_{H} CDR3 of the V_{H} set forth in SEQ ID NO:89, and
a variable light chain (V_{L}) polypeptide comprising
the V_{L} CDR1, V_{L} CDR2 and V_{L} CDR3 of the V_{L} set forth in SEQ ID NO:93;
a variable heavy chain (V_{H}) polypeptide comprising
the V_{H} CDR1, V_{H} CDR2 and V_{H} CDR3 of the V_{H} set forth in SEQ ID NO:97, and
a variable light chain (V_{L}) polypeptide comprising
the V_{L} CDR1, V_{L} CDR2 and V_{L} CDR3 of the V_{L} set forth in SEQ ID NO:101;
a variable heavy chain (V_{H}) polypeptide comprising
the V_{H} CDR1, V_{H} CDR2 and V_{H} CDR3 of the V_{H} set forth in SEQ ID NO:105, and
a variable light chain (V_{L}) polypeptide comprising
the V_{L} CDR1, V_{L} CDR2 and V_{L} CDR3 of the V_{L} set forth in SEQ ID NO:109;
a variable heavy chain (V_{H}) polypeptide comprising
the V_{H} CDR1, V_{H} CDR2 and V_{H} CDR3 of the V_{H} set forth in SEQ ID NO: 113, and
a variable light chain (V_{L}) polypeptide comprising
the V_{L} CDR1, V_{L} CDR2 and V_{L} CDR3 of the V_{L} set forth in SEQ ID NO:117;
a variable heavy chain (V_{H}) polypeptide comprising
the V_{H} CDR1, V_{H} CDR2 and V_{H} CDR3 of the V_{H} set forth in SEQ ID NO:121, and
a variable light chain (V_{L}) polypeptide comprising
the V_{L} CDR1, V_{L} CDR2 and V_{L} CDR3 of the V_{L} set forth in SEQ ID NO:125;
a variable heavy chain (V_{H}) polypeptide comprising
the V_{H} CDR1, V_{H} CDR2 and V_{H} CDR3 of the V_{H} set forth in SEQ ID NO:129, and
a variable light chain (V_{L}) polypeptide comprising
the V_{L} CDR1, V_{L} CDR2 and V_{L} CDR3 of the V_{L} set forth in SEQ ID NO:133;
a variable heavy chain (V_{H}) polypeptide comprising
the V_{H} CDR1, V_{H} CDR2 and V_{H} CDR3 of the V_{H} set forth in SEQ ID NO:137, and
a variable light chain (V_{L}) polypeptide comprising
the V_{L} CDR1, V_{L} CDR2 and V_{L} CDR3 of the V_{L} set forth in SEQ ID NO:141;
a variable heavy chain (V_{H}) polypeptide comprising
the V_{H} CDR1, V_{H} CDR2 and V_{H} CDR3 of the V_{H} set forth in SEQ ID NO:145, and
a variable light chain (V_{L}) polypeptide comprising
the V_{L} CDR1, V_{L} CDR2 and V_{L} CDR3 of the V_{L} set forth in SEQ ID NO:149;
a variable heavy chain (V_{H}) polypeptide comprising
the V_{H} CDR1, V_{H} CDR2 and V_{H} CDR3 of the V_{H} set forth in SEQ ID NO:153, and
a variable light chain (V_{L}) polypeptide comprising
the V_{L} CDR1, V_{L} CDR2 and V_{L} CDR3 of the V_{L} set forth in SEQ ID NO:157;
a variable heavy chain (V_{H}) polypeptide comprising
the V_{H} CDR1, V_{H} CDR2 and V_{H} CDR3 of the V_{H} set forth in SEQ ID NO:161, and
a variable light chain (V_{L}) polypeptide comprising
the V_{L} CDR1, V_{L} CDR2 and V_{L} CDR3 of the V_{L} set forth in SEQ ID NO:165;
a variable heavy chain (V_{H}) polypeptide comprising
the V_{H} CDR1, V_{H} CDR2 and V_{H} CDR3 of the V_{H} set forth in SEQ ID NO:169, and
a variable light chain (V_{L}) polypeptide comprising
the V_{L} CDR1, V_{L} CDR2 and V_{L} CDR3 of the V_{L} set forth in SEQ ID NO:173;
a variable heavy chain (V_{H}) polypeptide comprising
the V_{H} CDR1, V_{H} CDR2 and V_{H} CDR3 of the V_{H} set forth in SEQ ID NO:177, and
a variable light chain (V_{L}) polypeptide comprising
the V_{L} CDR1, V_{L} CDR2 and V_{L} CDR3 of the V_{L} set forth in SEQ ID NO:181;
a variable heavy chain (V_{H}) polypeptide comprising
the V_{H} CDR1, V_{H} CDR2 and V_{H} CDR3 of the V_{H} set forth in SEQ ID NO:185, and
a variable light chain (V_{L}) polypeptide comprising
the V_{L} CDR1, V_{L} CDR2 and V_{L} CDR3 of the V_{L} set forth in SEQ ID NO:189;
a variable heavy chain (V_{H}) polypeptide comprising
the V_{H} CDR1, V_{H} CDR2 and V_{H} CDR3 of the V_{H} set forth in SEQ ID NO:193, and
a variable light chain (V_{L}) polypeptide comprising
the V_{L} CDR1, V_{L} CDR2 and V_{L} CDR3 of the V_{L} set forth in SEQ ID NO:197;
a variable heavy chain (V_{H}) polypeptide comprising
the V_{H} CDR1, V_{H} CDR2 and V_{H} CDR3 of the V_{H} set forth in SEQ ID NO:201, and
a variable light chain (V_{L}) polypeptide comprising
the V_{L} CDR1, V_{L} CDR2 and V_{L} CDR3 of the V_{L} set forth in SEQ ID NO:205;
a variable heavy chain (V_{H}) polypeptide comprising
the V_{H} CDR1, V_{H} CDR2 and V_{H} CDR3 of the V_{H} set forth in SEQ ID NO:209, and
a variable light chain (V_{L}) polypeptide comprising
the V_{L} CDR1, V_{L} CDR2 and V_{L} CDR3 of the V_{L} set forth in SEQ ID NO:213;
a variable heavy chain (V_{H}) polypeptide comprising
the V_{H} CDR1, V_{H} CDR2 and V_{H} CDR3 of the V_{H} set forth in SEQ ID NO:217, and
a variable light chain (V_{L}) polypeptide comprising
the V_{L} CDR1, V_{L} CDR2 and V_{L} CDR3 of the V_{L} set forth in SEQ ID NO:221;
a variable heavy chain (V_{H}) polypeptide comprising
the V_{H} CDR1, V_{H} CDR2 and V_{H} CDR3 of the V_{H} set forth in SEQ ID NO:225, and
a variable light chain (V_{L}) polypeptide comprising
the V_{L} CDR1, V_{L} CDR2 and V_{L} CDR3 of the V_{L} set forth in SEQ ID NO:229;
a variable heavy chain (V_{H}) polypeptide comprising
the V_{H} CDR1, V_{H} CDR2 and V_{H} CDR3 of the V_{H} set forth in SEQ ID NO:233, and
a variable light chain (V_{L}) polypeptide comprising
the V_{L} CDR1, V_{L} CDR2 and V_{L} CDR3 of the V_{L} set forth in SEQ ID NO:237;
a variable heavy chain (V_{H}) polypeptide comprising
the V_{H} CDR1, V_{H} CDR2 and V_{H} CDR3 of the V_{H} set forth in SEQ ID NO:241, and
a variable light chain (V_{L}) polypeptide comprising
the V_{L} CDR1, V_{L} CDR2 and V_{L} CDR3 of the V_{L} set forth in SEQ ID NO:245;
a variable heavy chain (V_{H}) polypeptide comprising
the V_{H} CDR1, V_{H} CDR2 and V_{H} CDR3 of the V_{H} set forth in SEQ ID NO:249, and
a variable light chain (V_{L}) polypeptide comprising
the V_{L} CDR1, V_{L} CDR2 and V_{L} CDR3 of the V_{L} set forth in SEQ ID NO:253;
a variable heavy chain (V_{H}) polypeptide comprising
the V_{H} CDR1, V_{H} CDR2 and V_{H} CDR3 of the V_{H} set forth in SEQ ID NO:257, and
a variable light chain (V_{L}) polypeptide comprising
the V_{L} CDR1, V_{L} CDR2 and V_{L} CDR3 of the V_{L} set forth in SEQ ID NO:261;
a variable heavy chain (V_{H}) polypeptide comprising
the V_{H} CDR1, V_{H} CDR2 and V_{H} CDR3 of the V_{H} set forth in SEQ ID NO:265, and
a variable light chain (V_{L}) polypeptide comprising
the V_{L} CDR1, V_{L} CDR2 and V_{L} CDR3 of the V_{L} set forth in SEQ ID NO:269;
a variable heavy chain (V_{H}) polypeptide comprising
the V_{H} CDR1, V_{H} CDR2 and V_{H} CDR3 of the V_{H} set forth in SEQ ID NO:273, and
a variable light chain (V_{L}) polypeptide comprising
the V_{L} CDR1, V_{L} CDR2 and V_{L} CDR3 of the V_{L} set forth in SEQ ID NO:277;
a variable heavy chain (V_{H}) polypeptide comprising
the V_{H} CDR1, V_{H} CDR2 and V_{H} CDR3 of the V_{H} set forth in SEQ ID NO:281, and
a variable light chain (V_{L}) polypeptide comprising
the V_{L} CDR1, V_{L} CDR2 and V_{L} CDR3 of the V_{L} set forth in SEQ ID NO:285;
a variable heavy chain (V_{H}) polypeptide comprising
the V_{H} CDR1, V_{H} CDR2 and V_{H} CDR3 of the V_{H} set forth in SEQ ID NO:289, and
a variable light chain (V_{L}) polypeptide comprising
the V_{L} CDR1, V_{L} CDR2 and V_{L} CDR3 of the V_{L} set forth in SEQ ID NO:293;
a variable heavy chain (V_{H}) polypeptide comprising
the V_{H} CDR1, V_{H} CDR2 and V_{H} CDR3 of the V_{H} set forth in SEQ ID NO:297, and
a variable light chain (V_{L}) polypeptide comprising
the V_{L} CDR1, V_{L} CDR2 and V_{L} CDR3 of the V_{L} set forth in SEQ ID NO:301;
a variable heavy chain (V_{H}) polypeptide comprising
the V_{H} CDR1, V_{H} CDR2 and V_{H} CDR3 of the V_{H} set forth in SEQ ID NO:305, and
a variable light chain (V_{L}) polypeptide comprising
the V_{L} CDR1, V_{L} CDR2 and V_{L} CDR3 of the V_{L} set forth in SEQ ID NO:309;
a variable heavy chain (V_{H}) polypeptide comprising
the V_{H} CDR1, V_{H} CDR2 and V_{H} CDR3 of the V_{H} set forth in SEQ ID NO:313, and
a variable light chain (V_{L}) polypeptide comprising
the V_{L} CDR1, V_{L} CDR2 and V_{L} CDR3 of the V_{L} set forth in SEQ ID NO:317;
a variable heavy chain (V_{H}) polypeptide comprising
the V_{H} CDR1, V_{H} CDR2 and V_{H} CDR3 of the V_{H} set forth in SEQ ID NO:321, and
a variable light chain (V_{L}) polypeptide comprising
the V_{L} CDR1, V_{L} CDR2 and V_{L} CDR3 of the V_{L} set forth in SEQ ID NO:325;
a variable heavy chain (V_{H}) polypeptide comprising
the V_{H} CDR1, V_{H} CDR2 and V_{H} CDR3 of the V_{H} set forth in SEQ ID NO:329, and
a variable light chain (V_{L}) polypeptide comprising
the V_{L} CDR1, V_{L} CDR2 and V_{L} CDR3 of the V_{L} set forth in SEQ ID NO:333;
a variable heavy chain (V_{H}) polypeptide comprising
the V_{H} CDR1, V_{H} CDR2 and V_{H} CDR3 of the V_{H} set forth in SEQ ID NO:337, and
a variable light chain (V_{L}) polypeptide comprising
the V_{L} CDR1, V_{L} CDR2 and V_{L} CDR3 of the V_{L} set forth in SEQ ID NO:341;
a variable heavy chain (V_{H}) polypeptide comprising
the V_{H} CDR1, V_{H} CDR2 and V_{H} CDR3 of the V_{H} set forth in SEQ ID NO:345, and
a variable light chain (V_{L}) polypeptide comprising
the V_{L} CDR1, V_{L} CDR2 and V_{L} CDR3 of the V_{L} set forth in SEQ ID NO:349;
a variable heavy chain (V_{H}) polypeptide comprising
the V_{H} CDR1, V_{H} CDR2 and V_{H} CDR3 of the V_{H} set forth in SEQ ID NO:353, and
a variable light chain (V_{L}) polypeptide comprising
the V_{L} CDR1, V_{L} CDR2 and V_{L} CDR3 of the V_{L} set forth in SEQ ID NO:357;
a variable heavy chain (V_{H}) polypeptide comprising
the V_{H} CDR1, V_{H} CDR2 and V_{H} CDR3 of the V_{H} set forth in SEQ ID NO:361, and
a variable light chain (V_{L}) polypeptide comprising
the V_{L} CDR1, V_{L} CDR2 and V_{L} CDR3 of the V_{L} set forth in SEQ ID NO:365;
a variable heavy chain (V_{H}) polypeptide comprising
the V_{H} CDR1, V_{H} CDR2 and V_{H} CDR3 of the V_{H} set forth in SEQ ID NO:369, and
a variable light chain (V_{L}) polypeptide comprising
the V_{L} CDR1, V_{L} CDR2 and V_{L} CDR3 of the V_{L} set forth in SEQ ID NO:373;
a variable heavy chain (V_{H}) polypeptide comprising
the V_{H} CDR1, V_{H} CDR2 and V_{H} CDR3 of the V_{H} set forth in SEQ ID NO:377, and
a variable light chain (V_{L}) polypeptide comprising
the V_{L} CDR1, V_{L} CDR2 and V_{L} CDR3 of the V_{L} set forth in SEQ ID NO:381;
a variable heavy chain (V_{H}) polypeptide comprising
the V_{H} CDR1, V_{H} CDR2 and V_{H} CDR3 of the V_{H} set forth in SEQ ID NO:385, and
a variable light chain (V_{L}) polypeptide comprising
the V_{L} CDR1, V_{L} CDR2 and V_{L} CDR3 of the V_{L} set forth in SEQ ID NO:389;
a variable heavy chain (V_{H}) polypeptide comprising
the V_{H} CDR1, V_{H} CDR2 and V_{H} CDR3 of the V_{H} set forth in SEQ ID NO:393, and
a variable light chain (V_{L}) polypeptide comprising
the V_{L} CDR1, V_{L} CDR2 and V_{L} CDR3 of the V_{L} set forth in SEQ ID NO:397;
a variable heavy chain (V_{H}) polypeptide comprising
the V_{H} CDR1, V_{H} CDR2 and V_{H} CDR3 of the V_{H} set forth in SEQ ID NO:401, and
a variable light chain (V_{L}) polypeptide comprising
the V_{L} CDR1, V_{L} CDR2 and V_{L} CDR3 of the V_{L} set forth in SEQ ID NO:405;
a variable heavy chain (V_{H}) polypeptide comprising
the V_{H} CDR1, V_{H} CDR2 and V_{H} CDR3 of the V_{H} set forth in SEQ ID NO:409, and
a variable light chain (V_{L}) polypeptide comprising
the V_{L} CDR1, V_{L} CDR2 and V_{L} CDR3 of the V_{L} set forth in SEQ ID NO:413;
a variable heavy chain (V_{H}) polypeptide comprising
the V_{H} CDR1, V_{H} CDR2 and V_{H} CDR3 of the V_{H} set forth in SEQ ID NO:417, and
a variable light chain (V_{L}) polypeptide comprising
the V_{L} CDR1, V_{L} CDR2 and V_{L} CDR3 of the V_{L} set forth in SEQ ID NO:421;
a variable heavy chain (V_{H}) polypeptide comprising
the V_{H} CDR1, V_{H} CDR2 and V_{H} CDR3 of the V_{H} set forth in SEQ ID NO:425, and
a variable light chain (V_{L}) polypeptide comprising
the V_{L} CDR1, V_{L} CDR2 and V_{L} CDR3 of the V_{L} set forth in SEQ ID NO:429;
a variable heavy chain (V_{H}) polypeptide comprising
the V_{H} CDR1, V_{H} CDR2 and V_{H} CDR3 of the V_{H} set forth in SEQ ID NO:433, and
a variable light chain (V_{L}) polypeptide comprising
the V_{L} CDR1, V_{L} CDR2 and V_{L} CDR3 of the V_{L} set forth in SEQ ID NO:437;
a variable heavy chain (V_{H}) polypeptide comprising
the V_{H} CDR1, V_{H} CDR2 and V_{H} CDR3 of the V_{H} set forth in SEQ ID NO:441, and
a variable light chain (V_{L}) polypeptide comprising
the V_{L} CDR1, V_{L} CDR2 and V_{L} CDR3 of the V_{L} set forth in SEQ ID NO:445;
a variable heavy chain (V_{H}) polypeptide comprising
the V_{H} CDR1, V_{H} CDR2 and V_{H} CDR3 of the V_{H} set forth in SEQ ID NO:449, and
a variable light chain (V_{L}) polypeptide comprising
the V_{L} CDR1, V_{L} CDR2 and V_{L} CDR3 of the V_{L} set forth in SEQ ID NO:453;
a variable heavy chain (V_{H}) polypeptide comprising
the V_{H} CDR1, V_{H} CDR2 and V_{H} CDR3 of the V_{H} set forth in SEQ ID NO:457, and
a variable light chain (V_{L}) polypeptide comprising
the V_{L} CDR1, V_{L} CDR2 and V_{L} CDR3 of the V_{L} set forth in SEQ ID NO:461; or
a variable heavy chain (V_{H}) polypeptide comprising
the V_{H} CDR1, V_{H} CDR2 and V_{H} CDR3 of the V_{H} set forth in SEQ ID NO:465, and
a variable light chain (V_{L}) polypeptide comprising
the V_{L} CDR1, V_{L} CDR2 and V_{L} CDR3 of the V_{L} set forth in SEQ ID NO:469.

3. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:1; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:5.

4. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:9; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:13.

5. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:17; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:21.

6. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:25; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:29.

7. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:33; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:37.

8. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:41; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:45.

9. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:49; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:53.

10. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:57; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:61.

11. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:65; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:69.

12. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:73; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:77.

13. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:81; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:85.

14. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:89; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:93.

15. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:97; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:101.

16. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:105; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:109.

17. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO: 113; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO: 117.

18. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:121; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:125.

19. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:129; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:133.

20. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:137; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:141.

21. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:145; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:149.

22. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:153; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:157.

23. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:161; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:165.

24. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:169; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:173.

25. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:177; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:181.

26. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:185; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:189.

27. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:193; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:197.

28. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:201; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:205.

29. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:209; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:213.

30. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:217; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:221.

31. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:225; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:229.

32. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:233; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:237.

33. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:241; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:245.

34. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:249; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:253.

35. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:257; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:261.

36. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:265; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:269.

37. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:273; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:277.

38. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:281; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:285.

39. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:289; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:293.

40. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:297; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:301.

41. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:305; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:309.

42. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:313; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:317.

43. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:321; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:325.

44. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:329; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:333.

45. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:337; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:341.

46. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:345; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:349.

47. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:353; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:357.

48. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:361; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:365.

49. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:369; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:373.

50. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:377; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:381.

51. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:385; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:389.

52. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:393; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:397.

53. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:401; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:405.

54. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:409; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:413.

55. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:417; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:421.

56. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:425; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:429.

57. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:433; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:437.

58. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:441; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:445.

59. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:449; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:453.

60. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:457; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:461.

61. The antibody of claim 1 or claim 2, wherein the antibody comprises:
a variable heavy chain (V_{H}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:465; and
a variable light chain (V_{L}) polypeptide comprising an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% identity to the amino acid sequence set forth in SEQ ID NO:469.

62. The antibody of any one of claims 1 to 61, wherein the antibody is selected from the group consisting of: an IgG, Fv, single chain antibody, scFv, Fab, F(ab′)2, or Fab′.

63. The antibody of any one of claims 1 to 61, wherein the antibody is an IgG.

64. The antibody of claim 63, wherein the antibody is an IgG1.

65. The antibody of any one of claims 1 to 64, wherein the antibody comprises an Fc region, and the Fc region is heterologous to the V_{H} of the antibody.

66. The antibody of claim 65, wherein the Fc region is a variant Fc region.

67. The antibody of claim 66, wherein the variant Fc region comprises one or more amino acid substitutions, one or more amino acid insertions, one or more amino acid deletions, or any combination thereof, relative to a wild-type Fc region.

68. The antibody of any one of claims 1 to 61, wherein the antibody is a Fab.

69. The antibody of any one of claims 1 to 61, wherein the antibody is a single chain antibody.

70. The antibody of claim 69, wherein the antibody is an scFv.

71. The antibody of any one of claims 1 to 67, wherein the antibody is a recombinant antibody.

72. The antibody of any one of claims 1 to 67, wherein the antibody is a monoclonal antibody.

73. The antibody of any one of claims 1 to 72, wherein the antibody comprises an extent of glycosylation, a glycosylation pattern, or both, which is different from the extent of glycosylation, the glycosylation pattern, or both, of a naturally occurring antibody.

74. The antibody of any one of claims 1 to 73, wherein the antibody specifically binds a SARS-CoV-2 antigen selected from the group consisting of: the S1 subunit of a SARS-CoV-2 spike (S) protein, the receptor-binding domain (RBD) of the S1 subunit of a SARS-CoV-2 spike protein, the S2 subunit of a SARS-CoV-2 spike protein, a SARS-CoV-2 spike (S) protein trimer, a SARS-CoV-2 envelope (E) protein, a SARS-CoV-2 membrane (M) protein, and a SARS-CoV-2 nucleocapsid (N) protein.

75. The antibody of any one of claims 1 to 74, wherein the antibody is a bispecific antibody comprising a first antigen-binding domain that specifically binds SARS-CoV-2, and wherein the first antigen binding domain comprises a V_{H} polypeptide-V_{L} polypeptide pair as defined in any one of claims 1 to 61.

76. The antibody of claim 75, wherein the bispecific antibody comprises a second antigen-binding domain that specifically binds a SARS-CoV-2 antigen.

77. The antibody of claim 75, wherein the bispecific antibody comprises a second antigen-binding domain that specifically binds an antigen other than a SARS-CoV-2 antigen.

78. A fusion protein, comprising:
a chain of an antibody of any one of claims 1 to 61 fused to a heterologous sequence of amino acids.

79. The fusion protein of claim 78, wherein the heterologous sequence of amino acids is fused to the C-terminus of the chain of the antibody.

80. The fusion protein of claim 78 or claim 79, wherein the antibody is the single chain antibody of claim 69 or 70.

81. A conjugate, comprising:
an antibody of any one of claims 1 to 61 or a fusion protein of any one of claims 78 to 80; and
an agent conjugated to the antibody or fusion protein.

82. The conjugate of claim 81, wherein the agent is a detectable label or a half-life extending moiety.

83. The conjugate of claim 82, wherein the detectable label is a radiolabel.

84. The conjugate of claim 82, wherein the detectable label is an in vivo imaging agent.

85. The conjugate of any one of claims 81 to 84, wherein the agent is conjugated to the antibody or fusion protein via a non-cleavable linker.

86. The conjugate of any one of claims 81 to 84, wherein the agent is conjugated to the antibody or fusion protein via a cleavable linker.

87. A nucleic acid encoding a variable heavy chain (V_{H}) polypeptide, a variable light chain (V_{L}) polypeptide, or both, of the antibody of any one of claims 1 to 70.

88. The nucleic acid of claim 87, wherein the antibody is a single chain antibody, and wherein the nucleic acid encodes the single chain antibody.

89. The nucleic acid of claim 88, wherein the single chain antibody is an scFv.

90. An expression vector comprising the nucleic acid of any one of claims 87 to 89.

91. A cell comprising the nucleic acid of any one of claims 87 to 89 or the expression vector of claim 90.

92. The cell of claim 91, wherein the nucleic acid encodes the V_{H} polypeptide of the antibody and the V_{L} polypeptide of the antibody.

93. The cell of claim 92, wherein the antibody is a single chain antibody, and wherein the nucleic acid encodes the single chain antibody.

94. The cell of claim 93, wherein the single chain antibody is an scFv.

95. A cell comprising:
a first nucleic acid encoding a variable heavy chain (V_{H}) polypeptide of the antibody of any one of claims 1 to 68; and
a second nucleic acid encoding a variable light chain (V_{L}) polypeptide of the antibody.

96. The cell of claim 95, comprising:
a first expression vector comprising the first nucleic acid; and
a second expression vector comprising the second nucleic acid.

97. A method of making the antibody of any one of claims 1 to 77, comprising culturing the cell of any one of claims 91 to 96 under conditions suitable for the cell to express the antibody, wherein the antibody is produced.

98. The method according to claim 97, further comprising, prior to the culturing, transfecting the cell or an ancestor thereof with an expression vector encoding the V_{H}, the V_{L}, or both, of the antibody.

99. A pharmaceutical composition, comprising:
the antibody of any one of claims 1 to 77; and
a pharmaceutically acceptable carrier.

100. A pharmaceutical composition, comprising:
two or more different antibodies each having a V_{H} and V_{L} pair as defined in any one of claims 1 to 61; and
a pharmaceutically acceptable carrier.

101. The pharmaceutical composition of claim 100, wherein the two or more different antibodies comprise:
a first antibody that specifically binds the receptor-binding domain (RBD) of the S1 subunit of a SARS-CoV-2 spike (S) protein; and
a second antibody that specifically binds the S2 subunit of the SARS-CoV-2 spike (S) protein.

102. The pharmaceutical composition of claim 100, wherein the two or more different antibodies comprise:
a first antibody which is a class I RBD-binding antibody; and
a second antibody which is a class III RBD-binding antibody.

103. A pharmaceutical composition, comprising:
the fusion protein of any one of claims 78 to 80; and
a pharmaceutically acceptable carrier.

104. A pharmaceutical composition, comprising:
the conjugate of any one of claims 81 to 86; and
a pharmaceutically acceptable carrier.

105. The pharmaceutical composition of any one of claims 99 to 104, comprising:
a first antibody comprising:
a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:25, and
a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:29,
wherein the antibody comprises one or more, two or more, three or more, four or more, five or six of the complementarity determining regions (CDRs) of an antibody comprising the V_{H} and V_{L} set forth in SEQ ID NO:25 and SEQ ID NO:29, respectively; and
a second antibody comprising:
a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:33, and
a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:37,
wherein the antibody comprises one or more, two or more, three or more, four or more, five or six of the complementarity determining regions (CDRs) of an antibody comprising the V_{H} and V_{L} set forth in SEQ ID NO:33 and SEQ ID NO:37, respectively.

106. The pharmaceutical composition of any one of claims 99 to 104, comprising:
a first antibody comprising:
a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:25, and
a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:29,
wherein the antibody comprises one or more, two or more, three or more, four or more, five or six of the complementarity determining regions (CDRs) of an antibody comprising the V_{H} and V_{L} set forth in SEQ ID NO:25 and SEQ ID NO:29, respectively; and
a second antibody comprising:
a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:81, and
a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:85,
wherein the antibody comprises one or more, two or more, three or more, four or more, five or six of the complementarity determining regions (CDRs) of an antibody comprising the V_{H} and V_{L} set forth in SEQ ID NO:81 and SEQ ID NO:85, respectively.

107. The pharmaceutical composition of any one of claims 99 to 104, comprising:
a first antibody comprising:
a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:33, and
a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:37,
wherein the antibody comprises one or more, two or more, three or more, four or more, five or six of the complementarity determining regions (CDRs) of an antibody comprising the V_{H} and V_{L} set forth in SEQ ID NO:33 and SEQ ID NO:37, respectively; and
a second antibody comprising:
a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:81, and
a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:85,
wherein the antibody comprises one or more, two or more, three or more, four or more, five or six of the complementarity determining regions (CDRs) of an antibody comprising the V_{H} and V_{L} set forth in SEQ ID NO:81 and SEQ ID NO:85, respectively.

108. The pharmaceutical composition of any one of claims 99 to 107, wherein the pharmaceutical composition is formulated for parenteral administration.

109. The pharmaceutical composition of claim 108, wherein the pharmaceutical composition is formulated for intravenous, intramuscular, or subcutaneous administration.

110. The pharmaceutical composition of any one of claims 99 to 107, wherein the pharmaceutical composition is formulated for inhalational administration.

111. The pharmaceutical composition of any one of claims 99 to 107, wherein the pharmaceutical composition is formulated for intranasal administration.

112. The pharmaceutical composition of any one of claims 99 to 108, wherein the composition provides a unit dosage effective to neutralize a SARS-CoV-2 virus infection.

113. A kit, comprising:
the pharmaceutical composition of any one of claims 99 to 112; and
instructions for administering an effective amount of the pharmaceutical composition to an individual in need thereof.

114. The kit of claim 113, wherein the pharmaceutical composition is present in one or more unit dosages.

115. The kit of claim 113, wherein the pharmaceutical composition is present in two or more unit dosages.

116. The kit of any one of claims 113 to 115, wherein the individual in need thereof has or is suspected of having a SARS-CoV-2 infection.

117. The kit of any one of claims 113 to 115, wherein the individual in need thereof is known to be immunocompromised and is not suspected of having a SARS-CoV-2 infection.

118. A method comprising administering to an individual in need thereof a therapeutically effective amount of the antibody of any one of claims 1 to 77, the fusion protein of any one of claims 78 to 80, or the conjugate of any one of claims 81 to 86.

119. The method according to claim 118, wherein the individual has a SARS-CoV-2 infection, and wherein the method is effective in neutralizing the SARS-CoV-2 infection in the individual.

120. The method according to claim 118, wherein the antibody, fusion protein or conjugate is administered prophylactically to an individual who does not have a SARS-CoV-2 infection.

121. The method according to claim 120, wherein the individual who does not have a SARS-CoV-2 infection is immunocompromised.

122. The method according to claim 121, wherein the individual is immunocompromised by virtue of having cancer, taking one or more immunosuppressive drugs, being a transplant recipient, having HIV/AIDS, having an inherited disease that affects the immune system, having congenital agammaglobulinemia, having congenital IgA deficiency, being elderly, or any combination thereof.

123. A method of treating an individual having a SARS-CoV-2 infection, the method comprising:
administering to the individual a therapeutically effective amount of the antibody of any one of claims 1 to 77, the fusion protein of any one of claims 78 to 80, or the conjugate of any one of claims 81 to 86.

124. The method according to any one of claims 118 to 123, the method comprising administering to the individual a therapeutically effective amount of a combination of two or more different antibodies each having a V_{H} and V_{L} pair as defined in any one of claims 1 to 61.

125. The method according to claim 124, wherein the two or more different antibodies comprise:
a first antibody that specifically binds the receptor-binding domain (RBD) of the S1 subunit of a SARS-CoV-2 spike (S) protein; and
a second antibody that specifically binds the S2 subunit of the SARS-CoV-2 spike (S) protein.

126. The method according to claim 124, wherein the two or more different antibodies comprise:
a first antibody which is a class I RBD-binding antibody; and
a second antibody which is a class III RBD-binding antibody.

127. The method according to claim 123, the method comprising administering to the individual a therapeutically effective amount of a combination of two or more different antibodies, or fusion proteins or conjugates comprising same, the two or more different antibodies comprising:
a first antibody comprising:
a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:25, and
a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:29,
wherein the antibody comprises one or more, two or more, three or more, four or more, five or six of the complementarity determining regions (CDRs) of an antibody comprising the V_{H} and V_{L} set forth in SEQ ID NO:25 and SEQ ID NO:29, respectively; and
a second antibody comprising:
a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:33, and
a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:37,
wherein the antibody comprises one or more, two or more, three or more, four or more, five or six of the complementarity determining regions (CDRs) of an antibody comprising the V_{H} and V_{L} set forth in SEQ ID NO:33 and SEQ ID NO:37, respectively.

128. The method according to claim 123, the method comprising administering to the individual a therapeutically effective amount of a combination of two or more different antibodies, or fusion proteins or conjugates comprising same, the two or more different antibodies comprising:
a first antibody comprising:
a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:25, and
a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:29,
wherein the antibody comprises one or more, two or more, three or more, four or more, five or six of the complementarity determining regions (CDRs) of an antibody comprising the V_{H} and V_{L} set forth in SEQ ID NO:25 and SEQ ID NO:29, respectively; and
a second antibody comprising:
a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:81, and
a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:85,
wherein the antibody comprises one or more, two or more, three or more, four or more, five or six of the complementarity determining regions (CDRs) of an antibody comprising the V_{H} and V_{L} set forth in SEQ ID NO:81 and SEQ ID NO:85, respectively.

129. The method according to claim 123, the method comprising administering to the individual a therapeutically effective amount of a combination of two or more different antibodies, or fusion proteins or conjugates comprising same, the two or more different antibodies comprising:
a first antibody comprising:
a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:33, and
a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:37,
wherein the antibody comprises one or more, two or more, three or more, four or more, five or six of the complementarity determining regions (CDRs) of an antibody comprising the V_{H} and V_{L} set forth in SEQ ID NO:33 and SEQ ID NO:37, respectively; and
a second antibody comprising:
a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} set forth in SEQ ID NO:81, and
a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} set forth in SEQ ID NO:85,
wherein the antibody comprises one or more, two or more, three or more, four or more, five or six of the complementarity determining regions (CDRs) of an antibody comprising the V_{H} and V_{L} set forth in SEQ ID NO:81 and SEQ ID NO:85, respectively.

130. The method according to any one of claims 118 to 129, wherein the administering is by parenteral administration.

131. The method according to claim 130, wherein the administering is by intravenous, intramuscular, or subcutaneous administration.

132. The method according to any one of claims 118 to 129, wherein the administering is by inhalational administration.

133. The method according to any one of claims 118 to 129, wherein the administering is by intranasal administration.


SEQUENCE LISTING (501 sequences)

SEQ ID NO: 1 - protein, 120 aa, Artificial sequence
  synthetic sequence
QVQLQESGPRLVKPSETLSLTCTVSGDSISSYYWSWIRQPPGKGLEWIGYIYYSGSTNYNPSLKSRVTISVDTSKNQFSLKLSSVTAADTAVYYCARKTVAGPAGEFDYWGQGTLVTVSS

SEQ ID NO: 2 - protein, 8 aa, Artificial sequence
  synthetic sequence
GDSISSYY

SEQ ID NO: 3 - protein, 7 aa, Artificial sequence
  synthetic sequence
IYYSGST

SEQ ID NO: 4 - protein, 14 aa, Artificial sequence
  synthetic sequence
ARKTVAGPAGEFDY

SEQ ID NO: 5 - protein, 112 aa, Artificial sequence
  synthetic sequence
QSVLTQPPSVSGAPGQRVTISCTGSSSNIGAGYDVHWYQQFPGTAPKLLIYGNSNRPSGVPDRFSGSKSGTSASLAITGLQAEDEADYYCQSYDSSLSGPYVFGTGTKVTVL

SEQ ID NO: 6 - protein, 9 aa, Artificial sequence
  synthetic sequence
SSNIGAGYD

SEQ ID NO: 7 - protein, 3 aa, Artificial sequence
  synthetic sequence
GNS

SEQ ID NO: 8 - protein, 12 aa, Artificial sequence
  synthetic sequence
QSYDSSLSGPYV

SEQ ID NO: 9 - protein, 120 aa, Artificial sequence
  synthetic sequence
QVQVVQSGAEVKKPGASVKVSCTASGHTFTGYAIHWVRQAPGQRLEWMGLINAGGIYRTYSQRFQGRVTITRDTSASTAYMELTGLTSEDTAVYYCARANYGSGSFSDHWGQGTLVTVSS

SEQ ID NO: 10 - protein, 8 aa, Artificial sequence
  synthetic sequence
GHTFTGYA

SEQ ID NO: 11 - protein, 8 aa, Artificial sequence
  synthetic sequence
INAGGIYR

SEQ ID NO: 12 - protein, 13 aa, Artificial sequence
  synthetic sequence
ARANYGSGSFSDH

SEQ ID NO: 13 - protein, 107 aa, Artificial sequence
  synthetic sequence
DIQMTQSPSSLSASVGDRVTITCRASQSISSYLNWYQQKPGKAPKLLIYAASSLQSGVPSRFSGSGSGTDFTLTISSLQPEDFATYYCQQSSSTPYTFGQGTKLEIK

SEQ ID NO: 14 - protein, 6 aa, Artificial sequence
  synthetic sequence
QSISSY

SEQ ID NO: 15 - protein, 3 aa, Artificial sequence
  synthetic sequence
AAS

SEQ ID NO: 16 - protein, 9 aa, Artificial sequence
  synthetic sequence
QQSSSTPYT

SEQ ID NO: 17 - protein, 117 aa, Artificial sequence
  synthetic sequence
EVQLVESGGGLVQPGGSLRLSCAASGFTVSSNYMSWVRQAPGKGLEWVSVIYSGGSTFYADSVKGRFTISRDNSRNTLYLQMNSLRAEDTAVYYCARDWGEFFFDFWGQGALVTVSS

SEQ ID NO: 18 - protein, 8 aa, Artificial sequence
  synthetic sequence
GFTVSSNY

SEQ ID NO: 19 - protein, 7 aa, Artificial sequence
  synthetic sequence
IYSGGST

SEQ ID NO: 20 - protein, 11 aa, Artificial sequence
  synthetic sequence
ARDWGEFFFDF

SEQ ID NO: 21 - protein, 108 aa, Artificial sequence
  synthetic sequence
EIVLTQSPGTLSLSPGERATLSCRASQTISSSYLAWYQQKPGQAPRLLISGASSRATGIPDRFSGSGSGTDFTLTISRLEPEDFAVYYCQQYGSSPRTFGQGTNVEIK

SEQ ID NO: 22 - protein, 7 aa, Artificial sequence
  synthetic sequence
QTISSSY

SEQ ID NO: 23 - protein, 3 aa, Artificial sequence
  synthetic sequence
GAS

SEQ ID NO: 24 - protein, 9 aa, Artificial sequence
  synthetic sequence
QQYGSSPRT

SEQ ID NO: 25 - protein, 130 aa, Artificial sequence
  synthetic sequence
EVQLVESGGGLVQPGRSLRLSCAASGFTFDDYAMHWVRQAPGKGLEWVSGISWNSGSIGYADSVKGRFTISRDNAKNSLYLQMNSLKPEDTALYYCAKSTTSCYNGSQCPTKRPKPLESWGQGTLVIVSA

SEQ ID NO: 26 - protein, 8 aa, Artificial sequence
  synthetic sequence
GFTFDDYA

SEQ ID NO: 27 - protein, 8 aa, Artificial sequence
  synthetic sequence
ISWNSGSI

SEQ ID NO: 28 - protein, 23 aa, Artificial sequence
  synthetic sequence
AKSTTSCYNGSQCPTKRPKPLES

SEQ ID NO: 29 - protein, 107 aa, Artificial sequence
  synthetic sequence
DIQMTQSPSTLSAFVGDRVTITCRASQSINNWLAWYQQKPGEVPKLLIYKASSLENGVPSRFSGSGFGTEFTLTISSLQPDDFAIYYCQQYYSHSHTFGQGTKLEIK

SEQ ID NO: 30 - protein, 6 aa, Artificial sequence
  synthetic sequence
QSINNW

SEQ ID NO: 31 - protein, 3 aa, Artificial sequence
  synthetic sequence
KAS

SEQ ID NO: 32 - protein, 9 aa, Artificial sequence
  synthetic sequence
QQYYSHSHT

SEQ ID NO: 33 - protein, 129 aa, Artificial sequence
  synthetic sequence
QVQLVQSGAEVKKPGSSVKVSCKASGGTFSNSAINWVRQAPGQGLEWMGGIIPLFGAADYAQKFQARVTITADKSTTTSYMELSSLRSEDTAVYYCARDTLEHDDVWGNFRLSLPLSFWGQGTLVTVSS

SEQ ID NO: 34 - protein, 8 aa, Artificial sequence
  synthetic sequence
GGTFSNSA

SEQ ID NO: 35 - protein, 8 aa, Artificial sequence
  synthetic sequence
IIPLFGAA

SEQ ID NO: 36 - protein, 22 aa, Artificial sequence
  synthetic sequence
ARDTLEHDDVWGNFRLSLPLSF

SEQ ID NO: 37 - protein, 108 aa, Artificial sequence
  synthetic sequence
EIVMTQSPATLSVSPGERATLSCRASQSVSSKLAWYQQKPGQAPRLLFYGASTRATGLPARFSGSGSGTEFTLTISSLQSEDFAVYYCQQYNDWPMYTFGPGTRLEIK

SEQ ID NO: 38 - protein, 6 aa, Artificial sequence
  synthetic sequence
QSVSSK

SEQ ID NO: 39 - protein, 3 aa, Artificial sequence
  synthetic sequence
GAS

SEQ ID NO: 40 - protein, 10 aa, Artificial sequence
  synthetic sequence
QQYNDWPMYT

SEQ ID NO: 41 - protein, 127 aa, Artificial sequence
  synthetic sequence
QVQLVESGGGVVQPGRSLRLSCAASGFTFSRYGMHWVRQAPGKGLEWVALISSGGSNKYYADSVKGRFTISRDNSKNTLYLEMNSLRAEDTAVYYCAKDAIYDYIWGAYRENWFDPWGQGTLVTVSS

SEQ ID NO: 42 - protein, 8 aa, Artificial sequence
  synthetic sequence
GFTFSRYG

SEQ ID NO: 43 - protein, 8 aa, Artificial sequence
  synthetic sequence
ISSGGSNK

SEQ ID NO: 44 - protein, 20 aa, Artificial sequence
  synthetic sequence
AKDAIYDYIWGAYRENWFDP

SEQ ID NO: 45 - protein, 107 aa, Artificial sequence
  synthetic sequence
DIQMTQSPSSLSASVGDRVTITCRASQSISNYLNWYQQKPGKAPNLLIYAASSLQSGVPSRFSGSGSGTDFTLTISSLQPEDFATYYCQQTSSPPLTFGQGTKVEIK

SEQ ID NO: 46 - protein, 6 aa, Artificial sequence
  synthetic sequence
QSISNY

SEQ ID NO: 47 - protein, 3 aa, Artificial sequence
  synthetic sequence
AAS

SEQ ID NO: 48 - protein, 9 aa, Artificial sequence
  synthetic sequence
QQTSSPPLT

SEQ ID NO: 49 - protein, 126 aa, Artificial sequence
  synthetic sequence
QVQLVQSGAEVKKPGSSVKVSCKASGGTFSSYAISWVRQAPGQGLEWMGGIIPVFGTTNYAQEFRARVTITADESTNTAYMELRSLSSADTGVYYCATGAGEMIEVTIANDVFDIWGQGTRVTVSS

SEQ ID NO: 50 - protein, 8 aa, Artificial sequence
  synthetic sequence
GGTFSSYA

SEQ ID NO: 51 - protein, 8 aa, Artificial sequence
  synthetic sequence
IIPVFGTT

SEQ ID NO: 52 - protein, 19 aa, Artificial sequence
  synthetic sequence
ATGAGEMIEVTIANDVFDI

SEQ ID NO: 53 - protein, 111 aa, Artificial sequence
  synthetic sequence
QLVLTQSPSASASLGASVKLTCTLSSGHSSYAIAWHQQQPEKGPRYLMKLNSDGSHSKGDGIPDRFSGSSSGAERYLTISSLQSDDEADYYCQTWGTAIHVFGTGTKVTVL

SEQ ID NO: 54 - protein, 7 aa, Artificial sequence
  synthetic sequence
SGHSSYA

SEQ ID NO: 55 - protein, 7 aa, Artificial sequence
  synthetic sequence
LNSDGSH

SEQ ID NO: 56 - protein, 9 aa, Artificial sequence
  synthetic sequence
QTWGTAIHV

SEQ ID NO: 57 - protein, 122 aa, Artificial sequence
  synthetic sequence
QMQLVQSGAEVKKTGSSVKVSCEASENTFTNRYLHWVRQAPGQALEWMGWITPFQGNTHYAQKFQDRVTITRDRSMNTVYMELSSLRSEDTAIYYCASGGQYGPGSYYFEFWGQGTLVTVSS

SEQ ID NO: 58 - protein, 8 aa, Artificial sequence
  synthetic sequence
ENTFTNRY

SEQ ID NO: 59 - protein, 8 aa, Artificial sequence
  synthetic sequence
ITPFQGNT

SEQ ID NO: 60 - protein, 15 aa, Artificial sequence
  synthetic sequence
ASGGQYGPGSYYFEF

SEQ ID NO: 61 - protein, 108 aa, Artificial sequence
  synthetic sequence
EIVMTQSPATLSVSPGERATLSCRASQSIRNNLAWYRQKPGQAPRLLIYGASTRAAGVPARFSGSGSGTEFTLTISSLQSEDFAVYYCQQYINWPPLTFGGGTKVEIK

SEQ ID NO: 62 - protein, 6 aa, Artificial sequence
  synthetic sequence
QSIRNN

SEQ ID NO: 63 - protein, 3 aa, Artificial sequence
  synthetic sequence
GAS

SEQ ID NO: 64 - protein, 10 aa, Artificial sequence
  synthetic sequence
QQYINWPPLT

SEQ ID NO: 65 - protein, 122 aa, Artificial sequence
  synthetic sequence
QMQLVQSGAEVKKTGSSVKVSCEASGYTFTNRYLHWVRQAPGQALEWMGWITPFQGNTNYAQKFQDRVTITRDMSMNTVYMELSSLRSEDTAKYYCASGGQYGAGSYYLEDWGQGTLVTVSS

SEQ ID NO: 66 - protein, 8 aa, Artificial sequence
  synthetic sequence
GYTFTNRY

SEQ ID NO: 67 - protein, 8 aa, Artificial sequence
  synthetic sequence
ITPFQGNT

SEQ ID NO: 68 - protein, 15 aa, Artificial sequence
  synthetic sequence
ASGGQYGAGSYYLED

SEQ ID NO: 69 - protein, 108 aa, Artificial sequence
  synthetic sequence
EIVMTQSPATLSVSPGERATLSCRASQSVRSNLAWYRQKPGQAPRLLIYGASTRATGVPARFSGSGSGTEFTLTISSLQSEDFAVYYCQQYINWPPLTFGGGTRVEIK

SEQ ID NO: 70 - protein, 6 aa, Artificial sequence
  synthetic sequence
QSVRSN

SEQ ID NO: 71 - protein, 3 aa, Artificial sequence
  synthetic sequence
GAS

SEQ ID NO: 72 - protein, 10 aa, Artificial sequence
  synthetic sequence
QQYINWPPLT

SEQ ID NO: 73 - protein, 119 aa, Artificial sequence
  synthetic sequence
QVQLVQSGSELKKPGASVKVSCTGYGYTFTSYAMNWVRQAPGQGLEWMGWINTNTGHPAYAQGFTGRFVFSLDSSVSTAYLQISSLKAEDTAVYYCATTIRAGGYFDFWGQGTLVTVSS

SEQ ID NO: 74 - protein, 8 aa, Artificial sequence
  synthetic sequence
GYTFTSYA

SEQ ID NO: 75 - protein, 8 aa, Artificial sequence
  synthetic sequence
INTNTGHP

SEQ ID NO: 76 - protein, 12 aa, Artificial sequence
  synthetic sequence
ATTIRAGGYFDF

SEQ ID NO: 77 - protein, 107 aa, Artificial sequence
  synthetic sequence
DIQLTQSPSFLSASVGDRVTITCRASQGISSYLAWFQQKPGKAPKLLIYVVSILQSGVPSRFSGSGSGTEFTLTISSLQPEDFATYYCQQLNSYPYTFGQGTKLEIK

SEQ ID NO: 78 - protein, 6 aa, Artificial sequence
  synthetic sequence
QGISSY

SEQ ID NO: 79 - protein, 3 aa, Artificial sequence
  synthetic sequence
VVS

SEQ ID NO: 80 - protein, 9 aa, Artificial sequence
  synthetic sequence
QQLNSYPYT

SEQ ID NO: 81 - protein, 118 aa, Artificial sequence
  synthetic sequence
QVQLVQSGSELKKPGASVKVSCKASGYTFTNYGMNWVRQAPGQGLEWMGWINTNTGNPTNAQGFTGRFVFSLDTSVSTAYLQINSLKGEDTAVYYCARVADTGEMDVWGQGTTVTVSS

SEQ ID NO: 82 - protein, 8 aa, Artificial sequence
  synthetic sequence
GYTFTNYG

SEQ ID NO: 83 - protein, 8 aa, Artificial sequence
  synthetic sequence
INTNTGNP

SEQ ID NO: 84 - protein, 11 aa, Artificial sequence
  synthetic sequence
ARVADTGEMDV

SEQ ID NO: 85 - protein, 109 aa, Artificial sequence
  synthetic sequence
SYELTQPPSVSVSPGQTARITCSGDALPKQYAYWYQQKPGQAPVLVIYKDSERPSGIPERFSGSSSGTTVTLTISGVQAEDEADYYCQSADSSGSYNWVFGGGTKLTIL

SEQ ID NO: 86 - protein, 6 aa, Artificial sequence
  synthetic sequence
ALPKQY

SEQ ID NO: 87 - protein, 3 aa, Artificial sequence
  synthetic sequence
KDS

SEQ ID NO: 88 - protein, 12 aa, Artificial sequence
  synthetic sequence
QSADSSGSYNWV

SEQ ID NO: 89 - protein, 121 aa, Artificial sequence
  synthetic sequence
QVQLQESGPGLVKPSETLSLTCTVSGDSISTYYWSWIRQPPGKGLEWIGYIHYSGSTNYNPSLKSRVAVSVDTSKNQFSLKLSSVTAADTAVYYCARGGGVFGVVINFDYWGQGILVTVSS

SEQ ID NO: 90 - protein, 8 aa, Artificial sequence
  synthetic sequence
GDSISTYY

SEQ ID NO: 91 - protein, 7 aa, Artificial sequence
  synthetic sequence
IHYSGST

SEQ ID NO: 92 - protein, 15 aa, Artificial sequence
  synthetic sequence
ARGGGVFGVVINFDY

SEQ ID NO: 93 - protein, 111 aa, Artificial sequence
  synthetic sequence
NFMLTQPYSVSESPGKTVTISCTRSSGSIASNYVQWYQQRPGSAPTTVIYEDNQRPSGVPDRFSGSIDSSSNSASLTISGLKTEDEADYYCQSRDSSNPVVFGGGTKLTVL

SEQ ID NO: 94 - protein, 8 aa, Artificial sequence
  synthetic sequence
SGSIASNY

SEQ ID NO: 95 - protein, 3 aa, Artificial sequence
  synthetic sequence
EDN

SEQ ID NO: 96 - protein, 10 aa, Artificial sequence
  synthetic sequence
QSRDSSNPVV

SEQ ID NO: 97 - protein, 119 aa, Artificial sequence
  synthetic sequence
EVQLVESGGGLIQVGGSLRLSCAASGLTVTSNYMNWVRQGPGKGLEWVSLIYSGGTTYYADSVKGRFTISRDDSKNTLYLQMNSLRAEDTAVYYCARPIVGARSGMDVWGQGTAVTVSS

SEQ ID NO: 98 - protein, 8 aa, Artificial sequence
  synthetic sequence
GLTVTSNY

SEQ ID NO: 99 - protein, 7 aa, Artificial sequence
  synthetic sequence
IYSGGTT

SEQ ID NO: 100 - protein, 13 aa, Artificial sequence
  synthetic sequence
ARPIVGARSGMDV

SEQ ID NO: 101 - protein, 107 aa, Artificial sequence
  synthetic sequence
DIQMTQSPSSLSASVGDRVTITCQASQDINKYLNWYQQKPGKAPKLLIYDASNLETGVPSRFSGSGSGTDFTFTISSLQPEDLATYYCHQFDNLPGTFGGGTKVEIK

SEQ ID NO: 102 - protein, 6 aa, Artificial sequence
  synthetic sequence
QDINKY

SEQ ID NO: 103 - protein, 3 aa, Artificial sequence
  synthetic sequence
DAS

SEQ ID NO: 104 - protein, 9 aa, Artificial sequence
  synthetic sequence
HQFDNLPGT

SEQ ID NO: 105 - protein, 126 aa, Artificial sequence
  synthetic sequence
EVQLVESGGGLVKPGGSLRLSCAASGFTFSTYNMNWVRQAPGKGLEWVSSISSNSNYIYYADSMKGRFTISRDNAKNSLYLQMNSLRAEDTAVYYCARDMDPLPYFDWLLYAFDVWGQGTMVTVSS

SEQ ID NO: 106 - protein, 8 aa, Artificial sequence
  synthetic sequence
GFTFSTYN

SEQ ID NO: 107 - protein, 8 aa, Artificial sequence
  synthetic sequence
ISSNSNYI

SEQ ID NO: 108 - protein, 19 aa, Artificial sequence
  synthetic sequence
ARDMDPLPYFDWLLYAFDV

SEQ ID NO: 109 - protein, 108 aa, Artificial sequence
  synthetic sequence
EIVLTQSPGTLSLSPGERATLSCRASQSVSGSYLAWYQQKPGQAPRLLIYGASSRATGIPDRFSGSGSGTDFTLTISRLEPEDFAVYYCQQYGSPIFTFGPGTKVDIK

SEQ ID NO: 110 - protein, 7 aa, Artificial sequence
  synthetic sequence
QSVSGSY

SEQ ID NO: 111 - protein, 3 aa, Artificial sequence
  synthetic sequence
GAS

SEQ ID NO: 112 - protein, 9 aa, Artificial sequence
  synthetic sequence
QQYGSPIFT

SEQ ID NO: 113 - protein, 119 aa, Artificial sequence
  synthetic sequence
QITLKESGPTLVKPTQTLTLTCTFSGFSLSTVGVGVAWIRQPPGKALEWLALIYWDDDKRYSPSLKSRLTITKDTSKNHVVLTMTNMDPVDTATYYCAHHIIAALVDVWGKGTTVTVSS

SEQ ID NO: 114 - protein, 10 aa, Artificial sequence
  synthetic sequence
GFSLSTVGVG

SEQ ID NO: 115 - protein, 7 aa, Artificial sequence
  synthetic sequence
IYWDDDK

SEQ ID NO: 116 - protein, 11 aa, Artificial sequence
  synthetic sequence
AHHIIAALVDV

SEQ ID NO: 117 - protein, 111 aa, Artificial sequence
  synthetic sequence
QSALTQPASVSGSPGQSITISCTGTSSDVGGNNYVSWYQHHPGEAPKLMIYEVSNRPSGVSNRFSGSKSGNTASLTISGLQAEDEADYYCSSYTTSSTLVVFGGGSKLTVL

SEQ ID NO: 118 - protein, 9 aa, Artificial sequence
  synthetic sequence
SSDVGGNNY

SEQ ID NO: 119 - protein, 3 aa, Artificial sequence
  synthetic sequence
EVS

SEQ ID NO: 120 - protein, 11 aa, Artificial sequence
  synthetic sequence
SSYTTSSTLVV

SEQ ID NO: 121 - protein, 128 aa, Artificial sequence
  synthetic sequence
QVQLQESGPGLVKPSETLSLTCSVSGGSINYYYWSWIRQTPGQGLEWIGFIYSSGTTNYNPSLKSRVTMSKDTAKKQFSLKLTSVTAADSAVYYCARHSRSCTNGVCQTYYYYALDVWGHGTTVTVSS

SEQ ID NO: 122 - protein, 8 aa, Artificial sequence
  synthetic sequence
GGSINYYY

SEQ ID NO: 123 - protein, 7 aa, Artificial sequence
  synthetic sequence
IYSSGTT

SEQ ID NO: 124 - protein, 22 aa, Artificial sequence
  synthetic sequence
ARHSRSCTNGVCQTYYYYALDV

SEQ ID NO: 125 - protein, 112 aa, Artificial sequence
  synthetic sequence
QSVLTQPPSVSGAPGQRVTISCTGSGSNIGSGYDVHWYQQLPGRAPKLLIYRNRNRPSGVPDRFSGSKSGTSASLAIAGLQSEDEGDYFCQSYDGRLGESAVFGGGTRLTVL

SEQ ID NO: 126 - protein, 9 aa, Artificial sequence
  synthetic sequence
GSNIGSGYD

SEQ ID NO: 127 - protein, 3 aa, Artificial sequence
  synthetic sequence
RNR

SEQ ID NO: 128 - protein, 12 aa, Artificial sequence
  synthetic sequence
QSYDGRLGESAV

SEQ ID NO: 129 - protein, 128 aa, Artificial sequence
  synthetic sequence
QVQLQESGPGLVKPSETLSLTCSVSGGSINYYYWSWIRKSPGKGLEWIGFIYSSGTTNYNPSLKSRVSMSIGTSKRQFSLKLSSVTAADSAVYYCARHSRSCTNGVCQTYYYYALDVWGHGTTVTVSS

SEQ ID NO: 130 - protein, 8 aa, Artificial sequence
  synthetic sequence
GGSINYYY

SEQ ID NO: 131 - protein, 7 aa, Artificial sequence
  synthetic sequence
IYSSGTT

SEQ ID NO: 132 - protein, 22 aa, Artificial sequence
  synthetic sequence
ARHSRSCTNGVCQTYYYYALDV

SEQ ID NO: 133 - protein, 112 aa, Artificial sequence
  synthetic sequence
QSVLTQPPSVSGAPGQRVTISCTGSSSNIGAGYDVHWYQQLPGTAPKLLIYANTHRPSGVPDRFSASKSGTSASLAIAGLQAEDEGDYYCQSYDGSLSESAVFGGGTRLTVL

SEQ ID NO: 134 - protein, 9 aa, Artificial sequence
  synthetic sequence
SSNIGAGYD

SEQ ID NO: 135 - protein, 3 aa, Artificial sequence
  synthetic sequence
ANT

SEQ ID NO: 136 - protein, 12 aa, Artificial sequence
  synthetic sequence
QSYDGSLSESAV

SEQ ID NO: 137 - protein, 125 aa, Artificial sequence
  synthetic sequence
EVQLVESGGGLVKPGGSLRLSCVASGFSFGLYTMNWVRQAPGKGLEWVSYISSSTSYKYYADSVKGRVSVSRDNAKNSLYLQLNGLRVEDTAVYYCARDGYCPNGICTYYGMDVWGQGTTVTVSA

SEQ ID NO: 138 - protein, 8 aa, Artificial sequence
  synthetic sequence
GFSFGLYT

SEQ ID NO: 139 - protein, 8 aa, Artificial sequence
  synthetic sequence
ISSSTSYK

SEQ ID NO: 140 - protein, 18 aa, Artificial sequence
  synthetic sequence
ARDGYCPNGICTYYGMDV

SEQ ID NO: 141 - protein, 109 aa, Artificial sequence
  synthetic sequence
EIVMTQSPATLSVSPGERATLSCRASQSVSSNLAWYQQKPGQAPRLLIYGASTRATGIPARFSGSGSGTEFTLTITGLQSEDFAVYYCQQYDKWPPAYSFGQGTKVEIK

SEQ ID NO: 142 - protein, 6 aa, Artificial sequence
  synthetic sequence
QSVSSN

SEQ ID NO: 143 - protein, 3 aa, Artificial sequence
  synthetic sequence
GAS

SEQ ID NO: 144 - protein, 11 aa, Artificial sequence
  synthetic sequence
QQYDKWPPAYS

SEQ ID NO: 145 - protein, 128 aa, Artificial sequence
  synthetic sequence
QVQLQESGPGLVKPSETLSLTCSVTGGSTNYYYWSWIRQPPGKGLEWIGYIYYSGTTNYNPSLKDRVTMSVDKSKTQLSLKLNSVTAADTAVYYCARHARHCTNGVCQTYYYYGLDVWGLGTTVAVSA

SEQ ID NO: 146 - protein, 8 aa, Artificial sequence
  synthetic sequence
GGSTNYYY

SEQ ID NO: 147 - protein, 7 aa, Artificial sequence
  synthetic sequence
IYYSGTT

SEQ ID NO: 148 - protein, 22 aa, Artificial sequence
  synthetic sequence
ARHARHCTNGVCQTYYYYGLDV

SEQ ID NO: 149 - protein, 112 aa, Artificial sequence
  synthetic sequence
QSVLTQPPSVSAAPGQRVTISCSGSSSNIGAGYDVHWYQHLPGTAPKLLIYNNNNRPSGVPDRFSASRSGTSASLAITGVQTEDEADYYCQSFDGRLSESGVFGGGTKLTVL

SEQ ID NO: 150 - protein, 9 aa, Artificial sequence
  synthetic sequence
SSNIGAGYD

SEQ ID NO: 151 - protein, 3 aa, Artificial sequence
  synthetic sequence
NNN

SEQ ID NO: 152 - protein, 12 aa, Artificial sequence
  synthetic sequence
QSFDGRLSESGV

SEQ ID NO: 153 - protein, 128 aa, Artificial sequence
  synthetic sequence
QVQLQESGPGLVKPSETLSLTCTVSGGSINYYYWSWIRQSPGKGLEWIGFIYSSGTTNYNPSLKSRVTMSVDSSKSQFSLKLSSVTAADSAVYYCARHSRSCTNGVCQTYYYYALDVWGHGTTVTVSS

SEQ ID NO: 154 - protein, 8 aa, Artificial sequence
  synthetic sequence
GGSINYYY

SEQ ID NO: 155 - protein, 7 aa, Artificial sequence
  synthetic sequence
IYSSGTT

SEQ ID NO: 156 - protein, 22 aa, Artificial sequence
  synthetic sequence
ARHSRSCTNGVCQTYYYYALDV

SEQ ID NO: 157 - protein, 112 aa, Artificial sequence
  synthetic sequence
QSVLTQPPSVSGAPGQRVTISCTGSSSNIGAGYDVHWYQQLPGTAPKLLIYGNSNRPSGVPDRFSASKSGTSASLAIAGLQPEDEGDYYCQSYDGSLSESGVFGGGTRLTVL

SEQ ID NO: 158 - protein, 9 aa, Artificial sequence
  synthetic sequence
SSNIGAGYD

SEQ ID NO: 159 - protein, 3 aa, Artificial sequence
  synthetic sequence
GNS

SEQ ID NO: 160 - protein, 12 aa, Artificial sequence
  synthetic sequence
QSYDGSLSESGV

SEQ ID NO: 161 - protein, 125 aa, Artificial sequence
  synthetic sequence
EVRLVESGGGLVKPGGSLRLSCAASGFSFSLYTMNWVRQAPGKGLEWVSYISSSSSYRYYADSVKGRFSVSRDNAKNTLYLEMNGLRAEDTAVYYCARDGYCPRGVCTYYGMDVWGQGTTVTVSA

SEQ ID NO: 162 - protein, 8 aa, Artificial sequence
  synthetic sequence
GFSFSLYT

SEQ ID NO: 163 - protein, 8 aa, Artificial sequence
  synthetic sequence
ISSSSSYR

SEQ ID NO: 164 - protein, 18 aa, Artificial sequence
  synthetic sequence
ARDGYCPRGVCTYYGMDV

SEQ ID NO: 165 - protein, 109 aa, Artificial sequence
  synthetic sequence
EIVMTQSPATLSVSPGERATLSCRASQTIGIRLAWYQQKPGQAPRLLIYDATIRATGIPARFSGSGSGTDFTLTISGLQSEDFAVYYCQRYNNWPPVYTFGQGTKLEMK

SEQ ID NO: 166 - protein, 6 aa, Artificial sequence
  synthetic sequence
QTIGIR

SEQ ID NO: 167 - protein, 3 aa, Artificial sequence
  synthetic sequence
DAT

SEQ ID NO: 168 - protein, 11 aa, Artificial sequence
  synthetic sequence
QRYNNWPPVYT

SEQ ID NO: 169 - protein, 125 aa, Artificial sequence
  synthetic sequence
EVQLVESGGGLVKPGGSLRLSCAASGFSFNTYTMNWVRQAPGKGLEWVSYISSSSSYKYYSDSVKGRFSVSRDNAKKSLYLQMNGLRAEDTAVYYCARDGYCPNGVCTYYGMDVWGQGTTVTVSL

SEQ ID NO: 170 - protein, 8 aa, Artificial sequence
  synthetic sequence
GFSFNTYT

SEQ ID NO: 171 - protein, 8 aa, Artificial sequence
  synthetic sequence
ISSSSSYK

SEQ ID NO: 172 - protein, 18 aa, Artificial sequence
  synthetic sequence
ARDGYCPNGVCTYYGMDV

SEQ ID NO: 173 - protein, 109 aa, Artificial sequence
  synthetic sequence
EIVMTQSPATLSVSPGERATLSCRASQSVSSNLAWYQQKPGQAPRLLIYGASTRATGIPARFSGSGSGTEFTLTISGLQSEDFAVYFCQQYSKWPPAYTFGQGTKLEIK

SEQ ID NO: 174 - protein, 6 aa, Artificial sequence
  synthetic sequence
QSVSSN

SEQ ID NO: 175 - protein, 3 aa, Artificial sequence
  synthetic sequence
GAS

SEQ ID NO: 176 - protein, 11 aa, Artificial sequence
  synthetic sequence
QQYSKWPPAYT

SEQ ID NO: 177 - protein, 128 aa, Artificial sequence
  synthetic sequence
QVQLQESGPGLVKPSETLSLTCTVSGGSINYYYWSWIRQPPGKGLEWIGFIYSSGTTNYNPSLKSRVTMSVDSSKSQFSLKLSSVTAADSAVYYCARHSRSCTNGVCQTYYYYALDVWGHGTTVTVSS

SEQ ID NO: 178 - protein, 8 aa, Artificial sequence
  synthetic sequence
GGSINYYY

SEQ ID NO: 179 - protein, 7 aa, Artificial sequence
  synthetic sequence
IYSSGTT

SEQ ID NO: 180 - protein, 22 aa, Artificial sequence
  synthetic sequence
ARHSRSCTNGVCQTYYYYALDV

SEQ ID NO: 181 - protein, 112 aa, Artificial sequence; identical to SEQ ID NO: 157
  synthetic sequence

SEQ ID NO: 182 - protein, 9 aa, Artificial sequence
  synthetic sequence
SSNIGAGYD

SEQ ID NO: 183 - protein, 3 aa, Artificial sequence
  synthetic sequence
GNS

SEQ ID NO: 184 - protein, 12 aa, Artificial sequence
  synthetic sequence
QSYDGSLSESGV

SEQ ID NO: 185 - protein, 125 aa, Artificial sequence
  synthetic sequence
EVQLVESGGGLVKPGGSLRLSCAASGFSFSTYTMNWVRQAPGKGLEWVSYISSSSSYRYYADSVRGRFSVSRDNAKNSLYLQMNGLRVEDTAVYYCARDGYCPNGVCTYYGMDVWGQGTTVTVSS

SEQ ID NO: 186 - protein, 8 aa, Artificial sequence
  synthetic sequence
GFSFSTYT

SEQ ID NO: 187 - protein, 8 aa, Artificial sequence
  synthetic sequence
ISSSSSYR

SEQ ID NO: 188 - protein, 18 aa, Artificial sequence
  synthetic sequence
ARDGYCPNGVCTYYGMDV

SEQ ID NO: 189 - protein, 109 aa, Artificial sequence
  synthetic sequence
EIVMTQSPATLSVSPGERATLSCRASQSVSSNLAWYQQKPGQAPRLLIYGASTRATGIPARFSGSGSGTDFTLTISSLQSEDFAVYYCQQYSKWPPAYTFGQGTKLEIK

SEQ ID NO: 190 - protein, 6 aa, Artificial sequence
  synthetic sequence
QSVSSN

SEQ ID NO: 191 - protein, 3 aa, Artificial sequence
  synthetic sequence
GAS

SEQ ID NO: 192 - protein, 11 aa, Artificial sequence
  synthetic sequence
QQYSKWPPAYT

SEQ ID NO: 193 - protein, 128 aa, Artificial sequence
  synthetic sequence
QVQLQESGPGLVKPSETLSLTCSVSGGSINYYYWSWIRQPPGKGLEWIGFIYSSGTTNYNPSLKSRVTMSVDTSKNQFSLKLSSVTAADSAVYYCARHSRSCTNGVCQTYYYYALDVWGHGTTVTVSS

SEQ ID NO: 194 - protein, 8 aa, Artificial sequence
  synthetic sequence
GGSINYYY

SEQ ID NO: 195 - protein, 7 aa, Artificial sequence
  synthetic sequence
IYSSGTT

SEQ ID NO: 196 - protein, 22 aa, Artificial sequence
  synthetic sequence
ARHSRSCTNGVCQTYYYYALDV

SEQ ID NO: 197 - protein, 112 aa, Artificial sequence
  synthetic sequence
QSVLTQPPSVSGAPGQRVTISCTGSSSNIGAGYDVHWYQQLPGTAPKLLIYGNSNRPSGVPDRFSASKSGTSASLAIAGLQAEDEGDYYCQSYDGSLSESGVFGGGTRLTVL

SEQ ID NO: 198 - protein, 9 aa, Artificial sequence
  synthetic sequence
SSNIGAGYD

SEQ ID NO: 199 - protein, 3 aa, Artificial sequence
  synthetic sequence
GNS

SEQ ID NO: 200 - protein, 12 aa, Artificial sequence
  synthetic sequence
QSYDGSLSESGV

SEQ ID NO: 201 - protein, 125 aa, Artificial sequence
  synthetic sequence
EVQLVESGGGLVKPGGSLRLSCVASGFSFSIYSMNWVRQAPGKGLEWVSYISSSSSYKYYADSVKGRFSVSRDNAKNSLYLQLNGLRAEDTAVYYCARDGYCPKGVCTYYGMDVWGQGTTVTVSA

SEQ ID NO: 202 - protein, 8 aa, Artificial sequence
  synthetic sequence
GFSFSIYS

SEQ ID NO: 203 - protein, 8 aa, Artificial sequence
  synthetic sequence
ISSSSSYK

SEQ ID NO: 204 - protein, 18 aa, Artificial sequence
  synthetic sequence
ARDGYCPKGVCTYYGMDV

SEQ ID NO: 205 - protein, 109 aa, Artificial sequence
  synthetic sequence
EIVMTQSPATLSVSPGERVTLSCRASQSVRSRLAWFQQKPGQAPRLLIYDASIRATGIPARFSGSGSGTEFTLIISSLQSEDFAVYYCQQYDNWPPAYTFGQGTKLEIK

SEQ ID NO: 206 - protein, 6 aa, Artificial sequence
  synthetic sequence
QSVRSR

SEQ ID NO: 207 - protein, 3 aa, Artificial sequence
  synthetic sequence
DAS

SEQ ID NO: 208 - protein, 11 aa, Artificial sequence
  synthetic sequence
QQYDNWPPAYT

SEQ ID NO: 209 - protein, 125 aa, Artificial sequence
  synthetic sequence
EVQLVESGGGLVKPGGSLRLSCAASGFSFSLYTMNWVRQAPGKGLEWVSYISSSSSYRYYADSVKGRFSVSRDNAKNALYLQMNGLRAEDTAVYYCARDGYCPRGVCTYYGMDVWGQGTTVTVSA

SEQ ID NO: 210 - protein, 8 aa, Artificial sequence
  synthetic sequence
GFSFSLYT

SEQ ID NO: 211 - protein, 8 aa, Artificial sequence
  synthetic sequence
ISSSSSYR

SEQ ID NO: 212 - protein, 18 aa, Artificial sequence
  synthetic sequence
ARDGYCPRGVCTYYGMDV

SEQ ID NO: 213 - protein, 109 aa, Artificial sequence
  synthetic sequence
EIVMTQSPATLSVSPGERATLSCRASQSVGSRLAWYQQKPGQAPRLLIYDATIRATGIPARFSGSGSGTDFTLTISGLQSEDFAVYYCQRYNNWPPAYTFGQGTKLEIK

SEQ ID NO: 214 - protein, 6 aa, Artificial sequence
  synthetic sequence
QSVGSR

SEQ ID NO: 215 - protein, 3 aa, Artificial sequence
  synthetic sequence
DAT

SEQ ID NO: 216 - protein, 11 aa, Artificial sequence
  synthetic sequence
QRYNNWPPAYT

SEQ ID NO: 217 - protein, 125 aa, Artificial sequence
  synthetic sequence
EVQLVESGGGLVKPGGSLRLSCAASGFTFSSYSMNWVRQAPGKGLEWVSYISSSSSYKYYADSVKGRFSVSRDNAKNSLYLQLNGLRVEDTAVYYCARDGYCPKGVCTYYGMDVWGQGTTVTVSA

SEQ ID NO: 218 - protein, 8 aa, Artificial sequence
  synthetic sequence
GFTFSSYS

SEQ ID NO: 219 - protein, 8 aa, Artificial sequence
  synthetic sequence
ISSSSSYK

SEQ ID NO: 220 - protein, 18 aa, Artificial sequence
  synthetic sequence
ARDGYCPKGVCTYYGMDV

SEQ ID NO: 221 - protein, 109 aa, Artificial sequence
  synthetic sequence
EIVMTQSPATLSVSPGERATLSCRASQSVRSRLAWFQQKPGQPPRLLIYDASIRATGIPDRFSGSGSGTEFTLIISGLQSEDFAVYYCQQYDNWPPAYTFGQGTKLEIK

SEQ ID NO: 222 - protein, 6 aa, Artificial sequence
  synthetic sequence
QSVRSR

SEQ ID NO: 223 - protein, 3 aa, Artificial sequence
  synthetic sequence
DAS

SEQ ID NO: 224 - protein, 11 aa, Artificial sequence
  synthetic sequence
QQYDNWPPAYT

SEQ ID NO: 225 - protein, 124 aa, Artificial sequence
  synthetic sequence
QVQLVESGGGVVQPGRSLRLSCAASGFTFSSYAMHWVRQAPGKGLEWVAVISYDGSNKYYADSVKGRFTISRDNSKNTLYLQMDSLRAQDTAVYYCATPRDMFSSGWSSPFDYWGQGTLVTVSS

SEQ ID NO: 226 - protein, 8 aa, Artificial sequence
  synthetic sequence
GFTFSSYA

SEQ ID NO: 227 - protein, 8 aa, Artificial sequence
  synthetic sequence
ISYDGSNK

SEQ ID NO: 228 - protein, 17 aa, Artificial sequence
  synthetic sequence
ATPRDMFSSGWSSPFDY

SEQ ID NO: 229 - protein, 107 aa, Artificial sequence
  synthetic sequence
DIQMTQSPSSLSASVGDRVTITCQASQDISIYLTWYQQKPGKAPKLLIYGASNLEVEVPSRFSGSGSGTEFTLTISSLQPEDFATYFCQHSDDLPVTFGGGTKVEVK

SEQ ID NO: 230 - protein, 6 aa, Artificial sequence
  synthetic sequence
QDISIY

SEQ ID NO: 231 - protein, 3 aa, Artificial sequence
  synthetic sequence
GAS

SEQ ID NO: 232 - protein, 9 aa, Artificial sequence
  synthetic sequence
QHSDDLPVT

SEQ ID NO: 233 - protein, 120 aa, Artificial sequence
  synthetic sequence
QVQLVQSGTEVKKPGSSVRVSCQASGDTFTSHAIIWIRQAPGQGLEYLGRIIPVLDITNAAQRFLGRLTLTADKSTTTAYMELSSLRSEDTAVYYCARAPFGLIVMYDHWGQGTLVTVTA

SEQ ID NO: 234 - protein, 8 aa, Artificial sequence
  synthetic sequence
GDTFTSHA

SEQ ID NO: 235 - protein, 8 aa, Artificial sequence
  synthetic sequence
IIPVLDIT

SEQ ID NO: 236 - protein, 13 aa, Artificial sequence
  synthetic sequence
ARAPFGLIVMYDH

SEQ ID NO: 237 - protein, 108 aa, Artificial sequence
  synthetic sequence
EIVLTQSPGTLSLSPGERATLSCRASQSVSGNFLAWYQQKPGQAPRLLIHETSKRATGIPDRVSGGGSGTDFTLTISRLEPEDFAVYHCQQYGTTAVTFGQGTRLDMK

SEQ ID NO: 238 - protein, 7 aa, Artificial sequence
  synthetic sequence
QSVSGNF

SEQ ID NO: 239 - protein, 3 aa, Artificial sequence
  synthetic sequence
ETS

SEQ ID NO: 240 - protein, 9 aa, Artificial sequence
  synthetic sequence
QQYGTTAVT

SEQ ID NO: 241 - protein, 117 aa, Artificial sequence
  synthetic sequence
QVQLVQSGAEVKKPGASVKVSCKASGYTFTSNYIHWLRQAPGQGLEWMGIIKPSGTSTISAQKFRGRVAMTRDTSTSTVYMELSSLRFEDTAIYYCARDAKRALETWGQGTMVTVSS

SEQ ID NO: 242 - protein, 8 aa, Artificial sequence
  synthetic sequence
GYTFTSNY

SEQ ID NO: 243 - protein, 8 aa, Artificial sequence
  synthetic sequence
IKPSGTST

SEQ ID NO: 244 - protein, 10 aa, Artificial sequence
  synthetic sequence
ARDAKRALET

SEQ ID NO: 245 - protein, 110 aa, Artificial sequence
  synthetic sequence
QSVLTQPPSVSAAPGQKVTISCSGSSSNIGNNYVSWYQQLPGTAPKLLIYDNNKRPSGIPDRFSGSRSGTSATLGITGLQTGDEADYYCGTWDSSLSAWVFGGGTKLTVL

SEQ ID NO: 246 - protein, 8 aa, Artificial sequence
  synthetic sequence
SSNIGNNY

SEQ ID NO: 247 - protein, 3 aa, Artificial sequence
  synthetic sequence
DNN

SEQ ID NO: 248 - protein, 11 aa, Artificial sequence
  synthetic sequence
GTWDSSLSAWV

SEQ ID NO: 249 - protein, 117 aa, Artificial sequence
  synthetic sequence
QVQLVQSGAEVKKPGASVKVSCKASGYSFTDYYIHWVRQAPGQGLEWMGIIKPSAGATVYAQKFRGRVSMTSDTSTGTVYMELSGLKSEDTAVYYCARDYNRSFDFWGQGTLVTVSS

SEQ ID NO: 250 - protein, 8 aa, Artificial sequence
  synthetic sequence
GYSFTDYY

SEQ ID NO: 251 - protein, 8 aa, Artificial sequence
  synthetic sequence
IKPSAGAT

SEQ ID NO: 252 - protein, 10 aa, Artificial sequence
  synthetic sequence
ARDYNRSFDF

SEQ ID NO: 253 - protein, 110 aa, Artificial sequence
  synthetic sequence
QSVLTQPPSVSAAPGQTVTISCAGSTSNIGKNYVSWYQHLPGTAPKLLIYDNIKRPSGIPDRFSGSTSGTSATLGITELQTGDEADYYCGTWDSSLSAYVFGTGTKVTVL

SEQ ID NO: 254 - protein, 8 aa, Artificial sequence
  synthetic sequence
TSNIGKNY

SEQ ID NO: 255 - protein, 3 aa, Artificial sequence
  synthetic sequence
DNI

SEQ ID NO: 256 - protein, 11 aa, Artificial sequence
  synthetic sequence
GTWDSSLSAYV

SEQ ID NO: 257 - protein, 117 aa, Artificial sequence
  synthetic sequence
EVQLVESGGGLVQPGGSLRLSCAASGFTVSSNYMSWVRQAPGKGLEWVSVIYSGGGTYYADSVKGRFIISRDNSKNTLYLQMNSLRAEDTAVYYCARLSWWGDDNYWGQGTLVTVSS

SEQ ID NO: 258 - protein, 8 aa, Artificial sequence
  synthetic sequence
GFTVSSNY

SEQ ID NO: 259 - protein, 7 aa, Artificial sequence
  synthetic sequence
IYSGGGT

SEQ ID NO: 260 - protein, 11 aa, Artificial sequence
  synthetic sequence
ARLSWWGDDNY

SEQ ID NO: 261 - protein, 111 aa, Artificial sequence
  synthetic sequence
QAVVTQEPSLTVSPGGTVTLTCGSSTGAVTSGHYPYWFQQKPGQAPRTLIYDTSNKHSWTPARFSGSLLGGKAALTLSGAQPEDEADYYCLVSYSGARPGVFGGGTKLTVL

SEQ ID NO: 262 - protein, 9 aa, Artificial sequence
  synthetic sequence
TGAVTSGHY

SEQ ID NO: 263 - protein, 3 aa, Artificial sequence
  synthetic sequence
DTS

SEQ ID NO: 264 - protein, 11 aa, Artificial sequence
  synthetic sequence
LVSYSGARPGV

SEQ ID NO: 265 - protein, 119 aa, Artificial sequence
  synthetic sequence
QVQLVESGGGVVQPGGSLSLSCAASGLSFSSYGMHWVRQAPGKGLEWVAFIRYDVSNKYYADSVKGRFTISRDNSKNTLYLQMNSLRVEDTAVYYCATWAPLDDGMDVWGQGTTVTVSS

SEQ ID NO: 266 - protein, 8 aa, Artificial sequence
  synthetic sequence
GLSFSSYG

SEQ ID NO: 267 - protein, 8 aa, Artificial sequence
  synthetic sequence
IRYDVSNK

SEQ ID NO: 268 - protein, 12 aa, Artificial sequence
  synthetic sequence
ATWAPLDDGMDV

SEQ ID NO: 269 - protein, 109 aa, Artificial sequence
  synthetic sequence
SYELTQPPSVSVSPGQTARITCSGDALPKQYAYWYQQKPGQAPVLVIYKDTERPSGIPERFSGSSSGTTVTLTISGVQAEDEADYYCQSGDSSGTFYVVFGGGTKLTVL

SEQ ID NO: 270 - protein, 6 aa, Artificial sequence
  synthetic sequence
ALPKQY

SEQ ID NO: 271 - protein, 3 aa, Artificial sequence
  synthetic sequence
KDT

SEQ ID NO: 272 - protein, 12 aa, Artificial sequence
  synthetic sequence
QSGDSSGTFYVV

SEQ ID NO: 273 - protein, 122 aa, Artificial sequence
  synthetic sequence
QVQLVQSGAEVKKPGASVKVSCKASGYTFTDYFMHWVRQAPGQGLEWMGWVNPLSGGTNFAQKFQGRVTMTSDTSITTVYMELSRLRSDDTAVYYCARDPPLYSSTYGMDVWGQGTTVTVSS

SEQ ID NO: 274 - protein, 8 aa, Artificial sequence
  synthetic sequence
GYTFTDYF

SEQ ID NO: 275 - protein, 8 aa, Artificial sequence
  synthetic sequence
VNPLSGGT

SEQ ID NO: 276 - protein, 15 aa, Artificial sequence
  synthetic sequence
ARDPPLYSSTYGMDV

SEQ ID NO: 277 - protein, 110 aa, Artificial sequence
  synthetic sequence
QSALTQPASVSGSPGQSITISCTGTSSDVGAYNYVSWYQQHPGKAPKLMIYDVSDRPSGVSNRFSGSKSGNTASLTISGLQAEDEADYYCSSYTISSSVVFGGGTKLTVL

SEQ ID NO: 278 - protein, 9 aa, Artificial sequence
  synthetic sequence
SSDVGAYNY

SEQ ID NO: 279 - protein, 3 aa, Artificial sequence
  synthetic sequence
DVS

SEQ ID NO: 280 - protein, 10 aa, Artificial sequence
  synthetic sequence
SSYTISSSVV

SEQ ID NO: 281 - protein, 122 aa, Artificial sequence
  synthetic sequence
QVQLVQSGAEVKKPGASVKVSCKVSGYTLIELSMHWVRQAPGKGLEWMGGFDPEEGETIYAQKFQGRVTMTEDTSTDTAYMELSSLRSEDTAVYYCTTSPPVGATGAWFDPWGQGTLVTVSA

SEQ ID NO: 282 - protein, 8 aa, Artificial sequence
  synthetic sequence
GYTLIELS

SEQ ID NO: 283 - protein, 8 aa, Artificial sequence
  synthetic sequence
FDPEEGET

SEQ ID NO: 284 - protein, 15 aa, Artificial sequence
  synthetic sequence
TTSPPVGATGAWFDP

SEQ ID NO: 285 - protein, 107 aa, Artificial sequence
  synthetic sequence
SYVLTQPPSVSVAPGKTARITCGGNNIGSKSVHWYQQKPGQAPVLVVYDDSDRPSGIPERFSGSNSGNTATLTISGLQAEDEADYYCSSYTSSSTVVFGGGTKLTVL

SEQ ID NO: 286 - protein, 6 aa, Artificial sequence
  synthetic sequence
NIGSKS

SEQ ID NO: 287 - protein, 3 aa, Artificial sequence
  synthetic sequence
DDS

SEQ ID NO: 288 - protein, 10 aa, Artificial sequence
  synthetic sequence
SSYTSSSTVV

SEQ ID NO: 289 - protein, 121 aa, Artificial sequence
  synthetic sequence
QVQLVQSGAEVKKPGASVKVSCKVSGYTLIELSMHWVRQAPGKGLEWMGGFDPEDVETIYAQKFQGRVTMTEDTSTDTAYMELSSLRSEDTAVYYCATAYAVQWRGMIGYWGQGTLVTVSS

SEQ ID NO: 290 - protein, 8 aa, Artificial sequence
  synthetic sequence
GYTLIELS

SEQ ID NO: 291 - protein, 8 aa, Artificial sequence
  synthetic sequence
FDPEDVET

SEQ ID NO: 292 - protein, 14 aa, Artificial sequence
  synthetic sequence
ATAYAVQWRGMIGY

SEQ ID NO: 293 - protein, 112 aa, Artificial sequence
  synthetic sequence
QSALTQPASVSGSPGQSITISCTGTSSDVGSYNLVSWYQQHPGKAPKLMIYEVSKRPSGVSNRFSGSKSGNTASLTISGLQAEDEADYYCSSYAGSSTFSYVFGTGTKVTVL

SEQ ID NO: 294 - protein, 9 aa, Artificial sequence
  synthetic sequence
SSDVGSYNL

SEQ ID NO: 295 - protein, 3 aa, Artificial sequence
  synthetic sequence
EVS

SEQ ID NO: 296 - protein, 12 aa, Artificial sequence
  synthetic sequence
SSYAGSSTFSYV

SEQ ID NO: 297 - protein, 122 aa, Artificial sequence
  synthetic sequence
QVQLVQSGAEVKKPGASVKVPCKVSGYTFTDHFIHWVRQAPGQGLEWMGWISPNSGGRNYTQKFQGRVTLTRDTAITTVHMDLSSLTPDDTAVYYCARDVRWAQLQGGFDLWGQGTMVTVSS

SEQ ID NO: 298 - protein, 8 aa, Artificial sequence
  synthetic sequence
GYTFTDHF

SEQ ID NO: 299 - protein, 8 aa, Artificial sequence
  synthetic sequence
ISPNSGGR

SEQ ID NO: 300 - protein, 15 aa, Artificial sequence
  synthetic sequence
ARDVRWAQLQGGFDL

SEQ ID NO: 301 - protein, 110 aa, Artificial sequence
  synthetic sequence
QSALTQPASVSGSPGQSITISCTGTSSDVGSYNLVSWYQQHPGKAPKLMIYEVSKRPSGVSNRFSGSKSGNTASLTISGLQADDEADYYCCSYAGDNNFLFGGGTSLTVL

SEQ ID NO: 302 - protein, 9 aa, Artificial sequence
  synthetic sequence
SSDVGSYNL

SEQ ID NO: 303 - protein, 3 aa, Artificial sequence
  synthetic sequence
EVS

SEQ ID NO: 304 - protein, 10 aa, Artificial sequence
  synthetic sequence
CSYAGDNNFL

SEQ ID NO: 305 - protein, 125 aa, Artificial sequence
  synthetic sequence
QVQLVQSGAEVKKPGSSVKVSCTASGGTFSIYAFSWVRQAPGQGLEWMGGIIPISGTAGYAQKFQGRVTITADESTSTAYMELSSLRSEDTAIYYCARKYRYCSGSRCYTYFDYWGQGTLVTVSS

SEQ ID NO: 306 - protein, 8 aa, Artificial sequence
  synthetic sequence
GGTFSIYA

SEQ ID NO: 307 - protein, 8 aa, Artificial sequence
  synthetic sequence
IIPISGTA

SEQ ID NO: 308 - protein, 18 aa, Artificial sequence
  synthetic sequence
ARKYRYCSGSRCYTYFDY

SEQ ID NO: 309 - protein, 106 aa, Artificial sequence
  synthetic sequence
DIQMTQSPSSLSASVGDRVTITCRASQSISSFLNWYQQKPGKAPKLLIYAASSLQSGVPSRFSGSGSGTDFTLTISSLQPEDSATYYCQQSYSTITFGQGTRLEIK

SEQ ID NO: 310 - protein, 5 aa, Artificial sequence
  synthetic sequence
QSISS

SEQ ID NO: 311 - protein, 3 aa, Artificial sequence
  synthetic sequence
AAS

SEQ ID NO: 312 - protein, 8 aa, Artificial sequence
  synthetic sequence
QQSYSTIT

SEQ ID NO: 313 - protein, 119 aa, Artificial sequence
  synthetic sequence
QVQLQQWGAGLLKPSETLSLTCAVYGGSFSGYYWSWIRQPPGKGLEWIGRINDIGSTDYNPSLKSRVTISVDTSKNQFSLKLTSVTAADTAVYYCAREPFDSEGTFDYWGQGTLVIVSS

SEQ ID NO: 314 - protein, 8 aa, Artificial sequence
  synthetic sequence
GGSFSGYY

SEQ ID NO: 315 - protein, 7 aa, Artificial sequence
  synthetic sequence
INDIGST

SEQ ID NO: 316 - protein, 13 aa, Artificial sequence
  synthetic sequence
AREPFDSEGTFDY

SEQ ID NO: 317 - protein, 107 aa, Artificial sequence
  synthetic sequence
DIQMTQPPSTLSASVGDRVTITCRASQSISRWLAWYRQKPGKAPKLLIYDASSLQSGVPSRFSGSGSGTEFTLTISSLQPDDLATYYCQQYNTYPYTFGQGTKLEIK

SEQ ID NO: 318 - protein, 6 aa, Artificial sequence
  synthetic sequence
QSISRW

SEQ ID NO: 319 - protein, 3 aa, Artificial sequence
  synthetic sequence
DAS

SEQ ID NO: 320 - protein, 9 aa, Artificial sequence
  synthetic sequence
QQYNTYPYT

SEQ ID NO: 321 - protein, 125 aa, Artificial sequence
  synthetic sequence
QVQLVQSGAEVKKPGSSVKVSCKASGDTFSSYAISWVRQAPGQGLEWLGGIIPIFGSADYAQKFQGRVTITADEFTSTAYMELSSLRSEDTAVYFCAREKYGDYGEGPLYNFDYWGQGTLVTVSS

SEQ ID NO: 322 - protein, 8 aa, Artificial sequence
  synthetic sequence
GDTFSSYA

SEQ ID NO: 323 - protein, 8 aa, Artificial sequence
  synthetic sequence
IIPIFGSA

SEQ ID NO: 324 - protein, 18 aa, Artificial sequence
  synthetic sequence
AREKYGDYGEGPLYNFDY

SEQ ID NO: 325 - protein, 107 aa, Artificial sequence
  synthetic sequence
DIQMTQSPSSLSASVGDRVTITCRASQSISSYLNWYQQKPGKAPNLLIYAASSLQSGVPSRFSGSGSGTDFTLTISSLQPEDFATYYCQQSYSTPKTFGQGTKVEIK

SEQ ID NO: 326 - protein, 6 aa, Artificial sequence
  synthetic sequence
QSISSY

SEQ ID NO: 327 - protein, 3 aa, Artificial sequence
  synthetic sequence
AAS

SEQ ID NO: 328 - protein, 9 aa, Artificial sequence
  synthetic sequence
QQSYSTPKT

SEQ ID NO: 329 - protein, 125 aa, Artificial sequence
  synthetic sequence
QVQLVQSGAEVKKPGSSVRVSCKASGGTFISYTFNWVRQAPGQGLEWMGRIIPIFGIVNYAQKFQGRVTIAADKSTSTAYMELSSLRSEDTAMYYCATATVDYDSGEEQSSFDPWGQGTLVTVSS

SEQ ID NO: 330 - protein, 8 aa, Artificial sequence
  synthetic sequence
GGTFISYT

SEQ ID NO: 331 - protein, 8 aa, Artificial sequence
  synthetic sequence
IIPIFGIV

SEQ ID NO: 332 - protein, 18 aa, Artificial sequence
  synthetic sequence
ATATVDYDSGEEQSSFDP

SEQ ID NO: 333 - protein, 104 aa, Artificial sequence
  synthetic sequence
EIVLTQSPGTLSLSPGERATLSCRASQSVSSSYLAWYQQKAGQTPRLLIYAASSRATGVPDRFSGSGSGTDFTLTISRLEAEDFAVYYCQQSWTFGQGTKVEIK

SEQ ID NO: 334 - protein, 7 aa, Artificial sequence
  synthetic sequence
QSVSSSY

SEQ ID NO: 335 - protein, 3 aa, Artificial sequence
  synthetic sequence
AAS

SEQ ID NO: 336 - protein, 5 aa, Artificial sequence
  synthetic sequence
QQSWT

SEQ ID NO: 337 - protein, 125 aa, Artificial sequence
  synthetic sequence
QVQLQESGPGLVKPSGTLSLTCVVSGGSISSSNWWSWVRQPPGKGLEWIGETFHSGSFNYNPSLKSRVTISVDKSKNQFSLKLSSVTAADTAIYYCATTRVGYEGHFYYYGMDVWGQGTTVTVSS

SEQ ID NO: 338 - protein, 9 aa, Artificial sequence
  synthetic sequence
GGSISSSNW

SEQ ID NO: 339 - protein, 7 aa, Artificial sequence
  synthetic sequence
TFHSGSF

SEQ ID NO: 340 - protein, 18 aa, Artificial sequence
  synthetic sequence
ATTRVGYEGHFYYYGMDV

SEQ ID NO: 341 - protein, 107 aa, Artificial sequence
  synthetic sequence
DIQMTQSPSSVSASVGDRVTITCRASQGISSWLAWYQQKPGKAPKLLIYAASSLQSGVPSRFSGSGSGTDFTLTISSLQPEDFATYYCQQANRFPWTFGQGTKVEIK

SEQ ID NO: 342 - protein, 6 aa, Artificial sequence
  synthetic sequence
QGISSW

SEQ ID NO: 343 - protein, 3 aa, Artificial sequence
  synthetic sequence
AAS

SEQ ID NO: 344 - protein, 9 aa, Artificial sequence
  synthetic sequence
QQANRFPWT

SEQ ID NO: 345 - protein, 127 aa, Artificial sequence
  synthetic sequence
EVQLVESGGGLVKPGGSLRLSCAASGFTFSSYSMNWVRQAPGKGLEWVSSITSSSGYMYYADSVKGRFTISRDNAKNSLYLQLNSLRAEDTAVYYCAKDSAFDLWEVRSYYYVMDVWGQGTTVTVSS

SEQ ID NO: 346 - protein, 8 aa, Artificial sequence
  synthetic sequence
GFTFSSYS

SEQ ID NO: 347 - protein, 8 aa, Artificial sequence
  synthetic sequence
ITSSSGYM

SEQ ID NO: 348 - protein, 20 aa, Artificial sequence
  synthetic sequence
AKDSAFDLWEVRSYYYVMDV

SEQ ID NO: 349 - protein, 107 aa, Artificial sequence
  synthetic sequence
EIVMTQSPATLSVSPGERATLSCRASQSVSSNLAWYQQKPGQAPRLLIYGASTRATGIPARFSGSGSGTEFTLTISSLQSEDFALYYCQQYNNWPRTFGQGTKLEIK

SEQ ID NO: 350 - protein, 6 aa, Artificial sequence
  synthetic sequence
QSVSSN

SEQ ID NO: 351 - protein, 3 aa, Artificial sequence
  synthetic sequence
GAS

SEQ ID NO: 352 - protein, 9 aa, Artificial sequence
  synthetic sequence
QQYNNWPRT

SEQ ID NO: 353 - protein, 127 aa, Artificial sequence
  synthetic sequence
EVQLVESGGGLVQPGRSLRLSCAASGFTFDDYAMHWVRQAPGKGLEWVSGISWNSGSIGYADSVKGRFTLSRDNAKNSLFLQMDSLRAEDTALYYCAKDVSYRSGSYYRFWGGSGSWGQGTLVTVSS

SEQ ID NO: 354 - protein, 8 aa, Artificial sequence
  synthetic sequence
GFTFDDYA

SEQ ID NO: 355 - protein, 8 aa, Artificial sequence
  synthetic sequence
ISWNSGSI

SEQ ID NO: 356 - protein, 20 aa, Artificial sequence
  synthetic sequence
AKDVSYRSGSYYRFWGGSGS

SEQ ID NO: 357 - protein, 107 aa, Artificial sequence
  synthetic sequence
DIQMTQSPSSLSASVGDRVTITCRASLSIRSYLNWYQQKPGKPPKLLIYAASSLQSGVPSRFSGSGSGTDFTLTISSLQPEDFATYYCQQSYSTPLTFGGGTKVEIK

SEQ ID NO: 358 - protein, 6 aa, Artificial sequence
  synthetic sequence
LSIRSY

SEQ ID NO: 359 - protein, 3 aa, Artificial sequence
  synthetic sequence
AAS

SEQ ID NO: 360 - protein, 9 aa, Artificial sequence
  synthetic sequence
QQSYSTPLT

SEQ ID NO: 361 - protein, 128 aa, Artificial sequence
  synthetic sequence
QVQLVQSGAEVKKPGASVKVSCKVSGYTLTELSIHWVRQAPGKGLEWMGGFDPEDAETIYAQKFQGRVTMTEDTSTDTAYMELSSLRSEDTAVYYCATATAVAGTVFNYQYHYGLDFWGQGTTVTVSS

SEQ ID NO: 362 - protein, 8 aa, Artificial sequence
  synthetic sequence
GYTLTELS

SEQ ID NO: 363 - protein, 8 aa, Artificial sequence
  synthetic sequence
FDPEDAET

SEQ ID NO: 364 - protein, 21 aa, Artificial sequence
  synthetic sequence
ATATAVAGTVFNYQYHYGLDF

SEQ ID NO: 365 - protein, 112 aa, Artificial sequence
  synthetic sequence
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPHTFGRGTKLEIK

SEQ ID NO: 366 - protein, 11 aa, Artificial sequence
  synthetic sequence
QSLVHSDGNTY

SEQ ID NO: 367 - protein, 3 aa, Artificial sequence
  synthetic sequence
KIS

SEQ ID NO: 368 - protein, 9 aa, Artificial sequence
  synthetic sequence
MQATQFPHT

SEQ ID NO: 369 - protein, 131 aa, Artificial sequence
  synthetic sequence
QVQLVQSGAEVKKPGSSVKVSCKASGGTFSTYAISWVRQAPGQGLEWMGGIIPLFATANYAQNFQGRVTITADKSTSTAYMELSSLRSEDTAVYYCASVRLHLEELSLSHQGDYYYGLDVWGQGTTVTVSS

SEQ ID NO: 370 - protein, 8 aa, Artificial sequence
  synthetic sequence
GGTFSTYA

SEQ ID NO: 371 - protein, 8 aa, Artificial sequence
  synthetic sequence
IIPLFATA

SEQ ID NO: 372 - protein, 24 aa, Artificial sequence
  synthetic sequence
ASVRLHLEELSLSHQGDYYYGLDV

SEQ ID NO: 373 - protein, 107 aa, Artificial sequence
  synthetic sequence
DIQMTQSPSSLSASVADRVTITCQASQDISHYLNWYQQKPGKAPQLLIYDASKLETGAPSRFSGSGSGTDFTFTISSLQPEDIATYYCQQYDHLPLTFGGGSKVEIK

SEQ ID NO: 374 - protein, 6 aa, Artificial sequence
  synthetic sequence
QDISHY

SEQ ID NO: 375 - protein, 3 aa, Artificial sequence
  synthetic sequence
DAS

SEQ ID NO: 376 - protein, 9 aa, Artificial sequence
  synthetic sequence
QQYDHLPLT

SEQ ID NO: 377 - protein, 128 aa, Artificial sequence
  synthetic sequence
QVTLKESGPVLVKPTETLTLTCTVSGFSLSNARMGVSWIRQPPGKALEWLAHIFSNDEKSYSTYLKSRLTISKDSSKSQVVLTMTIMDPVDTATYYCARTAGRYSSRWGHYYYYMDVWGKGTTVTVSS

SEQ ID NO: 378 - protein, 10 aa, Artificial sequence
  synthetic sequence
GFSLSNARMG

SEQ ID NO: 379 - protein, 7 aa, Artificial sequence
  synthetic sequence
IFSNDEK

SEQ ID NO: 380 - protein, 20 aa, Artificial sequence
  synthetic sequence
ARTAGRYSSRWGHYYYYMDV

SEQ ID NO: 381 - protein, 113 aa, Artificial sequence
  synthetic sequence
NFMLTQPHSVSESPGKTVTISCTGSNGSIASNFMQWYQQRPGSAPTIVIYEDNQRPSGVPDRFSGSIDGSSNSASLTISGLKTEDEADYYCQSYDSSDSDLGVFGGGTKLTVL

SEQ ID NO: 382 - protein, 8 aa, Artificial sequence
  synthetic sequence
NGSIASNF

SEQ ID NO: 383 - protein, 3 aa, Artificial sequence
  synthetic sequence
EDN

SEQ ID NO: 384 - protein, 12 aa, Artificial sequence
  synthetic sequence
QSYDSSDSDLGV

SEQ ID NO: 385 - protein, 121 aa, Artificial sequence
  synthetic sequence
QVQLVQSGAEVKKPGASVKVSCKVSGYTLIELSMHWVRQAPGKGLEWMGGFDPEGAETIYAQKFQGRVTMTEDTSTDTAYMELSSLRSDDTAVYYCATGPAVFAANWFDPWGQGTLVTVSS

SEQ ID NO: 386 - protein, 8 aa, Artificial sequence
  synthetic sequence
GYTLIELS

SEQ ID NO: 387 - protein, 8 aa, Artificial sequence
  synthetic sequence
FDPEGAET

SEQ ID NO: 388 - protein, 14 aa, Artificial sequence
  synthetic sequence
ATGPAVFAANWFDP

SEQ ID NO: 389 - protein, 109 aa, Artificial sequence
  synthetic sequence
QSALTQPPSASGSPGQSVTISCTGTSSDIGNYNNVSWYQQHPGKAPKLMIYDVIKRPSGVPDRFSGSKSGNTASLTVSGLQAEDEADYYCSSYAGKSYVFGTGTKVTVL

SEQ ID NO: 390 - protein, 9 aa, Artificial sequence
  synthetic sequence
SSDIGNYNN

SEQ ID NO: 391 - protein, 3 aa, Artificial sequence
  synthetic sequence
DVI

SEQ ID NO: 392 - protein, 9 aa, Artificial sequence
  synthetic sequence
SSYAGKSYV

SEQ ID NO: 393 - protein, 122 aa, Artificial sequence
  synthetic sequence
QVQLVQSGAEVKKPGASVKVSCKVSGFTLPELSIHWVRQAPGKGLEWMGGFDPEDAKTIYAQKFQGRVTMTEDTSTDIAYMELNSLRSDDTAVYYCATGSPFGVVGNWLDPWGQGTLVTVSS

SEQ ID NO: 394 - protein, 8 aa, Artificial sequence
  synthetic sequence
GFTLPELS

SEQ ID NO: 395 - protein, 8 aa, Artificial sequence
  synthetic sequence
FDPEDAKT

SEQ ID NO: 396 - protein, 15 aa, Artificial sequence
  synthetic sequence
ATGSPFGVVGNWLDP

SEQ ID NO: 397 - protein, 110 aa, Artificial sequence
  synthetic sequence
QSALTQPASVSGSPGQSITISCTGTSSDVGDFSYVSWYQQHPGKAPKLMIYEVTKRPSGVSNRFSGSKSGNTASLTISGLQTEDEADYYCTSYTSSRLVLFGGGTKLTVL

SEQ ID NO: 398 - protein, 9 aa, Artificial sequence
  synthetic sequence
SSDVGDFSY

SEQ ID NO: 399 - protein, 3 aa, Artificial sequence
  synthetic sequence
EVT

SEQ ID NO: 400 - protein, 10 aa, Artificial sequence
  synthetic sequence
TSYTSSRLVL

SEQ ID NO: 401 - protein, 120 aa, Artificial sequence
  synthetic sequence
QVQLVQSGAEVKKPGSSLKVSCKASGGTFNNFAISWVRQAPGQGPEWMGRINPILSAAKYAQKFQGRLTITADKSTTTAYMELSSLRSEDTAVYYCAPTGTGESWWFDPWGQGTLVTVSS

SEQ ID NO: 402 - protein, 8 aa, Artificial sequence
  synthetic sequence
GGTFNNFA

SEQ ID NO: 403 - protein, 8 aa, Artificial sequence
  synthetic sequence
INPILSAA

SEQ ID NO: 404 - protein, 13 aa, Artificial sequence
  synthetic sequence
APTGTGESWWFDP

SEQ ID NO: 405 - protein, 110 aa, Artificial sequence
  synthetic sequence
QSVLTQPPSASGTPGQRVTISCSGSSSNIGTNYVYWYQQLPGTAPKVLIYGNNQRPSGVPDRFSGSKSGSSASLAISGLRSEDEADYYCAAWDDSLSGPVFGGGTKLTVL

SEQ ID NO: 406 - protein, 8 aa, Artificial sequence
  synthetic sequence
SSNIGTNY

SEQ ID NO: 407 - protein, 3 aa, Artificial sequence
  synthetic sequence
GNN

SEQ ID NO: 408 - protein, 11 aa, Artificial sequence
  synthetic sequence
AAWDDSLSGPV

SEQ ID NO: 409 - protein, 123 aa, Artificial sequence
  synthetic sequence
QVQLVQSGAEVKKPGSSVKVSCKASGGTFSSYAISWVRQAPGQGLEWMGGIIPIFHTANYAQKFQGRVTITADESTSTTYVELSSLRSEDTAMYYCATLPITIFGEEYSFDNWGQGTLVTVSS

SEQ ID NO: 410 - protein, 8 aa, Artificial sequence
  synthetic sequence
GGTFSSYA

SEQ ID NO: 411 - protein, 8 aa, Artificial sequence
  synthetic sequence
IIPIFHTA

SEQ ID NO: 412 - protein, 16 aa, Artificial sequence
  synthetic sequence
ATLPITIFGEEYSFDN

SEQ ID NO: 413 - protein, 113 aa, Artificial sequence
  synthetic sequence
DVVMTQSPLSLPVTLGQPASISCRSSQSLVHSDGNTYLSWFQQRPGQSPRRLIYKVSDRDSGVPDRFSGSGSGTDFTLKISRVEAEDVGIYYCMQGTHWPPLTFGGGTKVEIK

SEQ ID NO: 414 - protein, 11 aa, Artificial sequence
  synthetic sequence
QSLVHSDGNTY

SEQ ID NO: 415 - protein, 3 aa, Artificial sequence
  synthetic sequence
KVS

SEQ ID NO: 416 - protein, 10 aa, Artificial sequence
  synthetic sequence
MQGTHWPPLT

SEQ ID NO: 417 - protein, 123 aa, Artificial sequence
  synthetic sequence
QVQLVQSGAEVKKPGSSVKVSCKASGGTFSSYSISWVRQAPGQGLEWMGGLAPIFHTPNYAQKFQGRVTITADESTSTAYMELSSLRSEDTAVYYCARVAGAGWGVYGAFDYWGQGTLVTVSS

SEQ ID NO: 418 - protein, 8 aa, Artificial sequence
  synthetic sequence
GGTFSSYS

SEQ ID NO: 419 - protein, 8 aa, Artificial sequence
  synthetic sequence
LAPIFHTP

SEQ ID NO: 420 - protein, 16 aa, Artificial sequence
  synthetic sequence
ARVAGAGWGVYGAFDY

SEQ ID NO: 421 - protein, 113 aa, Artificial sequence
  synthetic sequence
DIVMTQSPDSLAVSLGERATINCKSSQSVLHSSNNKNDLAWYQQKPGQPPKLLIYWASTRESGVPDRFSGSGSGTDFTLTISSLQAEDVAVYYCQQYYSSPGTFGPGTKVDIK

SEQ ID NO: 422 - protein, 12 aa, Artificial sequence
  synthetic sequence
QSVLHSSNNKND

SEQ ID NO: 423 - protein, 3 aa, Artificial sequence
  synthetic sequence
WAS

SEQ ID NO: 424 - protein, 9 aa, Artificial sequence
  synthetic sequence
QQYYSSPGT

SEQ ID NO: 425 - protein, 130 aa, Artificial sequence
  synthetic sequence
QVQLVQSGAEVKKPGSSVKVSCKASGDAFISYAISWVRQAPGQGLEWMGGIIPIFGTANYAQKFQGRVTISADESTSTAYMELSRLRSEDTAVYYCARGGPEPYGSGSGYTPRYNWFDPWGQGTLVTVSS

SEQ ID NO: 426 - protein, 8 aa, Artificial sequence
  synthetic sequence
GDAFISYA

SEQ ID NO: 427 - protein, 8 aa, Artificial sequence
  synthetic sequence
IIPIFGTA

SEQ ID NO: 428 - protein, 23 aa, Artificial sequence
  synthetic sequence
ARGGPEPYGSGSGYTPRYNWFDP

SEQ ID NO: 429 - protein, 110 aa, Artificial sequence
  synthetic sequence
QSALTQPRSVSGSPGQSVTISCTGTSSDVGGYNYVSWYQQHPGKAPKFMIYDVNRRPSGVPDRFSGSKSGNTASLTISGLQAEDEADYYCCSYAGTYTWVFGGGTKLTVL

SEQ ID NO: 430 - protein, 9 aa, Artificial sequence
  synthetic sequence
SSDVGGYNY

SEQ ID NO: 431 - protein, 3 aa, Artificial sequence
  synthetic sequence
DVN

SEQ ID NO: 432 - protein, 10 aa, Artificial sequence
  synthetic sequence
CSYAGTYTWV

SEQ ID NO: 433 - protein, 122 aa, Artificial sequence
  synthetic sequence
QVQLQESGPGLVKPSETLSLTCTVSGGSISGHYWSWIRQPPGKGLEWIGYIYYSGSTNYNPSLKSRVTISVDTSVNQFSLKLSSVTAADTAVYYCARQEARDAVGNYFFDSWGQGTLVTVSS

SEQ ID NO: 434 - protein, 8 aa, Artificial sequence
  synthetic sequence
GGSISGHY

SEQ ID NO: 435 - protein, 7 aa, Artificial sequence
  synthetic sequence
IYYSGST

SEQ ID NO: 436 - protein, 16 aa, Artificial sequence
  synthetic sequence
ARQEARDAVGNYFFDS

SEQ ID NO: 437 - protein, 110 aa, Artificial sequence
  synthetic sequence
QSALTQPASVSGSPGQSITISCSGTSSDIGSYDLVSWYQQHPGKAPKLLIYDVTKRPSGVSHRFSGSKSGNTASLTISGLQGEDEADYYCCSYVHFSTWVFGGGTKLTVL

SEQ ID NO: 438 - protein, 9 aa, Artificial sequence
  synthetic sequence
SSDIGSYDL

SEQ ID NO: 439 - protein, 3 aa, Artificial sequence
  synthetic sequence
DVT

SEQ ID NO: 440 - protein, 10 aa, Artificial sequence
  synthetic sequence
CSYVHFSTWV

SEQ ID NO: 441 - protein, 123 aa, Artificial sequence
  synthetic sequence
QMQLVQSGPEVKKPGTSVKVSCKASGFTFSSSAVQWVRQARGQGLEWIGWIVVGSGNANYAQKLQERVSITRDMSTSTAYMELSSLRPEDTAVYYCAAPHCSRTICHDGFDMWGQGTMVTVSS

SEQ ID NO: 442 - protein, 8 aa, Artificial sequence
  synthetic sequence
GFTFSSSA

SEQ ID NO: 443 - protein, 8 aa, Artificial sequence
  synthetic sequence
IVVGSGNA

SEQ ID NO: 444 - protein, 16 aa, Artificial sequence
  synthetic sequence
AAPHCSRTICHDGFDM

SEQ ID NO: 445 - protein, 108 aa, Artificial sequence
  synthetic sequence
EIVLTQSPGTLSLSPGERATLSCRASQSVRSSYLAWYQQKPGQAPRLLMFVASSRATGIPDRFSGSGSGTDFTLTISRLEPEDFAVYYCQQYDTSPWTFGQGTKVEIK

SEQ ID NO: 446 - protein, 7 aa, Artificial sequence
  synthetic sequence
QSVRSSY

SEQ ID NO: 447 - protein, 3 aa, Artificial sequence
  synthetic sequence
VAS

SEQ ID NO: 448 - protein, 9 aa, Artificial sequence
  synthetic sequence
QQYDTSPWT

SEQ ID NO: 449 - protein, 117 aa, Artificial sequence
  synthetic sequence
EVQLVQSGAEVKKPGSSVKVSCKASGGTFSMHTIRWVRQAPGQGLEWMGRIIPMLGIVNYAQKFQGRVTISADKSTSTAYMELSSLTSEDTAMYYCAKGSHDVFDIWGQGTMVTVSS

SEQ ID NO: 450 - protein, 8 aa, Artificial sequence
  synthetic sequence
GGTFSMHT

SEQ ID NO: 451 - protein, 8 aa, Artificial sequence
  synthetic sequence
IIPMLGIV

SEQ ID NO: 452 - protein, 10 aa, Artificial sequence
  synthetic sequence
AKGSHDVFDI

SEQ ID NO: 453 - protein, 108 aa, Artificial sequence
  synthetic sequence
DIQMTQSPSTLSASVGDRVTITCRASQSISSWLAWYQQKPGKAPKLLIYDASSLESGVPSRFSGSGSGTEFTLTISSLQPDDFATYYCQQYNSYSPITFGQGTRLEIK

SEQ ID NO: 454 - protein, 6 aa, Artificial sequence
  synthetic sequence
QSISSW

SEQ ID NO: 455 - protein, 3 aa, Artificial sequence
  synthetic sequence
DAS

SEQ ID NO: 456 - protein, 10 aa, Artificial sequence
  synthetic sequence
QQYNSYSPIT

SEQ ID NO: 457 - protein, 119 aa, Artificial sequence
  synthetic sequence
QITLKESGPTLVKPTQTLTLTCTFSGFSLSSGGVGVGWIRQPPGKALEWLALIYWDDDKRYRPSLKSRLTITRDTSTNQVVLTMTNMDPVDTATYFCARHQIATVFDHWGQGTLVTVSS

SEQ ID NO: 458 - protein, 10 aa, Artificial sequence
  synthetic sequence
GFSLSSGGVG

SEQ ID NO: 459 - protein, 7 aa, Artificial sequence
  synthetic sequence
IYWDDDK

SEQ ID NO: 460 - protein, 11 aa, Artificial sequence
  synthetic sequence
ARHQIATVFDH

SEQ ID NO: 461 - protein, 111 aa, Artificial sequence
  synthetic sequence
QSALTQPASVSGSPGQSITISCTGTSSDVGGYNYVSWYQQHPGKAPKLMIYEVSNRPSGVSSRFSGSKSGNTASLTISGLQAEDEADYYCSSYTRSSPLVAFGGGTKVTVL

SEQ ID NO: 462 - protein, 9 aa, Artificial sequence
  synthetic sequence
SSDVGGYNY

SEQ ID NO: 463 - protein, 3 aa, Artificial sequence
  synthetic sequence
EVS

SEQ ID NO: 464 - protein, 11 aa, Artificial sequence
  synthetic sequence
SSYTRSSPLVA

SEQ ID NO: 465 - protein, 127 aa, Artificial sequence
  synthetic sequence
EVQLVESGGGLVQPGRSLRLSCAASGLTFEDYAMHWVRQPPGKGLEWVSGVSWNSGTIGYADSVKGRFTISRDNAKNSLYLHMRSLGAEDTAMYYCAKDMGGRFSFFSLENDAFDIWGQGTMVIVSS

SEQ ID NO: 466 - protein, 8 aa, Artificial sequence
  synthetic sequence
GLTFEDYA

SEQ ID NO: 467 - protein, 8 aa, Artificial sequence
  synthetic sequence
VSWNSGTI

SEQ ID NO: 468 - protein, 20 aa, Artificial sequence
  synthetic sequence
AKDMGGRFSFFSLENDAFDI

SEQ ID NO: 469 - protein, 107 aa, Artificial sequence
  synthetic sequence
SYELTQPPSVSVSPGQTARITCSGDALPKQSTYWYQQKPGQAPVLVIYKDIERPSGIPERFSGSSSGTTVTLTISGVQAEDEADYYCQSADSSDTYVFGTGTKVTVL

SEQ ID NO: 470 - protein, 6 aa, Artificial sequence
  synthetic sequence
ALPKQS

SEQ ID NO: 471 - protein, 3 aa, Artificial sequence
  synthetic sequence
KDI

SEQ ID NO: 472 - protein, 10 aa, Artificial sequence
  synthetic sequence
QSADSSDTYV

SEQ ID NO: 473 - DNA, 7445 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgtatcatcctgttcctggtcgccacagccaccggagtgcacagccaagtgcagctgcaggagagcggacctagactggtcaagcctagcgagacactgtctctgacctgcaccgtgtctggagatagcatcagcagctactactggtcctggatcagacagcctcctggcaagggcctggaatggatcggctacatctactacagcggcagcaccaactacaacccctctctgaaaagcagagtgaccatcagcgtggacaccagcaagaaccagttcagcctgaagctgagcagcgtgacagccgctgataccgccgtgtactactgcgccagaaagaccgtggccggccctgctggcgagttcgactactggggccagggcaccctggtgaccgtgtccagcgccagcaccaagggcccctctgtctttcctctggccccttctagcaaatctacaagcggaggcaccgccgccctgggttgtctggtgaaagactacttcccagagcctgtgaccgtgtcttggaacagcggcgccctgaccagcggcgtgcacacattccccgctgtgctgcagagcagcggcctgtacagcctgagcagcgtggtcaccgtccccagcagctctctgggaacacagacctacatctgcaacgtgaaccacaagccttctaataccaaggtggataagaaggtggaacctaagagttgcgacaagacccacacctgtcctccgtgccccgcccctgagctgctgggcggccctagcgtgtttctgttccctccaaagcccaaggacaccctgatgatcagcagaacccctgaggtgacctgcgtggtggttgatgtgtcccacgaagatcctgaggtgaagttcaactggtacgtggacggcgttgaggtgcataatgccaagacaaagccaagagaggaacagtacaacagcacatacagagtggtgtccgtgctgaccgtgctgcaccaggactggctgaacggcaaagagtacaagtgcaaggtgtccaacaaggccctgcctgctcctatcgagaagaccatcagcaaggctaaaggacagcctcgggaaccccaggtctacaccctgccccccagccgggacgagctgacaaagaaccaggtgtccctgacatgcctggtgaagggcttctacccctccgacatcgccgtggaatgggagagcaatggccaacctgaaaacaactacaaaacgacccctcctgttctggacagcgacggcagcttcttcctttatagcaagctgacagtggacaagagcagatggcagcagggcaacgtgttcagctgcagcgtgatgcacgaggccctccacaaccactacacccagaagtccctgagcctgtctcctggcaagtgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 474 - DNA, 6749 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgcatcatcctgtttctggtcgccaccgccaccggcgtgcacagccaaagcgtgctgacccagcctcctagcgtgtccggtgctcctggccagagagtgaccatcagctgcaccggcagctctagcaacatcggcgccggctacgacgtgcactggtaccagcagttccccggcaccgcccctaagctgctgatctacggcaacagcaatagacccagcggcgtgcctgatagattcagcggatctaagagcggcacaagcgcctctctggccatcaccggcctgcaggccgaggacgaggccgactactactgccagagctacgacagcagcctgagcggtccttacgtgttcggcaccggcactaaggtgacagtgctgggccagcctaaggccgcccctagcgtgaccctgttcccccccagcagcgaggaactgcaggccaacaaggccacactggtgtgcctgatcagcgacttctaccccggcgccgtgaccgtggcctggaaggcagatagcagccctgtgaaggccggagttgagacaaccacacctagcaagcagagcaacaacaagtacgccgcttcttcttacctgagcctgacccctgagcagtggaaaagccacagaagctacagctgccaggtgacacacgagggcagcaccgtggaaaagaccgtggcccctaccgagtgcagctgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 475 - DNA, 7445 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgtatcatcctgttcctggtcgccacagccaccggagtgcacagccaagtgcaggtggtgcagagcggagccgaggtgaaaaagcccggcgccagcgtgaaggtgtcctgcaccgccagcggccacaccttcaccggctacgccatccactgggtcagacaggcccctggccagcggctggaatggatgggcctgatcaacgccggcggcatctaccggacctacagccagagattccagggcagagtgaccatcaccagagatacaagcgcctccaccgcctacatggaactgacaggcctgaccagcgaggacaccgccgtgtactactgcgccagagccaactacggctctggcagcttcagcgaccactggggccagggtacactggtgacagtgagttctgccagcaccaagggcccctctgtctttcctctggccccttctagcaaatctacaagcggaggcaccgccgccctgggttgtctggtgaaagactacttcccagagcctgtgaccgtgtcttggaacagcggcgccctgaccagcggcgtgcacacattccccgctgtgctgcagagcagcggcctgtacagcctgagcagcgtggtcaccgtccccagcagctctctgggaacacagacctacatctgcaacgtgaaccacaagccttctaataccaaggtggataagaaggtggaacctaagagttgcgacaagacccacacctgtcctccgtgccccgcccctgagctgctgggcggccctagcgtgtttctgttccctccaaagcccaaggacaccctgatgatcagcagaacccctgaggtgacctgcgtggtggttgatgtgtcccacgaagatcctgaggtgaagttcaactggtacgtggacggcgttgaggtgcataatgccaagacaaagccaagagaggaacagtacaacagcacatacagagtggtgtccgtgctgaccgtgctgcaccaggactggctgaacggcaaagagtacaagtgcaaggtgtccaacaaggccctgcctgctcctatcgagaagaccatcagcaaggctaaaggacagcctcgggaaccccaggtctacaccctgccccccagccgggacgagctgacaaagaaccaggtgtccctgacatgcctggtgaagggcttctacccctccgacatcgccgtggaatgggagagcaatggccaacctgaaaacaactacaaaacgacccctcctgttctggacagcgacggcagcttcttcctttatagcaagctgacagtggacaagagcagatggcagcagggcaacgtgttcagctgcagcgtgatgcacgaggccctccacaaccactacacccagaagtccctgagcctgtctcctggcaagtgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 476 - DNA, 6737 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgcatcatcctgttcctggtggccacagccaccggcgtgcacagcgacatccagatgacccagtctccatcttctctgagcgccagcgtgggcgacagagtgaccatcacctgtagagccagccagagcatcagcagctacctgaactggtaccagcaaaagcccggcaaggcccctaagctgctgatctacgccgcctctagcctgcagagcggcgtgcccagcagattcagcggcagcggatctgggaccgacttcaccctgaccatcagcagcctgcaacctgaggacttcgccacatactactgccagcagagcagctcaacaccttacaccttcggccagggcaccaagctggaaatcaagagaaccgtggccgcccctagcgtgttcatcttcccccccagcgacgagcagctgaagagcggtacagcttctgtggtgtgcctgctgaacaacttctacccgcgggaagccaaggtgcagtggaaggtggacaacgccctgcagagcggcaacagccaggagagcgtgacagagcaggacagcaaggacagcacctacagcctgagcagcaccctgaccctgagcaaggccgactacgagaagcacaaggtgtacgcctgtgaagtgacccaccagggcctgtctagccctgtgaccaagtcttttaacagaggcgagtgctgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 477 - DNA, 7436 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgtatcatcctgttcctggtcgccacagccaccggagtgcacagcgaggtgcagctggtggaaagcggcggcggactggtccagcccggcggcagcctgagactgagctgcgccgcttctggcttcaccgtgtctagcaactacatgagctgggtgcggcaggcccctggcaagggcctggaatgggtctccgtgatctacagcggcgggtcaacattctacgccgacagcgtgaagggcagattcaccatcagccgggacaacagcagaaacaccctgtacctgcagatgaacagcctgcgggccgaggacaccgccgtgtactactgcgccagagattggggcgagttcttcttcgacttctggggacagggcgccctggttacagtgtccagcgccagcaccaagggcccctctgtctttcctctggccccttctagcaaatctacaagcggaggcaccgccgccctgggttgtctggtgaaagactacttcccagagcctgtgaccgtgtcttggaacagcggcgccctgaccagcggcgtgcacacattccccgctgtgctgcagagcagcggcctgtacagcctgagcagcgtggtcaccgtccccagcagctctctgggaacacagacctacatctgcaacgtgaaccacaagccttctaataccaaggtggataagaaggtggaacctaagagttgcgacaagacccacacctgtcctccgtgccccgcccctgagctgctgggcggccctagcgtgtttctgttccctccaaagcccaaggacaccctgatgatcagcagaacccctgaggtgacctgcgtggtggttgatgtgtcccacgaagatcctgaggtgaagttcaactggtacgtggacggcgttgaggtgcataatgccaagacaaagccaagagaggaacagtacaacagcacatacagagtggtgtccgtgctgaccgtgctgcaccaggactggctgaacggcaaagagtacaagtgcaaggtgtccaacaaggccctgcctgctcctatcgagaagaccatcagcaaggctaaaggacagcctcgggaaccccaggtctacaccctgccccccagccgggacgagctgacaaagaaccaggtgtccctgacatgcctggtgaagggcttctacccctccgacatcgccgtggaatgggagagcaatggccaacctgaaaacaactacaaaacgacccctcctgttctggacagcgacggcagcttcttcctttatagcaagctgacagtggacaagagcagatggcagcagggcaacgtgttcagctgcagcgtgatgcacgaggccctccacaaccactacacccagaagtccctgagcctgtctcctggcaagtgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 478 - DNA, 6740 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgcatcatcctgttcctggtggccacagccaccggcgtgcacagcgagatcgtgctgacacagagccctggcacactgagcctgtctccaggcgagcgggccacactgagctgcagagccagccagaccatctccagcagctacctggcctggtaccagcaaaagcctggacaggcccctagactgctgatcagcggtgcttcttctagagccaccggcatccccgacagattcagcggcagcggctctggcaccgacttcaccctgaccatcagcagactggaaccggaagatttcgccgtgtactactgccagcagtacggcagcagccccagaaccttcggccagggcaccaacgtggaaatcaagagaaccgtggccgcccctagcgtgttcatcttcccccccagcgacgagcagctgaagagcggtacagcttctgtggtgtgcctgctgaacaacttctacccgcgggaagccaaggtgcagtggaaggtggacaacgccctgcagagcggcaacagccaggagagcgtgacagagcaggacagcaaggacagcacctacagcctgagcagcaccctgaccctgagcaaggccgactacgagaagcacaaggtgtacgcctgtgaagtgacccaccagggcctgtctagccctgtgaccaagtcttttaacagaggcgagtgctgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 479 - DNA, 7472 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgtatcatcctgttcctggtcgccacagccaccggagtgcacagccaagtgcagctggtccagtctggcgccgaggtgaaaaagcccggcagcagcgtgaaggtgtcatgcaaggccagcggcggcaccttcagcaacagcgccatcaactgggtgcggcaggcccctggccagggcctggaatggatgggcggaatcatccccctgttcggcgctgctgattacgcccagaagttccaggccagagtgacaatcaccgccgataagtccaccaccacaagctacatggaactgagcagcctgagaagcgaggacaccgccgtgtactactgcgcccgggacaccctggaacacgacgacgtgtggggcaacttcagactgtctctgcctctgagcttctggggacagggaacactggtgaccgtgtccagcgccagcaccaagggcccctctgtctttcctctggccccttctagcaaatctacaagcggaggcaccgccgccctgggttgtctggtgaaagactacttcccagagcctgtgaccgtgtcttggaacagcggcgccctgaccagcggcgtgcacacattccccgctgtgctgcagagcagcggcctgtacagcctgagcagcgtggtcaccgtccccagcagctctctgggaacacagacctacatctgcaacgtgaaccacaagccttctaataccaaggtggataagaaggtggaacctaagagttgcgacaagacccacacctgtcctccgtgccccgcccctgagctgctgggcggccctagcgtgtttctgttccctccaaagcccaaggacaccctgatgatcagcagaacccctgaggtgacctgcgtggtggttgatgtgtcccacgaagatcctgaggtgaagttcaactggtacgtggacggcgttgaggtgcataatgccaagacaaagccaagagaggaacagtacaacagcacatacagagtggtgtccgtgctgaccgtgctgcaccaggactggctgaacggcaaagagtacaagtgcaaggtgtccaacaaggccctgcctgctcctatcgagaagaccatcagcaaggctaaaggacagcctcgggaaccccaggtctacaccctgccccccagccgggacgagctgacaaagaaccaggtgtccctgacatgcctggtgaagggcttctacccctccgacatcgccgtggaatgggagagcaatggccaacctgaaaacaactacaaaacgacccctcctgttctggacagcgacggcagcttcttcctttatagcaagctgacagtggacaagagcagatggcagcagggcaacgtgttcagctgcagcgtgatgcacgaggccctccacaaccactacacccagaagtccctgagcctgtctcctggcaagtgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 480 - DNA, 6740 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgcatcatcctgttcctggtggccacagccaccggcgtgcacagcgagatcgtgatgacccagtctcctgccaccctgtctgtgtcccctggcgagagagccacactgagctgcagagcttctcagagcgtgtctagcaagctggcctggtaccagcagaaacctggacaggcccctagactgctgttctacggcgccagcaccagagccacaggcctgcctgctagattcagcggcagcggatctggcaccgagttcaccctgaccatcagcagcctgcagagtgaagatttcgccgtgtactactgccagcaatacaacgactggcctatgtacaccttcggccccggaacaagactggaaatcaagagaaccgtggccgcccctagcgtgttcatcttcccccccagcgacgagcagctgaagagcggtacagcttctgtggtgtgcctgctgaacaacttctacccgcgggaagccaaggtgcagtggaaggtggacaacgccctgcagagcggcaacagccaggagagcgtgacagagcaggacagcaaggacagcacctacagcctgagcagcaccctgaccctgagcaaggccgactacgagaagcacaaggtgtacgcctgtgaagtgacccaccagggcctgtctagccctgtgaccaagtcttttaacagaggcgagtgctgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 481 - DNA, 7466 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgtatcatcctgttcctggtcgccacagccaccggagtgcacagccaagtgcagctggtcgagagcggcggaggcgtggtgcagcctggaagaagcctgagactgagctgcgccgcttctggcttcacattcagcagatacggcatgcactgggtgcggcaggcccctggcaagggcctggaatgggtcgccctgatcagcagcggcggcagcaacaagtactacgccgacagcgtgaagggcagattcaccatctctagagataacagcaagaacaccctgtacctggaaatgaacagcctgcgggccgaggacaccgccgtgtactactgcgccaaggacgccatctacgactacatctggggcgcctacagagagaactggttcgacccctggggacagggcaccctggtgaccgtgtccagcgccagcaccaagggcccctctgtctttcctctggccccttctagcaaatctacaagcggaggcaccgccgccctgggttgtctggtgaaagactacttcccagagcctgtgaccgtgtcttggaacagcggcgccctgaccagcggcgtgcacacattccccgctgtgctgcagagcagcggcctgtacagcctgagcagcgtggtcaccgtccccagcagctctctgggaacacagacctacatctgcaacgtgaaccacaagccttctaataccaaggtggataagaaggtggaacctaagagttgcgacaagacccacacctgtcctccgtgccccgcccctgagctgctgggcggccctagcgtgtttctgttccctccaaagcccaaggacaccctgatgatcagcagaacccctgaggtgacctgcgtggtggttgatgtgtcccacgaagatcctgaggtgaagttcaactggtacgtggacggcgttgaggtgcataatgccaagacaaagccaagagaggaacagtacaacagcacatacagagtggtgtccgtgctgaccgtgctgcaccaggactggctgaacggcaaagagtacaagtgcaaggtgtccaacaaggccctgcctgctcctatcgagaagaccatcagcaaggctaaaggacagcctcgggaaccccaggtctacaccctgccccccagccgggacgagctgacaaagaaccaggtgtccctgacatgcctggtgaagggcttctacccctccgacatcgccgtggaatgggagagcaatggccaacctgaaaacaactacaaaacgacccctcctgttctggacagcgacggcagcttcttcctttatagcaagctgacagtggacaagagcagatggcagcagggcaacgtgttcagctgcagcgtgatgcacgaggccctccacaaccactacacccagaagtccctgagcctgtctcctggcaagtgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 482 - DNA, 6737 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgcatcatcctgttcctggtggccacagccaccggcgtgcacagcgacatccagatgacccagagccctagcagcctgagcgccagcgtgggagatagagtgaccatcacctgtagagcctctcagagcatcagcaactacctgaactggtaccagcagaagcccggcaaggcccctaacctgctgatctacgccgcttctagcctgcagtctggcgtgcccagcagattcagcggatctggcagcggcaccgacttcaccctgaccatcagcagcctgcaacctgaggacttcgccacatactactgccagcaaacaagctctccacctctgacattcggccagggcacaaaggtggaaatcaagagaaccgtggccgcccctagcgtgttcatcttcccccccagcgacgagcagctgaagagcggtacagcttctgtggtgtgcctgctgaacaacttctacccgcgggaagccaaggtgcagtggaaggtggacaacgccctgcagagcggcaacagccaggagagcgtgacagagcaggacagcaaggacagcacctacagcctgagcagcaccctgaccctgagcaaggccgactacgagaagcacaaggtgtacgcctgtgaagtgacccaccagggcctgtctagccctgtgaccaagtcttttaacagaggcgagtgctgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 483 - DNA, 7463 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgtatcatcctgttcctggtcgccacagccaccggagtgcacagccaagtgcagctggtgcagagcggcgctgaagtgaaaaagcctggcagcagcgtgaaggtgtcttgtaaagccagcggcggcacattcagcagctacgccatcagctgggtgcggcaggcccctggacagggcctggaatggatgggcggaatcatccccgtgttcggcacgaccaactacgcccaggagttcagagccagagtgaccatcaccgccgacgagagcaccaataccgcctacatggaactgagatctctgagcagcgccgatacaggcgtgtactactgcgccaccggcgccggcgagatgatcgaggtgacaatcgccaacgacgtgttcgacatctggggccagggcaccagagtgaccgtgtccagcgccagcaccaagggcccctctgtctttcctctggccccttctagcaaatctacaagcggaggcaccgccgccctgggttgtctggtgaaagactacttcccagagcctgtgaccgtgtcttggaacagcggcgccctgaccagcggcgtgcacacattccccgctgtgctgcagagcagcggcctgtacagcctgagcagcgtggtcaccgtccccagcagctctctgggaacacagacctacatctgcaacgtgaaccacaagccttctaataccaaggtggataagaaggtggaacctaagagttgcgacaagacccacacctgtcctccgtgccccgcccctgagctgctgggcggccctagcgtgtttctgttccctccaaagcccaaggacaccctgatgatcagcagaacccctgaggtgacctgcgtggtggttgatgtgtcccacgaagatcctgaggtgaagttcaactggtacgtggacggcgttgaggtgcataatgccaagacaaagccaagagaggaacagtacaacagcacatacagagtggtgtccgtgctgaccgtgctgcaccaggactggctgaacggcaaagagtacaagtgcaaggtgtccaacaaggccctgcctgctcctatcgagaagaccatcagcaaggctaaaggacagcctcgggaaccccaggtctacaccctgccccccagccgggacgagctgacaaagaaccaggtgtccctgacatgcctggtgaagggcttctacccctccgacatcgccgtggaatgggagagcaatggccaacctgaaaacaactacaaaacgacccctcctgttctggacagcgacggcagcttcttcctttatagcaagctgacagtggacaagagcagatggcagcagggcaacgtgttcagctgcagcgtgatgcacgaggccctccacaaccactacacccagaagtccctgagcctgtctcctggcaagtgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 484 - DNA, 6746 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgcatcatcctgtttctggtcgccaccgccaccggcgtgcacagccagctggtgctgacccagtctccatctgcttctgccagcctgggcgctagcgtgaagctgacctgcaccctgagcagcggccacagctcctacgccatcgcctggcaccagcagcaacctgagaagggccctagatacctgatgaaactgaacagcgacggcagccactcaaagggagatggcatccccgatagattcagcggaagcagcagcggcgccgagcggtacctgacaatcagctctctgcagagcgacgacgaggccgactactactgccagacctggggcaccgccatccacgtgttcggcacaggcaccaaggtgaccgtgctgggccagcctaaggccgcccctagcgtgaccctgttcccccccagcagcgaggaactgcaggccaacaaggccacactggtgtgcctgatcagcgacttctaccccggcgccgtgaccgtggcctggaaggcagatagcagccctgtgaaggccggagttgagacaaccacacctagcaagcagagcaacaacaagtacgccgcttcttcttacctgagcctgacccctgagcagtggaaaagccacagaagctacagctgccaggtgacacacgagggcagcaccgtggaaaagaccgtggcccctaccgagtgcagctgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 485 - DNA, 7085 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgtatcatcctgttcctggtcgccacagccaccggagtgcacagcgccagcaccaagggcccctctgtctttcctctggccccttctagcaaatctacaagcggaggcaccgccgccctgggttgtctggtgaaagactacttcccagagcctgtgaccgtgtcttggaacagcggcgccctgaccagcggcgtgcacacattccccgctgtgctgcagagcagcggcctgtacagcctgagcagcgtggtcaccgtccccagcagctctctgggaacacagacctacatctgcaacgtgaaccacaagccttctaataccaaggtggataagaaggtggaacctaagagttgcgacaagacccacacctgtcctccgtgccccgcccctgagctgctgggcggccctagcgtgtttctgttccctccaaagcccaaggacaccctgatgatcagcagaacccctgaggtgacctgcgtggtggttgatgtgtcccacgaagatcctgaggtgaagttcaactggtacgtggacggcgttgaggtgcataatgccaagacaaagccaagagaggaacagtacaacagcacatacagagtggtgtccgtgctgaccgtgctgcaccaggactggctgaacggcaaagagtacaagtgcaaggtgtccaacaaggccctgcctgctcctatcgagaagaccatcagcaaggctaaaggacagcctcgggaaccccaggtctacaccctgccccccagccgggacgagctgacaaagaaccaggtgtccctgacatgcctggtgaagggcttctacccctccgacatcgccgtggaatgggagagcaatggccaacctgaaaacaactacaaaacgacccctcctgttctggacagcgacggcagcttcttcctttatagcaagctgacagtggacaagagcagatggcagcagggcaacgtgttcagctgcagcgtgatgcacgaggccctccacaaccactacacccagaagtccctgagcctgtctcctggcaagtgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 486 - DNA, 6436 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgcatcatcctgttcctggtggccacagccaccggcgtgcacagcgctagcgcggccgcggtaccagaaccgtggccgcccctagcgtgttcatcttcccccccagcgacgagcagctgaagagcggtacagcttctgtggtgtgcctgctgaacaacttctacccgcgggaagccaaggtgcagtggaaggtggacaacgccctgcagagcggcaacagccaggagagcgtgacagagcaggacagcaaggacagcacctacagcctgagcagcaccctgaccctgagcaaggccgactacgagaagcacaaggtgtacgcctgtgaagtgacccaccagggcctgtctagccctgtgaccaagtcttttaacagaggcgagtgctgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 487 - DNA, 7085 bp, Artificial sequence; identical to SEQ ID NO: 485
  synthetic sequence

SEQ ID NO: 488 - DNA, 6436 bp, Artificial sequence; identical to SEQ ID NO: 486
  synthetic sequence

SEQ ID NO: 489 - DNA, 7085 bp, Artificial sequence; identical to SEQ ID NO: 485
  synthetic sequence

SEQ ID NO: 490 - DNA, 6436 bp, Artificial sequence; identical to SEQ ID NO: 486
  synthetic sequence

SEQ ID NO: 491 - DNA, 7448 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgtatcatcctgttcctggtcgccacagccaccggagtgcacagccaagtgcagctgcaggagagcggtcctggactggtgaagcctagcgagacactgagccttacatgcaccgtgtccggcgacagcatcagcacctactactggtcctggatcagacagcctcctggcaagggcctggaatggatcggctacatccactacagcggcagcacaaactacaaccccagcctgaaaagcagagtggccgtgtctgtggacaccagcaagaaccagttcagcctgaagctgtctagcgtgaccgccgctgataccgccgtgtactactgcgccagaggcggcggagtgttcggcgtggtgatcaacttcgactactggggccagggcatcctggtcaccgtgtccagcgccagcaccaagggcccctctgtctttcctctggccccttctagcaaatctacaagcggaggcaccgccgccctgggttgtctggtgaaagactacttcccagagcctgtgaccgtgtcttggaacagcggcgccctgaccagcggcgtgcacacattccccgctgtgctgcagagcagcggcctgtacagcctgagcagcgtggtcaccgtccccagcagctctctgggaacacagacctacatctgcaacgtgaaccacaagccttctaataccaaggtggataagaaggtggaacctaagagttgcgacaagacccacacctgtcctccgtgccccgcccctgagctgctgggcggccctagcgtgtttctgttccctccaaagcccaaggacaccctgatgatcagcagaacccctgaggtgacctgcgtggtggttgatgtgtcccacgaagatcctgaggtgaagttcaactggtacgtggacggcgttgaggtgcataatgccaagacaaagccaagagaggaacagtacaacagcacatacagagtggtgtccgtgctgaccgtgctgcaccaggactggctgaacggcaaagagtacaagtgcaaggtgtccaacaaggccctgcctgctcctatcgagaagaccatcagcaaggctaaaggacagcctcgggaaccccaggtctacaccctgccccccagccgggacgagctgacaaagaaccaggtgtccctgacatgcctggtgaagggcttctacccctccgacatcgccgtggaatgggagagcaatggccaacctgaaaacaactacaaaacgacccctcctgttctggacagcgacggcagcttcttcctttatagcaagctgacagtggacaagagcagatggcagcagggcaacgtgttcagctgcagcgtgatgcacgaggccctccacaaccactacacccagaagtccctgagcctgtctcctggcaagtgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 492 - DNA, 6746 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgcatcatcctgtttctggtcgccaccgccaccggcgtgcacagcaacttcatgctgacccagccttacagcgtgtccgagagccctggaaaaaccgtgaccatcagctgcaccagaagctccggcagcatcgcctctaattacgtgcagtggtaccagcagcggcctggcagcgcccctaccaccgtgatctacgaggacaaccagagacctagcggcgtgcctgatagattcagcggctctatcgacagcagctctaacagcgcttctctgaccatcagcggcctgaagaccgaggacgaggccgactactactgccagagcagagatagcagcaaccccgtggtgttcggcggcggaacaaagctgacagtgctgggccagcctaaggccgcccctagcgtgaccctgttcccccccagcagcgaggaactgcaggccaacaaggccacactggtgtgcctgatcagcgacttctaccccggcgccgtgaccgtggcctggaaggcagatagcagccctgtgaaggccggagttgagacaaccacacctagcaagcagagcaacaacaagtacgccgcttcttcttacctgagcctgacccctgagcagtggaaaagccacagaagctacagctgccaggtgacacacgagggcagcaccgtggaaaagaccgtggcccctaccgagtgcagctgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 493 - DNA, 7442 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgtatcatcctgttcctggtcgccacagccaccggagtgcacagcgaggtgcagctggtggaaagcggaggaggcctgatccaggtgggcggcagcctgagactgagctgcgccgcttctggactgaccgtgaccagcaactacatgaactgggtgcggcagggtcctggcaagggcctggaatgggtcagcctgatctacagcggcggcaccacctactacgccgacagcgtgaagggcagattcaccatcagcagagatgatagcaagaacaccctgtacctgcagatgaacagcctgcgggccgaggacaccgccgtgtactactgcgccagacccatcgtgggcgccagaagcggcatggacgtgtggggccagggcacagccgtgacagtgtccagcgccagcaccaagggcccctctgtctttcctctggccccttctagcaaatctacaagcggaggcaccgccgccctgggttgtctggtgaaagactacttcccagagcctgtgaccgtgtcttggaacagcggcgccctgaccagcggcgtgcacacattccccgctgtgctgcagagcagcggcctgtacagcctgagcagcgtggtcaccgtccccagcagctctctgggaacacagacctacatctgcaacgtgaaccacaagccttctaataccaaggtggataagaaggtggaacctaagagttgcgacaagacccacacctgtcctccgtgccccgcccctgagctgctgggcggccctagcgtgtttctgttccctccaaagcccaaggacaccctgatgatcagcagaacccctgaggtgacctgcgtggtggttgatgtgtcccacgaagatcctgaggtgaagttcaactggtacgtggacggcgttgaggtgcataatgccaagacaaagccaagagaggaacagtacaacagcacatacagagtggtgtccgtgctgaccgtgctgcaccaggactggctgaacggcaaagagtacaagtgcaaggtgtccaacaaggccctgcctgctcctatcgagaagaccatcagcaaggctaaaggacagcctcgggaaccccaggtctacaccctgccccccagccgggacgagctgacaaagaaccaggtgtccctgacatgcctggtgaagggcttctacccctccgacatcgccgtggaatgggagagcaatggccaacctgaaaacaactacaaaacgacccctcctgttctggacagcgacggcagcttcttcctttatagcaagctgacagtggacaagagcagatggcagcagggcaacgtgttcagctgcagcgtgatgcacgaggccctccacaaccactacacccagaagtccctgagcctgtctcctggcaagtgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 494 - DNA, 6737 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgcatcatcctgttcctggtggccacagccaccggcgtgcacagcgatatccagatgacccagagccctagcagcctgagcgccagcgtgggagatagagtgacaatcacctgtcaggcctctcaggacatcaacaagtacctgaactggtaccagcagaagcccggcaaggcccctaagctgctgatctacgacgccagcaacctggaaaccggcgtgcccagcagattcagcggcagcggatctggcaccgacttcacctttaccatcagctctctgcagcctgaggacctggccacctactactgccaccagttcgacaacctgcctggcaccttcggcggcggaacaaaggtggaaatcaagagaaccgtggccgcccctagcgtgttcatcttcccccccagcgacgagcagctgaagagcggtacagcttctgtggtgtgcctgctgaacaacttctacccgcgggaagccaaggtgcagtggaaggtggacaacgccctgcagagcggcaacagccaggagagcgtgacagagcaggacagcaaggacagcacctacagcctgagcagcaccctgaccctgagcaaggccgactacgagaagcacaaggtgtacgcctgtgaagtgacccaccagggcctgtctagccctgtgaccaagtcttttaacagaggcgagtgctgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 495 - DNA, 7442 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgtatcatcctgttcctggtcgccacagccaccggagtgcacagccaaatcaccctgaaggaaagcggtcctacactggtcaagcctacccagaccctgacactgacatgcaccttcagcggcttcagcctgagcaccgtgggagtgggcgtggcctggatcagacagcctcctggcaaggccctggaatggctggccctgatctactgggacgacgataagcggtacagccccagcctgaaaagcagactgaccatcacaaaggacaccagcaagaaccacgtggtgctgaccatgaccaacatggaccccgtggataccgccacctactactgcgcccaccacatcatcgccgccctggtggacgtgtggggaaagggcaccaccgttacagtgtccagcgccagcaccaagggcccctctgtctttcctctggccccttctagcaaatctacaagcggaggcaccgccgccctgggttgtctggtgaaagactacttcccagagcctgtgaccgtgtcttggaacagcggcgccctgaccagcggcgtgcacacattccccgctgtgctgcagagcagcggcctgtacagcctgagcagcgtggtcaccgtccccagcagctctctgggaacacagacctacatctgcaacgtgaaccacaagccttctaataccaaggtggataagaaggtggaacctaagagttgcgacaagacccacacctgtcctccgtgccccgcccctgagctgctgggcggccctagcgtgtttctgttccctccaaagcccaaggacaccctgatgatcagcagaacccctgaggtgacctgcgtggtggttgatgtgtcccacgaagatcctgaggtgaagttcaactggtacgtggacggcgttgaggtgcataatgccaagacaaagccaagagaggaacagtacaacagcacatacagagtggtgtccgtgctgaccgtgctgcaccaggactggctgaacggcaaagagtacaagtgcaaggtgtccaacaaggccctgcctgctcctatcgagaagaccatcagcaaggctaaaggacagcctcgggaaccccaggtctacaccctgccccccagccgggacgagctgacaaagaaccaggtgtccctgacatgcctggtgaagggcttctacccctccgacatcgccgtggaatgggagagcaatggccaacctgaaaacaactacaaaacgacccctcctgttctggacagcgacggcagcttcttcctttatagcaagctgacagtggacaagagcagatggcagcagggcaacgtgttcagctgcagcgtgatgcacgaggccctccacaaccactacacccagaagtccctgagcctgtctcctggcaagtgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 496 - DNA, 6746 bp, Artificial sequence
  synthetic sequence
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgaattcccgccgccaccatgggctggtcctgcatcatcctgtttctggtcgccaccgccaccggcgtgcacagccaaagcgccctgacacagcctgctagcgtgtccggcagccctggccagagcatcaccatcagctgcaccggcaccagcagtgatgtgggcggcaacaactacgtgtcctggtaccagcaccaccccggcgaggcccctaagctgatgatctacgaggtgtccaaccgtccatctggcgtgtctaatagattcagcggctctaaaagcggaaataccgccagcctgaccatcagcggcctgcaggccgaggacgaggccgactactactgcagcagctacaccaccagctcaacactggtggtgttcggcggaggcagcaagctgaccgtgctgggccagcctaaggccgcccctagcgtgaccctgttcccccccagcagcgaggaactgcaggccaacaaggccacactggtgtgcctgatcagcgacttctaccccggcgccgtgaccgtggcctggaaggcagatagcagccctgtgaaggccggagttgagacaaccacacctagcaagcagagcaacaacaagtacgccgcttcttcttacctgagcctgacccctgagcagtggaaaagccacagaagctacagctgccaggtgacacacgagggcagcaccgtggaaaagaccgtggcccctaccgagtgcagctgataagcttaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 497 - protein, 15 aa, Artificial sequence
  synthetic sequence
GGGGSGGGGSGGGGS

SEQ ID NO: 498 - protein, 223 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 499 - protein, 673 aa, SARS-CoV-2
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 500 - protein, 588 aa, SARS-CoV-2
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 501 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT